(19) United States
(12) Patent Application Publication
(10) Pub. No.: US 2023/0400766 A1
(43) Pub. Date: Dec. 14, 2023

(54) ONIUM SALT, RESIST COMPOSITION AND PATTERN FORMING PROCESS

(71) Applicant: Shin-Etsu Chemical Co., Ltd., Tokyo (JP)

(72) Inventor: Masahiro Fukushima, Joetsu-shi (JP)

(73) Assignee: Shin-Etsu Chemical Co., Ltd., Tokyo (JP)

(21) Appl. No.: 18/207,250

(22) Filed: Jun. 8, 2023

(30) Foreign Application Priority Data
Jun. 14, 2022 (JP) 2022-095416

(51) Int. Cl. G03F 7/029; G03F 7/004; G03F 7/20

(52) U.S. Cl. CPC G03F 7/029; G03F 7/0045; G03F 7/2006

(57) ABSTRACT

A resist composition is provided comprising as a quencher an onium salt having an anion moiety whose conjugate acid is decomposed under the action of acid and heat into carbon dioxide and an organic compound of up to 12 carbon atoms. When processed by deep-UV, EB or EUV lithography, the resist composition exhibits an improved LWR and resolution and prevents the resist pattern from collapsing.

### CROSS-REFERENCE TO RELATED APPLICATIONS

This non-provisional application claims priority under 35 U.S.C. § 119(a) on Patent Application No. 2022-095416 filed in Japan on Jun. 14, 2022, the entire contents of which are hereby incorporated by reference.

### TECHNICAL FIELD

This invention relates to an onium salt, a resist composition comprising the same, and a patterning process using the composition.

### BACKGROUND ART

To meet the demand for higher integration density and operating speed of LSIs, the effort to reduce the pattern rule is in rapid progress. As the use of 5G high-speed communications and artificial intelligence (AI) is widely spreading, high-performance devices are needed for their processing. As the advanced miniaturization technology, manufacturing of microelectronic devices at the 5-nm node by the lithography using EUV of wavelength 13.5 nm has been implemented in a mass scale. Studies are made on the application of EUV lithography to 3-nm node devices of the next generation and 2-nm node devices of the next-but-one generation.

As the feature size reduces, image blurs due to acid diffusion become a problem. To insure resolution for fine patterns with a size of 45 nm et seq., not only an improvement in dissolution contrast is important as previously reported, but the control of acid diffusion is also important as reported in Non-Patent Document 1. Since chemically amplified resist compositions are designed such that sensitivity and contrast are enhanced by acid diffusion, an attempt to minimize acid diffusion by reducing the temperature and/or time of post-exposure bake (PEB) fails, resulting in drastic reductions of sensitivity and contrast.

A triangular tradeoff relationship among sensitivity, resolution, and edge roughness (LER, LWR) has been pointed out. Specifically, a resolution improvement requires to suppress acid diffusion whereas a short acid diffusion distance leads to a decline of sensitivity.

The addition of an acid generator capable of generating a bulky acid is an effective means for suppressing acid diffusion. It was then proposed to incorporate repeat units derived from an onium salt having a polymerizable unsaturated bond in a polymer. Since this polymer functions as an acid generator, it is referred to as polymer-bound acid generator. Patent Document 1 discloses a sulfonium or iodonium salt having a polymerizable unsaturated bond, capable of generating a specific sulfonic acid. Patent Document 2 discloses a sulfonium salt having a sulfonic acid directly attached to the backbone.

Studies have also been made on quenchers or acid diffusion inhibitors. Amines are typically used as the quencher. Many problems associated with line width roughness (LWR) as an index of pattern roughness and pattern profile are left unsolved. Also the use of weak acid onium salts as the quencher is under study. For example, Patent Document 1 describes that patterns with minimal roughness can be formed using a compound capable of generating a carboxylic acid having a boiling point of at least 150° C. Patent Document 2 reports improvements in sensitivity, resolution and exposure margin by the addition of ammonium salts of sulfonic acids or carboxylic acids. Also, Patent Document 3 describes that a resist composition for KrF or EB lithography comprising a PAG capable of generating a fluorinated carboxylic acid is improved in resolution and process latitude such as exposure margin and depth of focus. Further, Patent Document 4 describes a positive photosensitive composition for ArF excimer laser lithography comprising a carboxylic acid onium salt.

Patent Document 5 describes an onium salt of fluoroalkanesulfonamide as the weak acid onium salt. When this onium salt is applied to the upcoming generation of ultrafine processing using ArF lithography or ArF immersion lithography, the LWR as an index of pattern roughness and resolution are yet insufficient. There is still a need for a weak acid onium salt having better quencher function. Also Patent Documents 6 to 8 describe an onium salt of α,α-difluorocarboxylic acid and an onium salt having an oxalic acid structure as the carboxylic acid onium salt. On use of these onium salts, they can also act as an acid generator in some cases because the carboxylic acid resulting from proton exchange with strong acid has an acidity which is not fully low. Because of such low quencher function, LWR and resolution are unsatisfactory. Patent Document 9 describes an attempt to use an onium salt of aromatic carboxylic acid, which has not been positively applied in the ArF lithography, in the EUV lithography on which development efforts are recently concentrated.

These series of weak acid onium salts are based on the mechanism that a salt exchange occurs between a weak acid onium salt and a strong acid (sulfonic acid) which is generated by another PAG upon exposure, to form a weak acid and a strong acid onium salt. That is, the strong acid (α,α-difluorosulfonic acid) having high acidity is replaced by a weak acid (alkanesulfonic acid or carboxylic acid), thereby suppressing acid-aided elimination reaction of acid labile group and reducing or controlling the distance of acid diffusion. The onium salt apparently functions as a quencher. However, as the microfabrication technology is currently further advanced, the resist compositions using such weak acid onium salts become unsatisfactory with respect to resolution, roughness, depth of focus (DOF) and the like, particularly when processed by the EUV lithography. The alkanesulfonic acid salts have a low quencher capability because their acidity is not fully low. The carboxylic acid salts are not only insufficient in the above-referred properties, but also suffer from a swell problem because they are highly hydrophilic and thus have a high affinity to alkaline developer so that the developer is sucked in the exposed area. Particularly in forming small size line patterns, the swell causes the resist pattern to collapse down. To comply with the requirement for further miniaturization, it is desired to have a quencher which has a fully low acidity and excellent quenching function, and prevents the resist patterns from collapsing as a result of swelling in alkaline developer.

### CITATION LIST

Patent Document 1: JP-A H11-125907
Patent Document 2: JP-A H11-327143
Patent Document 3: JP-A 2001-281849
Patent Document 4: JP 4226803
Patent Document 5: JP-A 2012-108447
Patent Document 6: JP-A 2015-054833 (U.S. Pat. No. 9,221,742)
Patent Document 7: WO 2021/199789
Patent Document 8: JP 6304246
Patent Document 9: JP 6561731
Non-Patent Document 1: SPIE Vol. 6520 65203L-1 (2007)

### SUMMARY OF THE INVENTION

An object of the invention is to provide a resist composition which is processed by DUV, EUV or EB lithography to form a resist pattern with improved resolution, reduced LWR, and collapse resistance, an onium salt for use therein, and a pattern forming process using the resist composition.

The inventor has found that a resist composition comprising as the quencher an onium salt having an anion moiety whose conjugate acid is decomposed under the action of acid and heat into carbon dioxide and an organic compound of up to 12 carbon atoms can be processed by lithography to form a resist pattern with improved resolution and reduced LWR. Since the swell during development is suppressed, which leads to collapse resistance, the resist composition is quite useful in high accuracy micropatterning.

In one aspect, the invention provides an onium salt having an anion moiety whose conjugate acid is decomposed under the action of acid and heat into carbon dioxide and an organic compound of up to 12 carbon atoms.

The preferred onium salt has the formula (1).

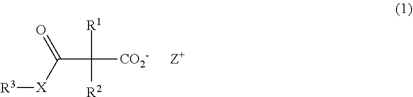

Herein X is a single bond, —O— or —S—,

R¹ and R² are each independently hydrogen or a C₁-C₁₀ hydrocarbyl group in which some constituent —CH₂— may be replaced by —O— or —C(═O)—, R¹ and R² may bond together to form a ring with the carbon atom to which they are attached,

R³ is hydrogen or a C₁-C₁₀ hydrocarbyl group when X is a single bond or —S—, and hydrogen, a C₁-C₁₀ hydrocarbyl group other than an acid labile group, or an acid labile group when X is —O—, some or all of the hydrogen atoms in the hydrocarbyl group may be substituted by halogen, some constituent —CH₂— in the hydrocarbyl group may be replaced by —O— or —C(═O)—. R¹ and R³ may bond together to form a ring with the atoms to which they are attached and intervenient atom, with the proviso that the number of carbon atoms within R¹ to R³ is up to 10 when R³ is other than the acid labile group, and

Z⁺ is an onium cation.

In a preferred embodiment, X is —O—.

In a preferred embodiment, R³ is an acid labile group.

More preferably, the acid labile group has the formula (AL-1) or (AL-2).

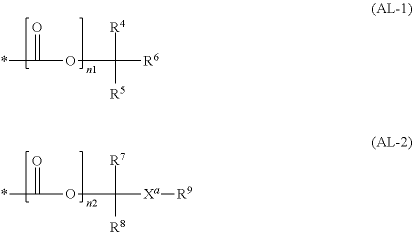

Herein X^{a} is —O— or —S—,

R⁴, R⁵ and R⁶ are each independently a C₁-C₁₂ hydrocarbyl group, some constituent —CH₂— in the hydrocarbyl group may be replaced by —O— or —S—, and when the hydrocarbyl group contains an aromatic ring, some or all of the hydrogen atoms on the aromatic ring may be substituted by halogen, cyano, nitro, optionally halogenated C₁-C₄ alkyl moiety, or optionally halogenated C₁-C₄ alkoxy moiety, any two of R⁴, R⁵ and R⁶ may bond together to form a ring, some constituent —CH₂— in the ring may be replaced by —O— or —S—,

R⁷ and R⁸ are each independently hydrogen or a C₁-C₁₀ hydrocarbyl group, R⁹ is a C₁-C₂₀ hydrocarbyl group in which some constituent —CH₂— may be replaced by —O— or —S—, R¹ and R⁹ may bond together to form a C₃-C₂₀ heterocycle with the carbon atom and X to which they are attached, some constituent —CH₂— in the heterocycle may be replaced by —O— or —S—,

n1 and n2 are each independently 0 or 1, and

* designates a point of attachment to the adjacent —O—.

In a preferred embodiment, Z⁺ is an onium cation having any one of the formulae (cation-1) to (cation-3):

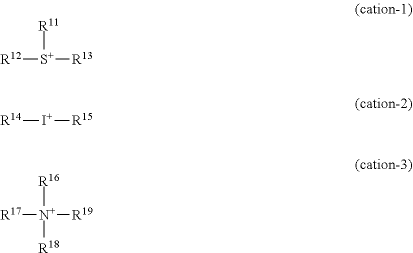

wherein R¹¹ to R¹⁹ are each independently a C₁-C₃₀ hydrocarbyl group which may contain a heteroatom, R¹¹ and R¹² may bond together to form a ring with the sulfur atom to which they are attached.

In another aspect, the invention provides a quencher comprising the onium salt defined herein.

In a further aspect, the invention provides a resist composition comprising the quencher.

The resist composition may further comprise an organic solvent.

Most often, the resist composition further comprises a base polymer comprising repeat units having the formula (a1).

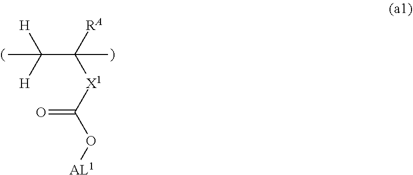

Herein R^{A} is hydrogen, fluorine, methyl or trifluoromethyl,

X¹ is a single bond, phenylene group, naphthylene group or*—C(═O)—O—X¹¹—, the phenylene group and naphthylene group may be substituted with an optionally fluorinated C₁-C₁₀ alkoxy moiety or halogen, X¹¹ is a C₁-C₁₀ saturated hydrocarbylene group which may contain a hydroxy moiety, ether bond, ester bond or lactone ring, a phenylene group or naphthylene group, * designates a point of attachment to the carbon atom in the backbone, and

AL¹ is an acid labile group.

In a preferred embodiment, the base polymer further comprises repeat units having the formula (a2).

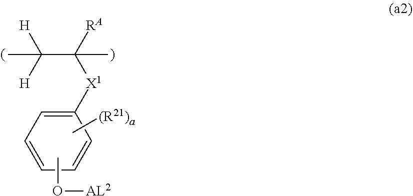

Herein R^{A} is hydrogen, fluorine, methyl or trifluoromethyl,

X² is a single bond or*—C(═O)—O—, wherein * designates a point of attachment to the carbon atom in the backbone,

R²¹ is halogen, cyano, a C₁-C₂₀ hydrocarbyl group which may contain a heteroatom, C₁-C₂₀ hydrocarbyloxy group which may contain a heteroatom, C₂-C₂₀ hydrocarbylcarbonyl group which may contain a heteroatom, C₂-C₂₀ hydrocarbylcarbonyloxy group which may contain a heteroatom, or C₂-C₂₀ hydrocarbyloxycarbonyl group which may contain a heteroatom.

AL² is an acid labile group, and

a is an integer of 0 to 4.

In a more preferred embodiment, the base polymer further comprises repeat units having the formula (b1) or (b2).

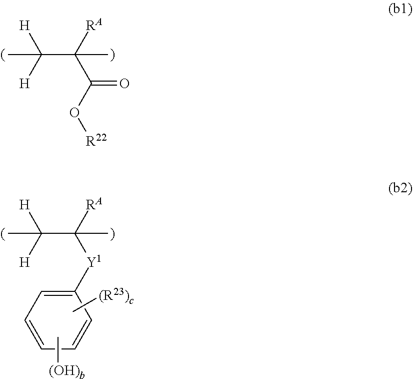

Herein R^{A} is each independently hydrogen, fluorine, methyl or trifluoromethyl,

Y¹ is a single bond or*—C(═O)—O—,

R²² is hydrogen, or a C₁-C²⁰ group containing at least one moiety selected from hydroxy moiety other than phenolic hydroxy, cyano moiety, carbonyl moiety, carboxy moiety, ether bond, ester bond, sulfonic ester bond, carbonate bond, lactone ring, sultone ring, and carboxylic anhydride (—C(═O)—O—C(═O)—),

R²³ is halogen, hydroxy, nitro, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom.

b is an integer of 1 to 4, c is an integer of 0 to 4, and b+c is from 1 to 5.

The base polymer may further comprise repeat units of at least one type selected from repeat units having the formulae (c1) to (c4).

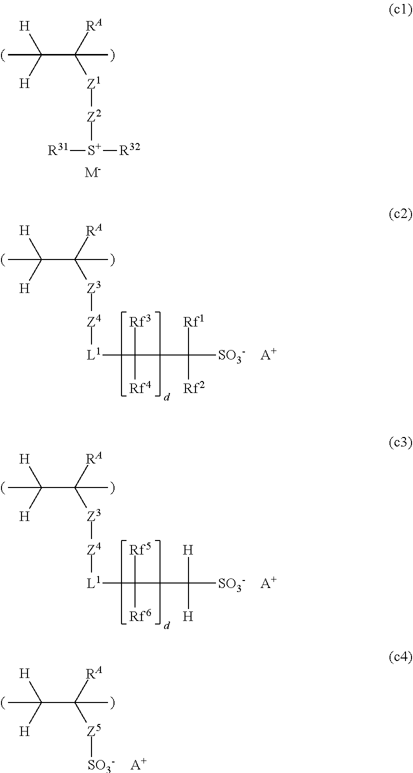

Herein R^{A} is each independently hydrogen, fluorine, methyl or trifluoromethyl,

Z¹ is a single bond or phenylene group,

Z² is *—C(═O)—O—Z²¹—, *C(═O)—NH—Z²¹—, or*—O—Z²¹—, wherein Z²¹ is a C₁-C₆ aliphatic hydrocarbylene group, phenylene, or divalent group obtained by combining the foregoing, which may contain a carbonyl moiety, ester bond, ether bond or hydroxy moiety,

Z³ is a single bond, phenylene group, naphthylene group or*—C(═O)—O—Z³¹—, wherein Z³¹ is a C₁-C₁₀ aliphatic hydrocarbylene group which may contain a hydroxy moiety, ether bond, ester bond or lactone ring, or a phenylene group or naphthylene group,

Z⁴ is a single bond or*—Z⁴¹—C(═O)—O—, wherein Z⁴¹ is a C₁-C₂₀ hydrocarbylene group which may contain a heteroatom.

Z⁵ is a single bond, methylene, ethylene, phenylene, fluorinated phenylene, trifluoromethyl-substituted phenylene, *—C(═O)—O—Z⁵¹—. *—C(═O)—N(H)—Z⁵¹— or*—O—Z⁵¹, wherein Z⁵¹ is a C₁-C₆ aliphatic hydrocarbylene group, phenylene, fluorinated phenylene or trifluoromethyl-substituted phenylene group, which may contain a carbonyl moiety, ester bond, ether bond or hydroxy moiety,

the asterisk (*) designates a point of attachment to the carbon atom in the backbone,

R³¹ and R³² are each independently a C₁-C₂₀ hydrocarbyl group which may contain a heteroatom, R³¹ and R³² may bond together to form a ring with the sulfur atom to which they are attached,

L¹ is a single bond, ether bond, ester bond, carbonyl group, sulfonic ester bond, carbonate bond or carbamate bond,

Rf¹ and Rf² are each independently fluorine or a C₁-C₆ fluorinated alkyl group.

Rf³ and Rf⁴ are each independently hydrogen, fluorine or a C₁-C₆ fluorinated alkyl group,

Rf⁵ and Rf⁶ are each independently hydrogen, fluorine or a C₁-C₆ fluorinated alkyl group, excluding that all Rf⁵ and Rf⁶ are hydrogen at the same time,

M⁻ is a non-nucleophilic counter ion,

A⁺ is an onium cation, and

d is an integer of 0 to 3.

The resist composition may further comprise a photoacid generator, an amine compound, and/or a surfactant.

In a still further aspect, the invention provides a process for forming a pattern comprising the steps of applying the resist composition defined above to a substrate to form a resist film thereon, exposing the resist film to high-energy radiation, baking the resist film, and developing the PEB resist film in a developer.

The high-energy radiation is typically KrF excimer laser, ArF excimer laser, EB, or EUV of wavelength 3 to 15 nm.

### Advantageous Effects of Invention

Since the onium salt exerts a satisfactory quencher function in a resist composition, a pattern of good profile with a high resolution, reduced LWR, and good rectangularity can be formed from the resist composition. The salt is effective for preventing the resist pattern from swelling during alkaline development. The resist pattern which is fully collapse resistant can be formed. The resist composition is thus useful in micropatterning.

### DETAILED DESCRIPTION OF THE INVENTION

As used herein, the singular forms “a,” “an” and “the” include plural referents unless the context clearly dictates otherwise. “Optional” or “optionally” means that the subsequently described event or circumstances may or may not occur, and that description includes instances where the event or circumstance occurs and instances where it does not. The notation (Cn-Cm) means a group containing from n to m carbon atoms per group. In chemical formulae, Me stands for methyl, Ac for acetyl, and the broken line designates a valence bond. As used herein, the term “fluorinated” refers to a fluorine-substituted or fluorine-containing compound or group. The terms “group” and “moiety” are interchangeable.

The abbreviations and acronyms have the following meaning.
EB: electron beam
EUV: extreme ultraviolet
Mw: weight average molecular weight
Mn: number average molecular weight
Mw/Mn: molecular weight distribution or dispersity
GPC: gel permeation chromatography
PEB: post-exposure bake
PAG: photoacid generator
LWR: line width roughness
EL: exposure latitude
DOF: depth of focus

Onium Salt

One embodiment of the invention is an onium salt having an anion moiety whose conjugate acid is decomposed under the action of acid and heat into carbon dioxide and an organic compound of up to 12 carbon atoms. Specifically, the onium salt has the formula (1).

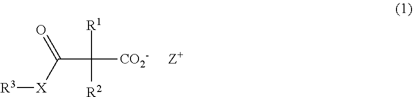

In formal (1), X is a single bond, —O— or —S—, preferably a single bond or —O—, more preferably —O—.

In formula (1), R¹ and R² are each independently hydrogen or a C₁-C₁₀ hydrocarbyl group in which some constituent —CH₂— may be replaced by —O— or —C(═O)—. The hydrocarbyl group may be saturated or unsaturated and straight, branched or cyclic. Examples thereof include C₁-C₁₀ alkyl groups such as methyl, ethyl, n-propyl, isopropyl, n-butyl, and tert-butyl; C₃-C₁₀ cyclic saturated hydrocarbyl groups such as cyclopropyl, cyclopentyl, cyclohexyl, cyclopropylmethyl, 4-methylcyclohexyl, cyclohexylmethyl, norbornyl and adamantyl; C₂-C₁₀ alkenyl groups such as vinyl, allyl, propenyl, butenyl and hexenyl; C₃-C₁₀ cyclic unsaturated hydrocarbyl groups such as cyclohexenyl; C₆-C₁₀ aryl groups such as phenyl and naphthyl; C₇-C₁₀ aralkyl groups such as benzyl, 1-phenylethyl and 2-phenylethyl, and combinations thereof. In the foregoing hydrocarbyl group, some constituent —CH₂— may be replaced by —O— or —C(═O)—.

Also, R¹ and R² may bond together to form a ring with the carbon atom to which they are attached. The ring is preferably of 3 to 10 carbon atoms and more preferably saturated. Suitable examples include cyclopropane, cyclobutane, cyclopentane, cyclohexane, norbornane, and adamantane rings. In the ring, some constituent —CH₂— may be replaced by —O— or —C(═O)—.

In a preferred embodiment, R¹ and R² each are hydrogen or a C₁-C₆ saturated hydrocarbyl group, or R¹ and R², taken together, form a C₃-C₅ saturated ring with the carbon atom to which they are attached. More preferably. R¹ and R² each are hydrogen or a C₁-C₄ saturated hydrocarbyl group, or R¹ and R², taken together, form a C₃-C₆ saturated ring with the carbon atom to which they are attached.

In formula (1), R³ is hydrogen or a C₁-C₁₀ hydrocarbyl group when X is a single bond or —S—, and hydrogen, a C₁-C₁₀ hydrocarbyl group other than an acid labile group, or an acid labile group when X is —O—. The hydrocarbyl group may be saturated or unsaturated and straight, branched or cyclic. Examples thereof include C₁-C₁₀ alkyl groups such as methyl, ethyl, n-propyl, isopropyl, n-butyl, and tert-butyl; C₃-C₁₀ cyclic saturated hydrocarbyl groups such as cyclopropyl, cyclopentyl, cyclohexyl, cyclopropyhnethyl, 4-methylcyclohexyl, cyclohexylmethyl, norbornyl and adamantyl; C₂-C₁₀ alkenyl groups such as vinyl, allyl, propenyl, butenyl and hexenyl: C₃-C₁₀ cyclic unsaturated hydrocarbyl groups such as cyclohexenyl; C₆-C₁₀ aryl groups such as phenyl and naphthyl; C₇-C₁₀ aralkyl groups such as benzyl, 1-phenylethyl and 2-phenylethyl, and combinations thereof. In the foregoing hydrocarbyl group, some or all of the hydrogen atoms may be substituted by halogen such as fluorine, chlorine, bromine or iodine, and some constituent —CH₂— may be replaced by —O— or —C(═O)—.

Also, R¹ and R³ may bond together to form a ring with the atoms to which they are attached and intervenient atom. The ring thus formed is a cycloalkyl ketone when X is a single bond, a lactone ring when X is —O—, and a thiolactone ring when X is —S—. The ring is preferably a 3 to 8-membered ring, more preferably 5 to 7-membered ring. In the ring, some or all of the hydrogen atoms may be substituted by halogen, and some constituent —CH₂— may be replaced by —O— or —C(═O)—.

It is noted that the number of carbon atoms within R¹ to R³ is up to 10 when R³ is other than the acid labile group.

The acid labile group R³ preferably has the formula (AL-1) or (AL-2).

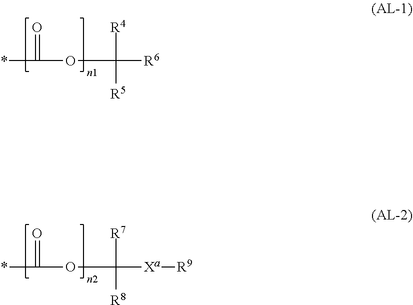

In formula (AL-1), R⁴, R⁵ and R⁶ are each independently a C₁-C₁₂ hydrocarbyl group. Some constituent —CH₂— in the hydrocarbyl group may be replaced by —O— or —S—. When the hydrocarbyl group contains an aromatic ring, some or all of the hydrogen atoms on the aromatic ring may be substituted by halogen, cyano, nitro, optionally halogenated C₁-C₄ alkyl moiety, or optionally halogenated C₁-C₄ alkoxy moiety. The subscript n1 is 0 or 1, and the asterisk (*) designates a point of attachment to the adjacent —O—.

The C₁-C₁₂ hydrocarbyl group represented by R⁴, R⁵ and R⁶ may be saturated or unsaturated and straight, branched or cyclic. Examples thereof include C₁-C₁₂ alkyl groups such as methyl, ethyl, n-propyl, isopropyl, n-butyl, sec-butyl, tert-butyl, n-pentyl, tert-pentyl, n-hexyl, n-octyl, 2-ethylhexyl, n-nonyl, n-decyl, n-undecyl, and n-dodecyl; C₃-C₁₂ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, norbornylmethyl, adamantyl, adamantylmethyl, tricyclo[5.2.1.0^{2,6}]decyl, and tetracyclo[6.2.1.1^{3,6}0^{2,7}]dodecyl; C₂-C₁₂ alkenyl groups such as vinyl, allyl, propenyl, butenyl, pentenyl and hexenyl; C₂-C₁₂ alkynyl groups such as ethynyl, propynyl, butynyl, pentynyl and hexynyl; C₃-C₁₂ cyclic unsaturated aliphatic hydrocarbyl groups such as cyclopentenyl and cyclohexenyl; C₆-C₁₂ aryl groups such as phenyl, naphthyl and indanyl; C₇-C₁₂ aralkyl groups such as benzyl, 1-phenylethyl and 2-phenylethyl, and combinations thereof.

Any two of R⁴, R⁵ and R⁶ may bond together to form a ring. Examples of the thus formed ring include cyclopropane, cyclobutane, cyclopentane, cyclohexane, cycloheptane, cyclooctane, norbornane, adamantane, tricyclo[5.2.1.0^{2,6}]decane, and tetracyclo[6.2.1.1^{3,6}0^{2,7}]dodecane rings. Some constituent —CH₂— in the ring may be replaced by —O— or —S—.

In formula (AL-2), X^{a} is —O— or —S—. R⁷ and R⁸ are each independently hydrogen or a C₁-C₁₀ hydrocarbyl group. The C₁-C₁₀ hydrocarbyl group represented by R⁷ and R⁸ may be saturated or unsaturated and straight, branched or cyclic, and examples thereof are as exemplified above for the C₁-C₁₀ hydrocarbyl group represented by R¹ and R².

In formula (AL-2), R⁹ is a C₁-C₂₀ hydrocarbyl group. Some constituent —CH₂— in the hydrocarbyl group may be replaced by —O— or —S—. The hydrocarbyl group may be saturated or unsaturated and straight, branched or cyclic. Examples thereof include C₁-C₂₀ alkyl groups such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, pentadecyl, heptadecyl, octadecyl, nonadecyl and icosyl; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl, norbornylmethyl, adamantyl, adamantylmethyl, tricyclo[5.2.1.0^{2,6}]decyl, and tetracyclo[6.2.1.1^{3,6}0^{2,7}]dodecyl; C₂-C₂₀ alkenyl groups such as vinyl, propenyl, butenyl, pentenyl and hexenyl; C₂-C₂₀ alkynyl groups such as ethynyl, propynyl, butynyl, pentynyl and hexynyl: C₃-C₂₀ cyclic unsaturated aliphatic hydrocarbyl groups such as cyclopentenyl, cyclohexenyl and norbornenyl; C₆-C₂₀ aryl groups such as phenyl, methylphenyl, ethylphenyl, n-propylphenyl, isopropylphenyl, n-butylphenyl, isobutylphenyl, sec-butylphenyl, tert-butylphenyl, naphthyl, methylnaphthyl, ethylnaphthyl, n-propylnaphthyl, isopropylnaphthyl, n-butylnaphthyl, isobutylnaphthyl, sec-butylnaphthyl, and tert-butylnaphthyl; C₇-C₁₂ aralkyl groups such as benzyl and phenethyl, and combinations thereof. Also, R⁸ and R⁹ may bond together to form a C₃-C₂₀ heterocycle with the carbon atom and X^{a} to which they are attached. Some constituent —CH₂— in the heterocycle may be replaced by —O— or —S—. The subscript n2 is 0 or 1 and * designates a point of attachment to the adjacent —O—.

Examples of the acid labile group having formula (AL-1) are shown below, but not limited thereto. Herein, * designates a point of attachment to the adjacent —O—.

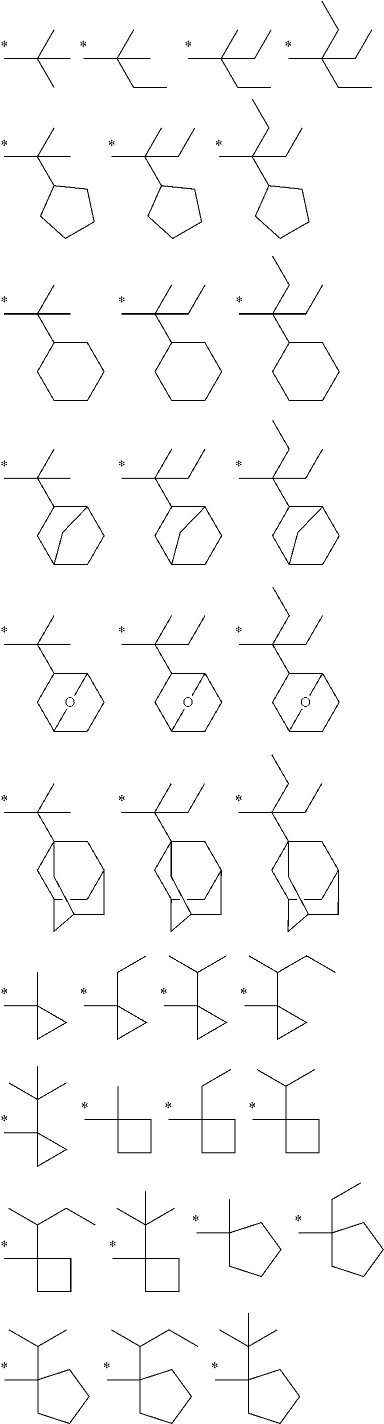

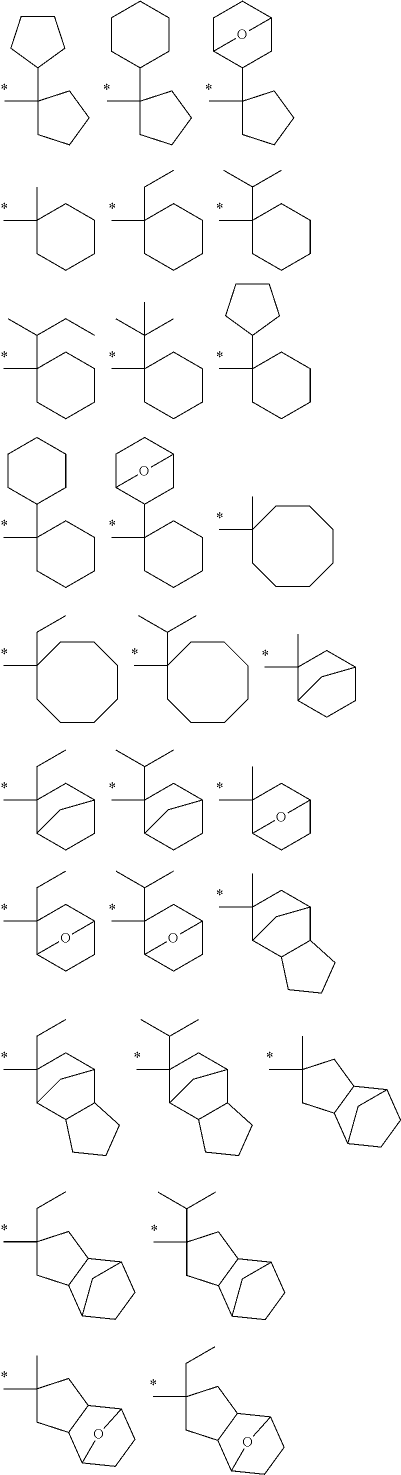

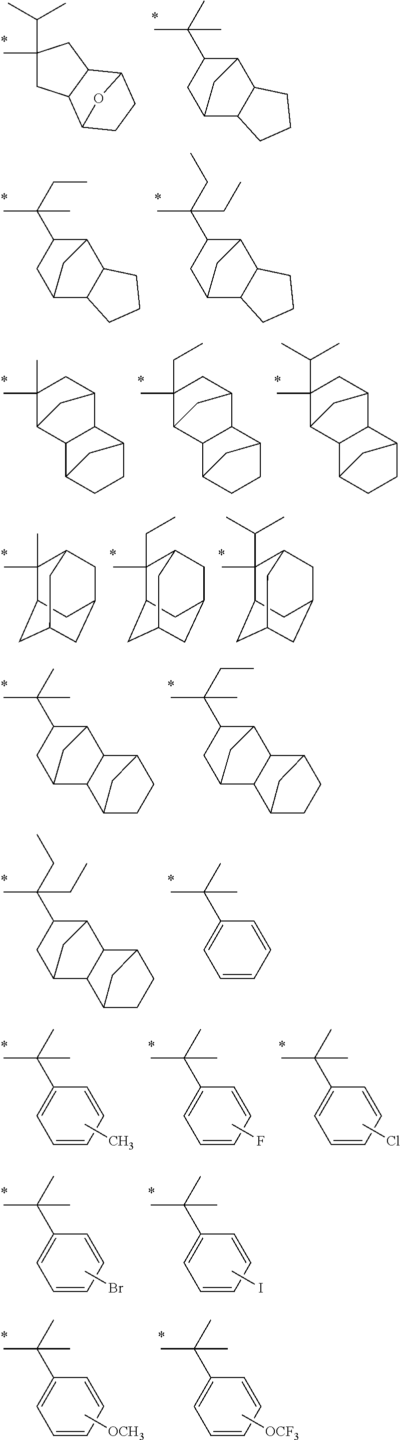

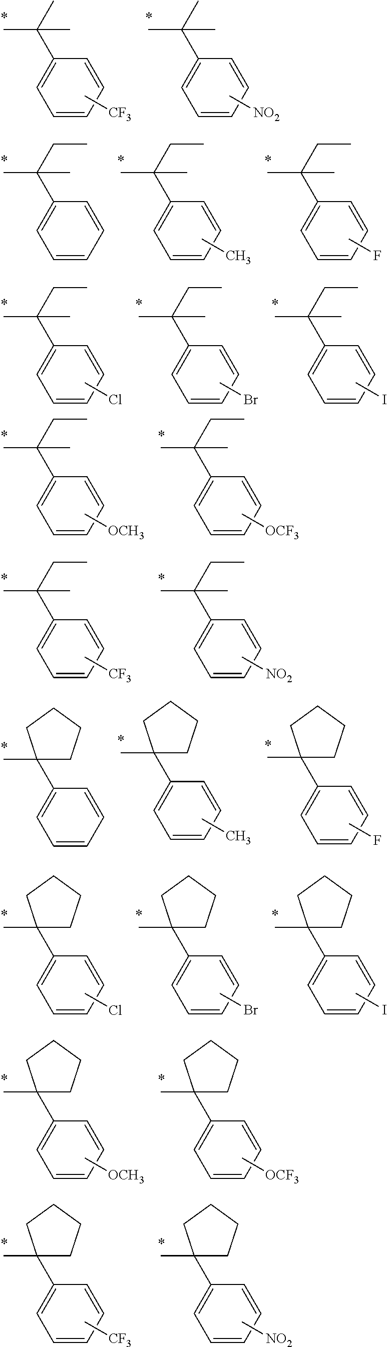

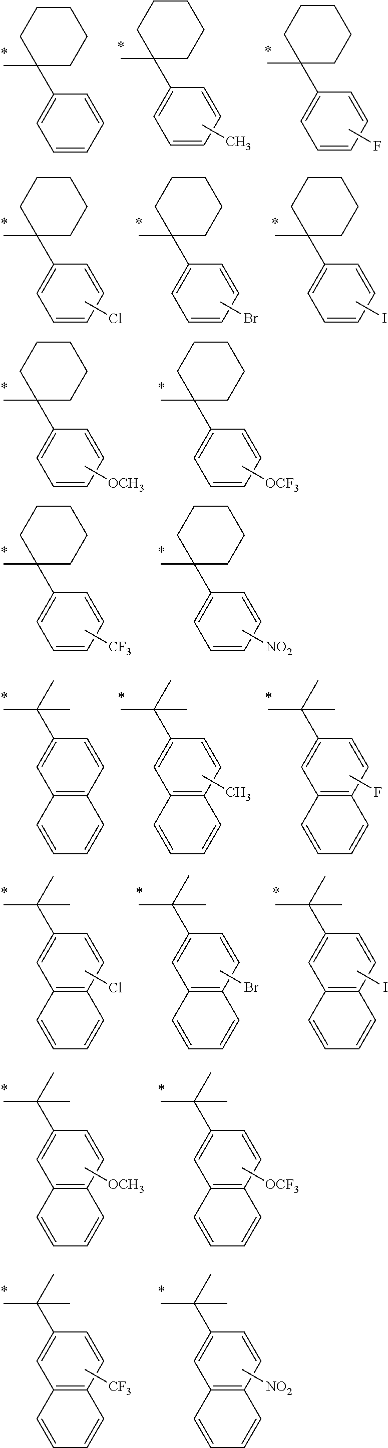

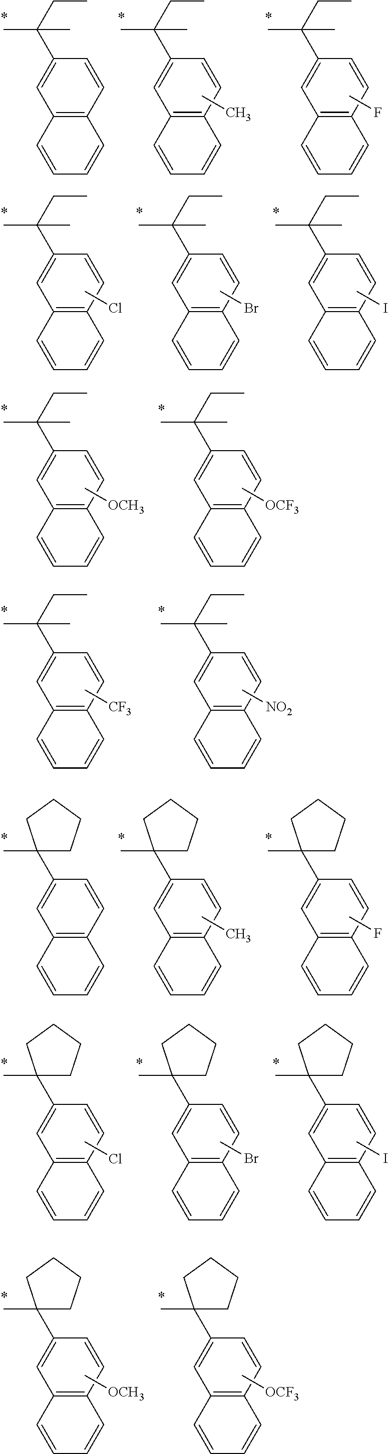

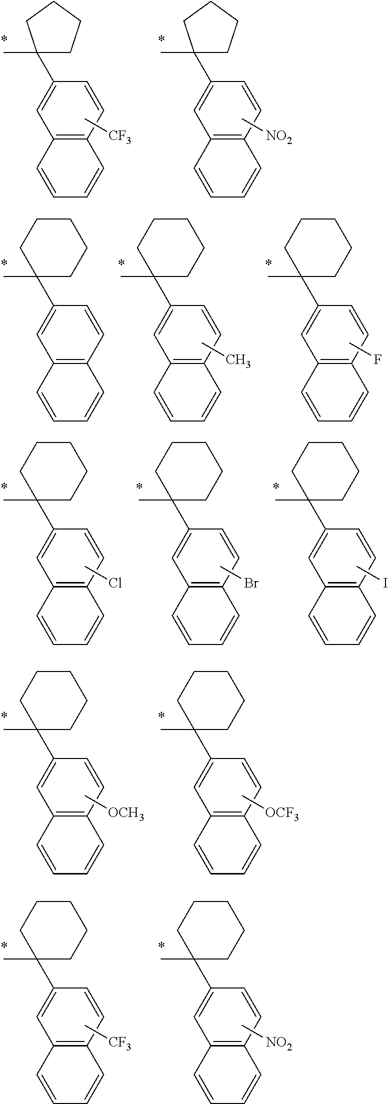

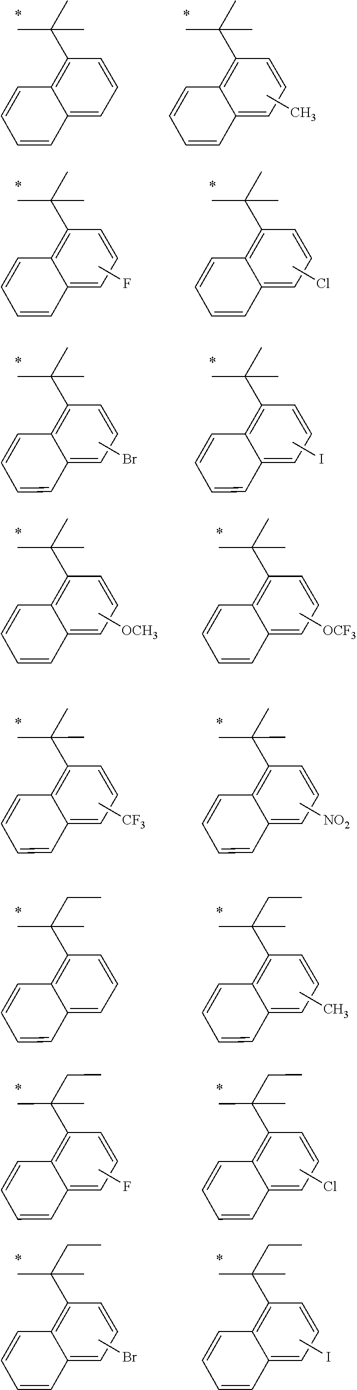

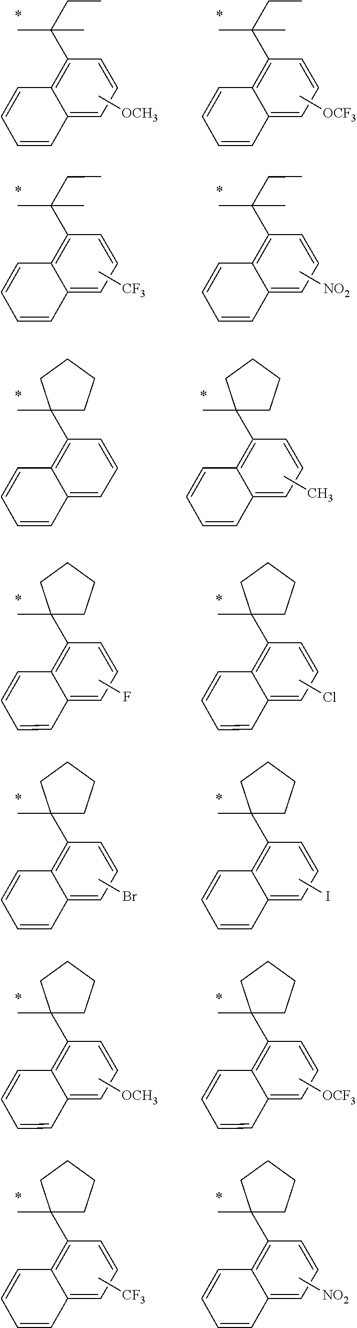

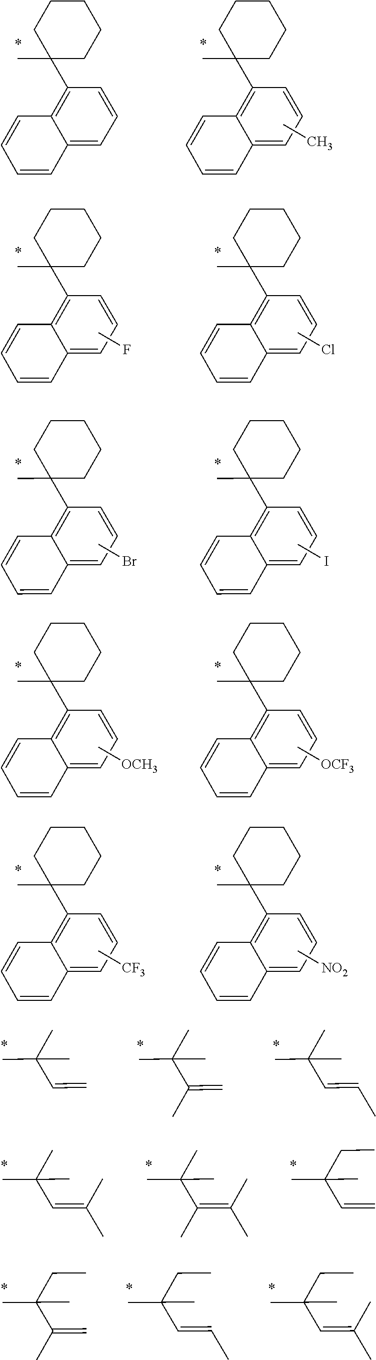

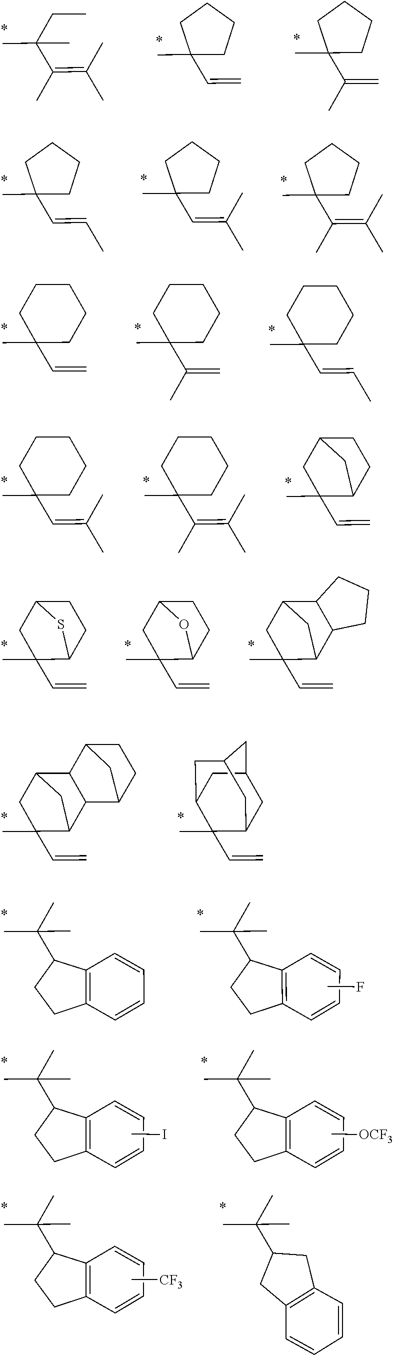

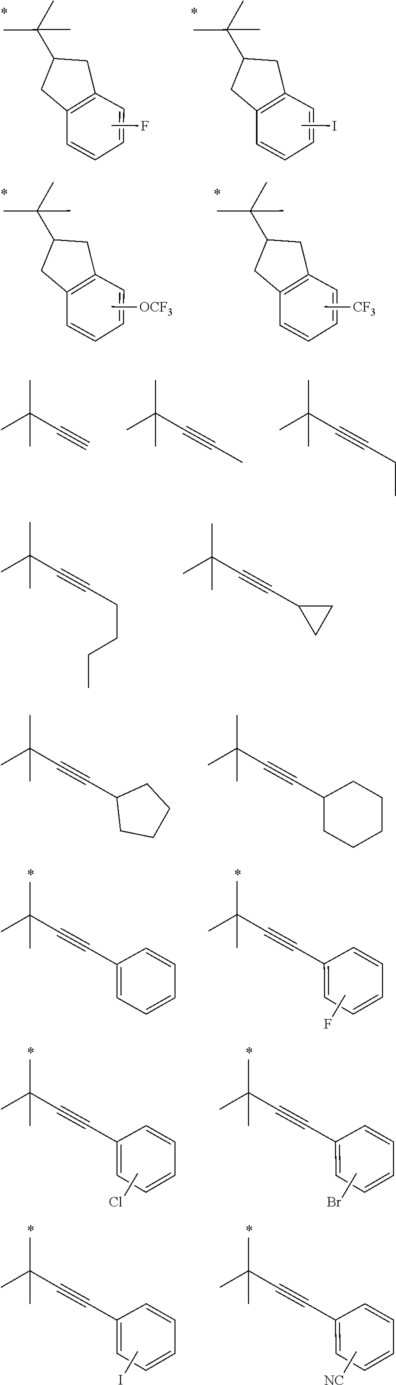

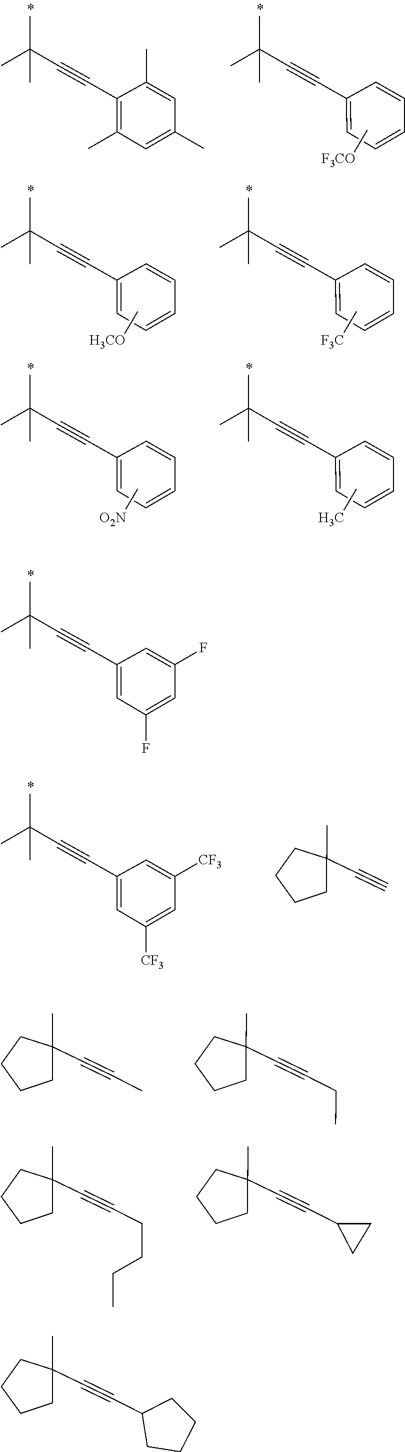

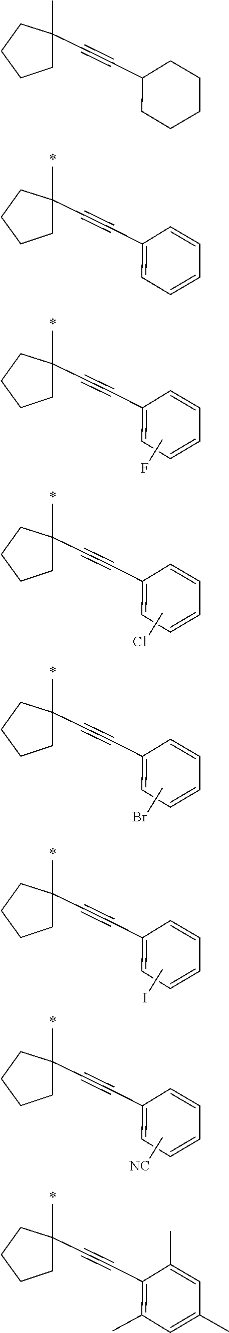

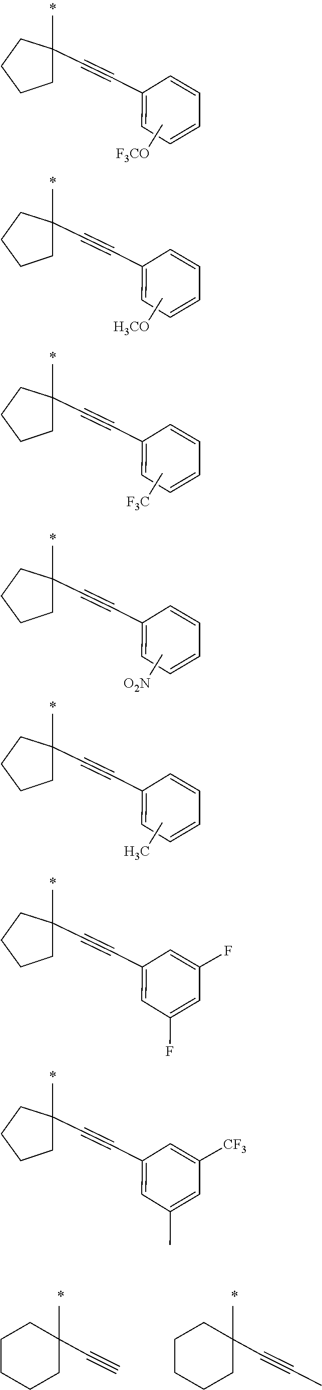

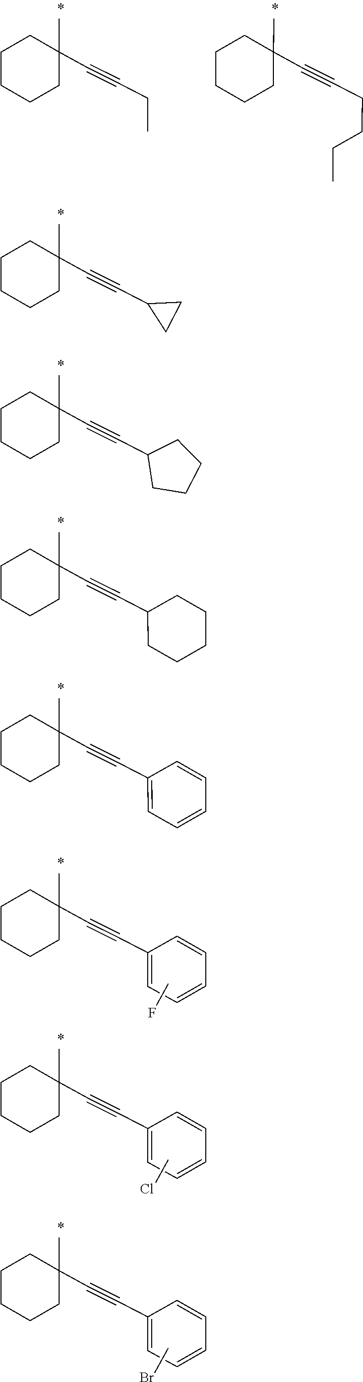

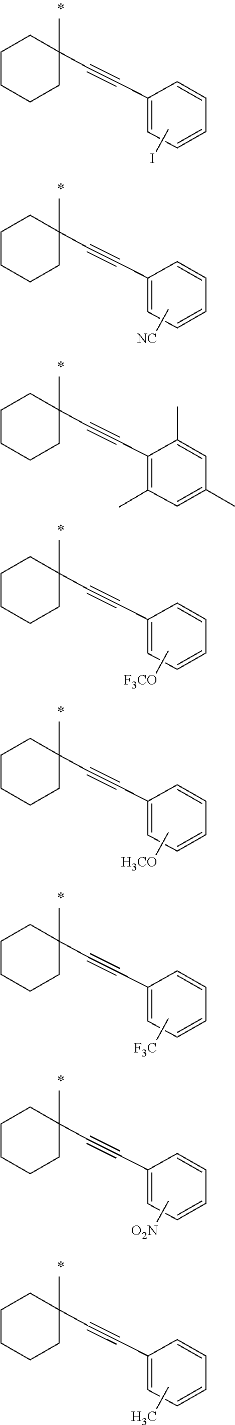

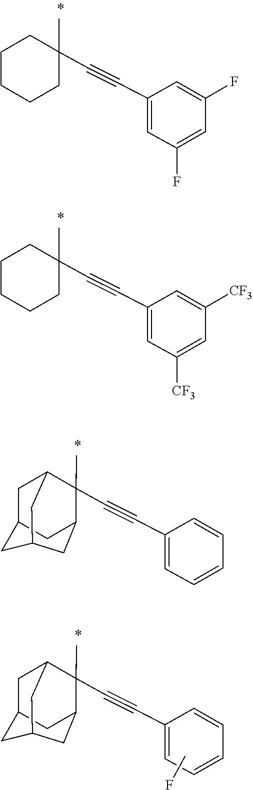

Examples of the acid labile group having formula (AL-2) are shown below, but not limited thereto. Herein, *designates a point of attachment to the adjacent —O—.

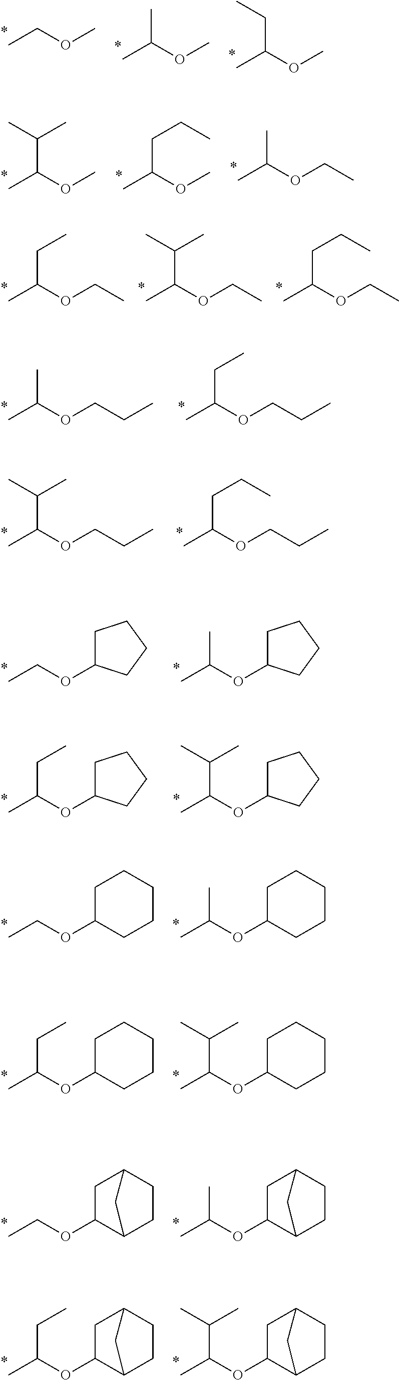

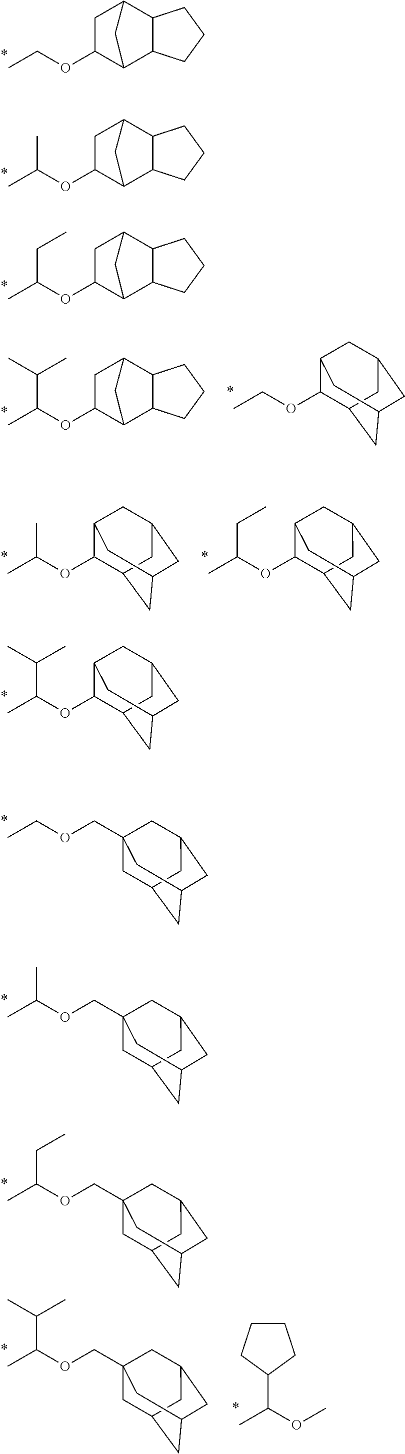

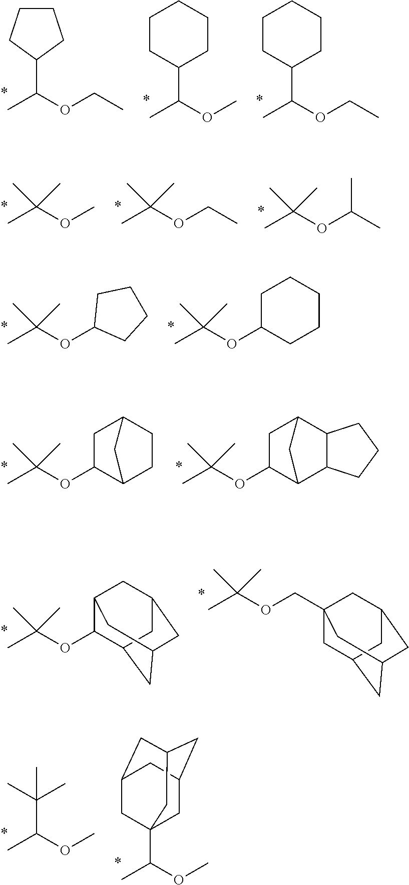

When X is a single bond or —S—, R³ is preferably hydrogen, a C₁-C₄ alkyl group, C₁-C₄ halogenated alkyl group, or C₃-C₆ cyclic saturated hydrocarbyl group, and more preferably hydrogen, a C₁-C₃ alkyl group, C₁-C₃ halogenated alkyl group, or C₃-C₆ cyclic saturated hydrocarbyl group. When X is —O—. R³ is preferably hydrogen, a C₁-C₄ alkyl group other than an acid labile group, a C₁-C₄ halogenated alkyl group other than an acid labile group, or an acid labile group having formula (AL-1) or (AL-2), and more preferably hydrogen, a C₁-C₃ hydrocarbyl group other than an acid labile group, a C₁-C₃ halogenated alkyl group other than an acid labile group, or an acid labile group having formula (AL-1) or (AL-2).

Preferred examples of the anion in the onium salt having formula (1) are shown below, but not limited thereto.

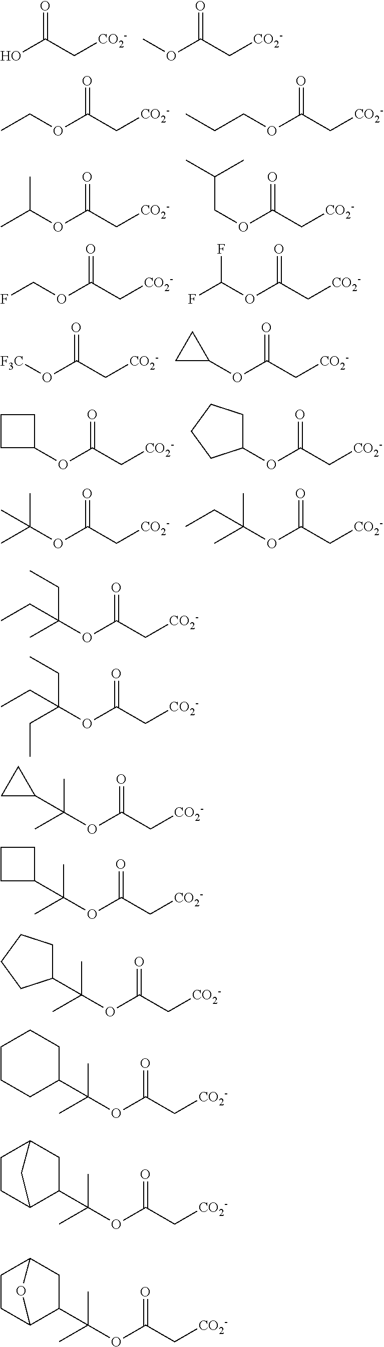

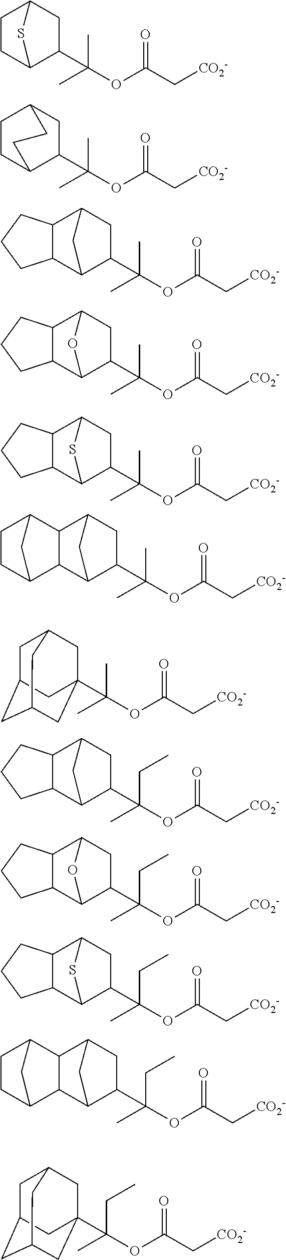

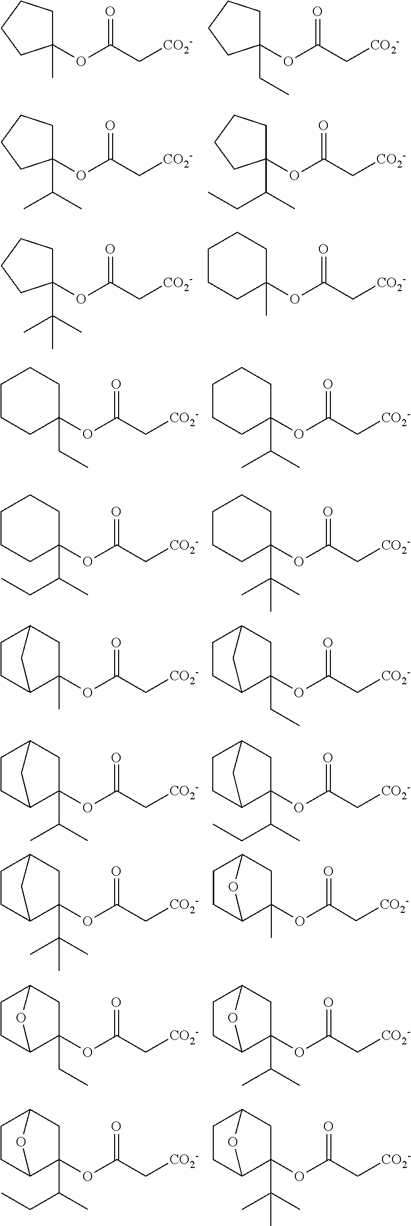

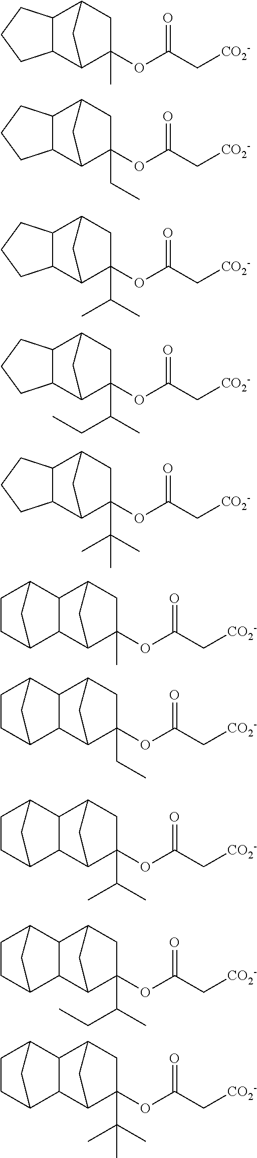

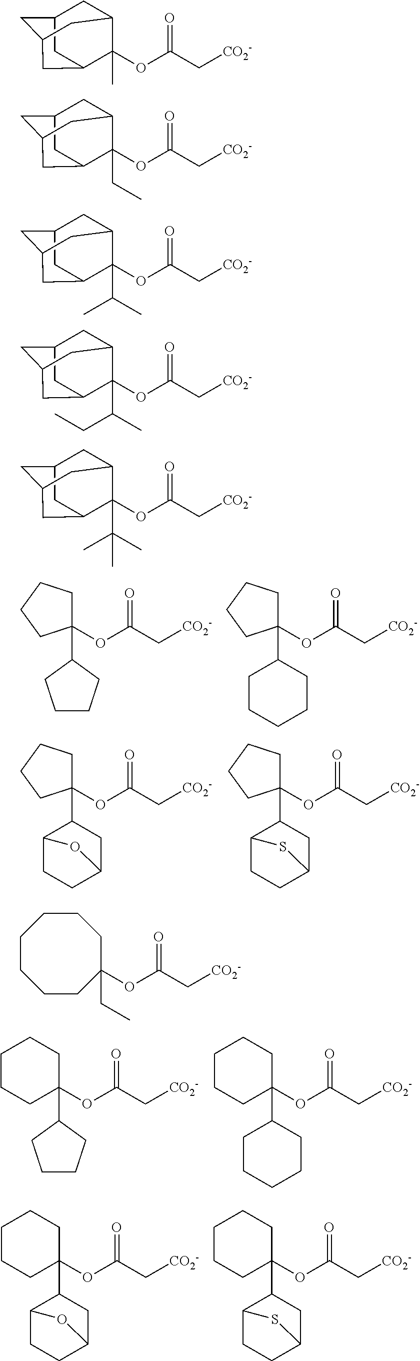

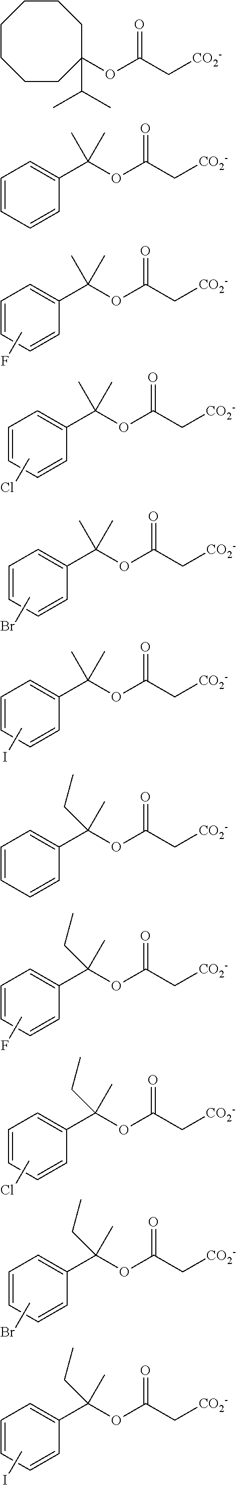

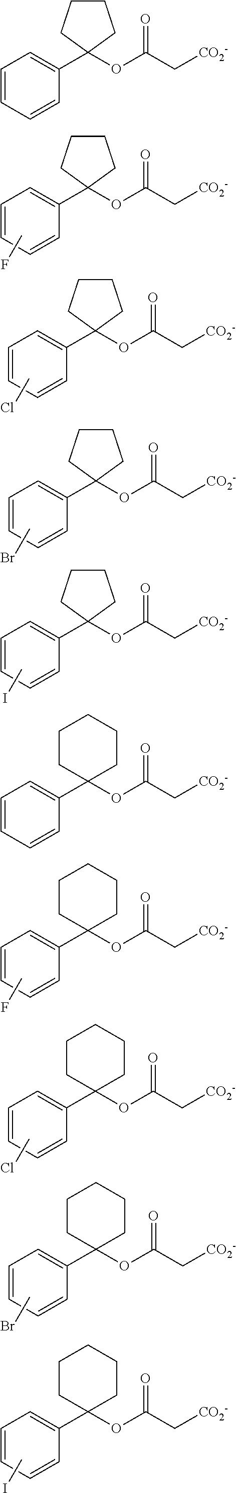

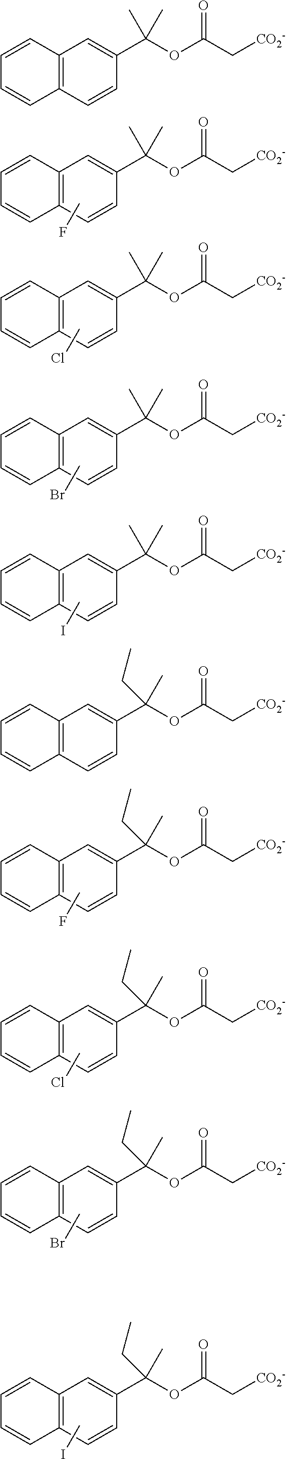

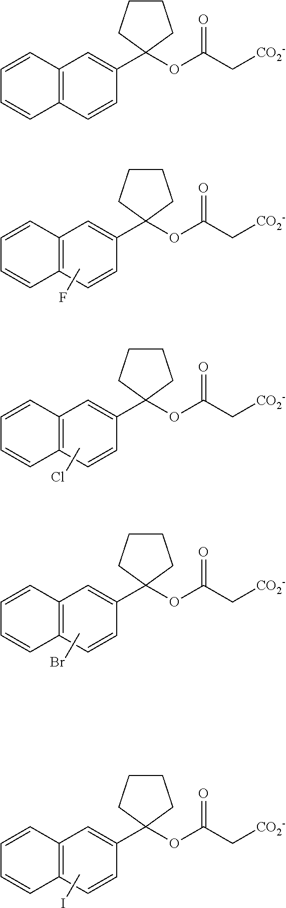

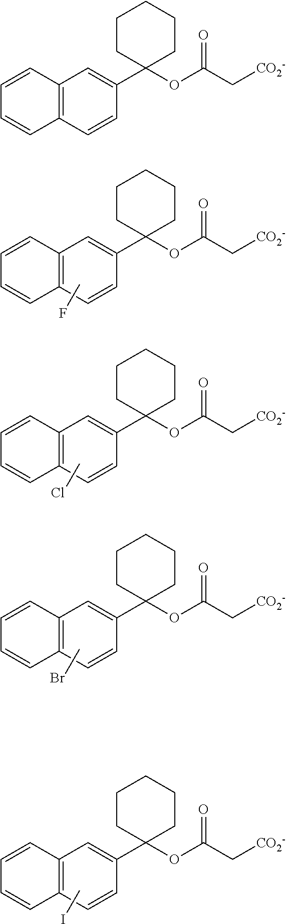

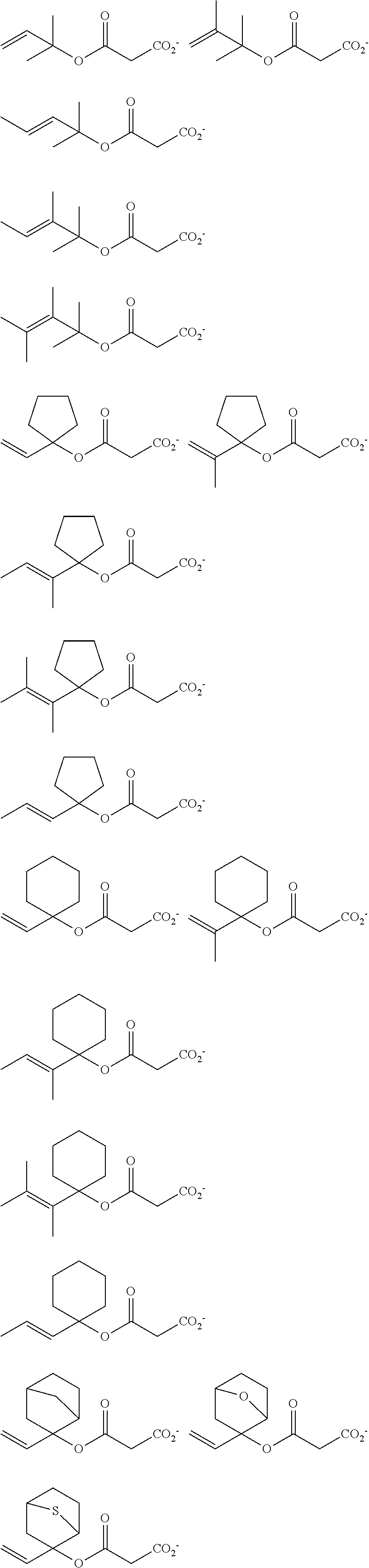

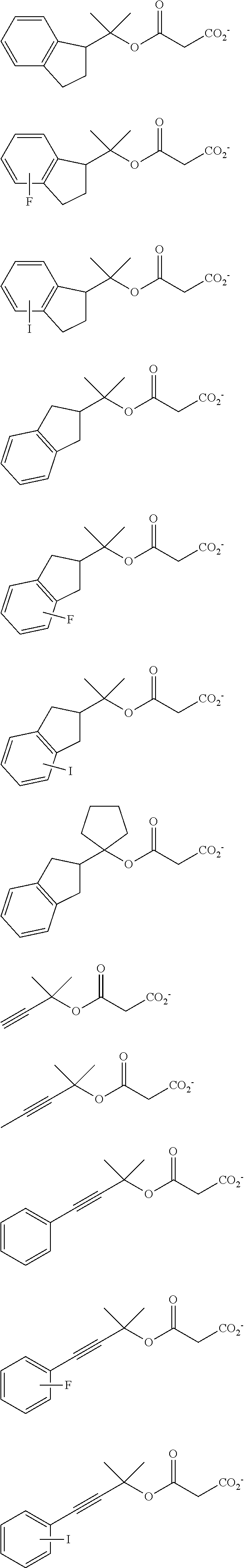

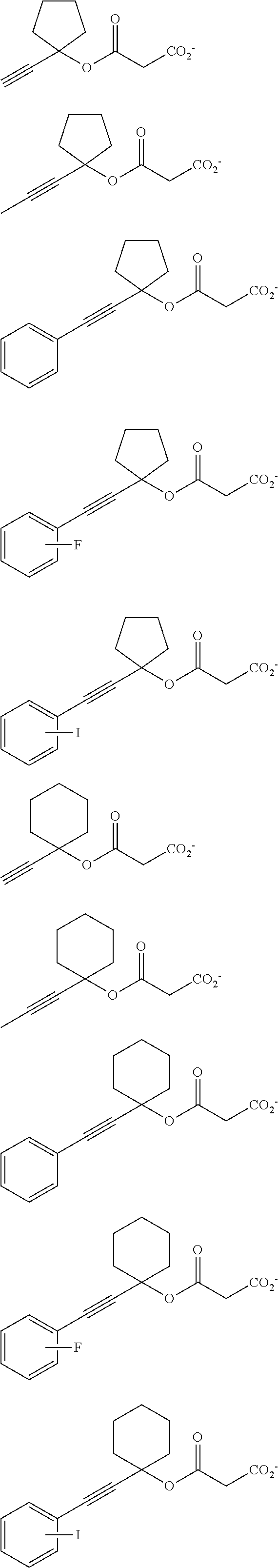

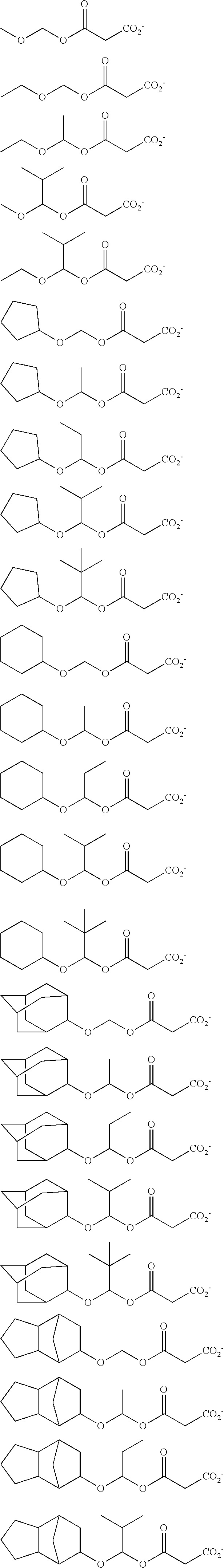

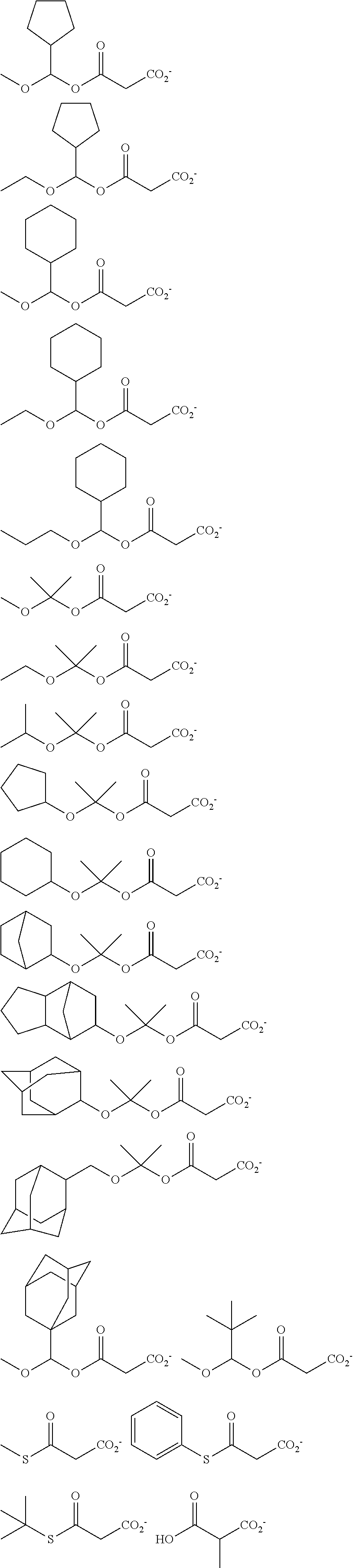

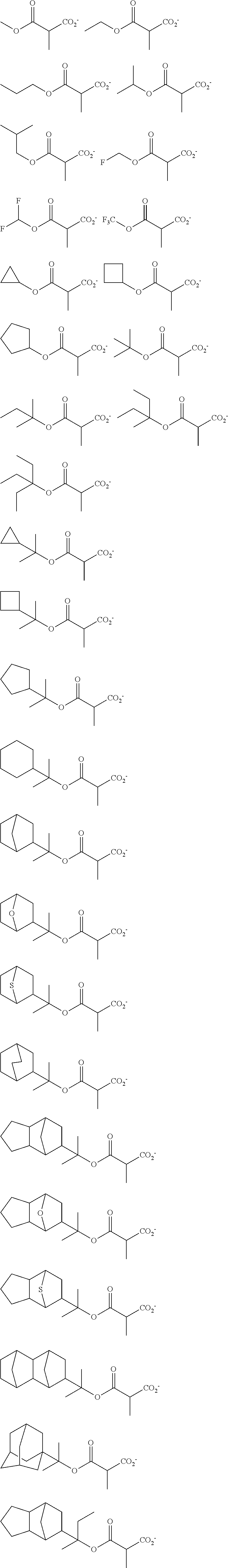

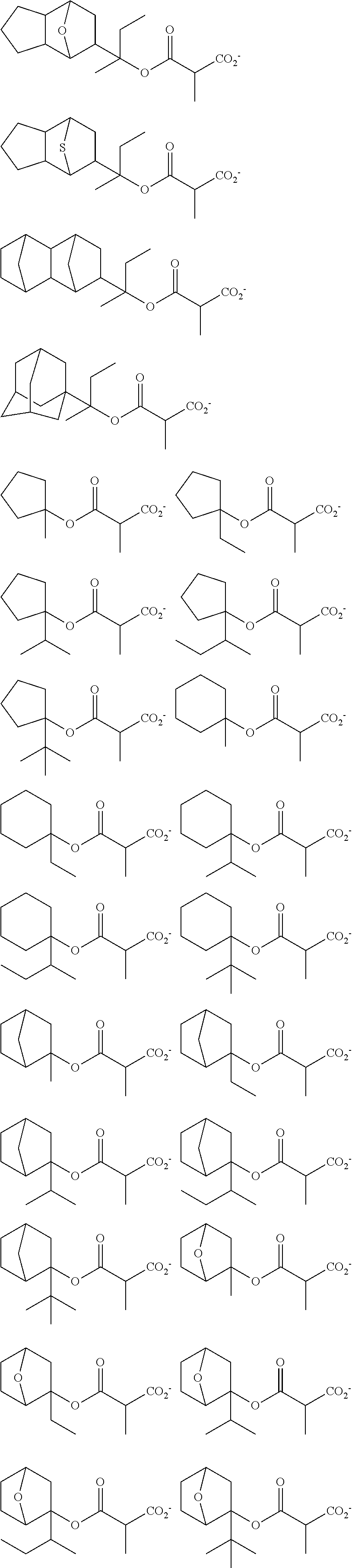

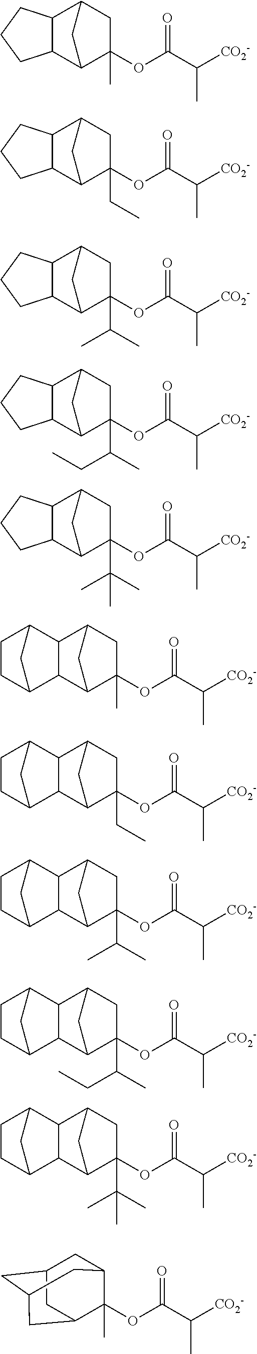

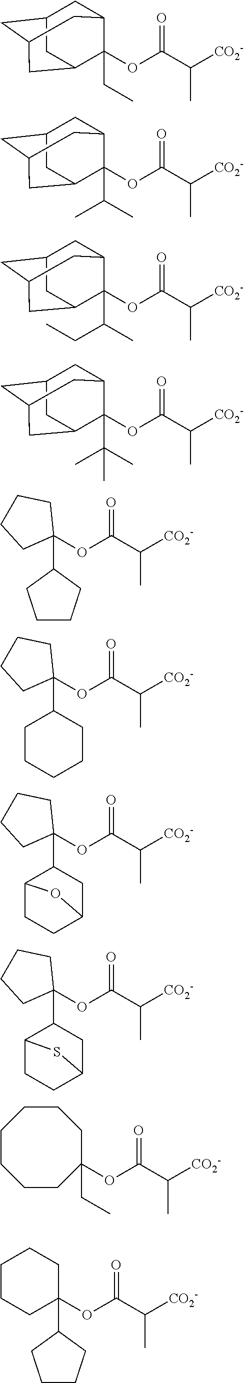

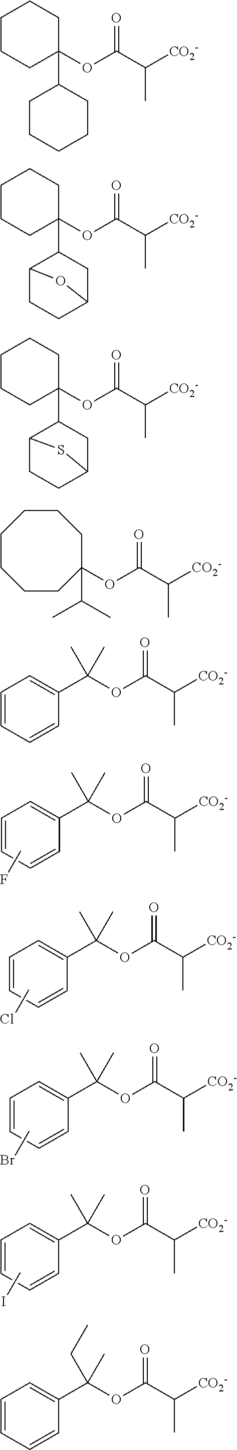

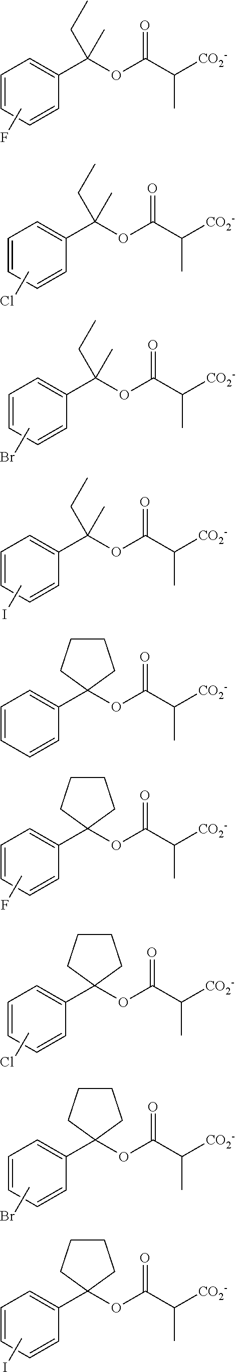

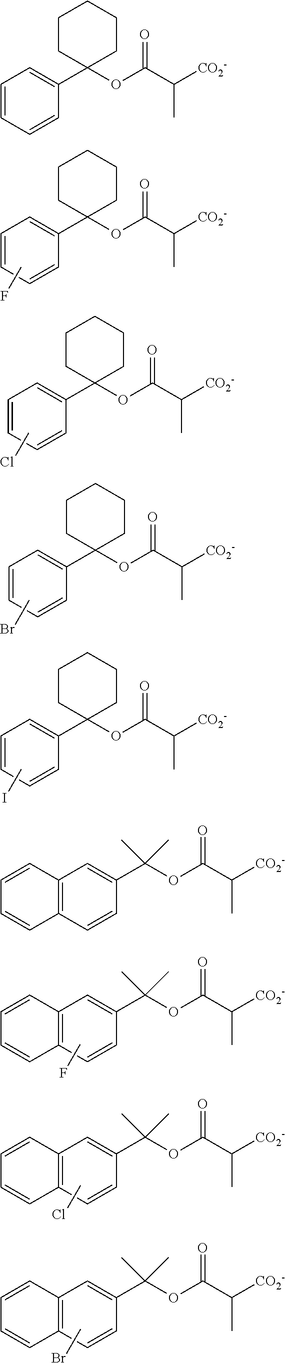

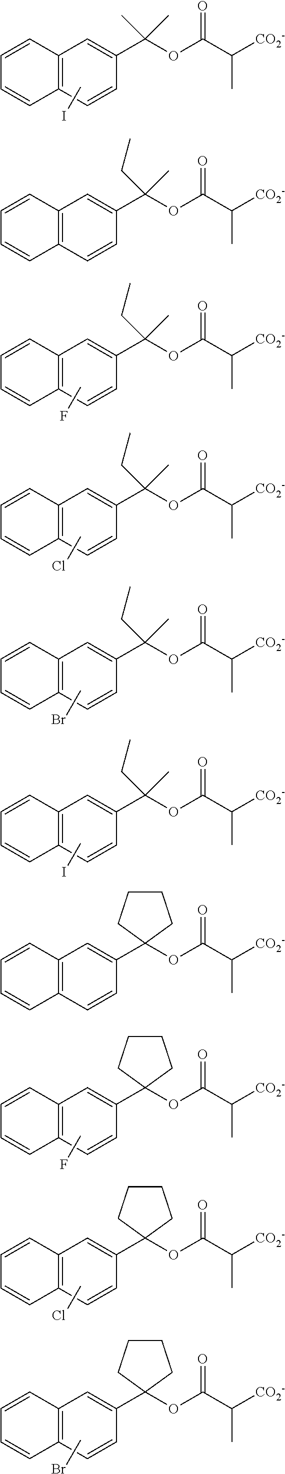

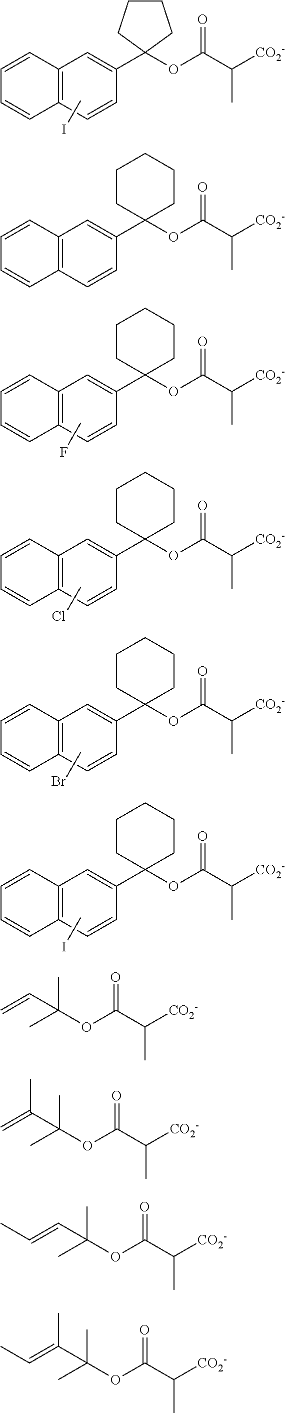

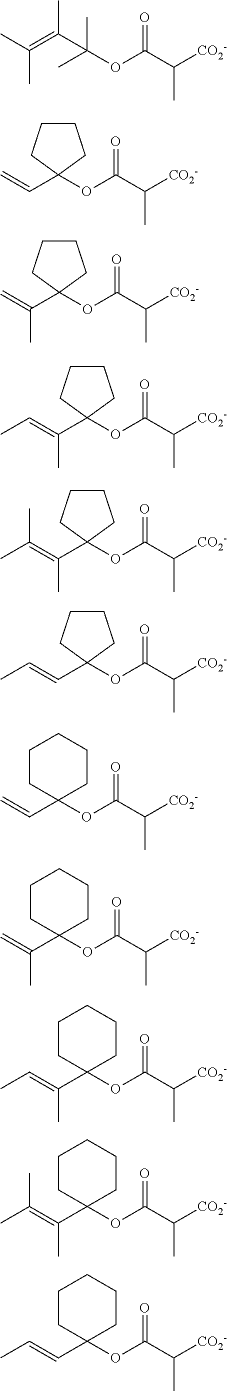

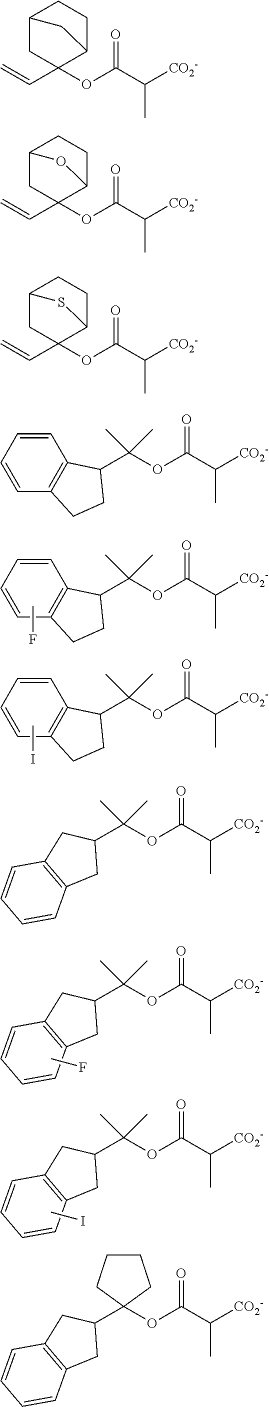

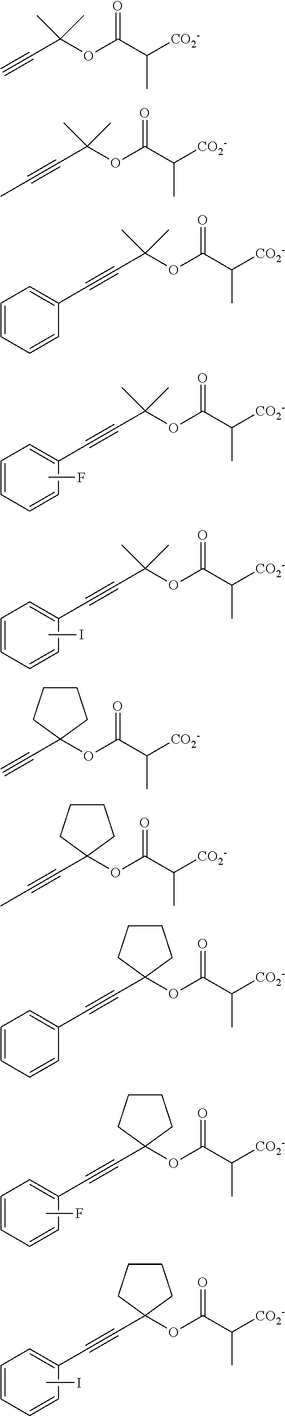

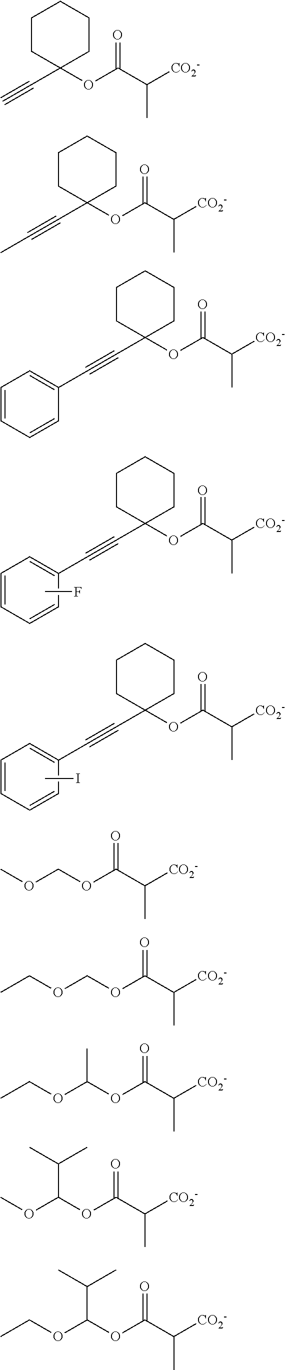

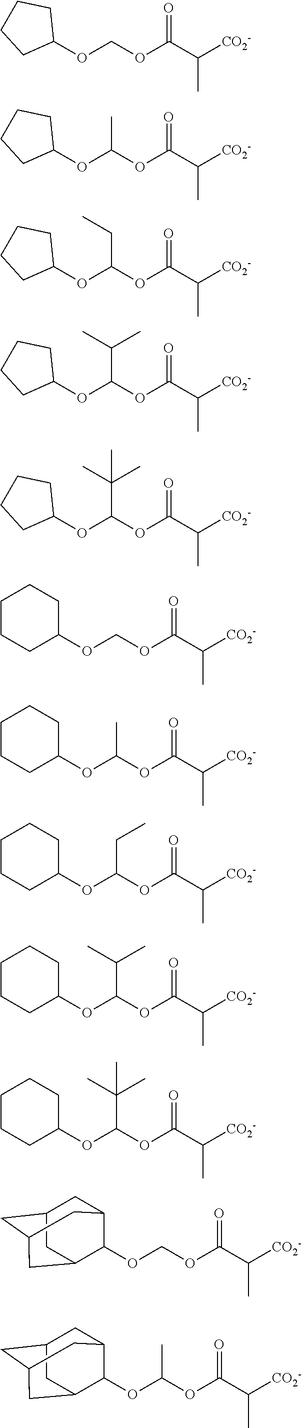

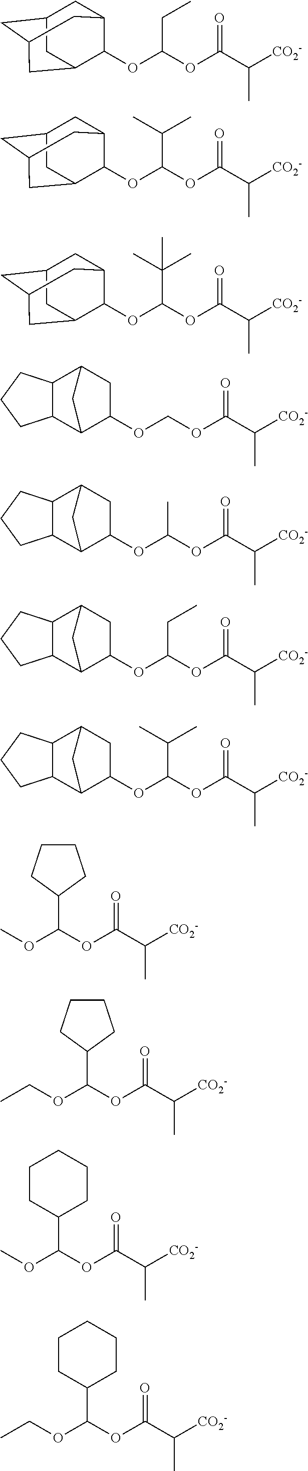

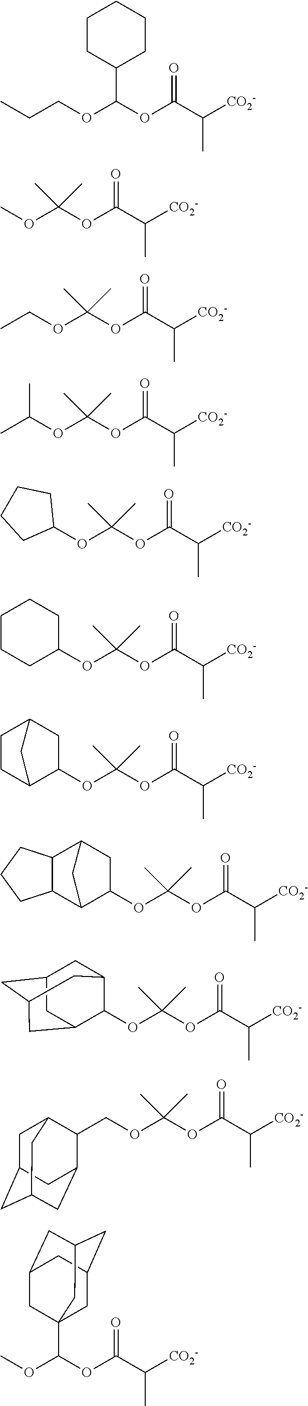

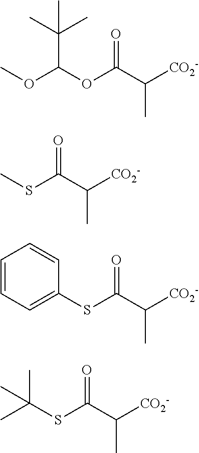

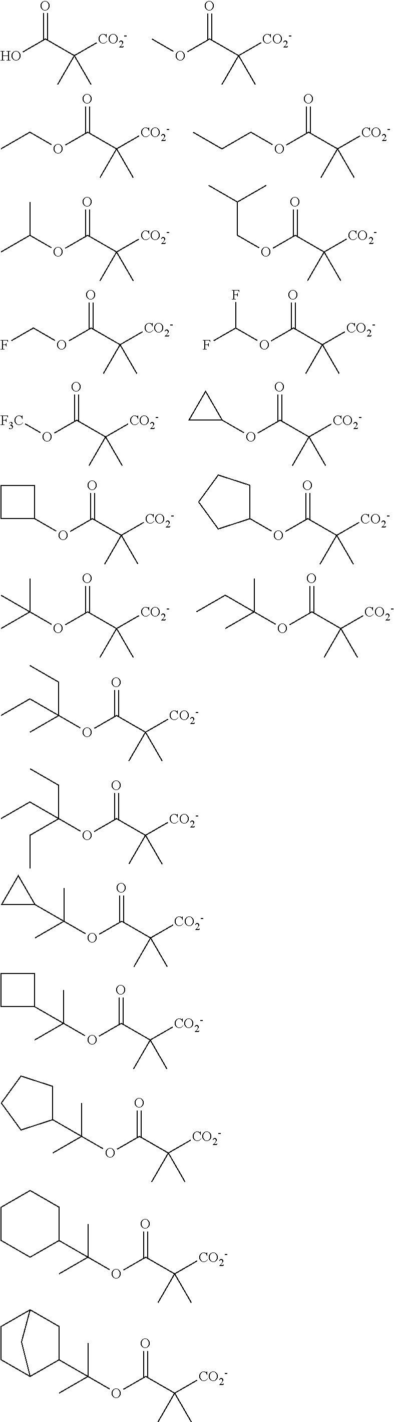

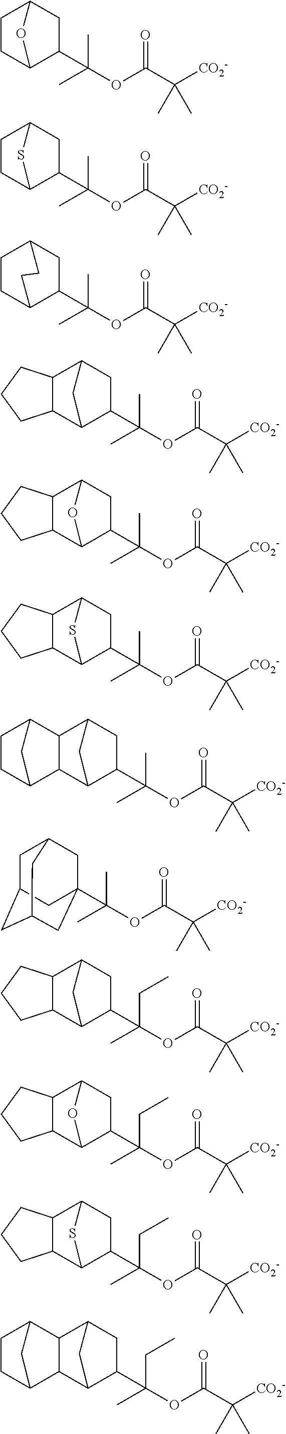

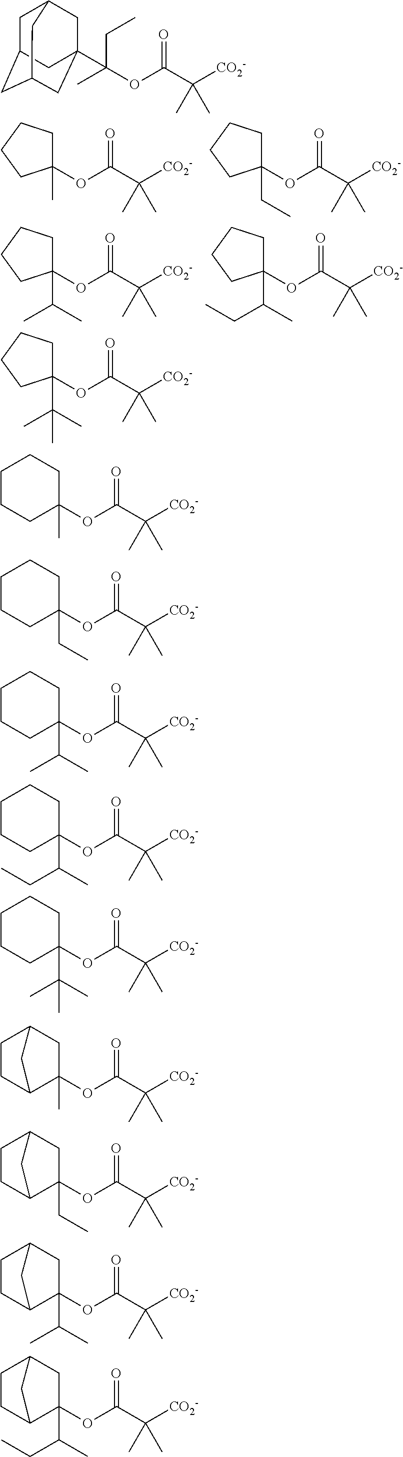

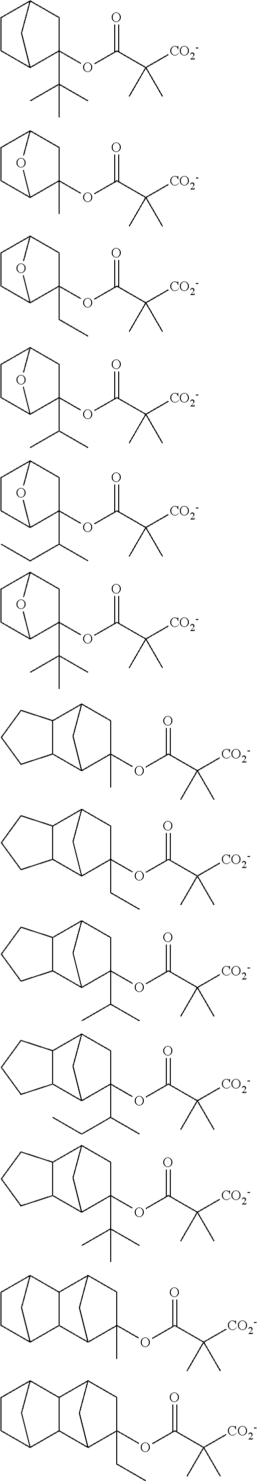

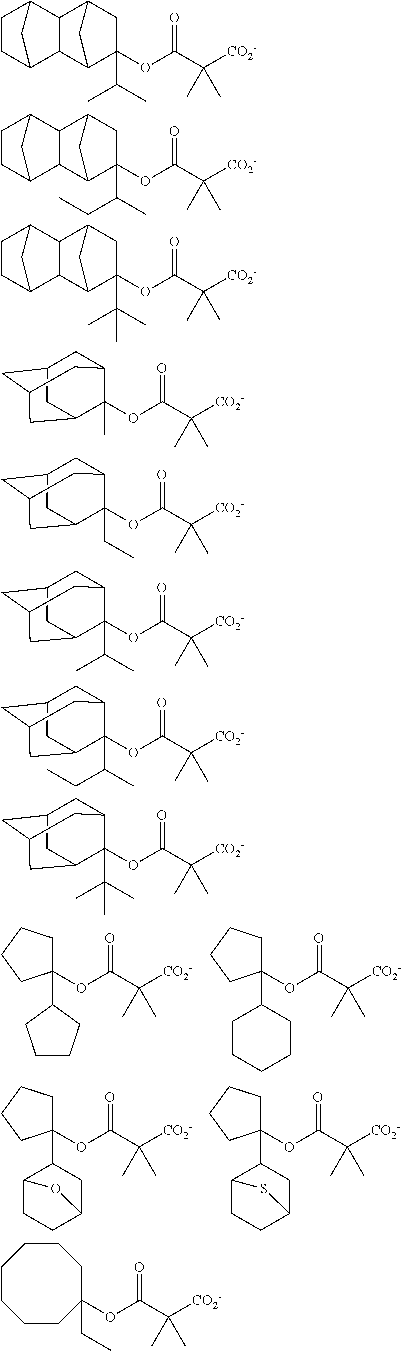

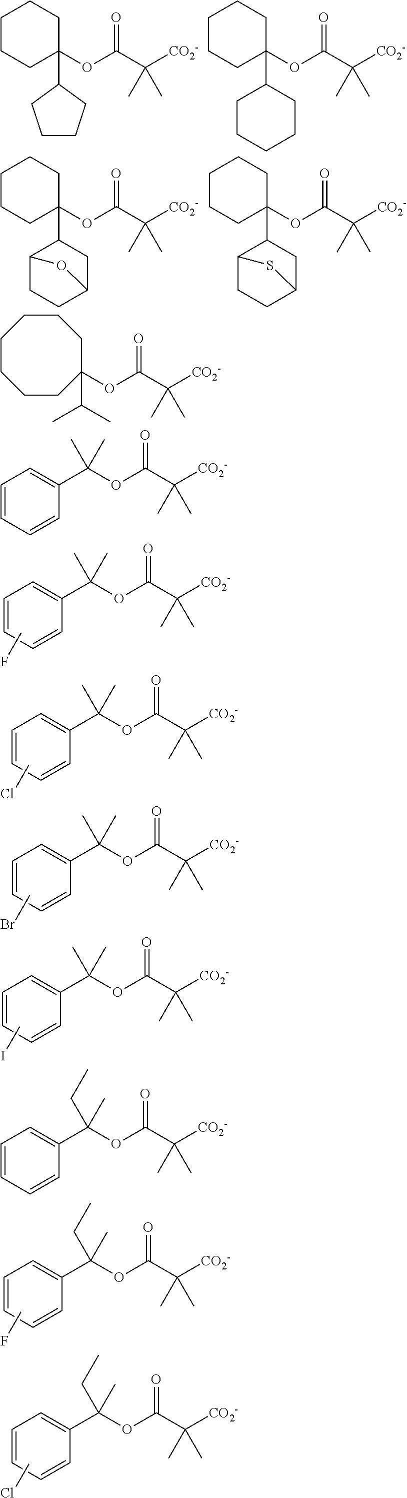

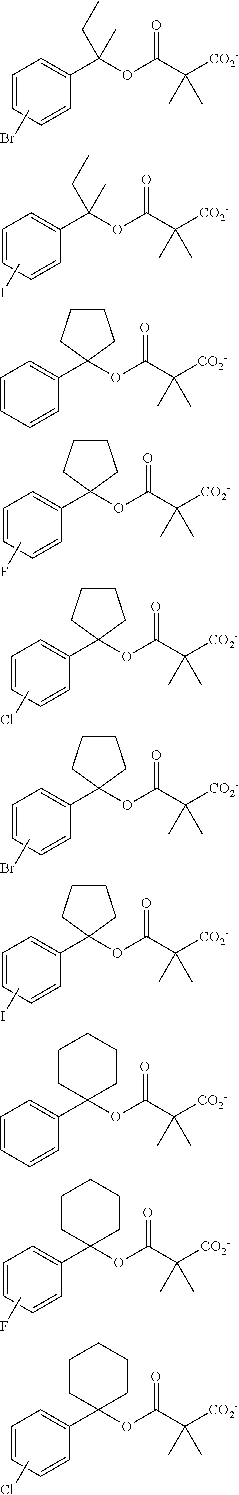

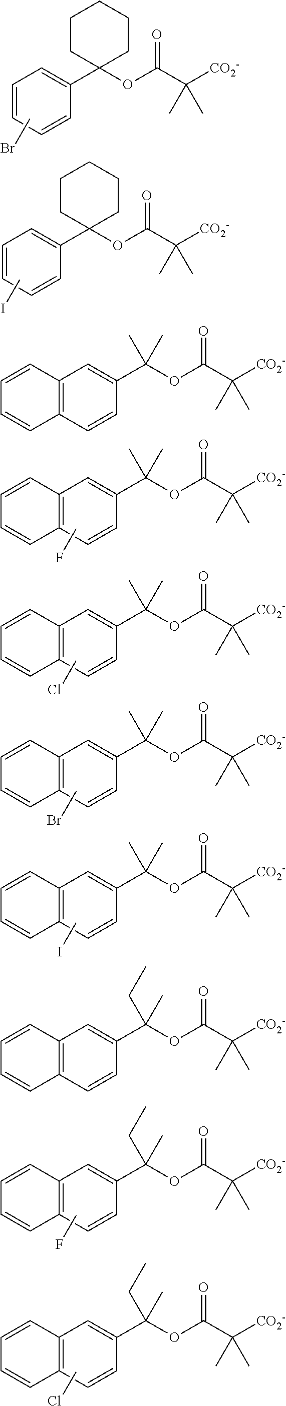

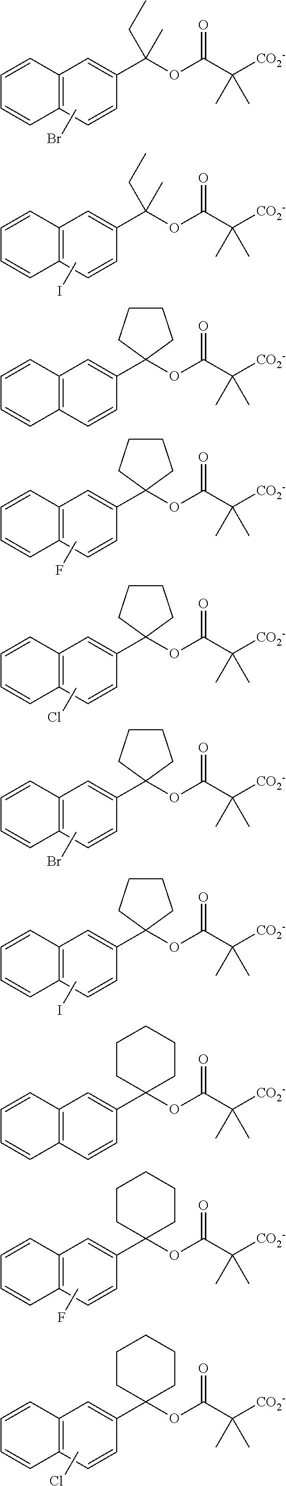

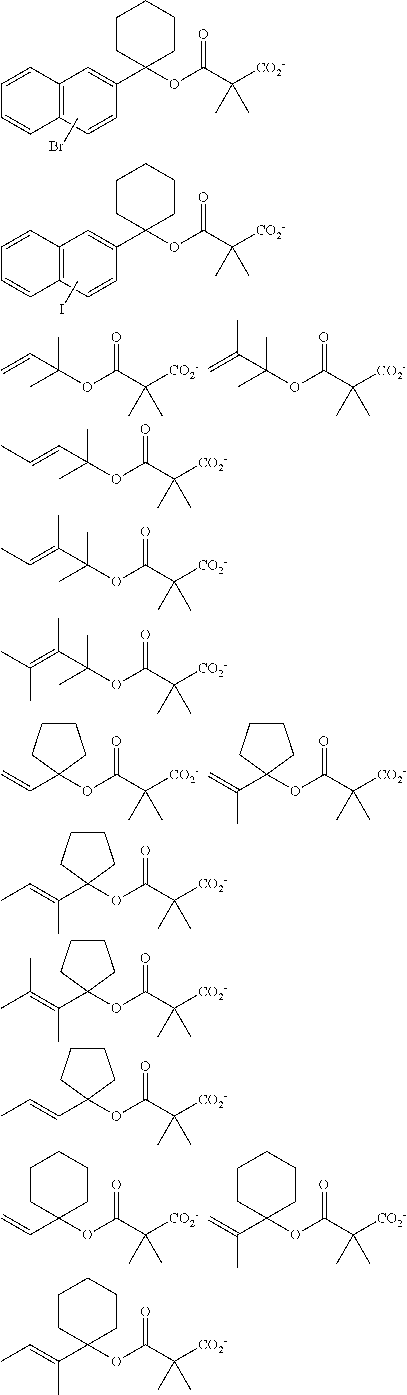

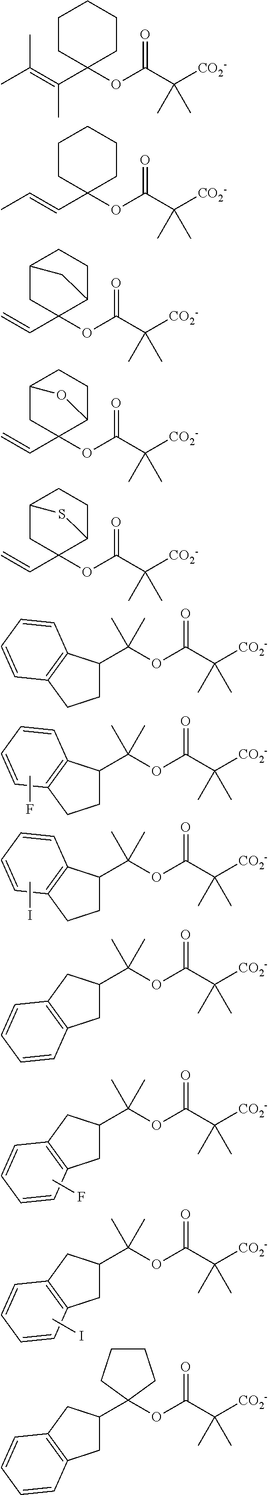

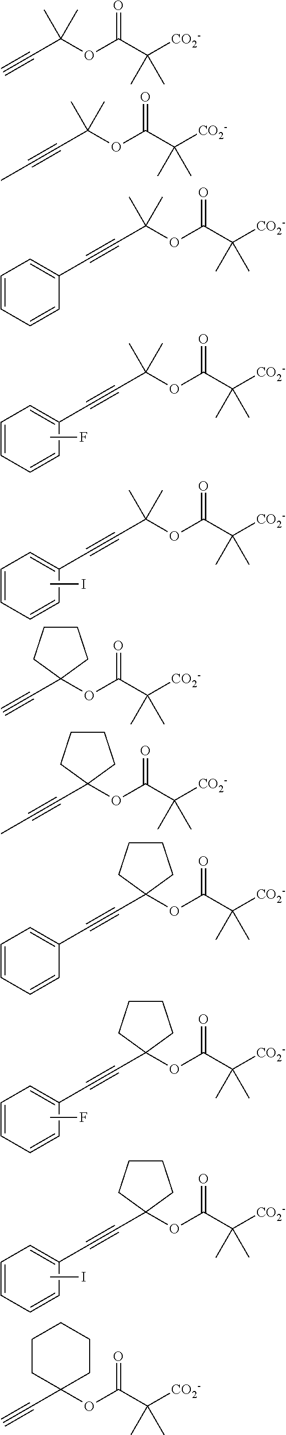

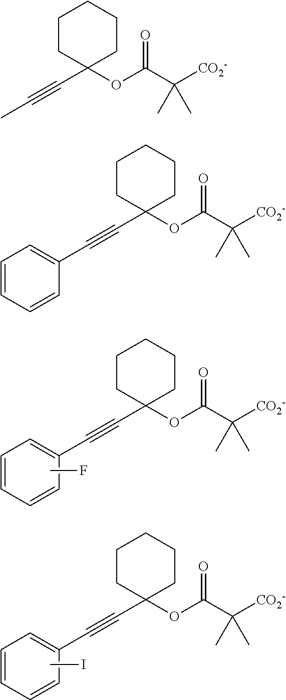

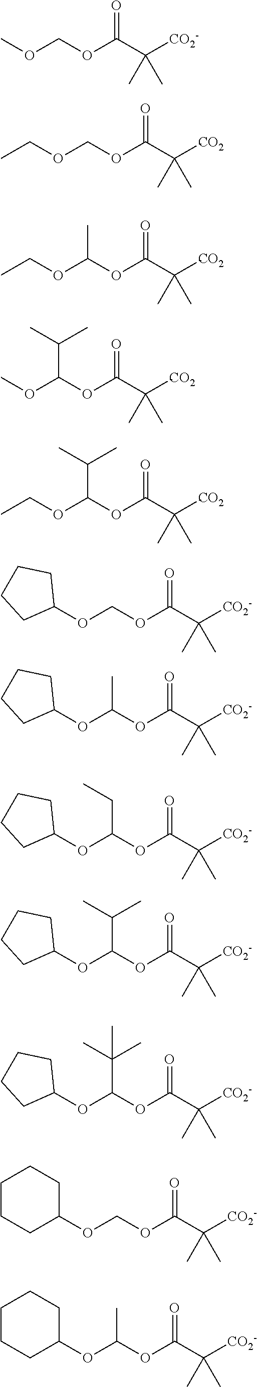

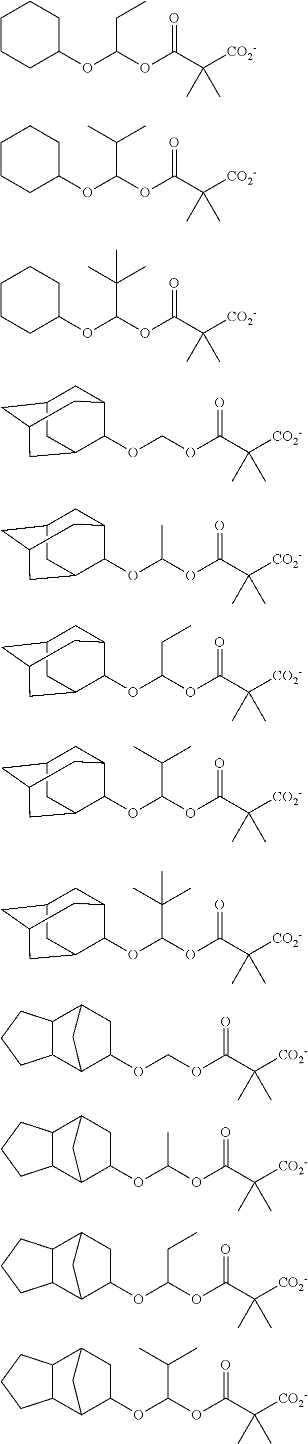

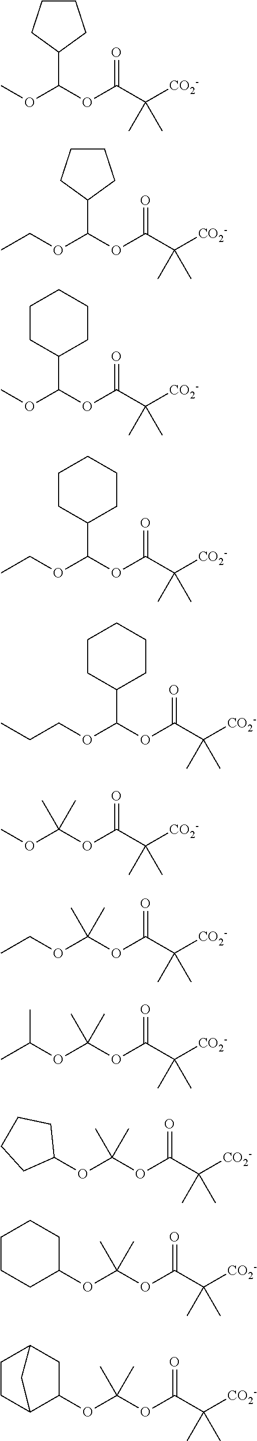

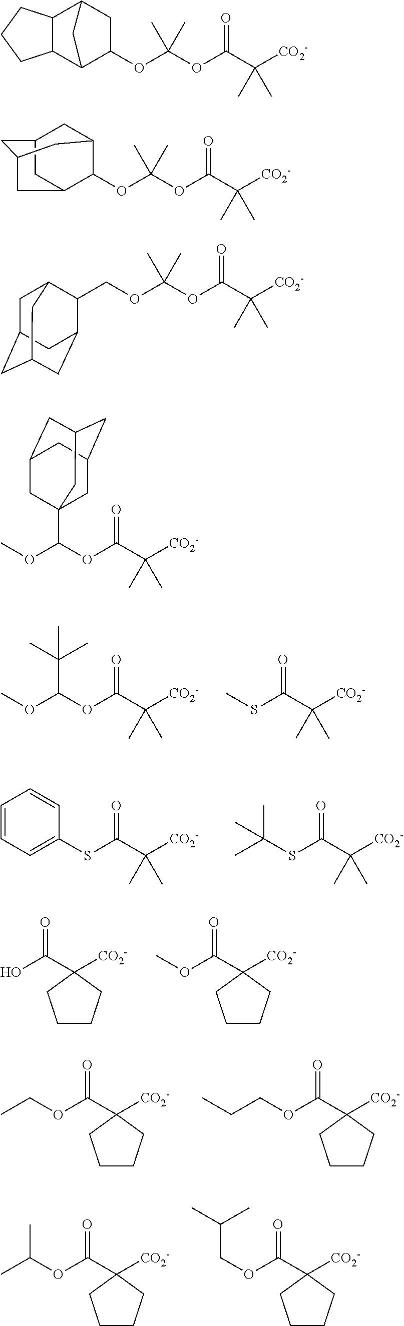

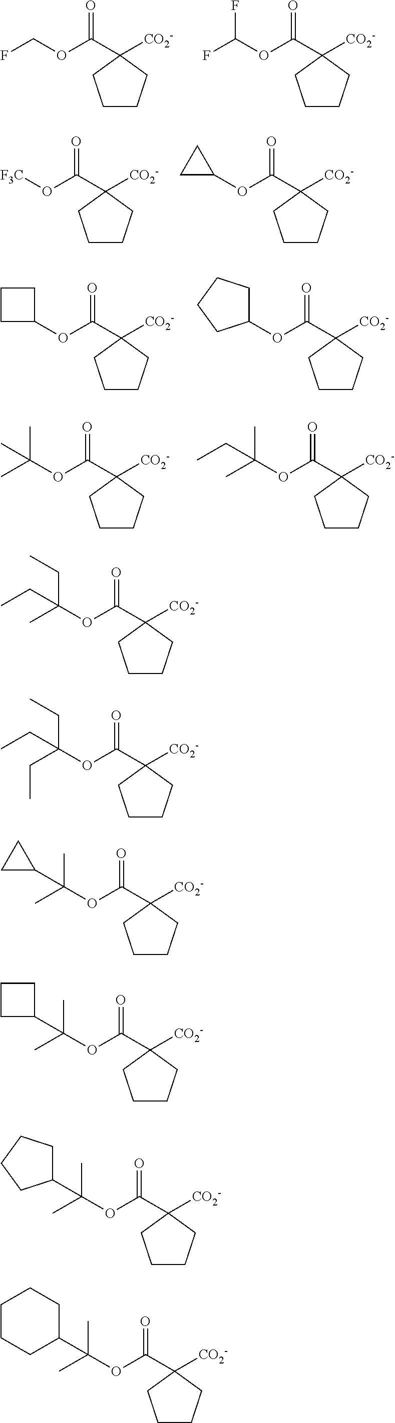

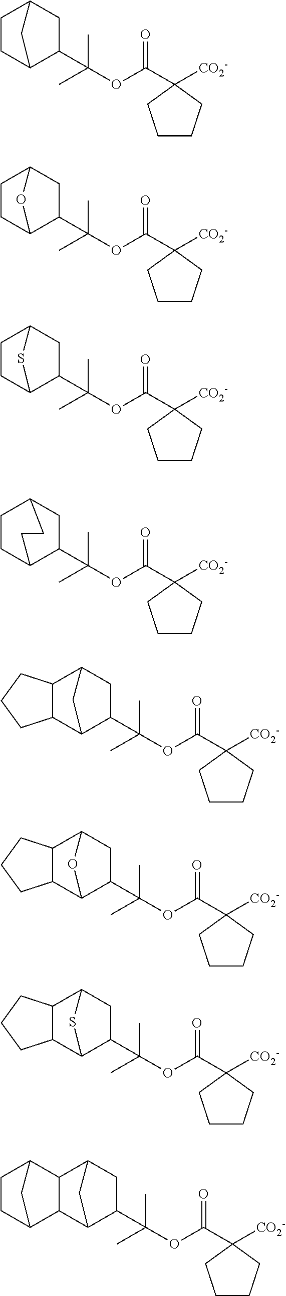

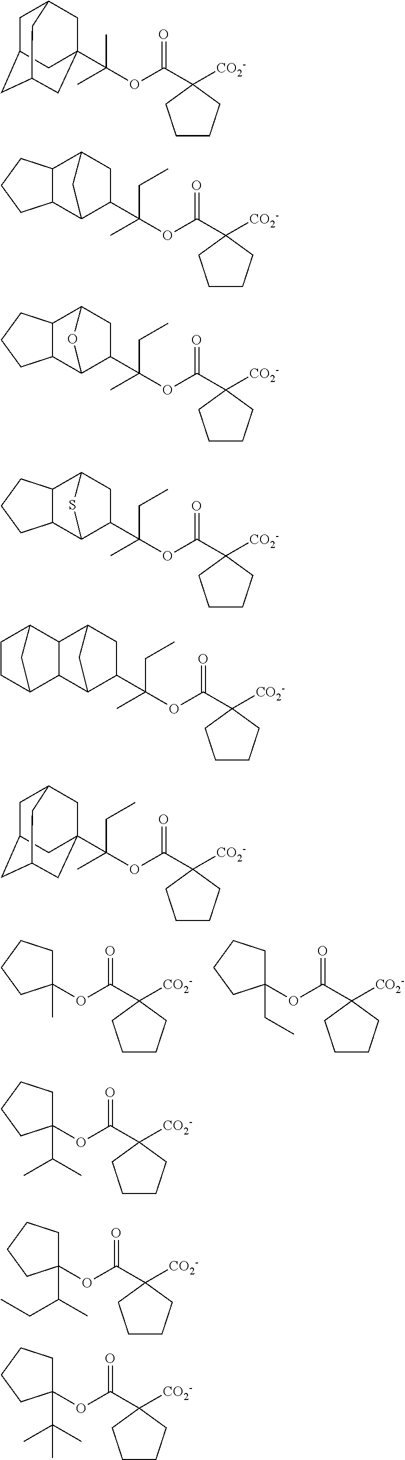

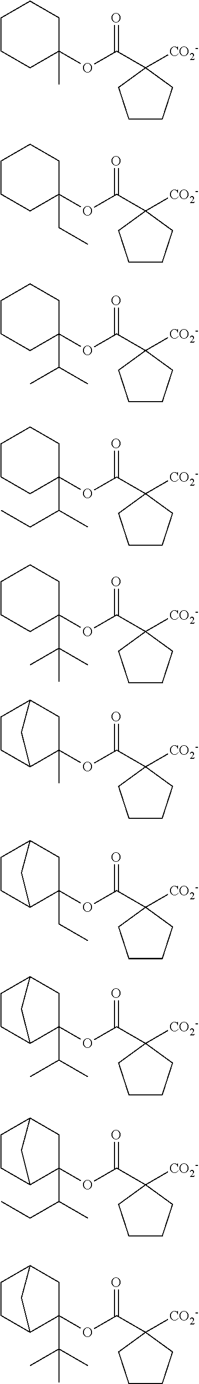

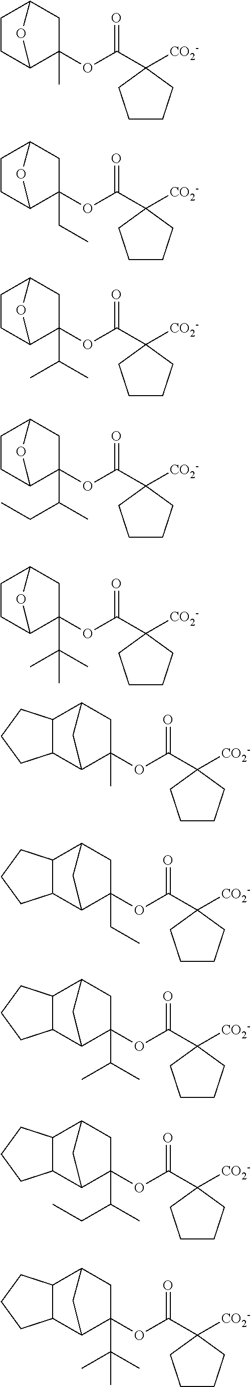

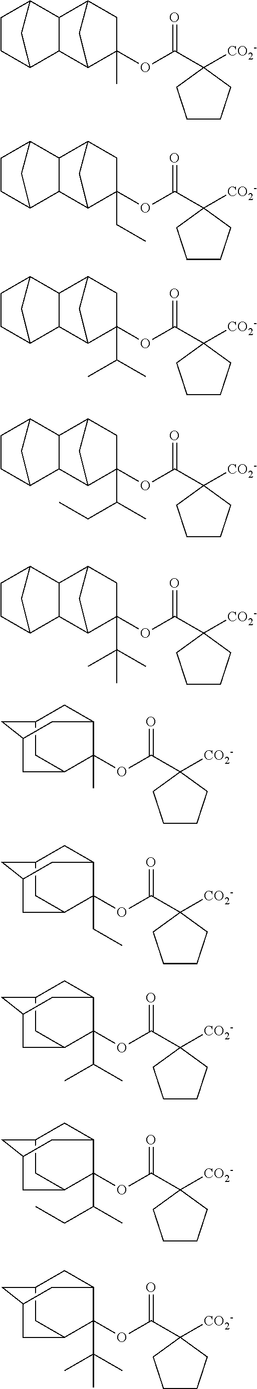

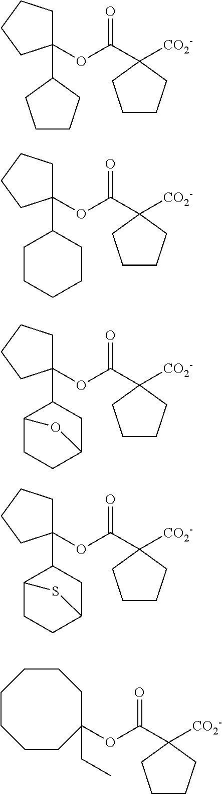

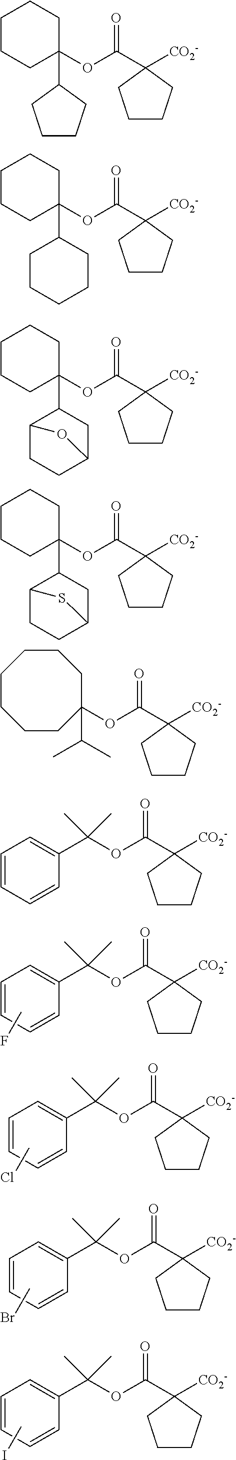

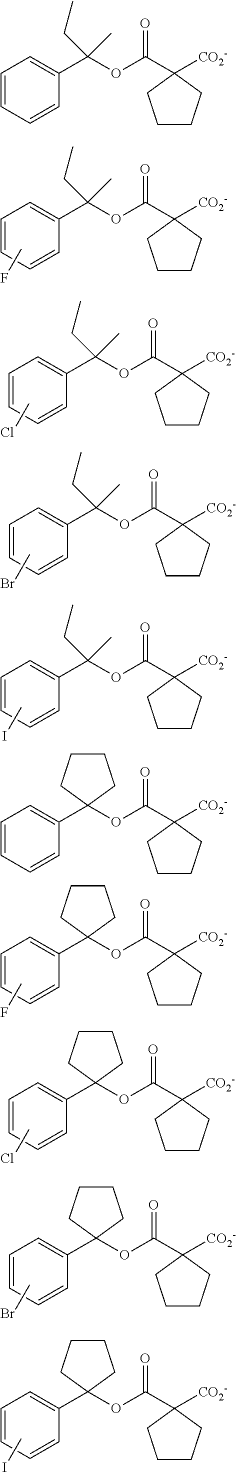

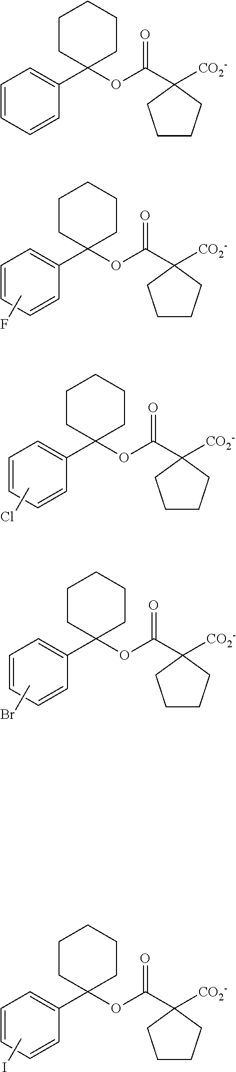

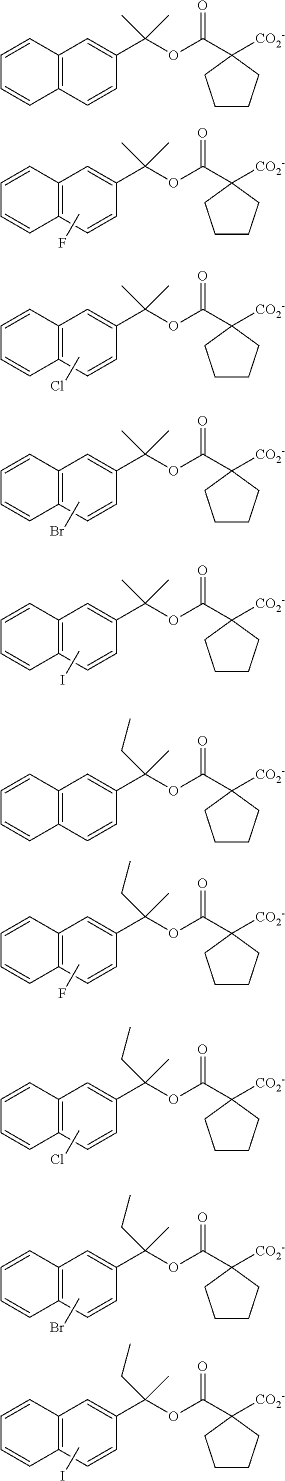

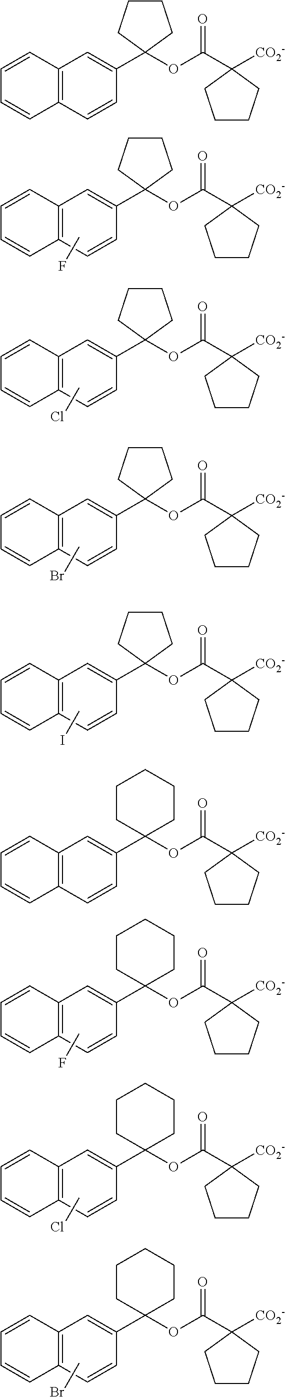

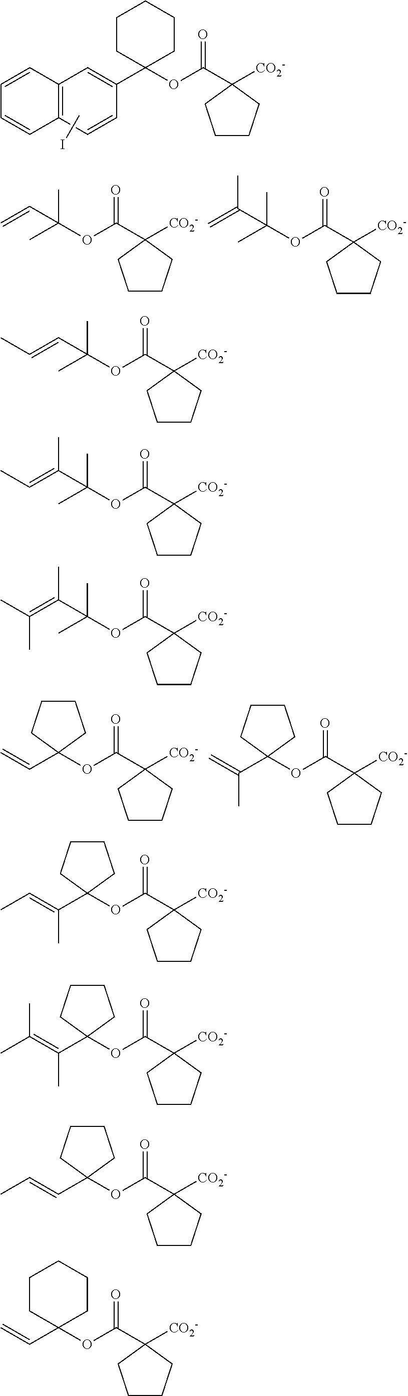

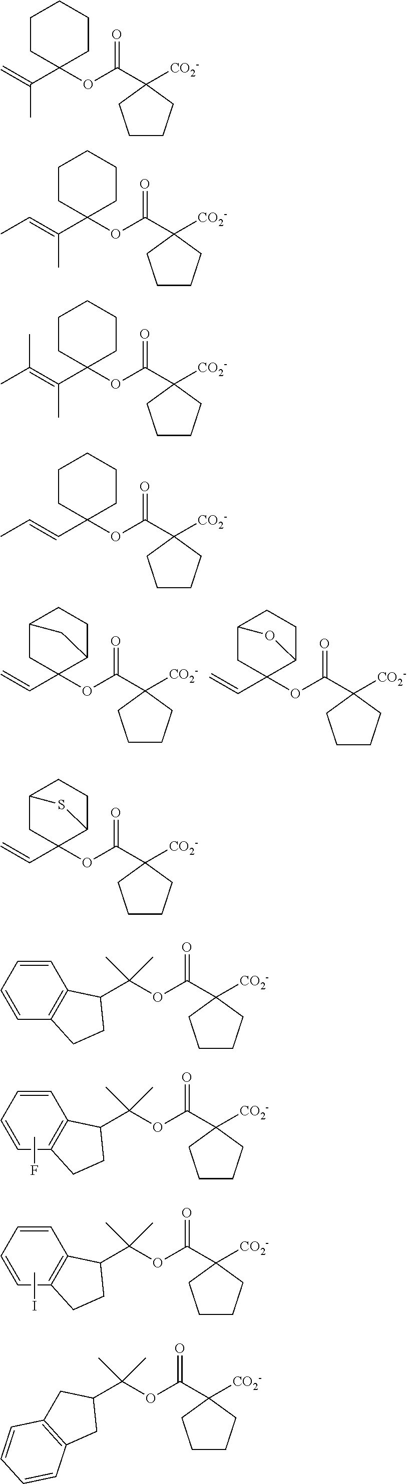

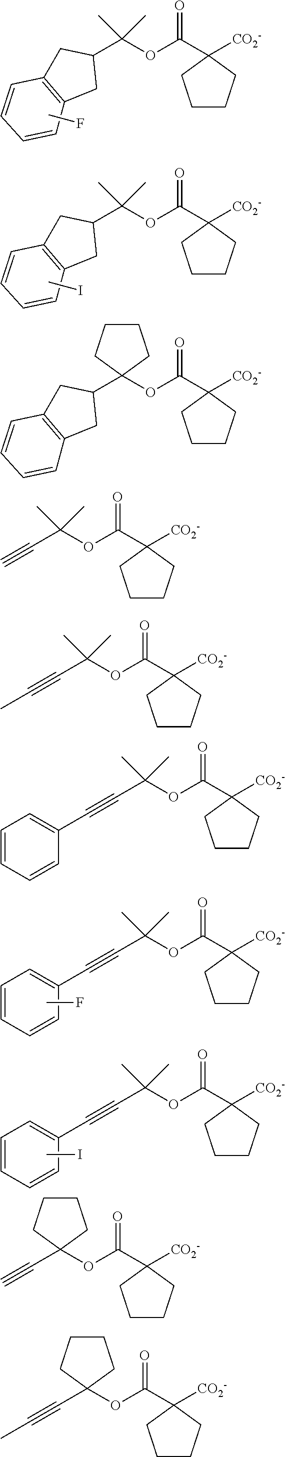

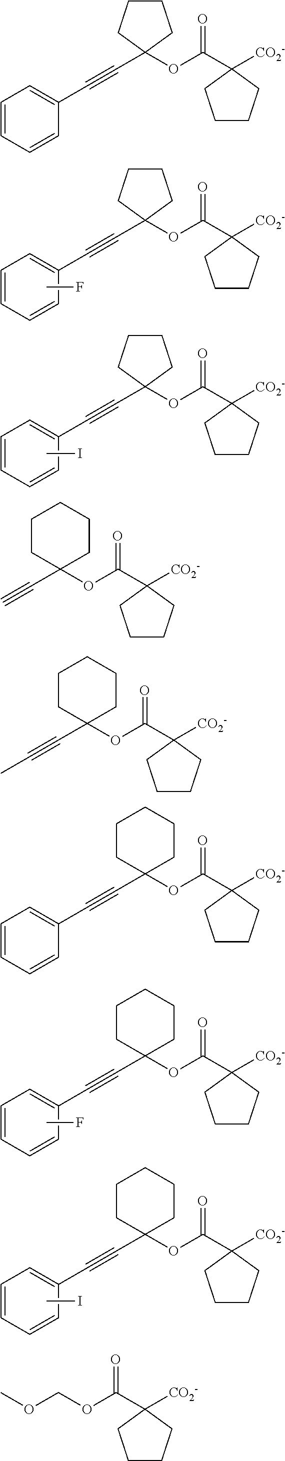

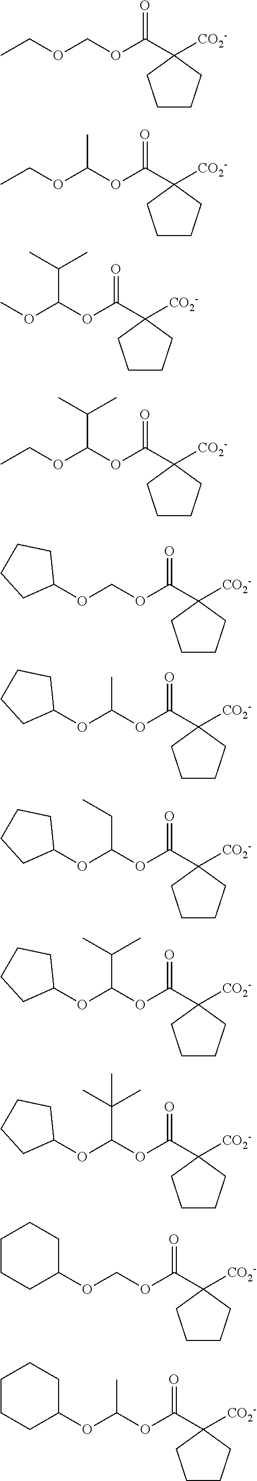

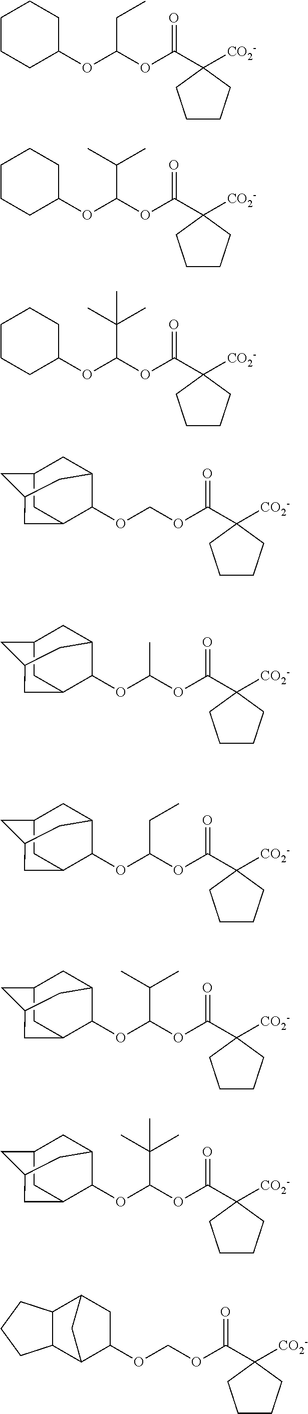

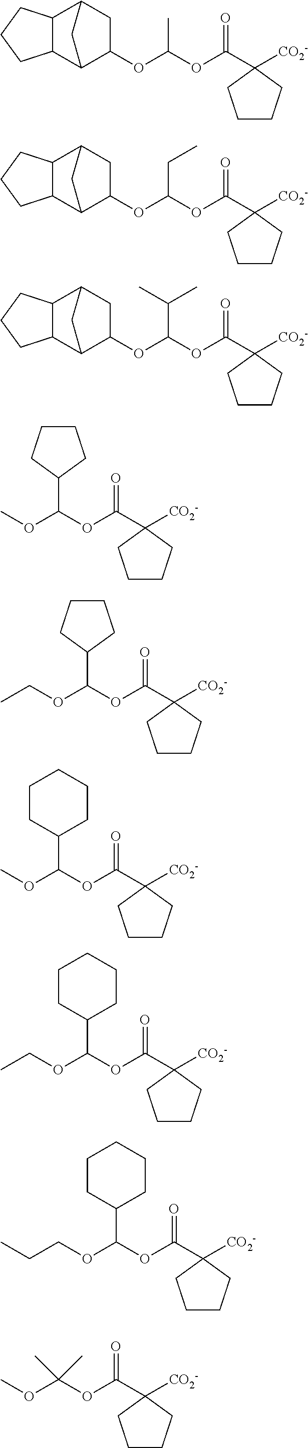

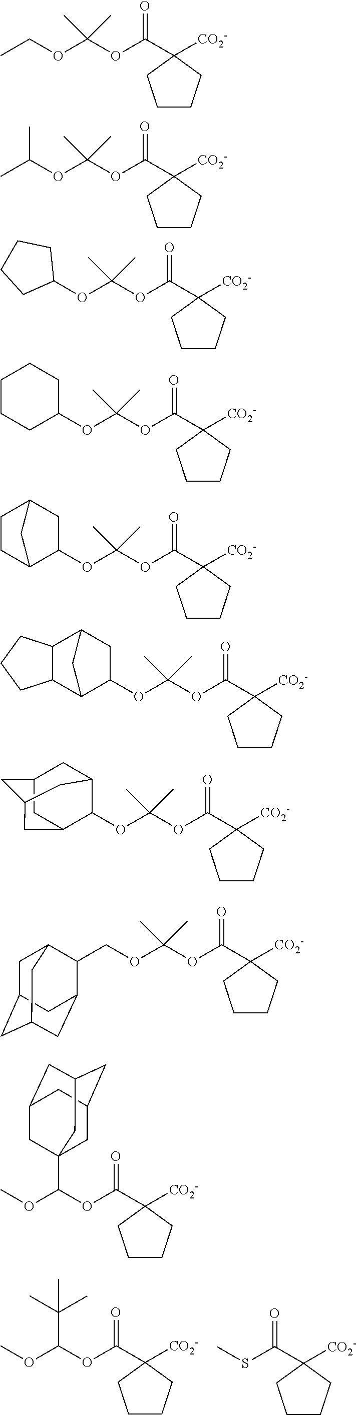

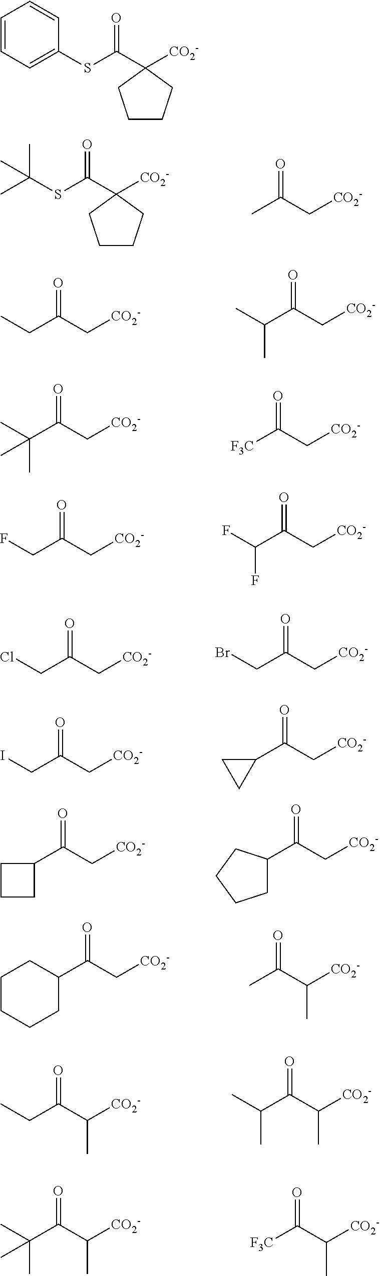

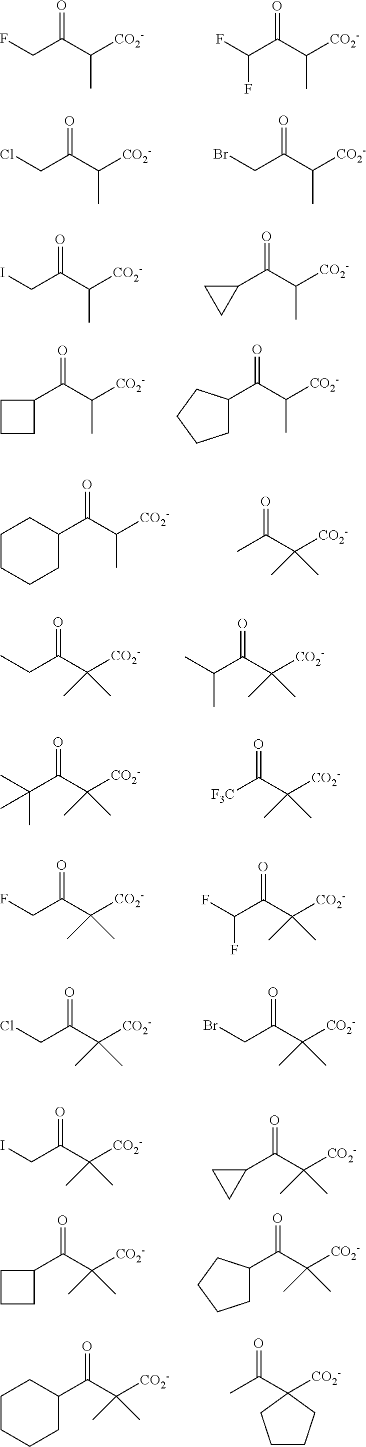

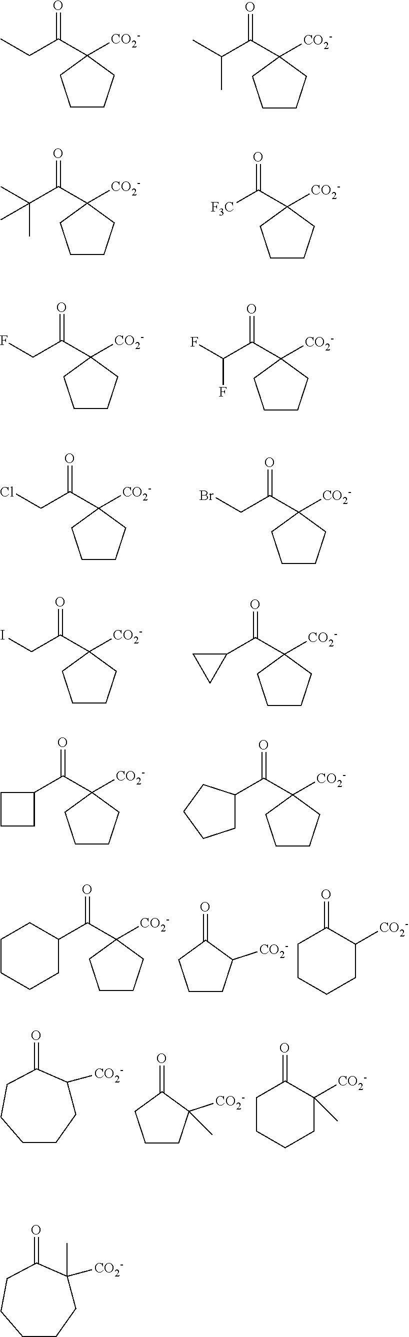

In formula (1), Z⁺ is an onium cation having any one of the formulae (cation-1) to (cation-3).

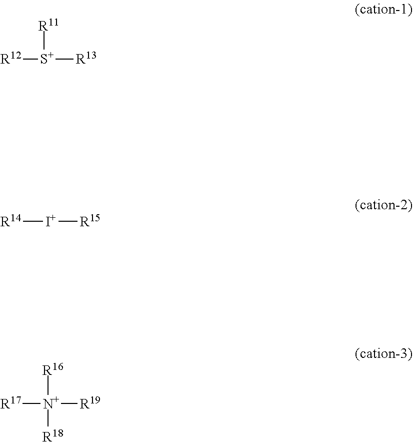

In formulae (cation-1) to (cation-3), R¹¹ to R¹⁹ are each independently a C₁-C₃₀ hydrocarbyl group which may contain a heteroatom. The hydrocarbyl group may be saturated or unsaturated and straight, branched or cyclic. Examples thereof include C₁-C₃₀ alkyl groups such as methyl, ethyl, n-propyl, isopropyl, n-butyl, and tert-butyl; C₃-C₃₀ cyclic saturated hydrocarbyl groups such as cyclopropyl, cyclopentyl, cyclohexyl, cyclopropylmethyl, 4-methylcyclohexyl, cyclohexylmethyl, norbornyl, and adamantyl; C₂-C₃₀ alkenyl groups such as vinyl, allyl, propenyl, butenyl, and hexenyl; C₃-C₃₀ cyclic unsaturated hydrocarbyl groups such as cyclohexenyl; C₆-C₃₀ aryl groups such as phenyl, naphthyl and thienyl; C₇-C₃₀ aralkyl groups such as benzyl, 1-phenylethyl and 2-phenylethyl, and combinations thereof. Inter alia, aryl groups are preferred. In the hydrocarbyl group, some or all of the hydrogen atoms may be substituted by a moiety containing a heteroatom such as oxygen, sulfur, nitrogen or halogen and some constituent —CH₂— may be replaced by a moiety containing a heteroatom such as oxygen, sulfur or nitrogen, so that the group may contain a hydroxy, cyano, carbonyl, ether bond, ester bond, sulfonic ester bond, carbonate bond, lactone ring, sultone ring, carboxylic anhydride (—C(═O)—O—C(═O)—), or haloalkyl moiety.

Also, R¹¹ and R¹² may bond together to form a ring with the sulfur atom to which they are attached. Examples of the sulfonium cation having formula (cation-1) wherein R¹¹ and R¹² form a ring are shown below.

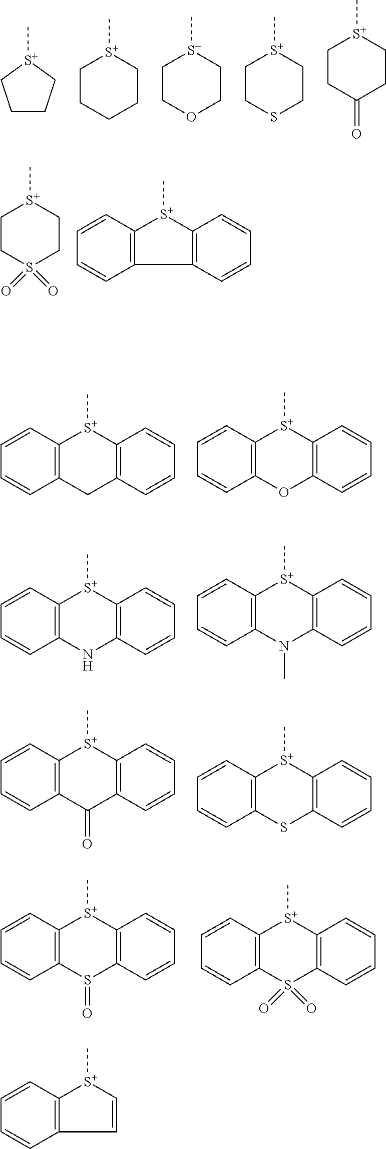

Herein the broken line designates a point of attachment to R¹³.

Examples of the sulfonium cation having formula (cation-1) are shown below, but not limited thereto.

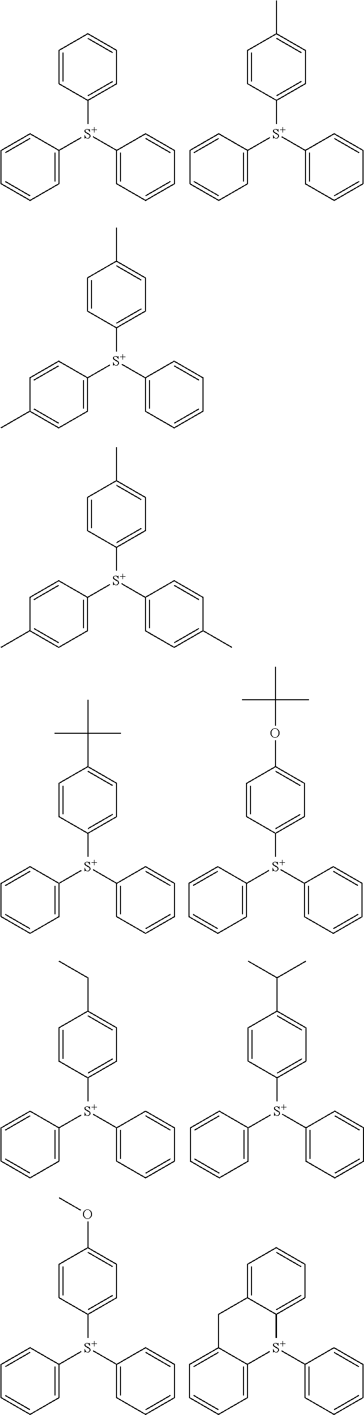

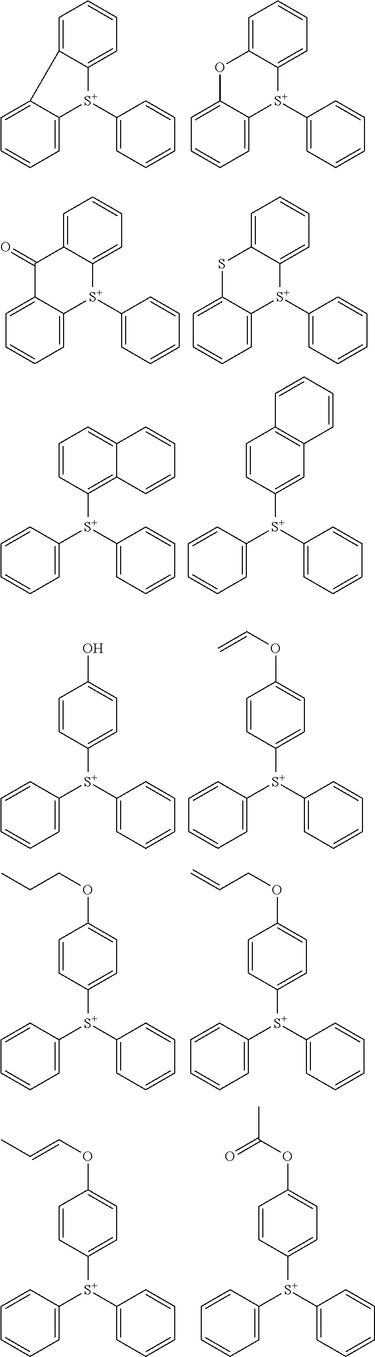

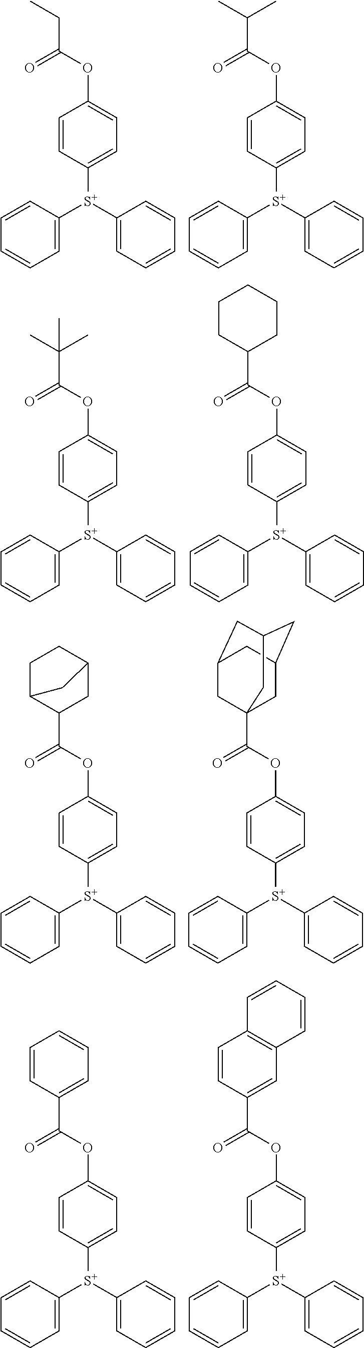

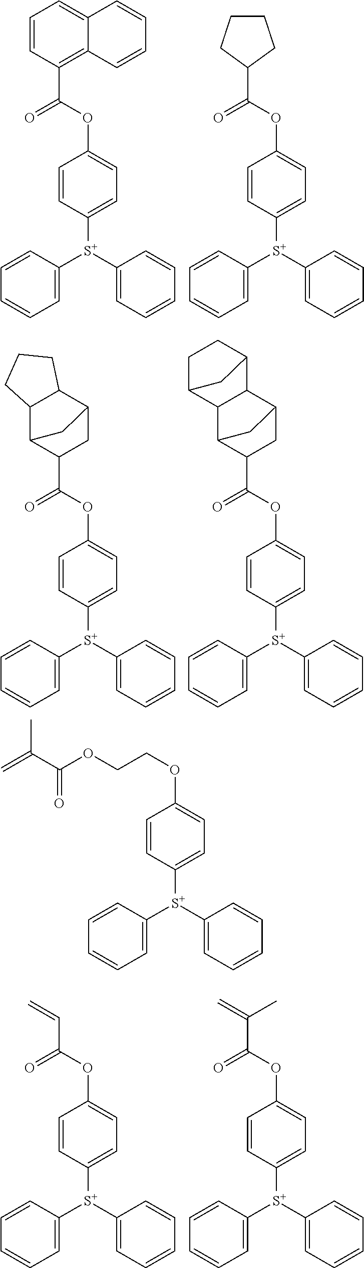

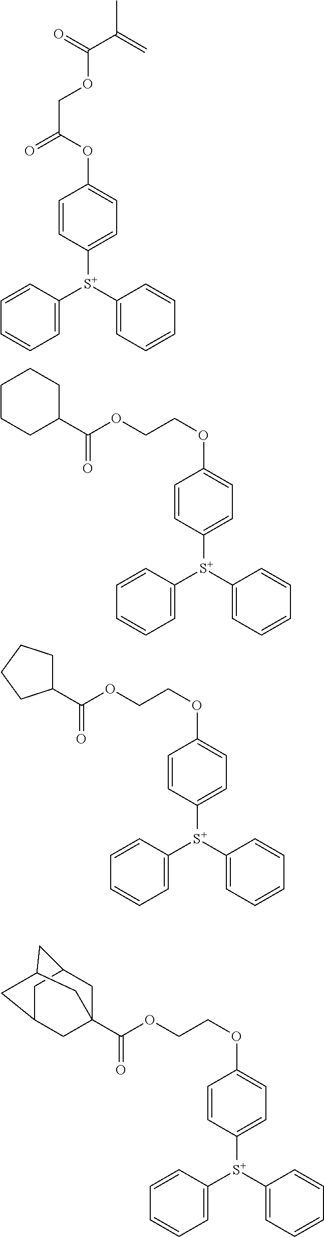 [SMILES: C=C(C)C(=O)OCC(=O)Oc1ccc([S+](c2ccccc2)c2ccccc2)cc1 ; O=C(OCCOc1ccc([S+](c2ccccc2)c2ccccc2)cc1)C1CCCCC1 ; O=C(OCCOc1ccc([S+](c2ccccc2)c2ccccc2)cc1)C1CCCC1 ; O=C(OCCOc1ccc([S+](c2ccccc2)c2ccccc2)cc1)C12CC3CC(CC(C3)C1)C2]

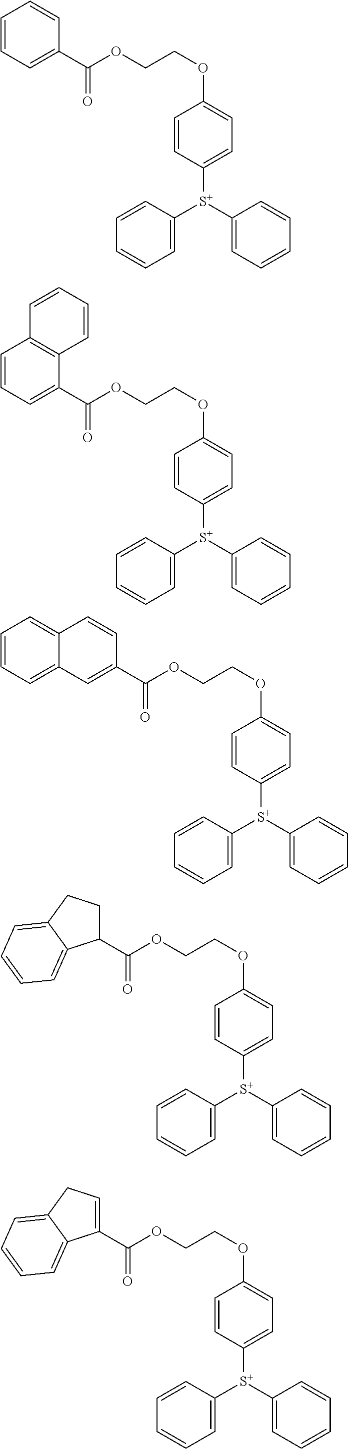 [SMILES: O=C(OCCOc1ccc([S+](c2ccccc2)c2ccccc2)cc1)c1ccccc1 ; O=C(OCCOc1ccc([S+](c2ccccc2)c2ccccc2)cc1)c1cccc2ccccc12 ; O=C(OCCOc1ccc([S+](c2ccccc2)c2ccccc2)cc1)c1ccc2ccccc2c1 ; O=C(OCCOc1ccc([S+](c2ccccc2)c2ccccc2)cc1)C1CCc2ccccc21 ; O=C(OCCOc1ccc([S+](c2ccccc2)c2ccccc2)cc1)C1=CCc2ccccc21]

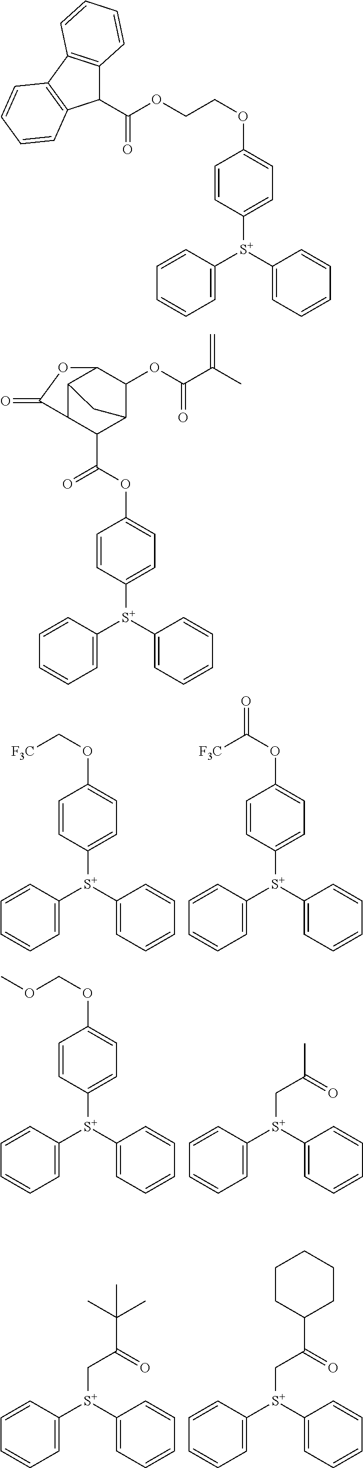

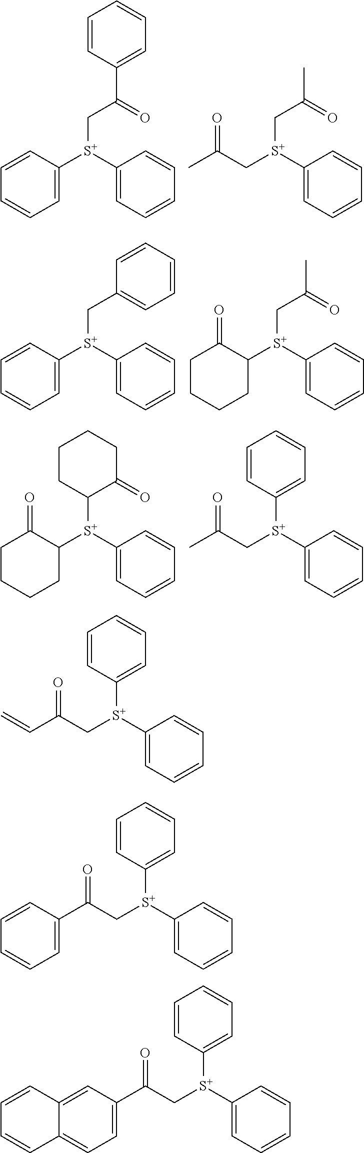

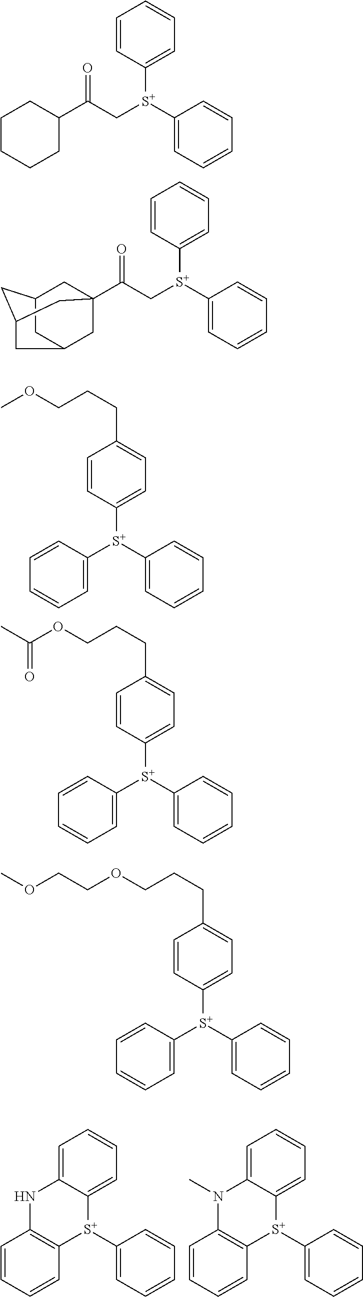

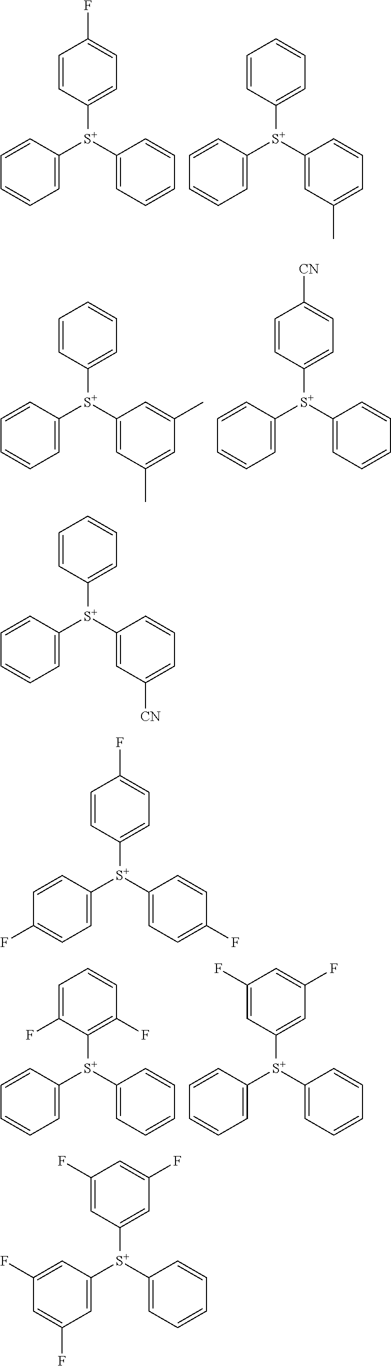

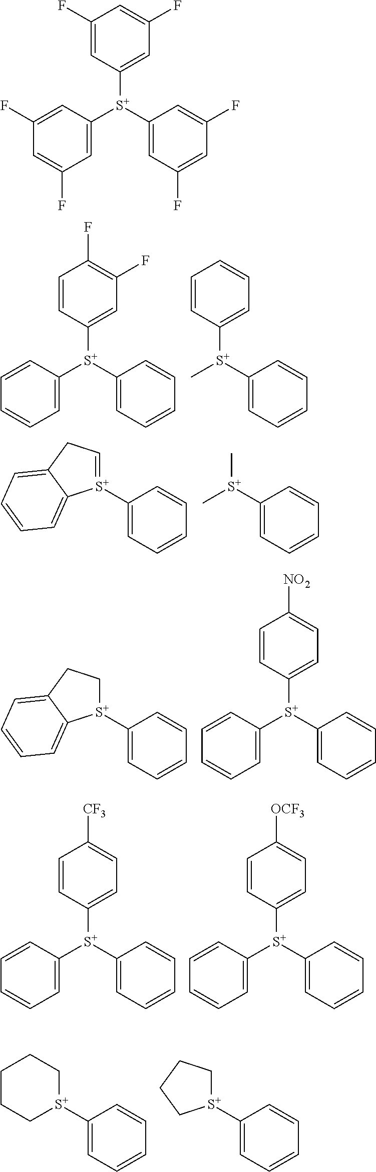

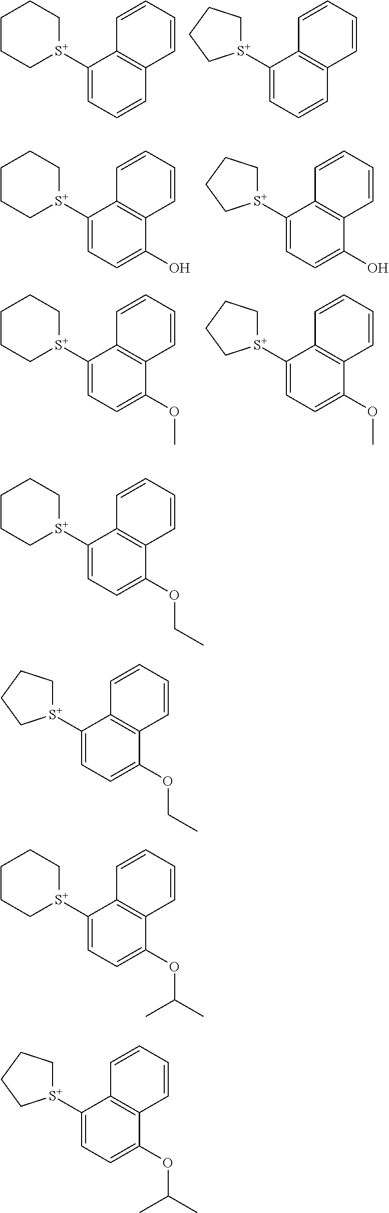

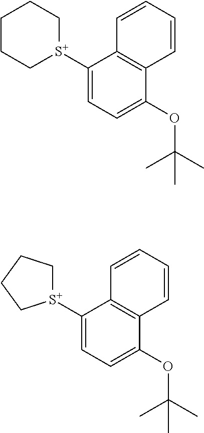 [SMILES: CC(C)(C)Oc1ccc([S+]2CCCCC2)c2ccccc12 ; CC(C)(C)Oc1ccc([S+]2CCCC2)c2ccccc12]

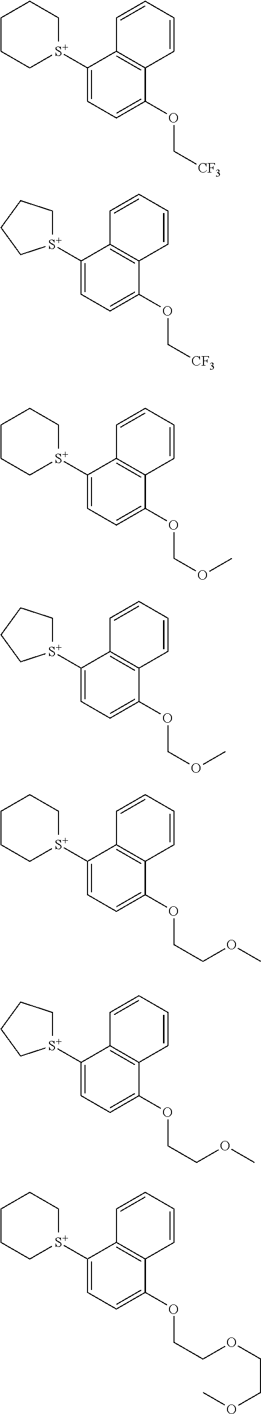

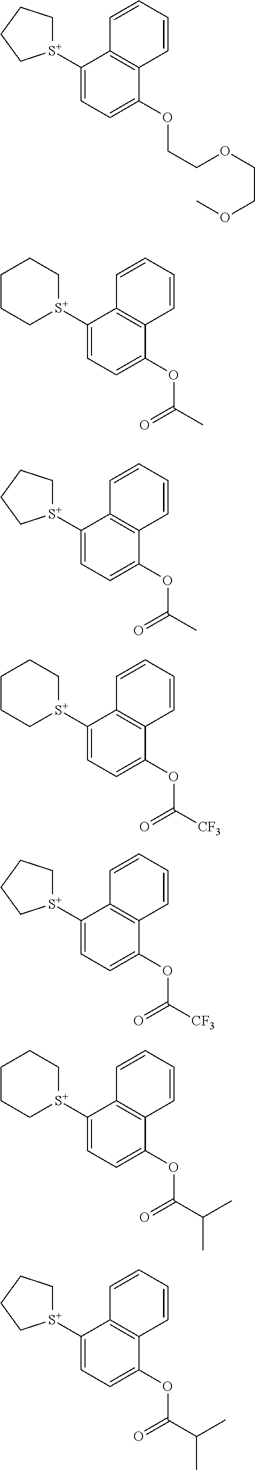

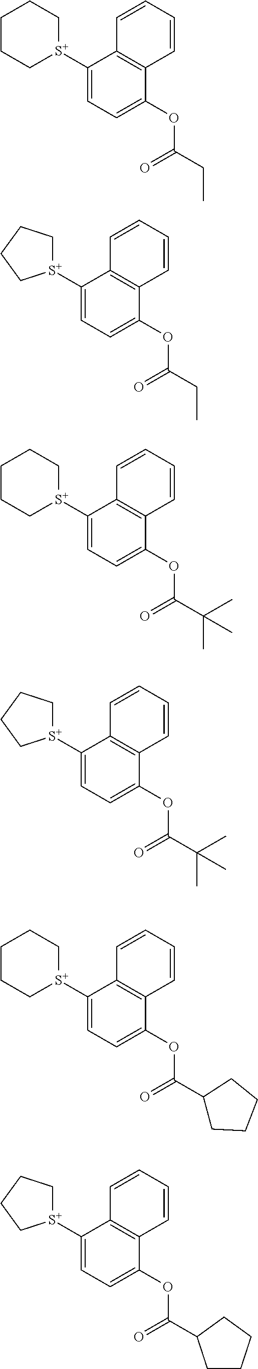 [SMILES: CCC(=O)Oc1ccc([S+]2CCCCC2)c2ccccc12 ; CCC(=O)Oc1ccc([S+]2CCCC2)c2ccccc12 ; CC(C)(C)C(=O)Oc1ccc([S+]2CCCCC2)c2ccccc12 ; CC(C)(C)C(=O)Oc1ccc([S+]2CCCC2)c2ccccc12 ; O=C(Oc1ccc([S+]2CCCCC2)c2ccccc12)C1CCCC1 ; O=C(Oc1ccc([S+]2CCCC2)c2ccccc12)C1CCCC1]

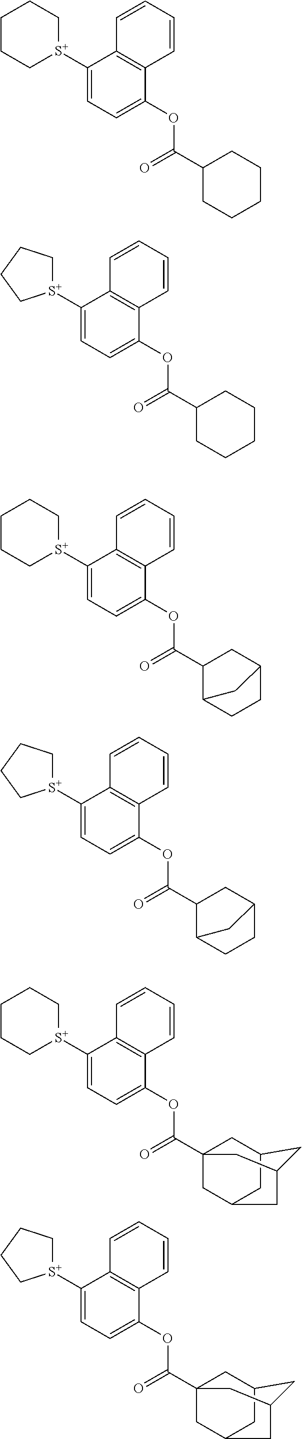 [SMILES: O=C(Oc1ccc([S+]2CCCCC2)c2ccccc12)C1CCCCC1 ; O=C(Oc1ccc([S+]2CCCC2)c2ccccc12)C1CCCCC1 ; O=C(Oc1ccc([S+]2CCCCC2)c2ccccc12)C1CC2CCC1C2 ; O=C(Oc1ccc([S+]2CCCC2)c2ccccc12)C1CC2CCC1C2 ; O=C(Oc1ccc([S+]2CCCCC2)c2ccccc12)C12CC3CC(CC(C3)C1)C2 ; O=C(Oc1ccc([S+]2CCCC2)c2ccccc12)C12CC3CC(CC(C3)C1)C2]

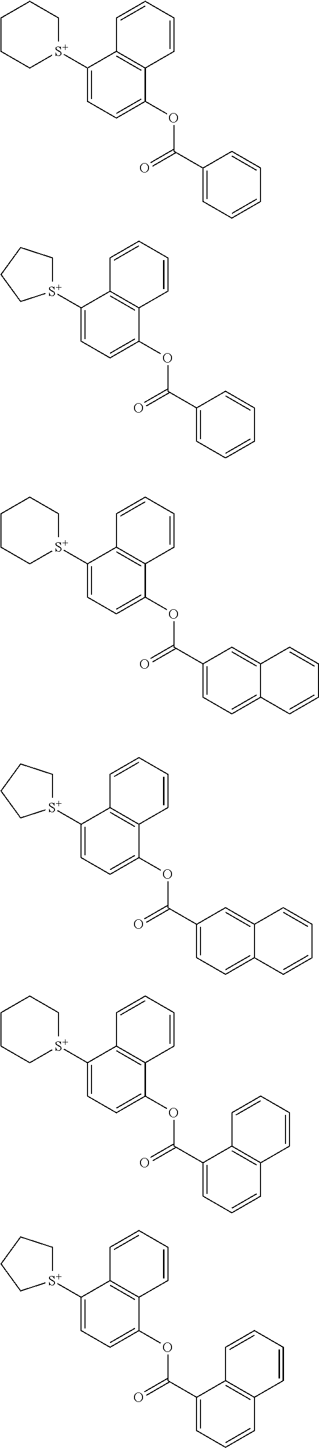 [SMILES: O=C(Oc1ccc([S+]2CCCCC2)c2ccccc12)c1ccccc1 ; O=C(Oc1ccc([S+]2CCCC2)c2ccccc12)c1ccccc1 ; O=C(Oc1ccc([S+]2CCCCC2)c2ccccc12)c1ccc2ccccc2c1 ; O=C(Oc1ccc([S+]2CCCC2)c2ccccc12)c1ccc2ccccc2c1 ; O=C(Oc1ccc([S+]2CCCCC2)c2ccccc12)c1cccc2ccccc12 ; O=C(Oc1ccc([S+]2CCCC2)c2ccccc12)c1cccc2ccccc12]

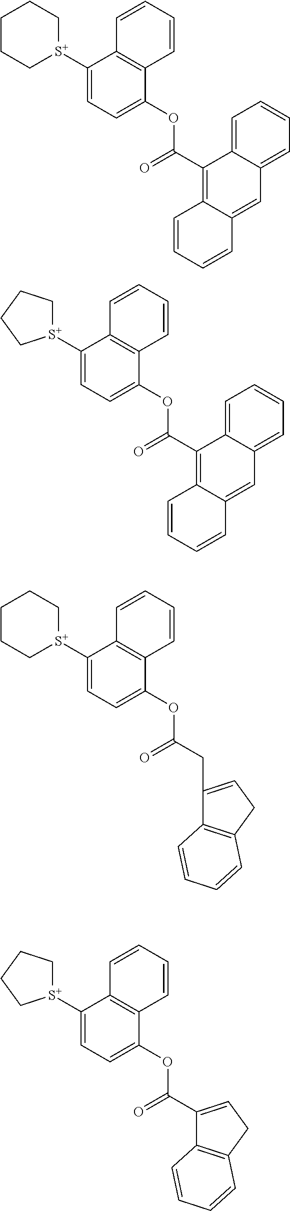 [SMILES: O=C(Oc1ccc([S+]2CCCCC2)c2ccccc12)c1c2ccccc2cc2ccccc12 ; O=C(Oc1ccc([S+]2CCCC2)c2ccccc12)c1c2ccccc2cc2ccccc12 ; O=C(CC1=CCc2ccccc21)Oc1ccc([S+]2CCCCC2)c2ccccc12 ; O=C(Oc1ccc([S+]2CCCC2)c2ccccc12)C1=CCc2ccccc21]

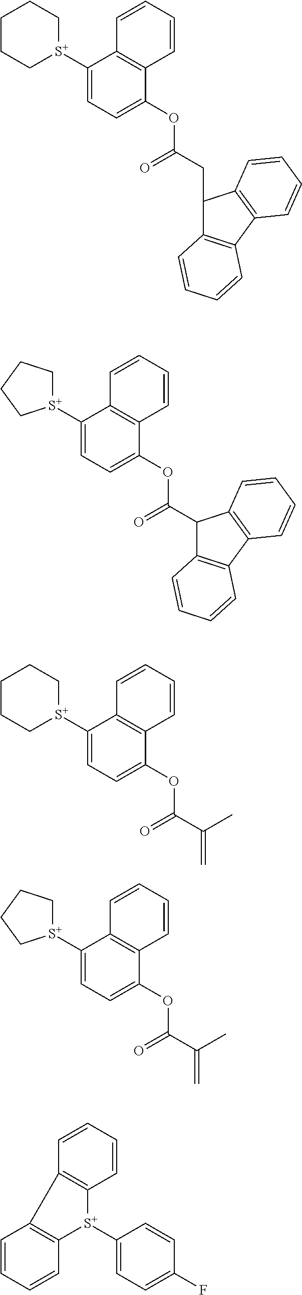 [SMILES: O=C(CC1c2ccccc2-c2ccccc21)Oc1ccc([S+]2CCCCC2)c2ccccc12 ; O=C(Oc1ccc([S+]2CCCC2)c2ccccc12)C1c2ccccc2-c2ccccc21 ; C=C(C)C(=O)Oc1ccc([S+]2CCCCC2)c2ccccc12 ; C=C(C)C(=O)Oc1ccc([S+]2CCCC2)c2ccccc12 ; Fc1ccc(-[s+]2c3ccccc3c3ccccc32)cc1]

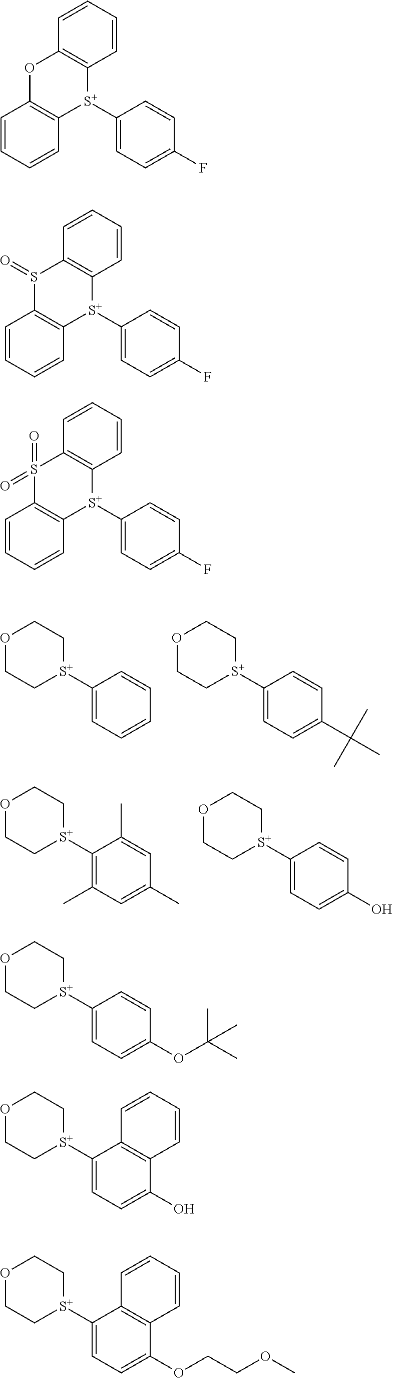

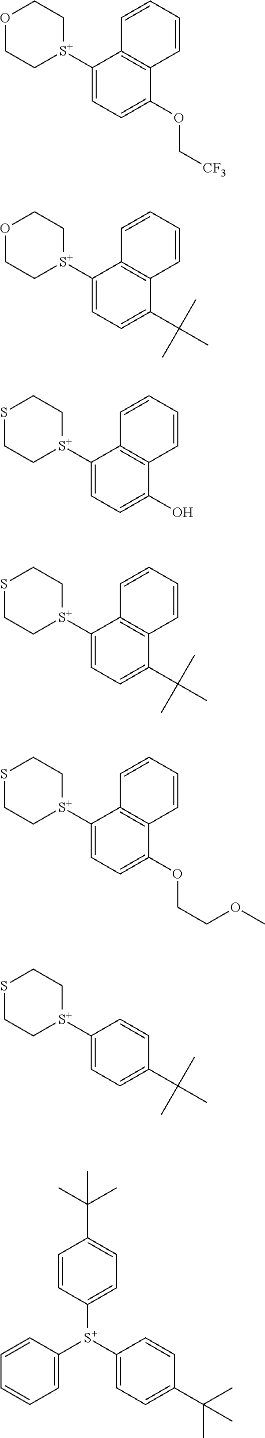

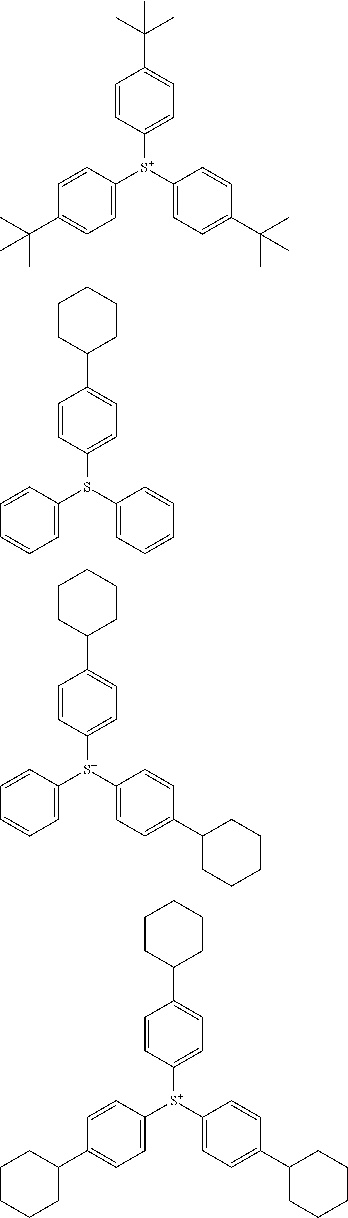 [SMILES: CC(C)(C)c1ccc([S+](c2ccc(C(C)(C)C)cc2)c2ccc(C(C)(C)C)cc2)cc1 ; c1ccc([S+](c2ccccc2)c2ccc(C3CCCCC3)cc2)cc1 ; c1ccc([S+](c2ccc(C3CCCCC3)cc2)c2ccc(C3CCCCC3)cc2)cc1 ; c1cc([S+](c2ccc(C3CCCCC3)cc2)c2ccc(C3CCCCC3)cc2)ccc1C1CCCCC1]

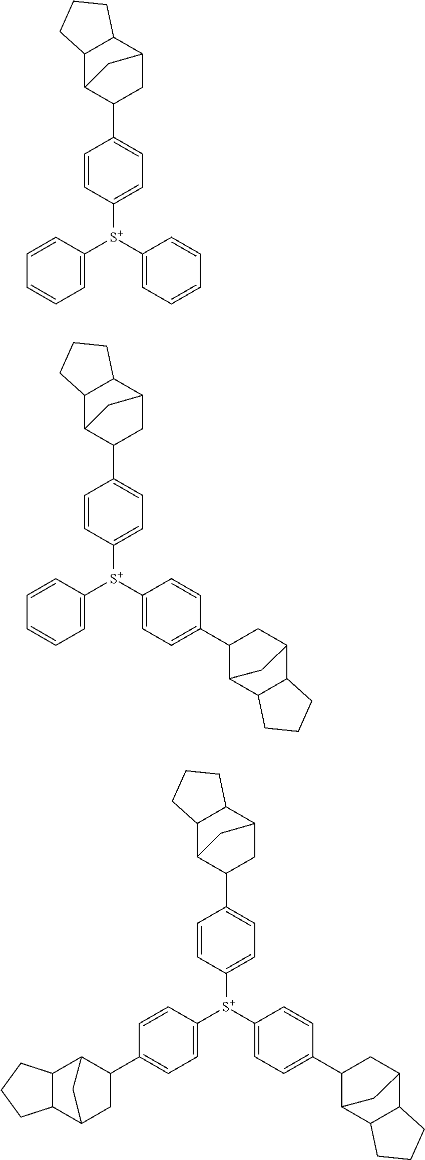 [SMILES: c1ccc([S+](c2ccccc2)c2ccc(C3CC4CC3C3CCCC43)cc2)cc1 ; c1ccc([S+](c2ccc(C3CC4CC3C3CCCC43)cc2)c2ccc(C3CC4CC3C3CCCC43)cc2)cc1 ; c1cc([S+](c2ccc(C3CC4CC3C3CCCC43)cc2)c2ccc(C3CC4CC3C3CCCC43)cc2)ccc1C1CC2CC1C1CCCC21]

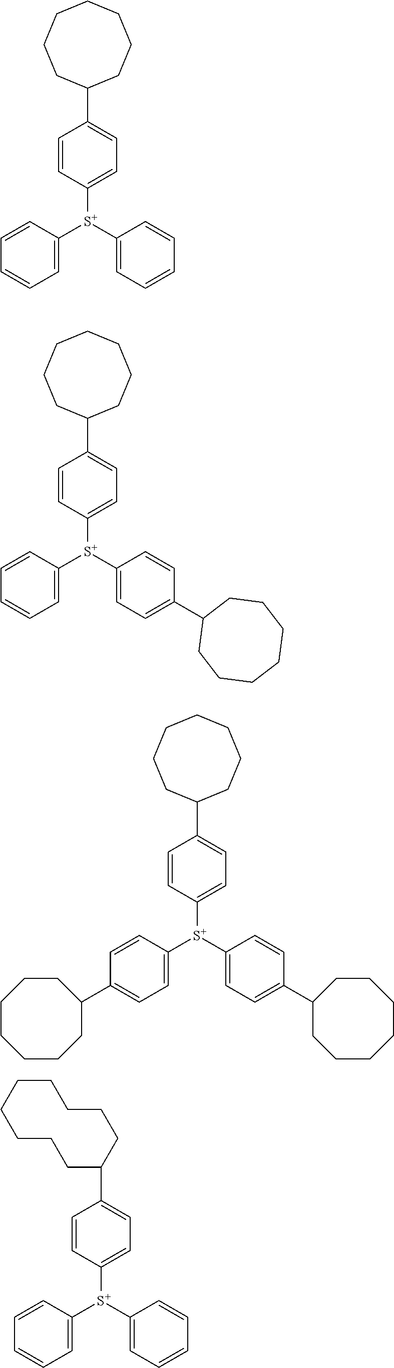 [SMILES: c1ccc([S+](c2ccccc2)c2ccc(C3CCCCCCC3)cc2)cc1 ; c1ccc([S+](c2ccc(C3CCCCCCC3)cc2)c2ccc(C3CCCCCCC3)cc2)cc1 ; c1cc([S+](c2ccc(C3CCCCCCC3)cc2)c2ccc(C3CCCCCCC3)cc2)ccc1C1CCCCCCC1 ; c1ccc([S+](c2ccccc2)c2ccc(C3CCCCCCCCC3)cc2)cc1]

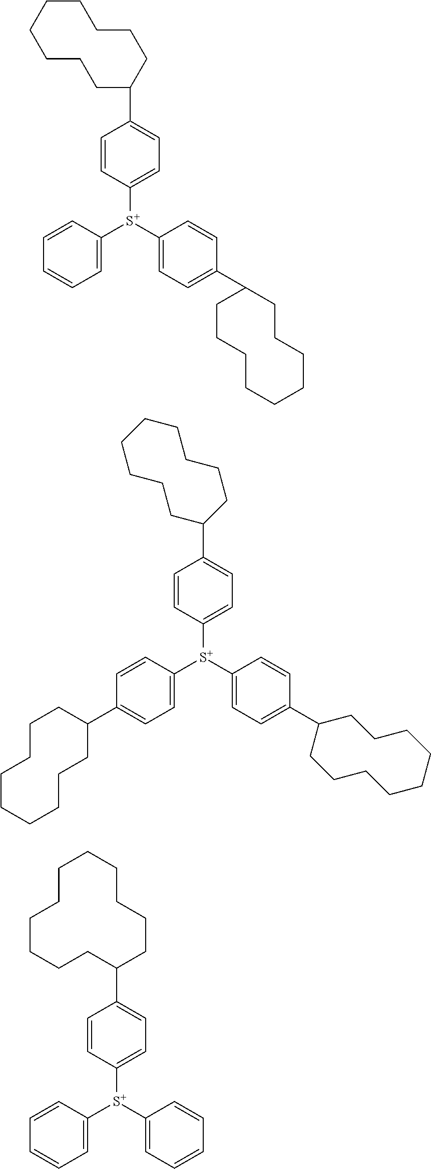 [SMILES: c1ccc([S+](c2ccc(C3CCCCCCCCC3)cc2)c2ccc(C3CCCCCCCCC3)cc2)cc1 ; c1cc([S+](c2ccc(C3CCCCCCCCC3)cc2)c2ccc(C3CCCCCCCCC3)cc2)ccc1C1CCCCCCCCC1 ; c1ccc([S+](c2ccccc2)c2ccc(C3CCCCCCCCCCC3)cc2)cc1]

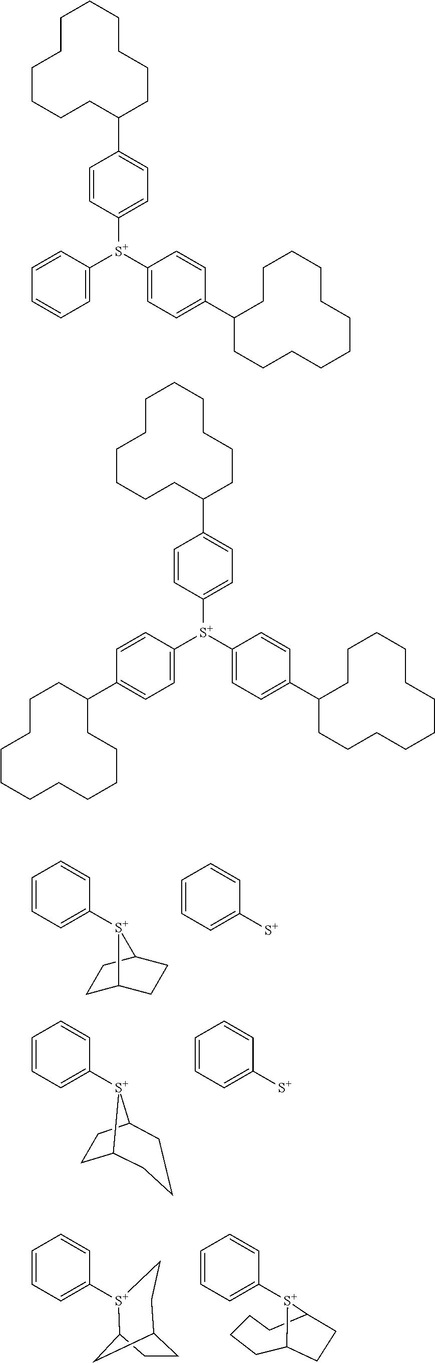

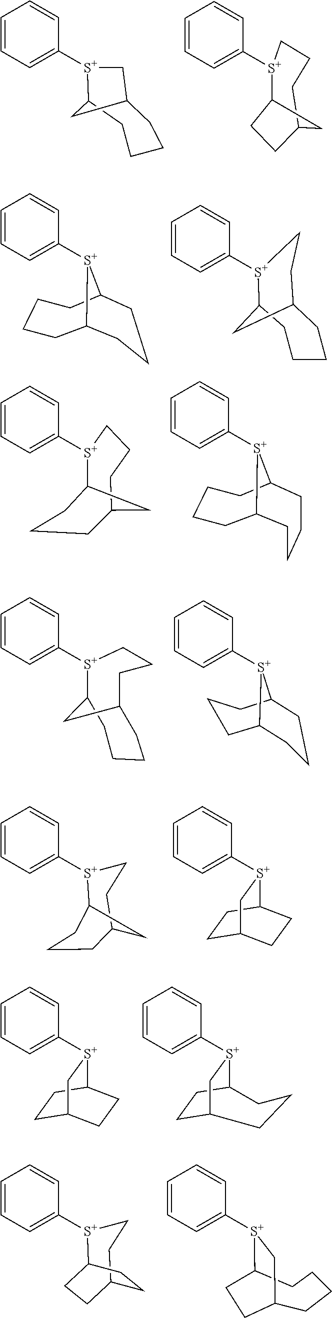

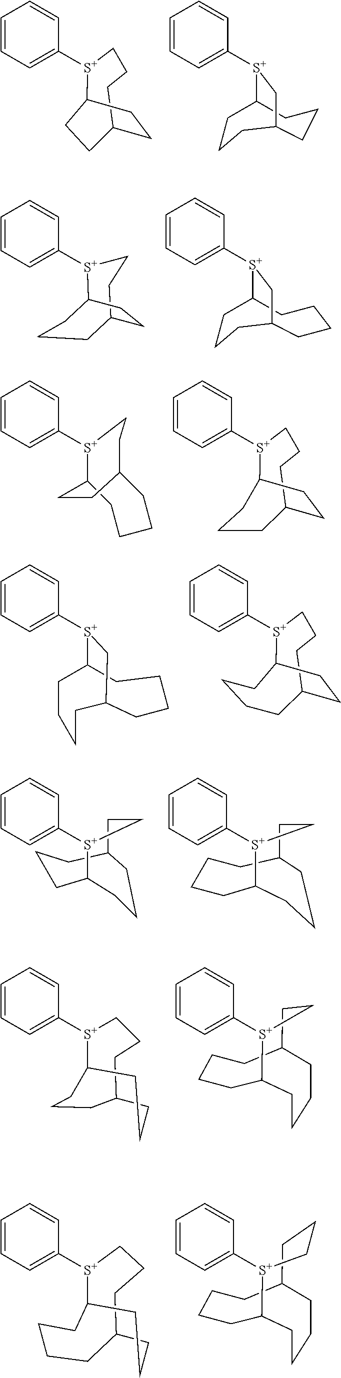

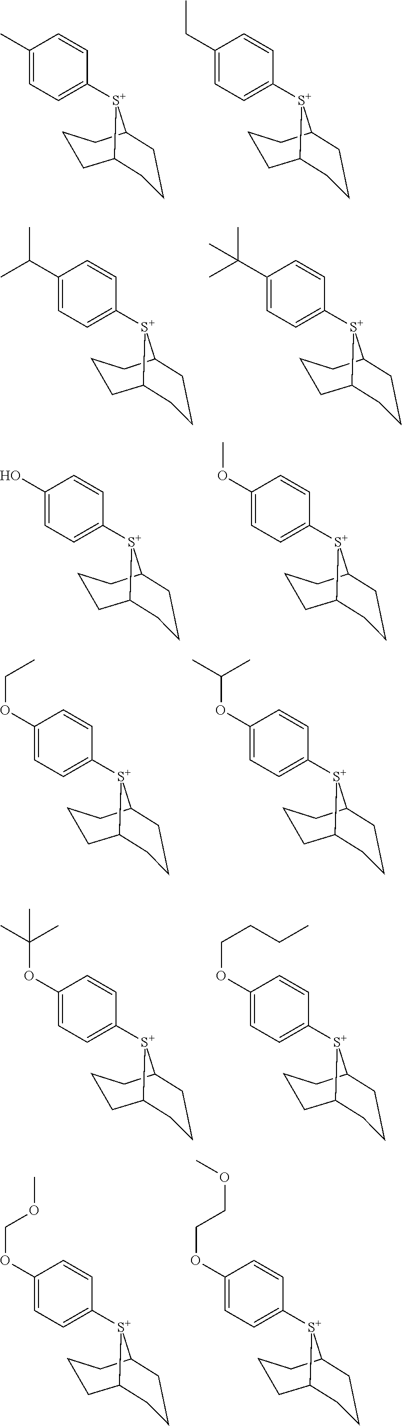

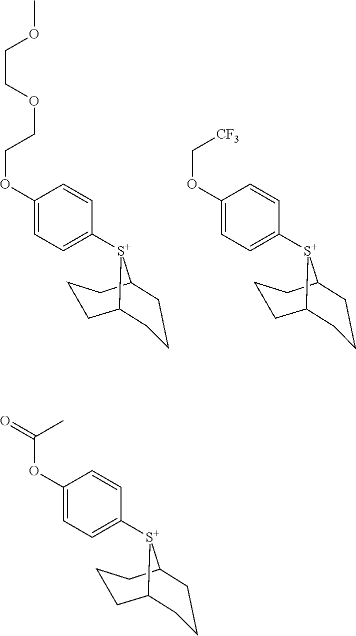 [SMILES: COCCOCCOc1ccc([S+]2C3CCCC2CCC3)cc1 ; FC(F)(F)COc1ccc([S+]2C3CCCC2CCC3)cc1 ; CC(=O)Oc1ccc([S+]2C3CCCC2CCC3)cc1]

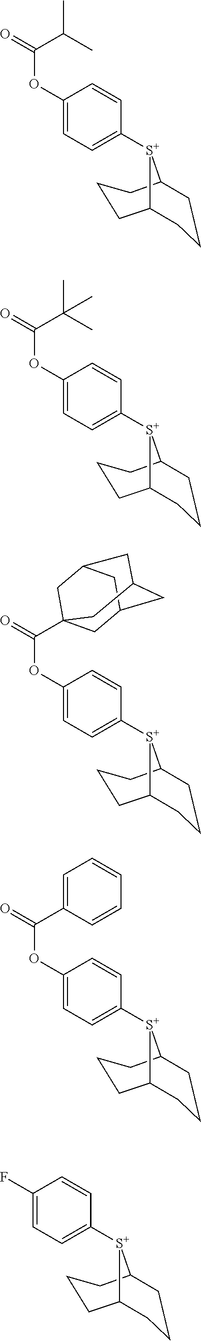 [SMILES: CC(C)C(=O)Oc1ccc([S+]2C3CCCC2CCC3)cc1 ; CC(C)(C)C(=O)Oc1ccc([S+]2C3CCCC2CCC3)cc1 ; O=C(Oc1ccc([S+]2C3CCCC2CCC3)cc1)C12CC3CC(CC(C3)C1)C2 ; O=C(Oc1ccc([S+]2C3CCCC2CCC3)cc1)c1ccccc1 ; Fc1ccc([S+]2C3CCCC2CCC3)cc1]

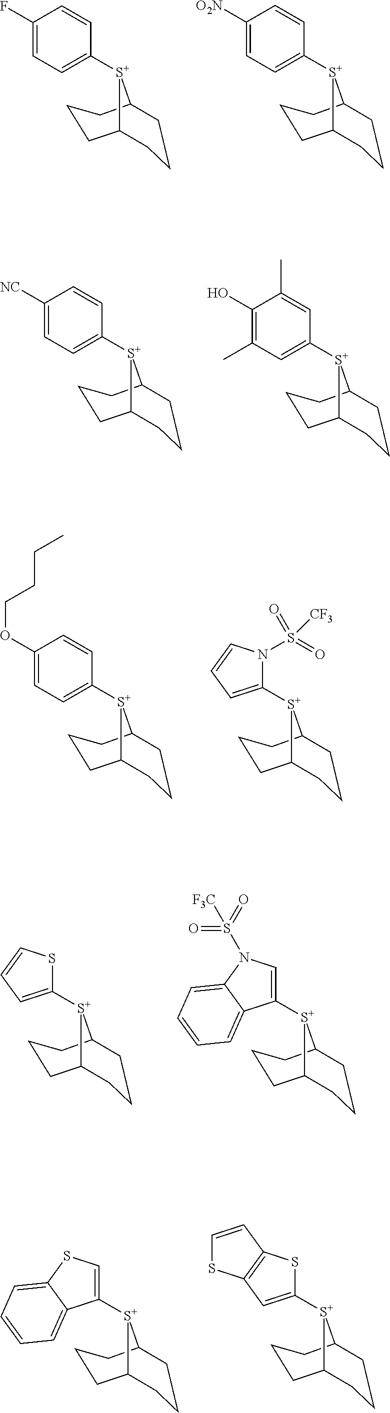

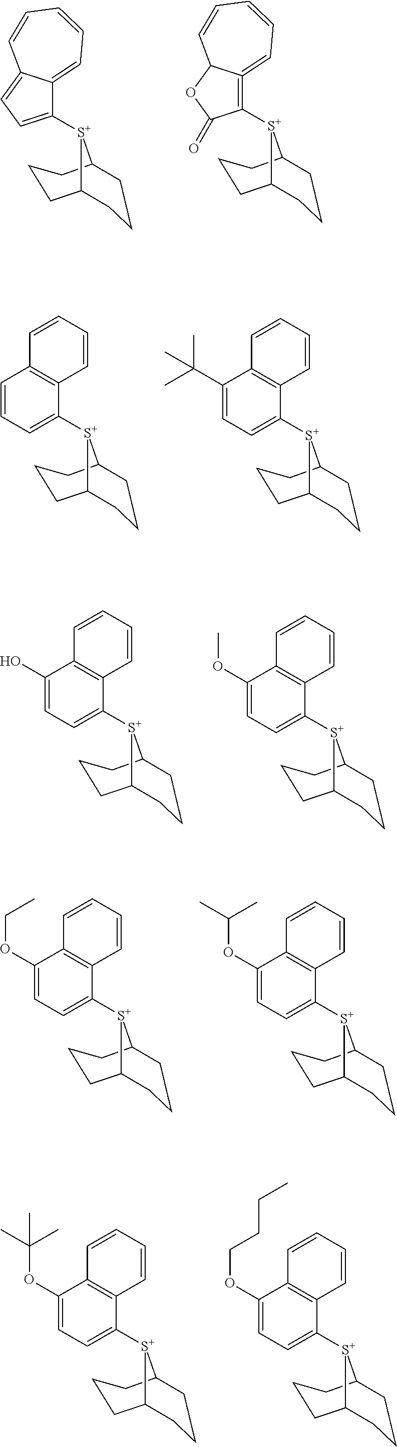

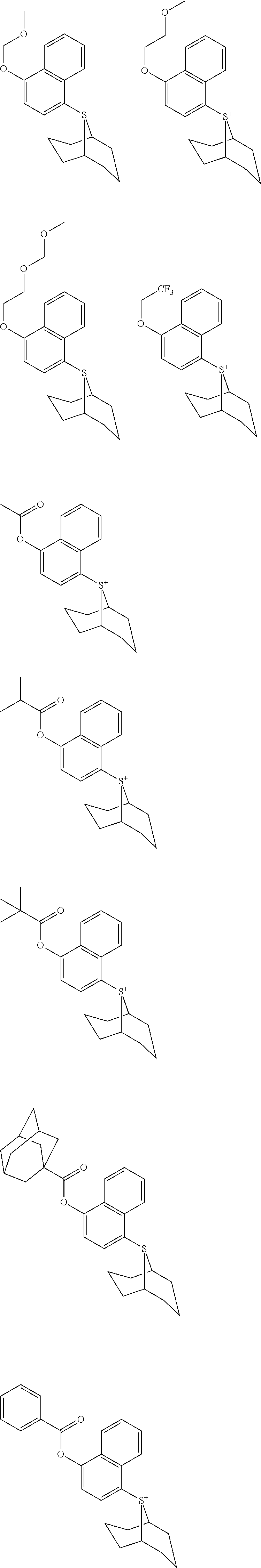

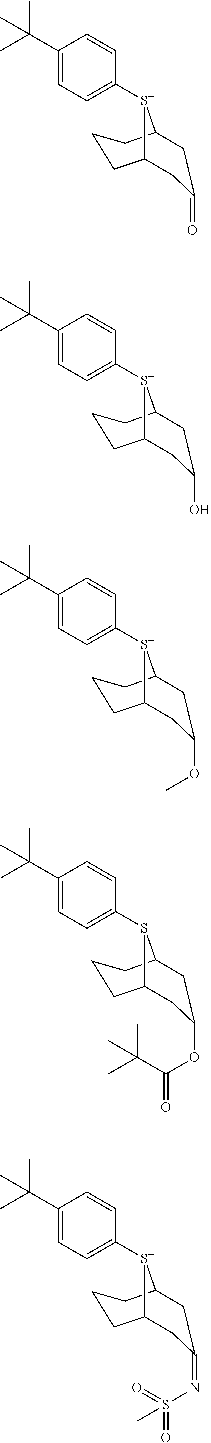 [SMILES: CC(C)(C)c1ccc([S+]2C3CCCC2CC(=O)C3)cc1 ; CC(C)(C)c1ccc([S+]2C3CCCC2CC(O)C3)cc1 ; COC1CC2CCCC(C1)[S+]2c1ccc(C(C)(C)C)cc1 ; CC(C)(C)C(=O)OC1CC2CCCC(C1)[S+]2c1ccc(C(C)(C)C)cc1 ; CC(C)(C)c1ccc([S+]2C3CCCC2CC(=NS(C)(=O)=O)C3)cc1]

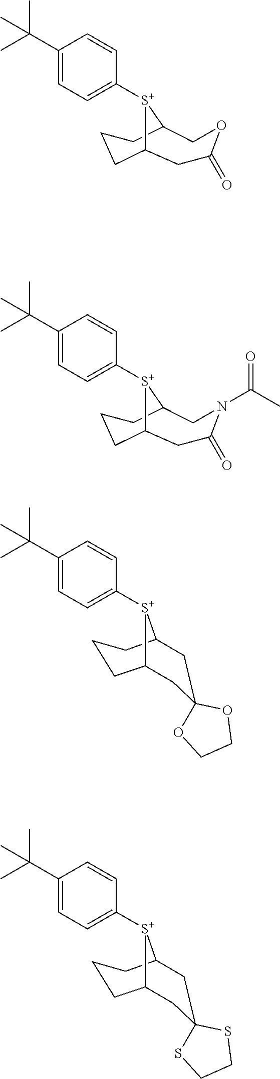

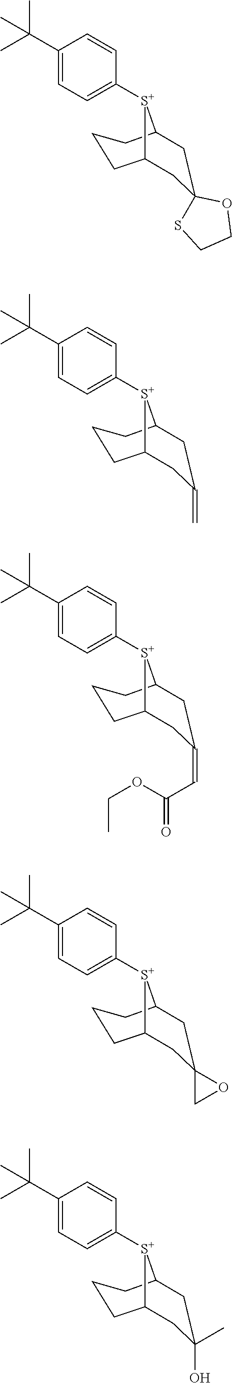

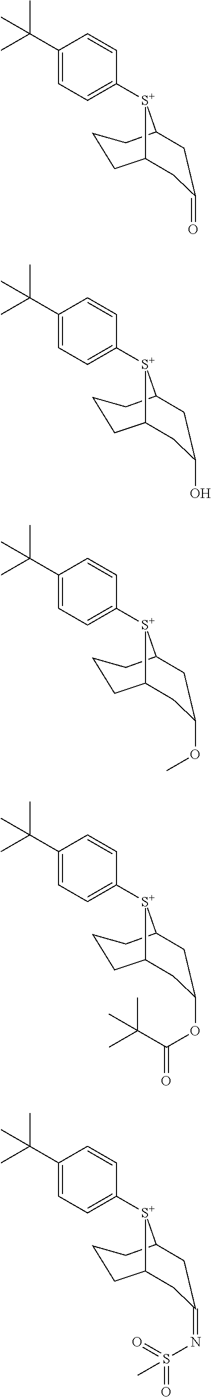 [SMILES: CC(C)(C)c1ccc([S+]2C3CCCC2CC(=O)C3)cc1 ; CC(C)(C)c1ccc([S+]2C3CCCC2CC(O)C3)cc1 ; COC1CC2CCCC(C1)[S+]2c1ccc(C(C)(C)C)cc1 ; CC(C)(C)C(=O)OC1CC2CCCC(C1)[S+]2c1ccc(C(C)(C)C)cc1 ; CC(C)(C)c1ccc([S+]2C3CCCC2CC(=NS(C)(=O)=O)C3)cc1]

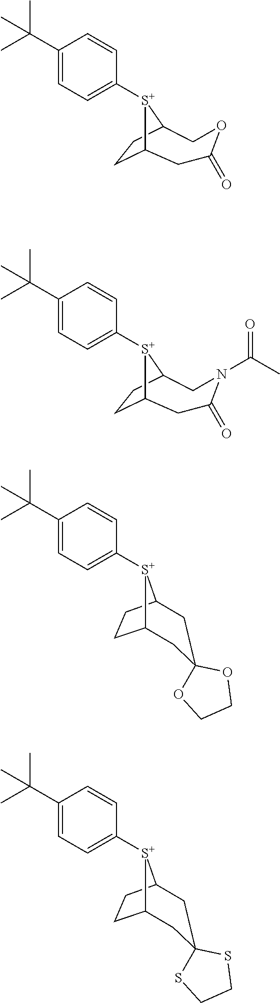

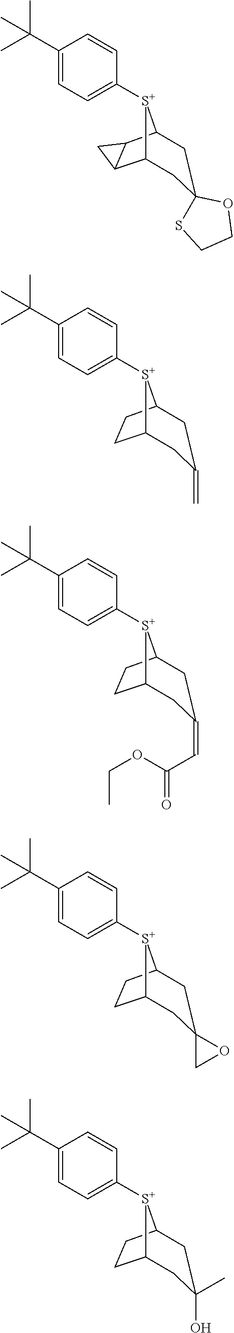

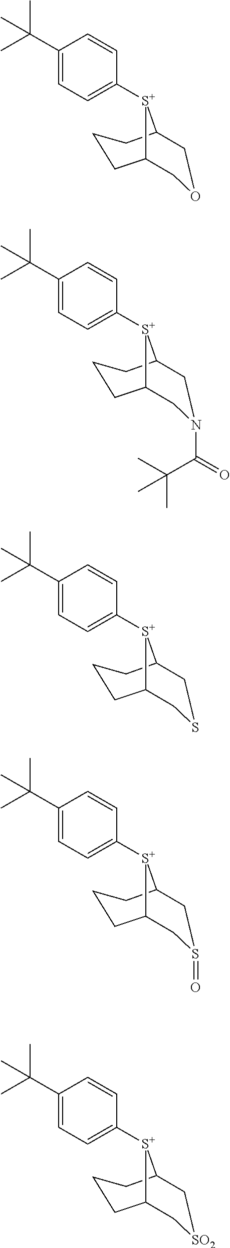 [SMILES: CC(C)(C)c1ccc([S+]2C3CCCC2COC3)cc1 ; CC(C)(C)C(=O)N1CC2CCCC(C1)[S+]2c1ccc(C(C)(C)C)cc1 ; CC(C)(C)c1ccc([S+]2C3CCCC2CSC3)cc1 ; CC(C)(C)c1ccc([S+]2C3CCCC2CS(=O)C3)cc1 ; CC(C)(C)c1ccc([S+]2C3CCCC2CS(=O)(=O)C3)cc1]

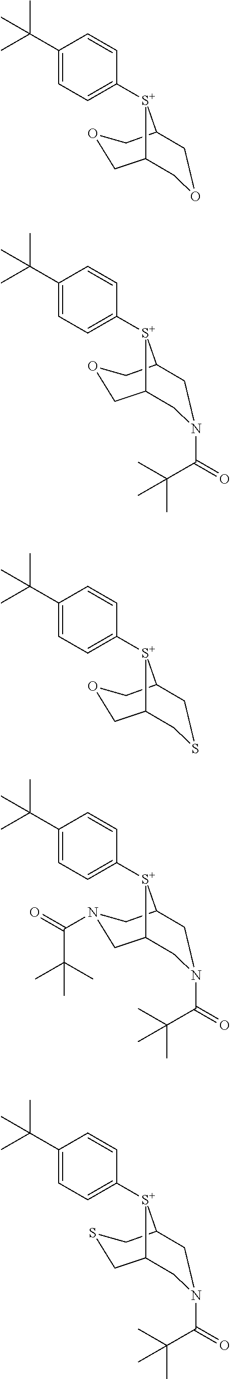 [SMILES: CC(C)(C)c1ccc([S+]2C3COCC2COC3)cc1 ; CC(C)(C)C(=O)N1CC2COCC(C1)[S+]2c1ccc(C(C)(C)C)cc1 ; CC(C)(C)c1ccc([S+]2C3COCC2CSC3)cc1 ; CC(C)(C)C(=O)N1CC2CN(C(=O)C(C)(C)C)CC(C1)[S+]2c1ccc(C(C)(C)C)cc1 ; CC(C)(C)C(=O)N1CC2CSCC(C1)[S+]2c1ccc(C(C)(C)C)cc1]

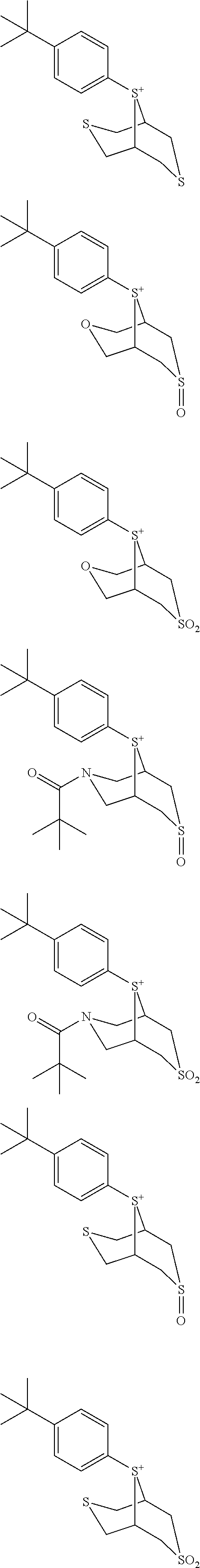

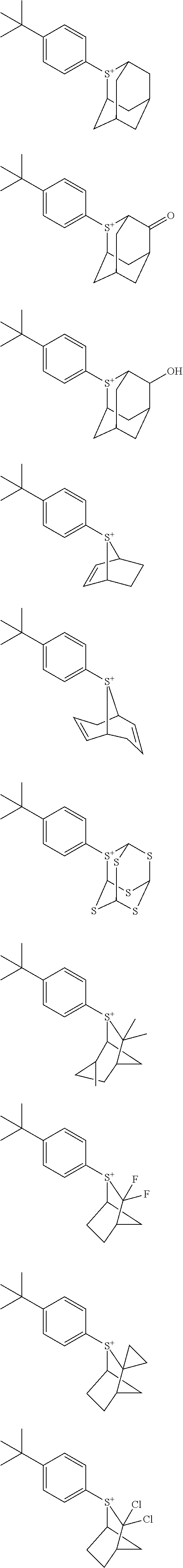

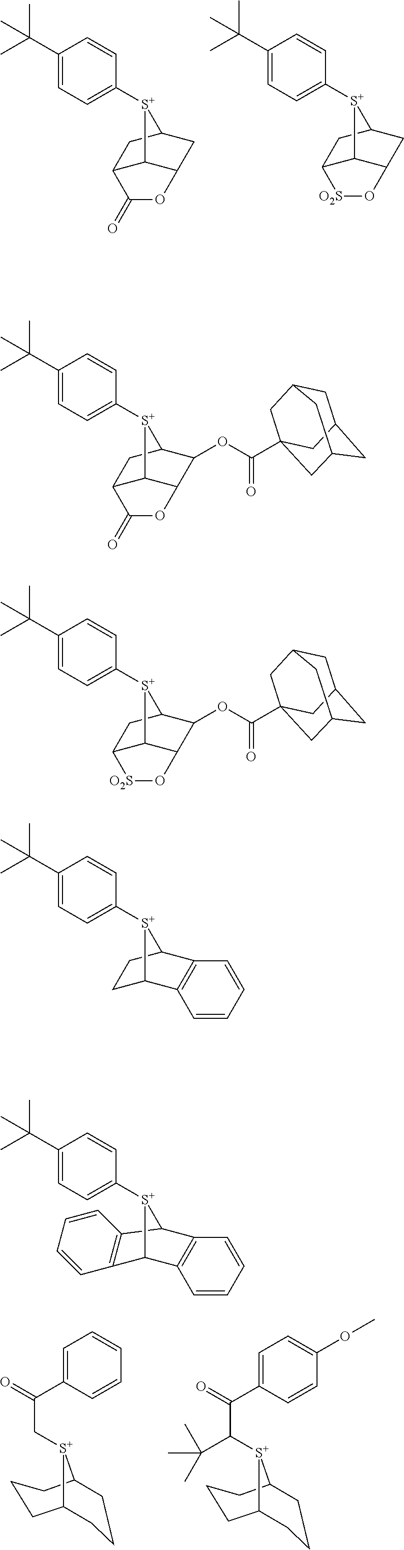

Examples of the iodonium cation having formula (cation-2) are shown below, but not limited thereto.

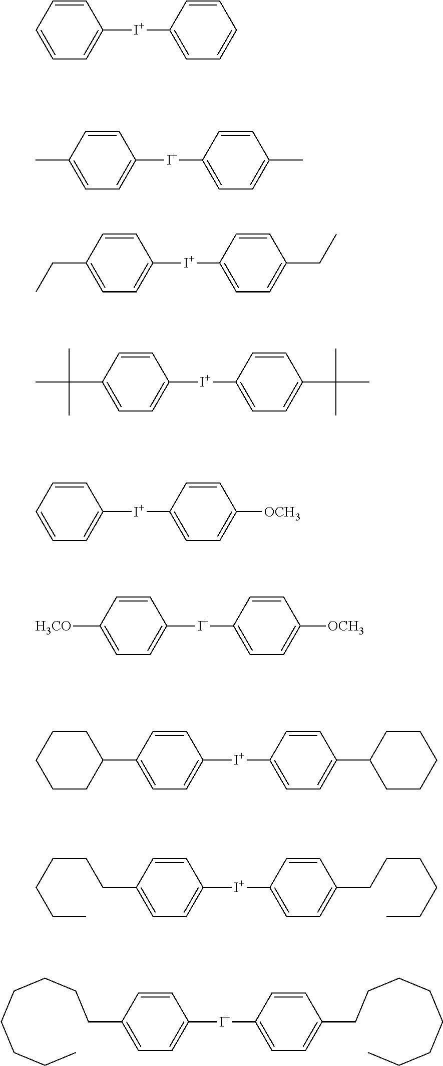

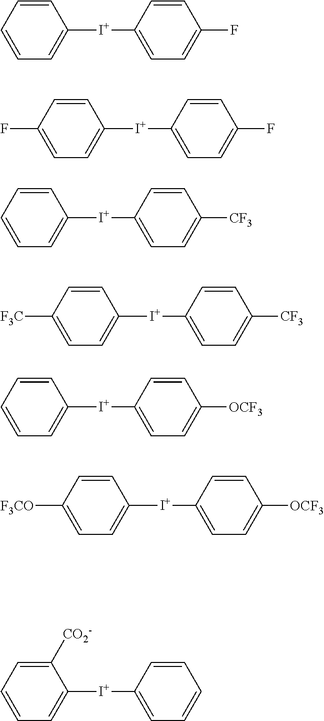

Examples of the ammonium cation having formula (cation-3) are shown below, but not limited thereto.

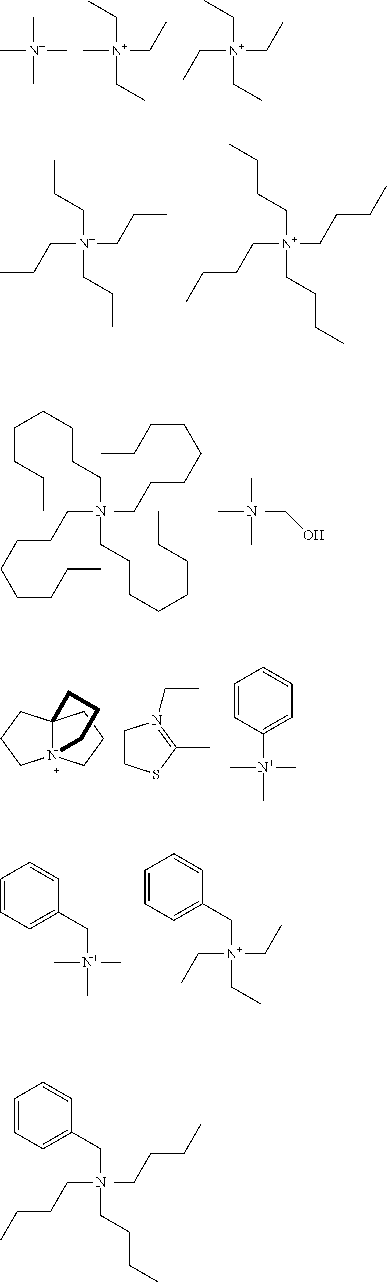

Specific structures of the inventive onium salt include arbitrary combinations of the anion with the cation, both as exemplified above.

The inventive onium salt may be synthesized, for example, according to the following scheme. Although reference is now made to the synthesis of onium salt (1′) wherein X is oxygen, the synthesis method is not limited thereto.

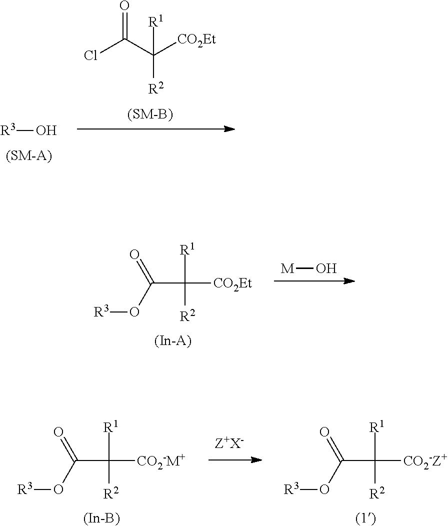

Herein, R¹ to R³ and Z⁺ are as defined above, Et stands for ethyl, M⁺ is a metal cation, and X⁻ is an anion.

First, a starting alcohol (SM-A) is esterified by reacting it with an acid chloride (SM-B). The starting alcohol (SM-A) is dissolved in a solvent such as tetrahydrofuran (THF) or acetonitrile, to which the acid chloride (SM-B) is added dropwise in the presence of a base such as pyridine or 2,6-lutidine. The reaction may be promoted by heating if necessary. While it is desirable in view of yield to monitor the reaction by gas chromatography (GC) or silica gel thin layer chromatography (TLC) until the reaction is complete, the reaction time is typically about 2 to 24 hours. The intermediate (In-A) may be collected from the reaction mixture by standard aqueous work-up. If necessary, the intermediate is purified by a standard technique such as distillation, chromatography or recrystallization.

Next, the intermediate (In-A) is subjected to alkaline hydrolysis using a metal hydroxide: M-OH, to synthesize an intermediate (In—B). The intermediate (In-A) is dissolved in a solvent such as THF or acetonitrile, to which an aqueous solution of metal hydroxide: M-OH is added dropwise for alkaline hydrolysis. Examples of the metal hydroxide used herein include sodium hydroxide, potassium hydroxide, and lithium hydroxide. The reaction may be promoted by heating if necessary. While it is desirable in view of yield to monitor the reaction by silica gel TLC until the reaction is complete, the reaction time is typically about 2 to 24 hours. The intermediate (In—B) may be collected from the reaction mixture by standard aqueous work-up. If necessary, the intermediate is purified by a standard technique such as chromatography or recrystallization.

The final step is a salt exchange between the intermediate (In—B) and an onium salt: Z⁺X⁻ to synthesize an onium salt (1′). Preferably X⁻ is a hydrogencarbonate ion, chloride ion or bromide ion because the exchange reaction runs quantitatively.

In the above-illustrated scheme, the third step of ion exchange may be readily carried out by any well-known procedure, for example, with reference to JP-A 2007-145797.

It is noted that the preparation method according to the above scheme is merely exemplary and the method of preparing the inventive onium salt is not limited thereto.

Quencher

The inventive onium salt is useful as a quencher. As used herein, the quencher refers to a compound capable of trapping the acid, which is generated by the acid generator in the resist composition upon light exposure, to prevent the acid from diffusing to the unexposed region and to assist in forming the desired pattern.

In a system where the inventive onium salt and an onium salt capable of generating strong acid such as α-fluorinated sulfonic acid, imide acid or methide acid are co-present, a corresponding carboxylic acid and strong acid generate upon light exposure. On the other hand, in the region receiving a reduced dose of exposure, much onium salt remains undecomposed. The strong acid functions as a catalyst for inducing deprotection reaction to the base resin whereas the inventive onium salt induces little deprotection reaction. The strong acid undergoes ion exchange with the residual carboxylic acid sulfonium salt. It is converted to a strong acid onium salt and instead, carboxylic acid is released. Differently stated, through ion exchange, the strong acid is neutralized with the carboxylic acid sulfonium salt. That is, the inventive onium salt functions as a quencher. This onium salt type quencher tends to form a resist pattern with a reduced LWR as compared with the conventional quenchers in the form of amine compounds.

Salt exchange between strong acid and carboxylic acid onium salt is infinitely repeated. The site where strong acid is generated at the end of exposure shifts from the site where the onium salt of strong acid generation type is initially present. It is believed that since the cycle of photo-acid generation and salt exchange is repeated many times, the points of acid generation are averaged, which leads to a resist pattern with reduced LWR after development.

As the compound that exerts a quencher effect by a similar mechanism, Patent Documents 1 to 6 describe carboxylic acid onium salts, alkanesulfonic acid onium salts, arenesulfonic acid onium salts, and α,α-difluorocarboxylic acid onium salts. With respect to the type of onium salt, sulfonium, iodonium and ammonium salts are included. However, on use of an alkanesulfonic acid onium salt or arenesulfonic acid onium salt, the generated acid has a certain acid strength so that part thereof may induce deprotection reaction as the acid generator rather than as the quencher, leading to a lowering of resolution and an increase of acid diffusion, which invite losses of resist performance factors like exposure latitude (EL) and mask error factor (MEF). Also, the α,α-difluorocarboxylic acid onium salt as described in Patent Document 6, despite a carboxylic acid onium salt, has a possibility to provoke deprotection reaction depending on a choice of acid labile group on the base polymer, for the reason that the generated acid has a relatively high acidity like the sulfonic acid onium salt, due to the inclusion of fluorine at α-position of the carboxylate anion. Fluorocarboxylic acid onium salts obtained by simply extending the straight chain similarly allow for substantial acid diffusion and undergo salt exchange with strong acid in the unexposed region, probably leading to losses of resolution, EL and MEF. Further, the alkanecarboxylic acid onium salt is highly hydrophilic though it functions as a quencher. The fluoroalkanecarboxylic acid onium salt as described in Patent Document 3 has a somewhat controlled level of hydrophilicity as compared with the non-fluorinated type, but the control of hydrophilicity is insufficient when the carbon count is small. Although some onium salts of perfluoroalkanecarboxylic acid having a larger carbon count are known, they are deemed incompatible with resist compositions because the carboxylic acids have surfactant-like physical properties. Incompatibility with resist compositions can cause defect formation. Additionally, perfluoroalkanecarboxylic acids are unfavorable from the biotic and environmental aspects.

The inventive onium salt solves the outstanding problem. The onium salt having such a structure in the anion functions as a quencher, that is, turns to a 1,3-dicarboxylic acid monoester or 1,3-ketocarboxylic acid structure by effectively trapping the strong acid generated from the acid generator. In the onium salt of formula (1) wherein R³ forms an acid labile group with the adjacent oxygen atom, the acid labile group is eliminated as a result of reaction with strong acid whereby resist sensitivity is improved and a 1,3-dicarboxylic acid (e.g., malonic acid) structure forms. The 1,3-dicarboxylic acid monoester, 1,3-ketocarboxylic acid and 1,3-dicarboxylic acid undergo thermal decarbonation reaction during the subsequent step of PEB whereby it is decomposed into carbon dioxide and a corresponding acetic acid derivative or ketone derivative, i.e., volatilizes from the resist film. In the subsequent development step in alkaline developer, the carboxylic acid having high affinity to the alkaline developer has been eliminated. This prevents the resist film from being swollen. The problem of resist pattern collapse which has been unsolved during formation of small-size patterns is overcome.

Resist Composition

Another embodiment of the invention is a resist composition comprising (A) a quencher in the form of the onium salt having formula (1) as an essential component.

In the resist composition, the quencher (A) is preferably used in an amount of 0.1 to 40 parts by weight, more preferably 1 to 20 parts by weight per 80 parts by weight of the base polymer (C) described below. An amount of quencher (A) in the range ensures a satisfactory quenching function, eliminating the risk of lowering sensitivity or leaving foreign particles due to shortage of solubility.

(B) Organic Solvent

The resist composition may comprise (B) an organic solvent. The organic solvent used herein is not particularly limited as long as the foregoing and other components are soluble therein. Suitable solvents include ketones such as cyclopentanone, cyclohexanone, and methyl-2-n-pentyl ketone; alcohols such as 3-methoxybutanol, 3-methyl-3-methoxybutanol, 1-methoxy-2-propanol, and 1-ethoxy-2-propanol; keto-alcohols such as diacetone alcohol (DAA); ethers such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether; esters such as propylene glycol monomethyl ether acetate (PGMEA), propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; and lactones such as γ-butyrolactone (GBL), and mixtures thereof.

Of the foregoing organic solvents, it is recommended to use PGME, PGMEA, cyclohexanone, GBL, DAA, ethyl lactate, and mixtures thereof because the base polymer (C) is most soluble therein.

The organic solvent (B) is preferably added in an amount of 200 to 5,000 parts by weight, and more preferably 400 to 3,500 parts by weight per 80 parts by weight of the base polymer (C). The organic solvent may be used alone or in admixture.

(C) Base Polymer

The resist composition may further comprise (C) a base polymer. The base polymer preferably contains repeat units having the formula (a1), which are also referred to as repeat units (a1).

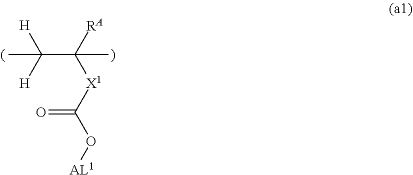

In formula (a1), R^{A} is hydrogen, fluorine, methyl or trifluoromethyl. X¹ is a single bond, phenylene group, naphthylene group, or*—C(═O)—O—X¹¹—. The phenylene and naphthylene groups may be substituted with an optionally fluorinated C₁-C₁₀ alkyl moiety or halogen. X¹¹ is a C₁-C₁₀ saturated hydrocarbylene group which may contain a hydroxy moiety, ether bond, ester bond or lactone ring, a phenylene group or naphthylene group. The asterisk (*) designates a point of attachment to the carbon atom in the backbone. AL¹ is an acid labile group.

The acid labile group represented by AL¹ may be selected from a variety of such groups, for example, those groups described in JP-A 2013-080033 (U.S. Pat. No. 8,574,817) and JP-A 2013-083821 (U.S. Pat. No. 8,846,303).

Typical of the acid labile group are groups of the following formulae (AL-3) to (AL-5).

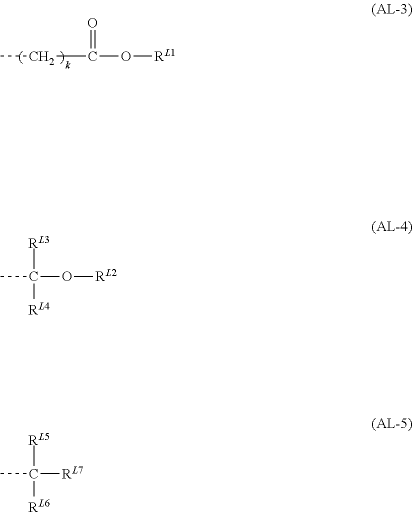

In formulae (AL-3) and (AL-4), R^{L1} and R^{L2} are each independently a C₁-C₄₀ saturated hydrocarbyl group which may contain a heteroatom such as oxygen, sulfur, nitrogen or fluorine. The saturated hydrocarbyl group may be straight, branched or cyclic. Inter alia, C₁-C₂₀ saturated hydrocarbyl groups are preferred.

In formula (AL-3), k is an integer of 0 to 10, preferably 1 to 5.

In formula (AL-4), R^{L3} and R^{L4} are each independently hydrogen or a C₁-C₄₀ saturated hydrocarbyl group which may contain a heteroatom such as oxygen, sulfur, nitrogen or fluorine. The saturated hydrocarbyl group may be straight, branched or cyclic. Any two of R^{L2}, R^{L3} and R^{L4} may bond together to form a C₃-C₂₀ ring with the carbon atom or carbon and oxygen atoms to which they are attached. The ring preferably contains 4 to 16 carbon atoms and is typically alicyclic.

In formula (AL-5), R^{L5}, R^{L6} and R^{L7} are each independently a C₁-C₂₀ saturated hydrocarbyl group which may contain a heteroatom such as oxygen, sulfur, nitrogen or fluorine. The saturated hydrocarbyl group may be straight, branched or cyclic. Any two of R^{L5}, R^{L6} and R^{L7} may bond together to form a C₃-C₂₀ ring with the carbon atom to which they are attached. The ring preferably contains 4 to 16 carbon atoms and is typically alicyclic.

Examples of the repeat unit (a1) are shown below, but not limited thereto. Herein R^{A} and AL¹ are as defined above.

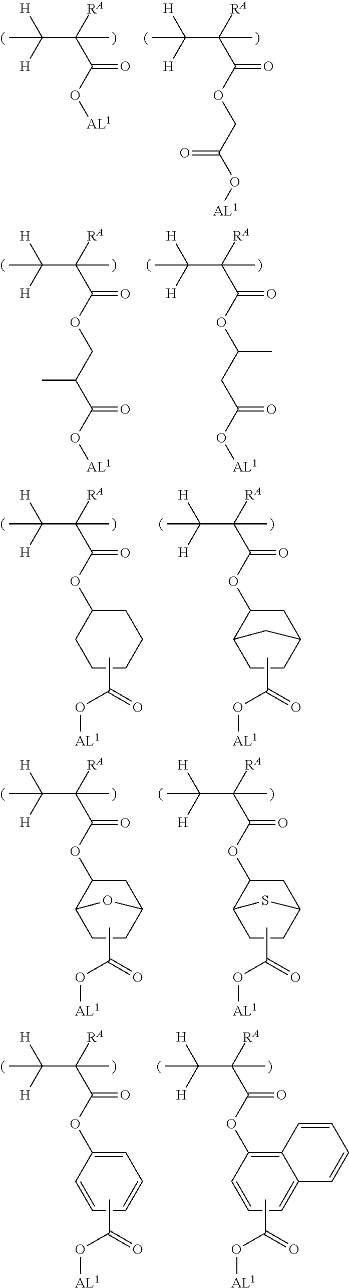

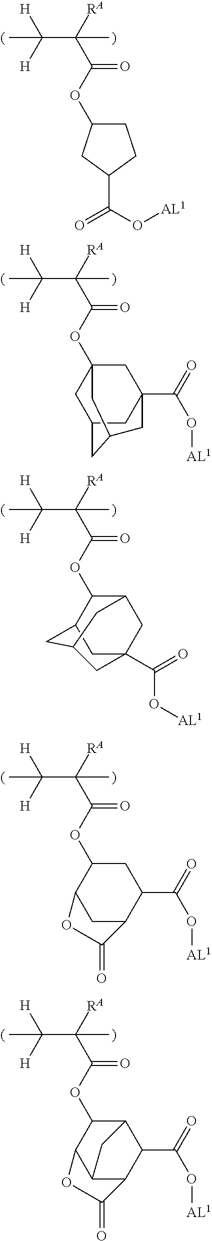

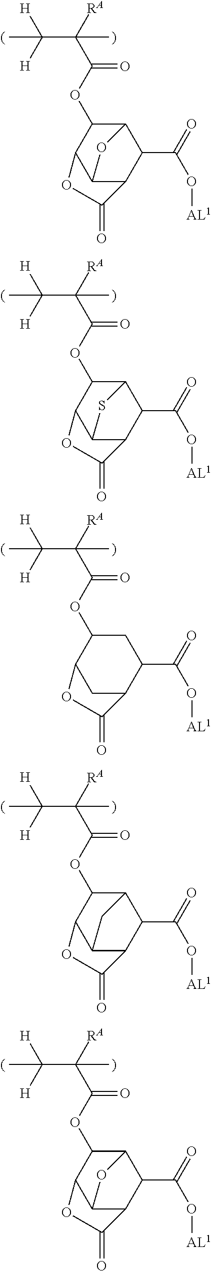

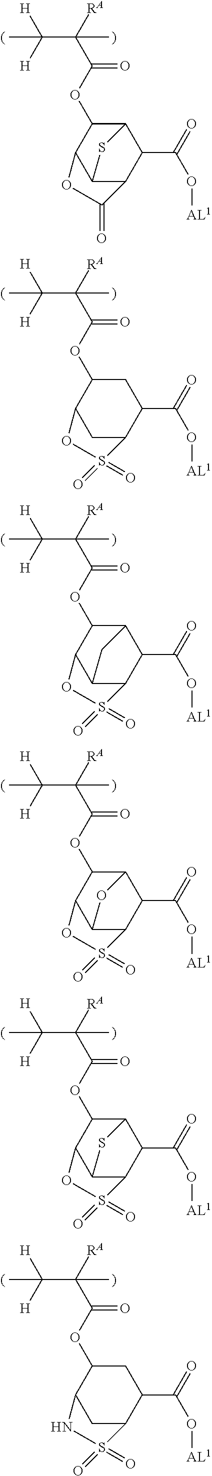

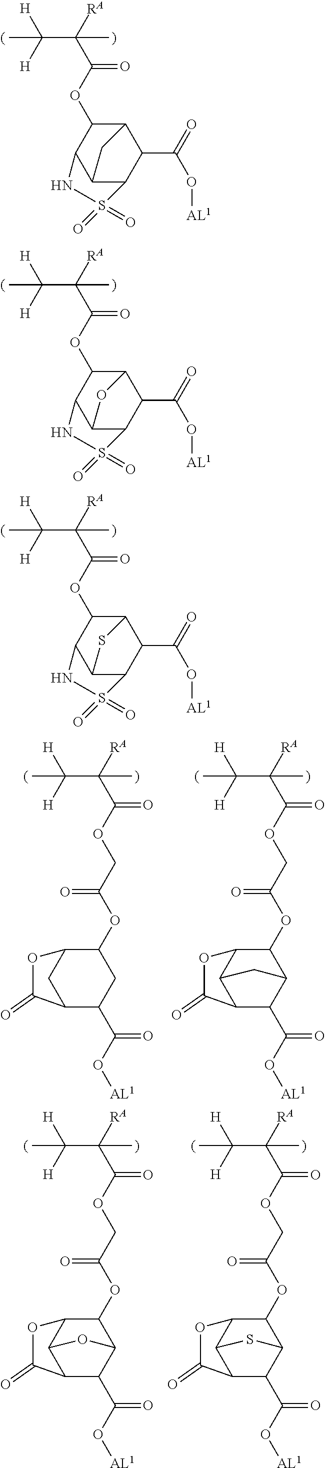

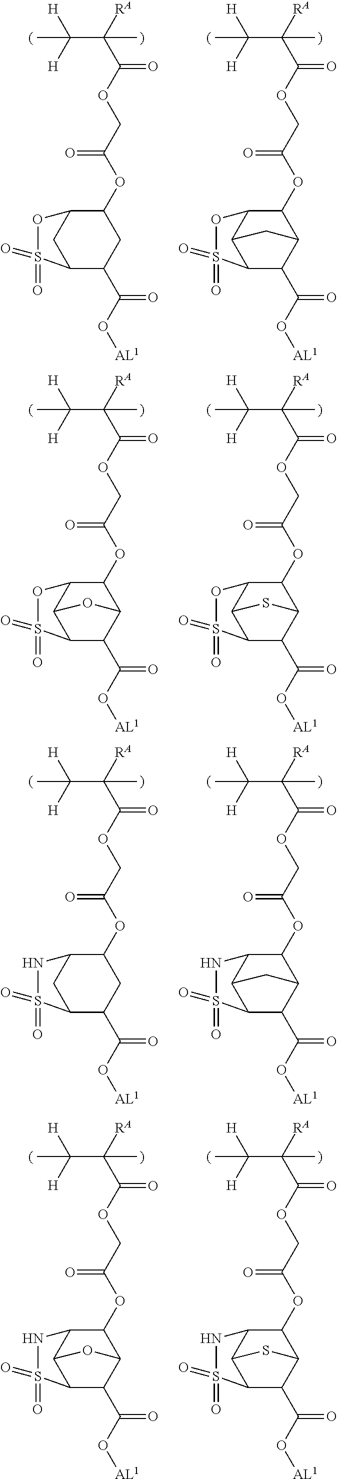

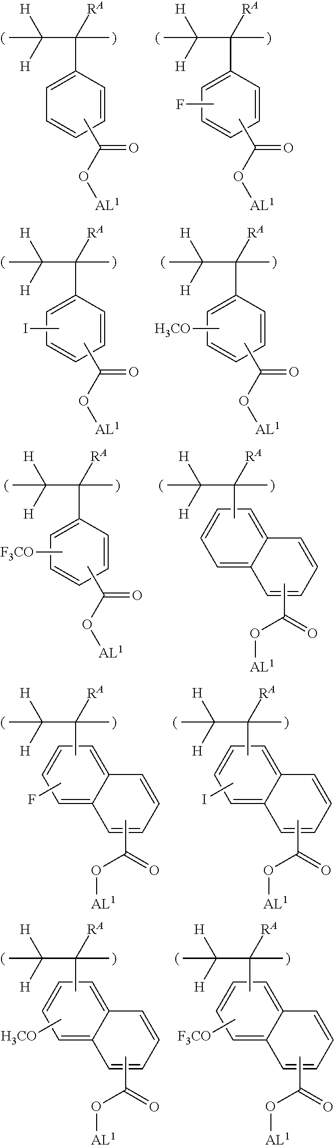

The base polymer may further comprise repeat units having the formula (a2), which are also referred to as repeat units (a2).

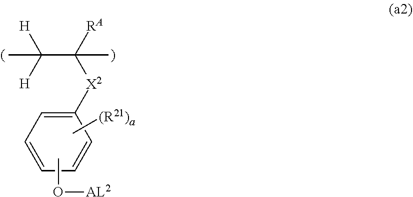

In formula (a2), R^{A} is hydrogen, fluorine, methyl or trifluoromethyl. X² is a single bond or*—C(═O)—O—. The asterisk (*) designates a point of attachment to the carbon atom in the backbone. R²¹ is halogen, cyano, a C₁-C₂₀ hydrocarbyl group which may contain a heteroatom. C₁-C₂₀ hydrocarbyloxy group which may contain a heteroatom, C₂-C₂ hydrocarbylcarbonyl group which may contain a heteroatom, C₂-C₂₀ hydrocarbylcarbonyloxy group which may contain a heteroatom, or C₂-C₂₀ hydrocarbyloxycarbonyl group which may contain a heteroatom. AL² is an acid labile group, examples of which are the same as the acid labile group AL¹. The subscript “a” is an integer of 0 to 4, preferably 0 or 1.

Examples of the repeat unit (a2) are shown below, but not limited thereto. Herein R^{A} and AL² are as defined above.

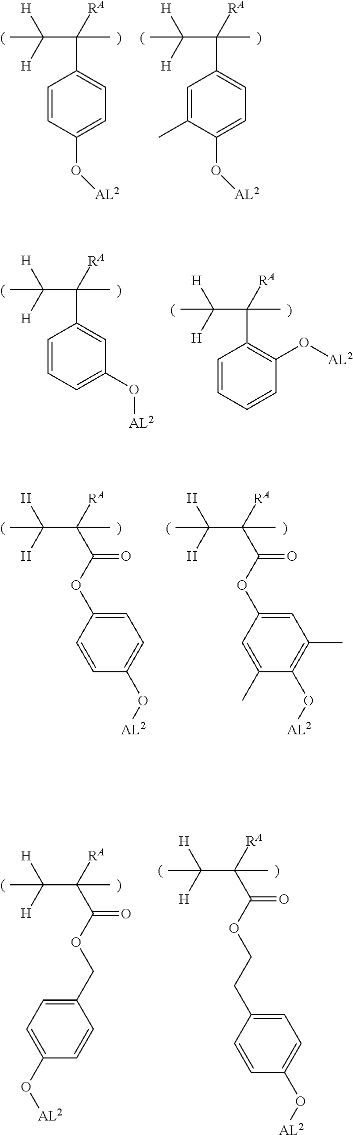

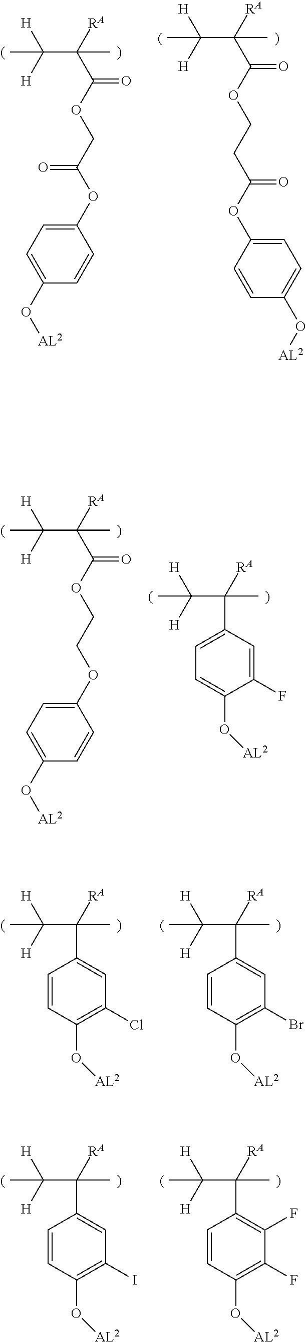

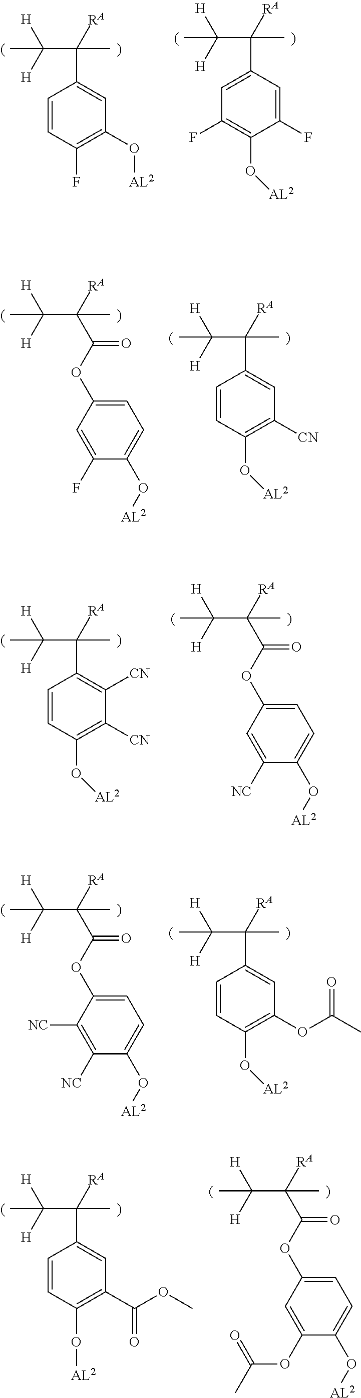

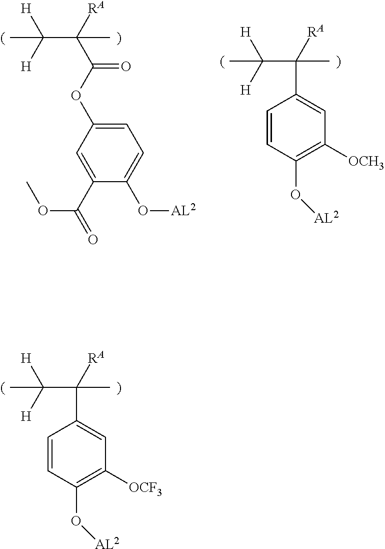

The base polymer may further comprise repeat units having the formula (b1) or repeat units having the formula (b2), which are also referred to as repeat units (b1) or (b2).

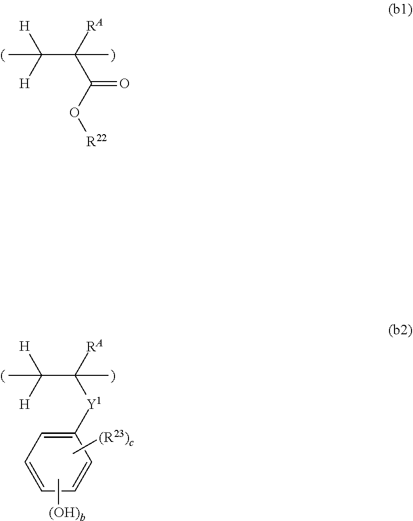

In formulae (b1) and (b2), R^{A} is each independently hydrogen, fluorine, methyl or trifluoromethyl. Y¹ is a single bond or*—C(═O)—O—. R²² is hydrogen or a C₁-C₂₀ group containing at least one structure selected from hydroxy other than phenolic hydroxy, cyano, carbonyl, carboxy, ether bond, ester bond, sulfonic ester bond, carbonate bond, lactone ring, sultone ring, and carboxylic anhydride (—C(═O)—O—C(═O)—). R²³ is halogen, hydroxy, nitro, a C₁-C₂₀ hydrocarbyl group which may contain a heteroatom, C₁-C₂₀ hydrocarbyloxy group which may contain a heteroatom, C₂-C₂₀ hydrocarbylcarbonyl group which may contain a heteroatom. C₂-C₂₀ hydrocarbylcarbonyloxy group which may contain a heteroatom, or C₂-C₂₀ hydrocarbyloxycarbonyl group which may contain a heteroatom. The subscript “b” is an integer of 1 to 4, “c” is an integer of 0 to 4, and the sum of b+c is from 1 to 5.

Examples of the repeat unit (b1) are shown below, but not limited thereto. Herein R^{A} is as defined above.

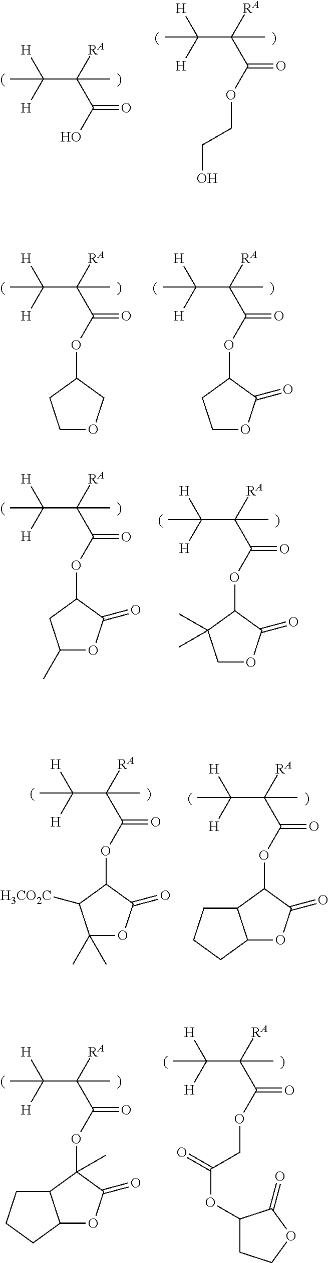

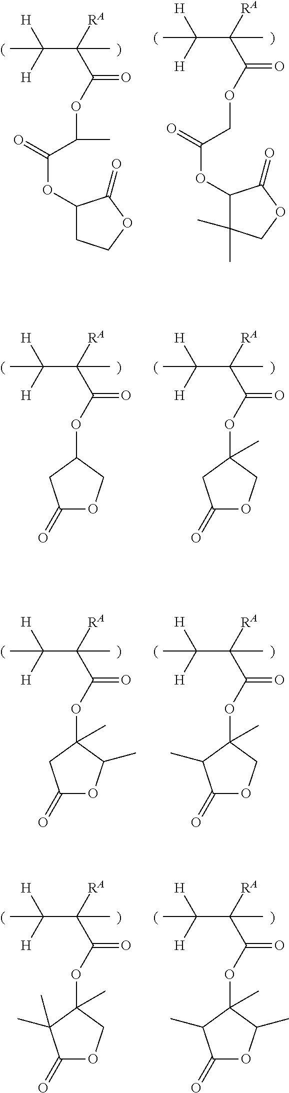

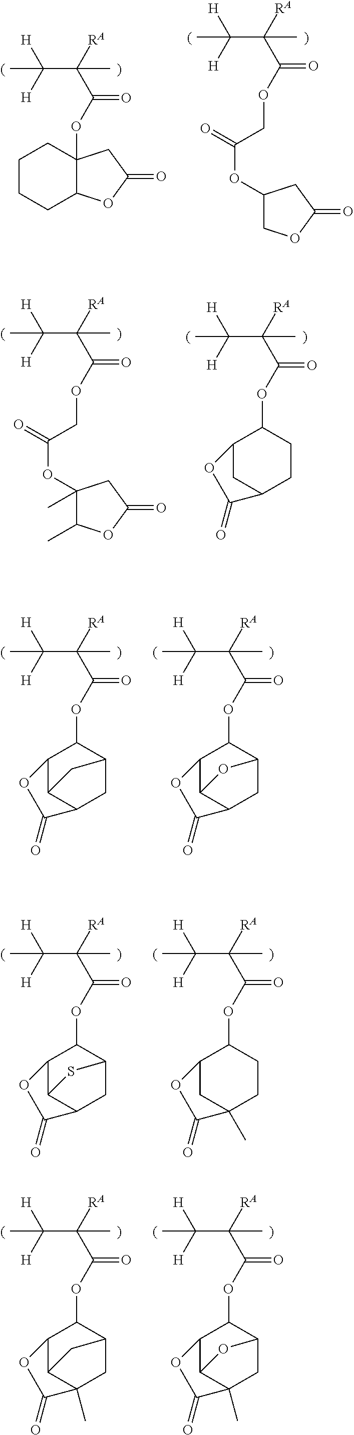

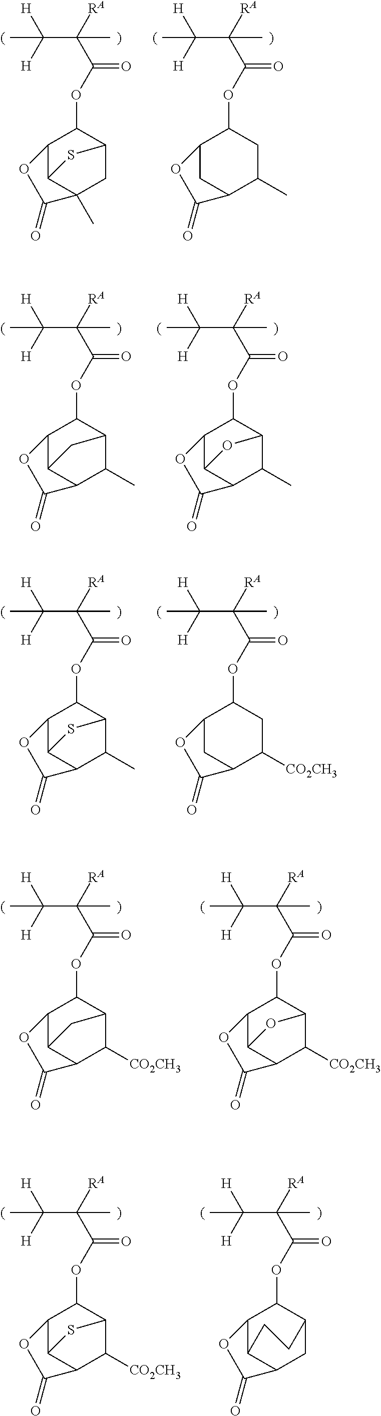

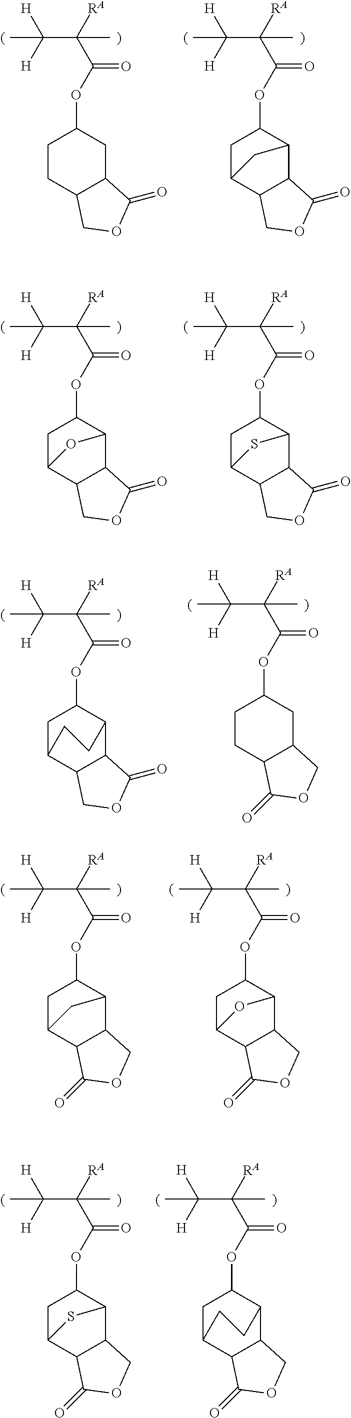

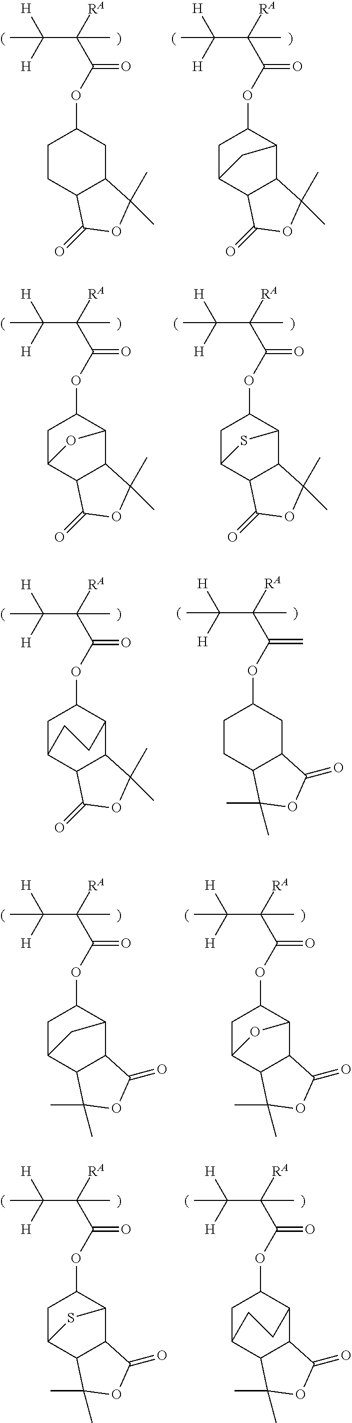

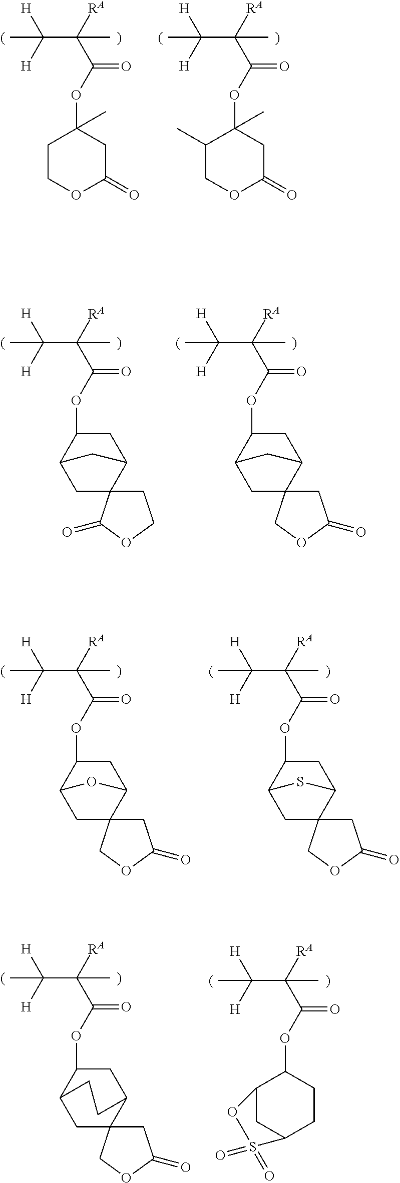

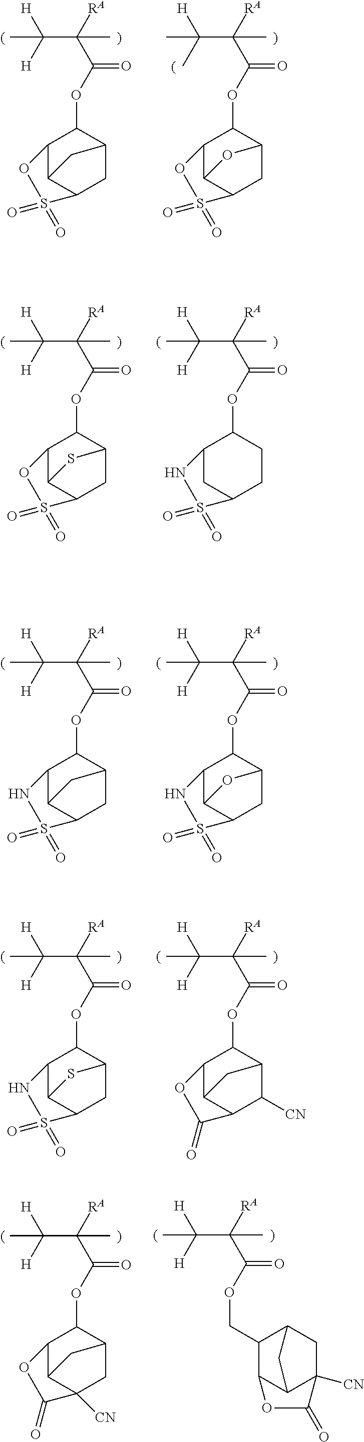

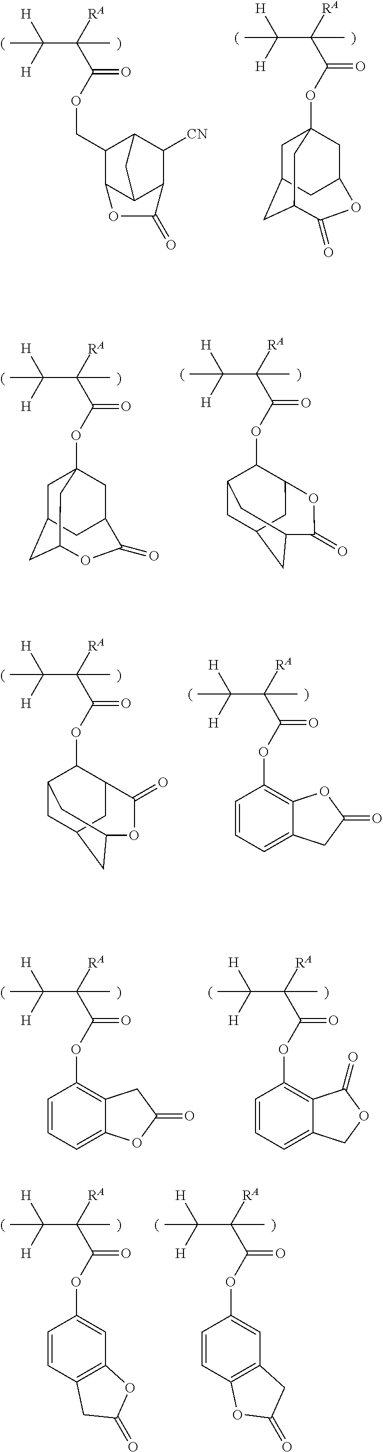

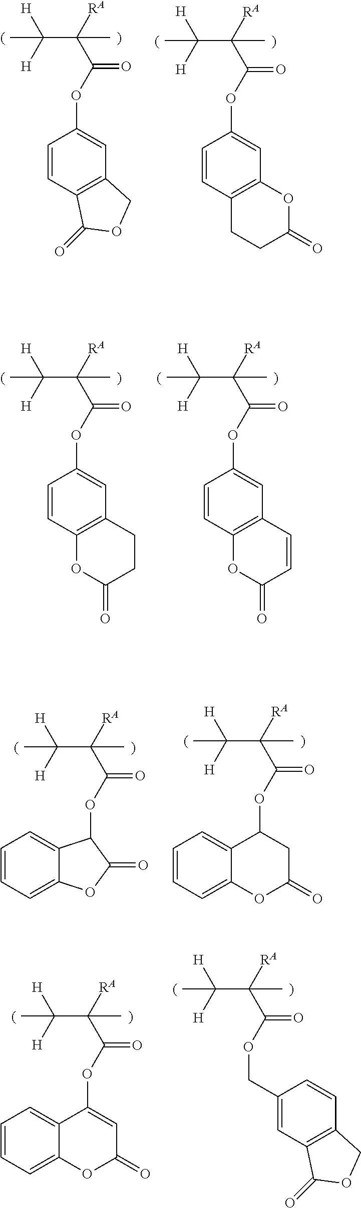

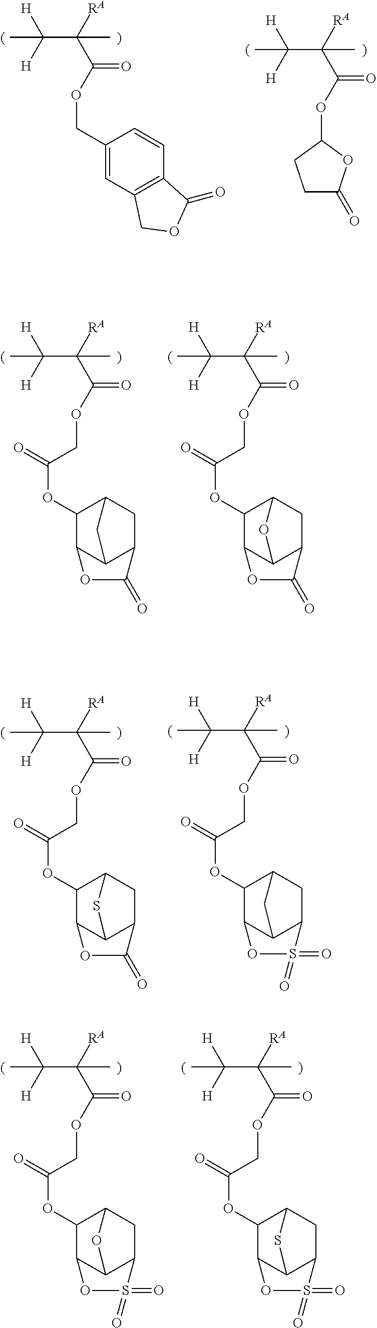

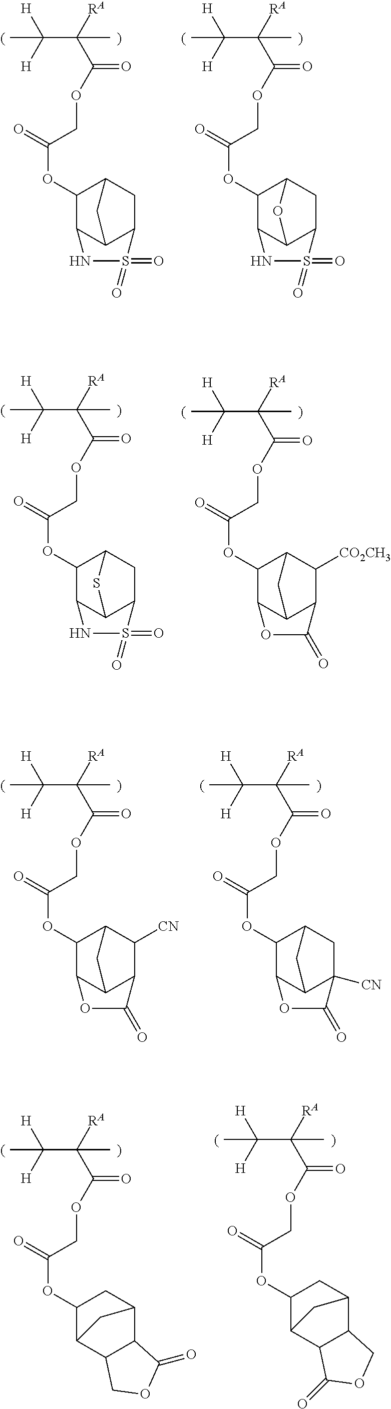

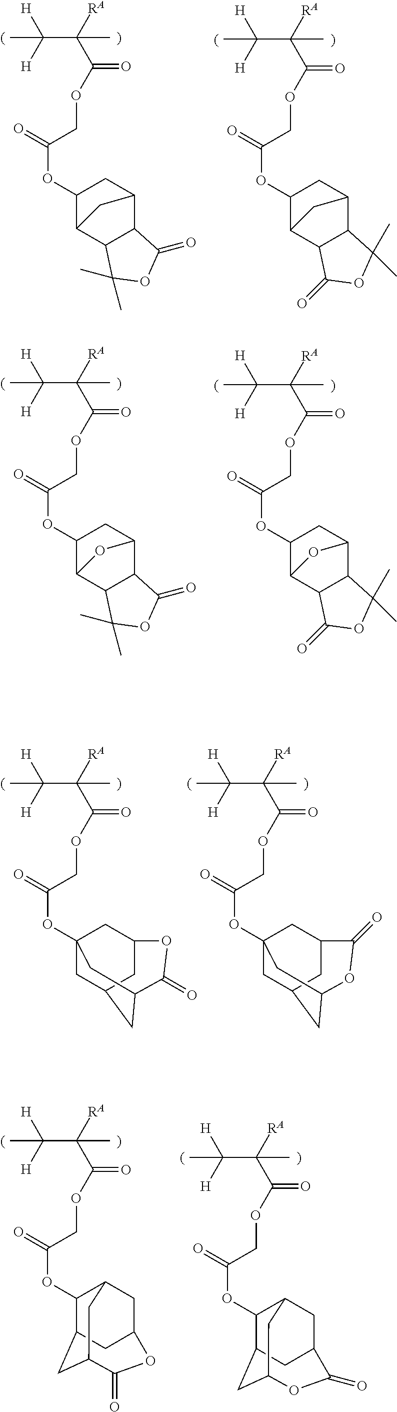

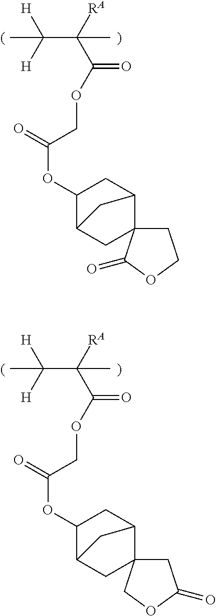

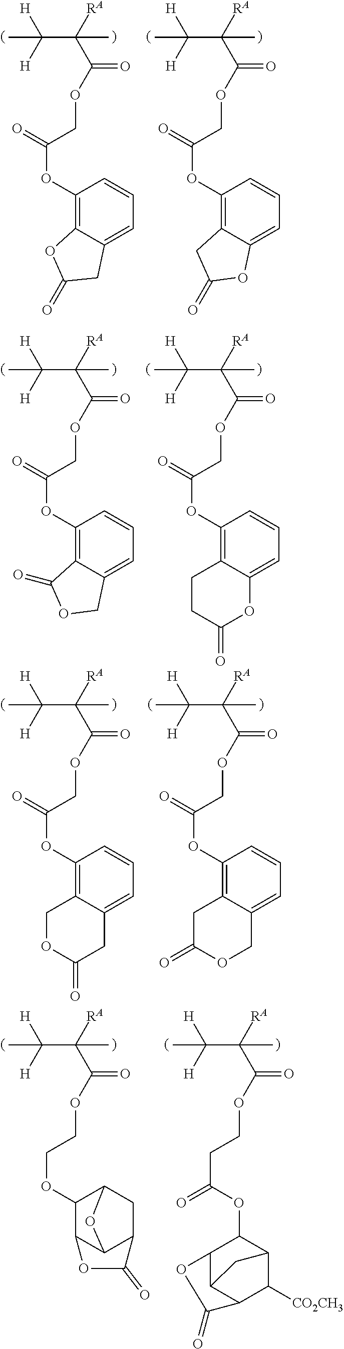

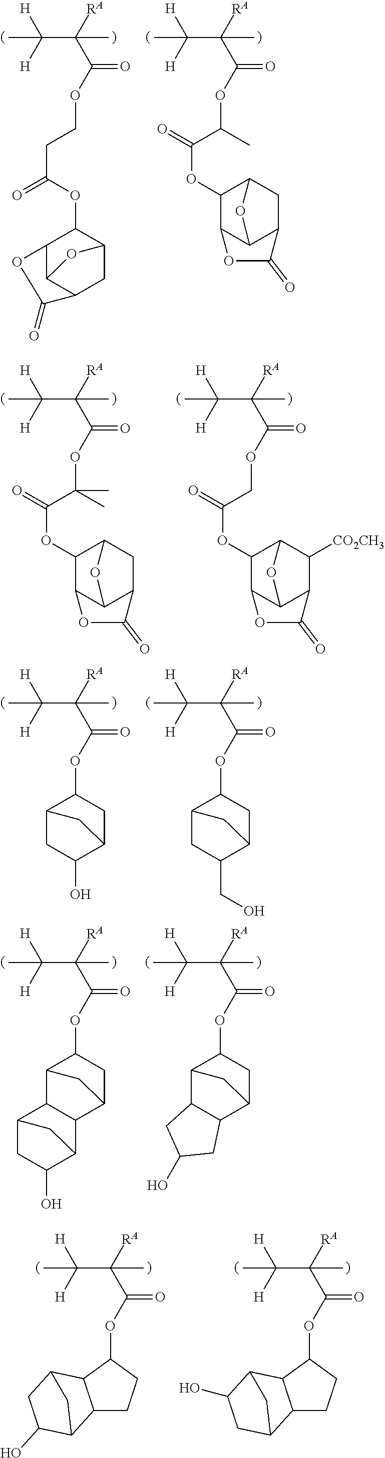

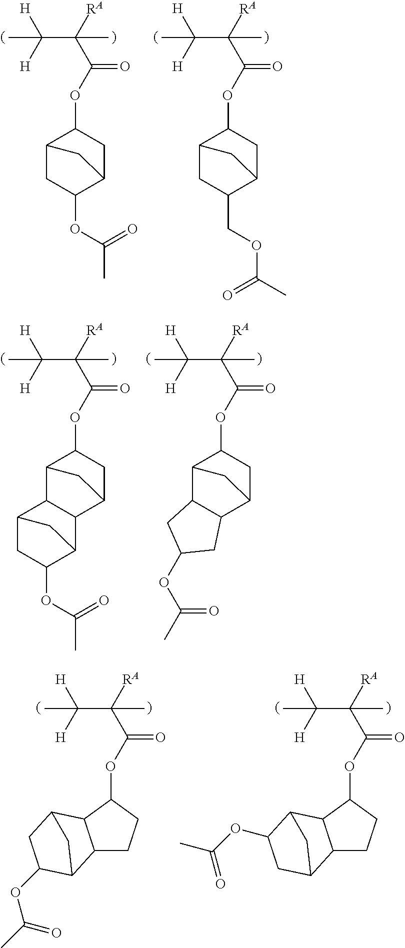

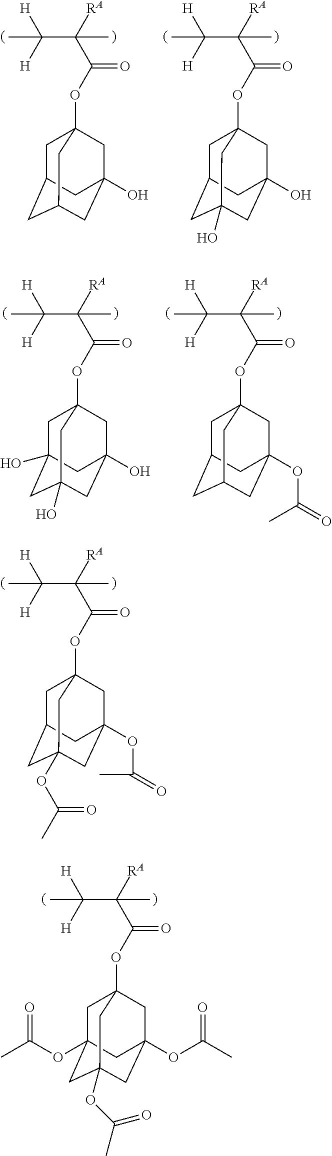

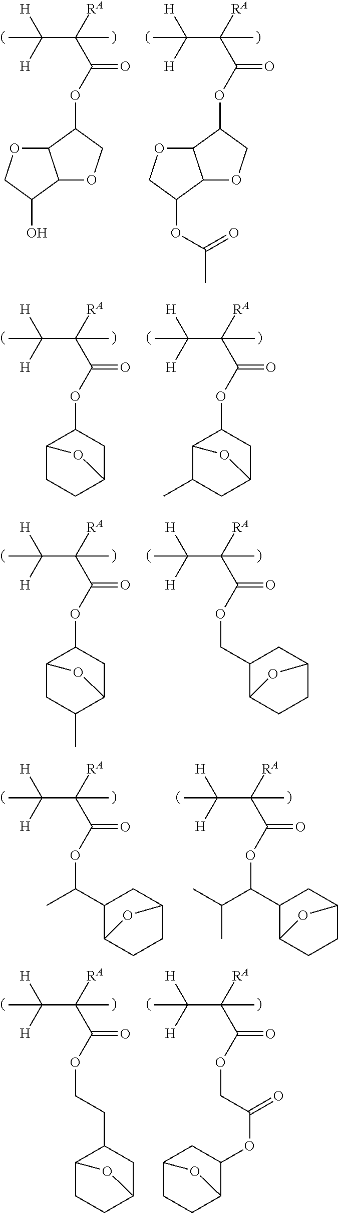

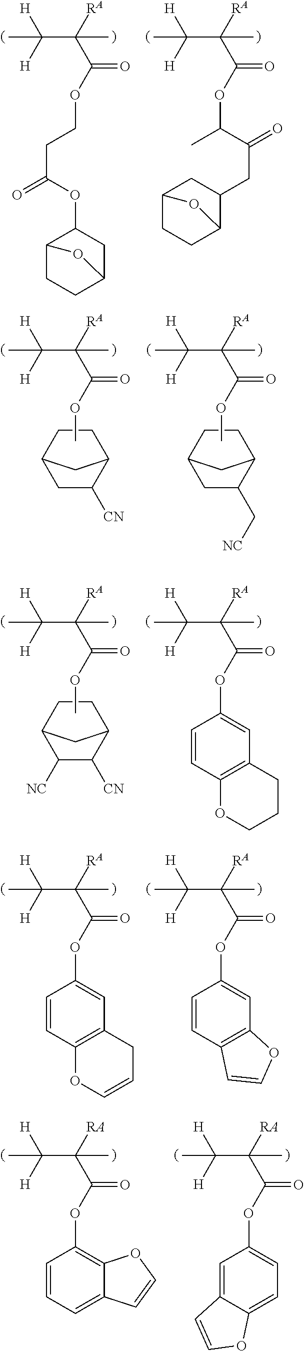

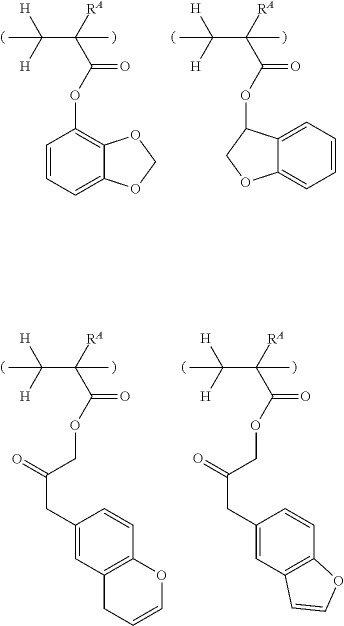

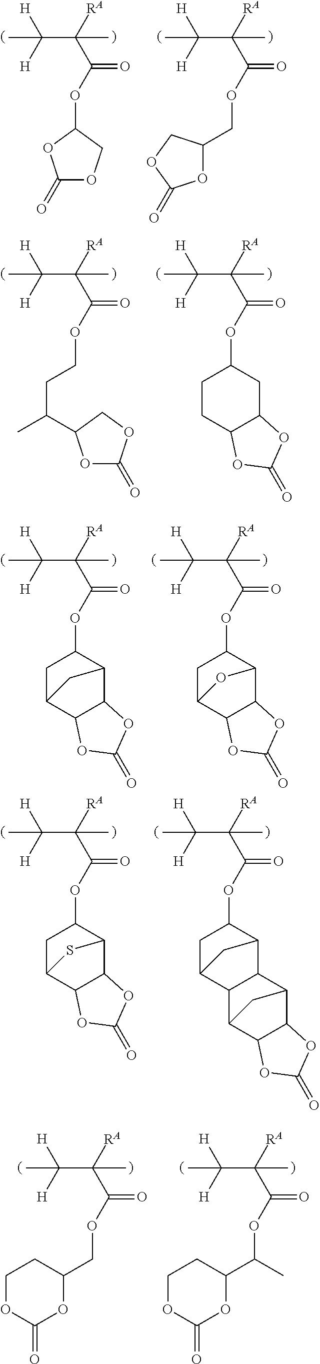

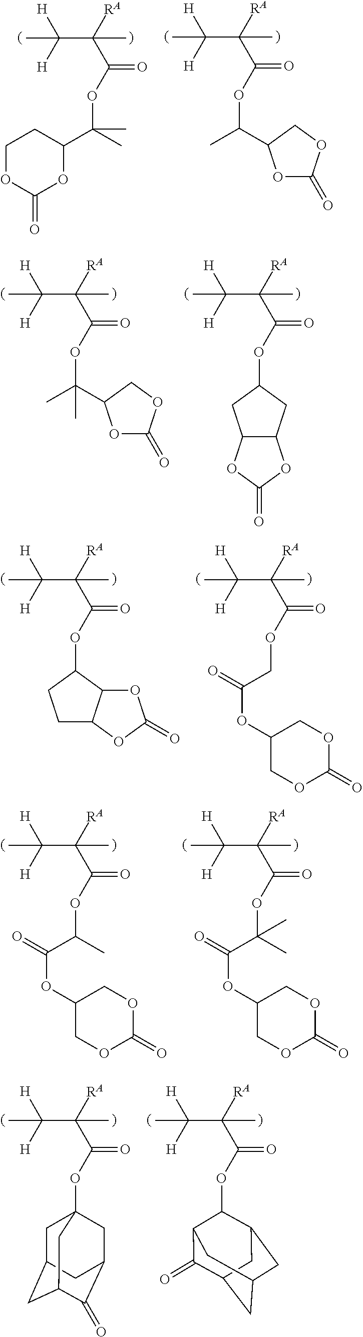

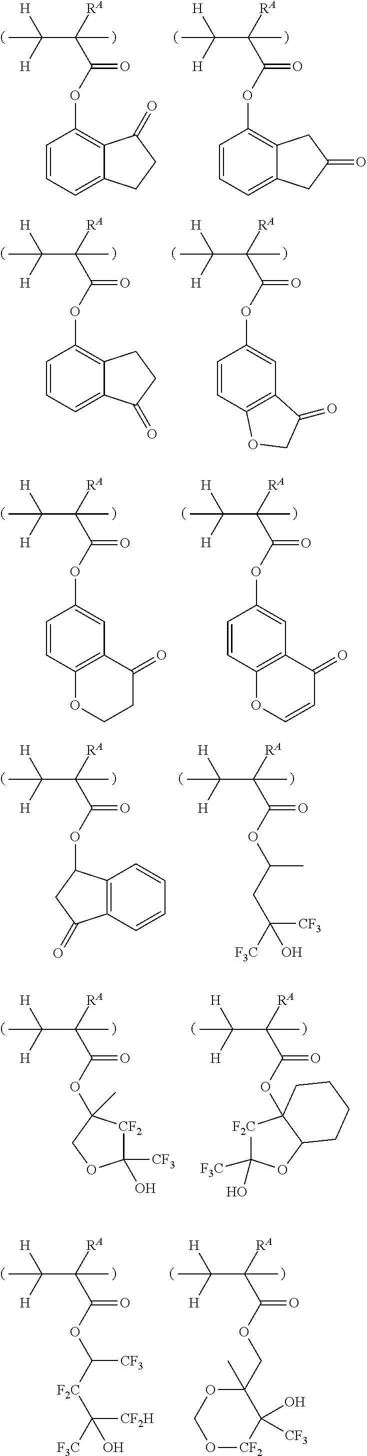

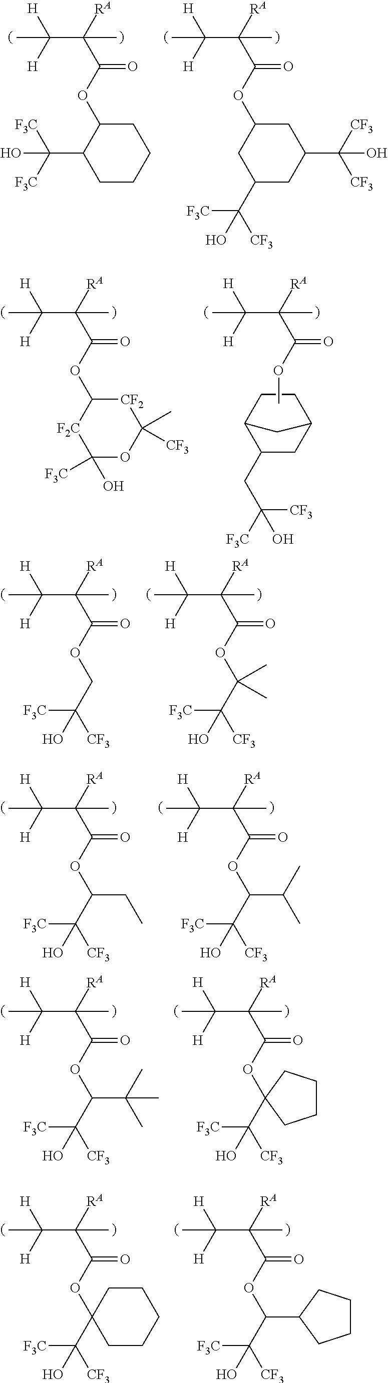

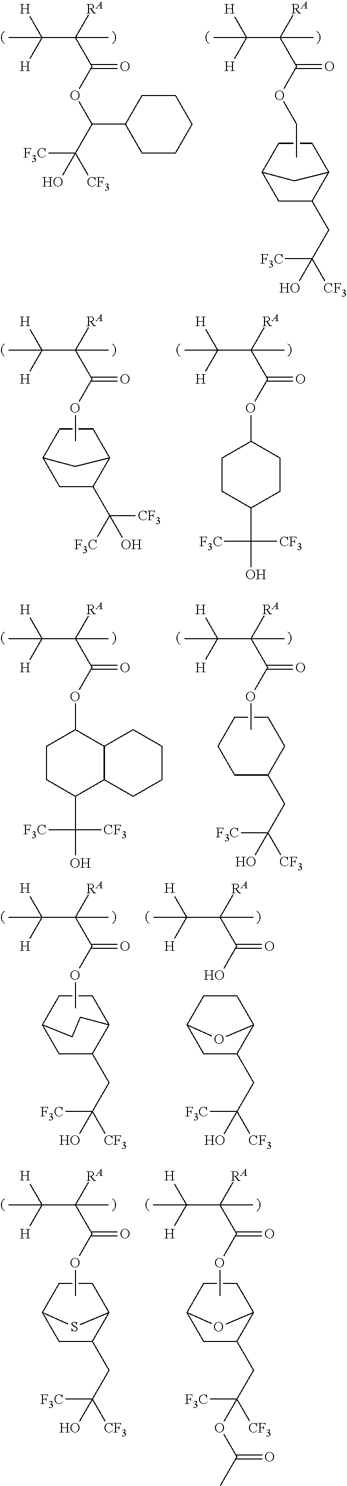

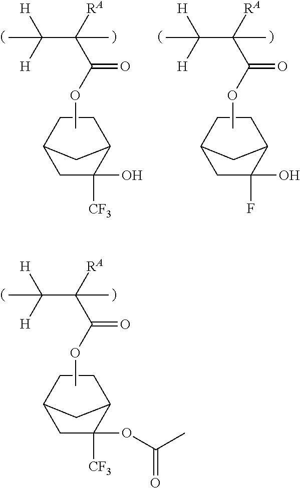

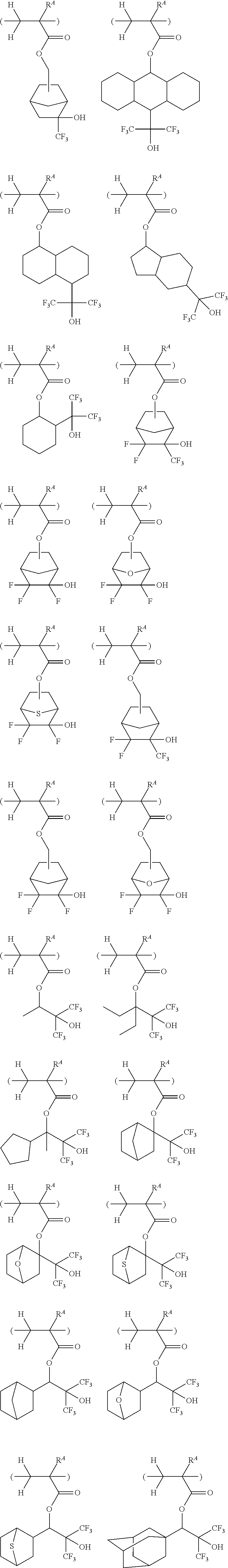

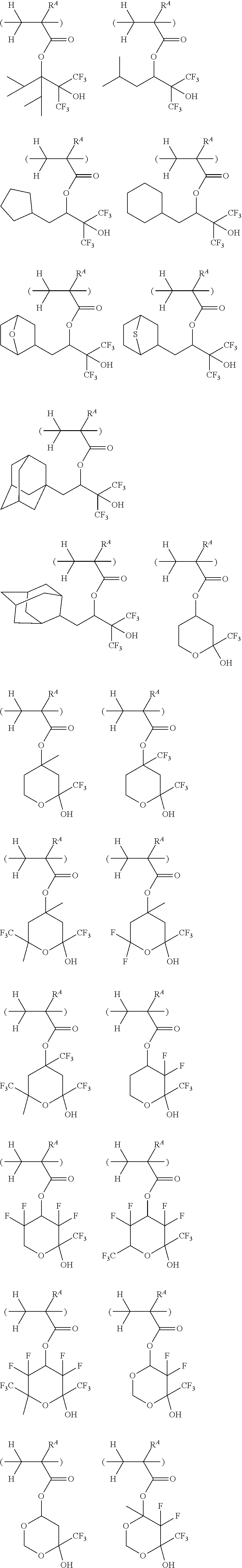

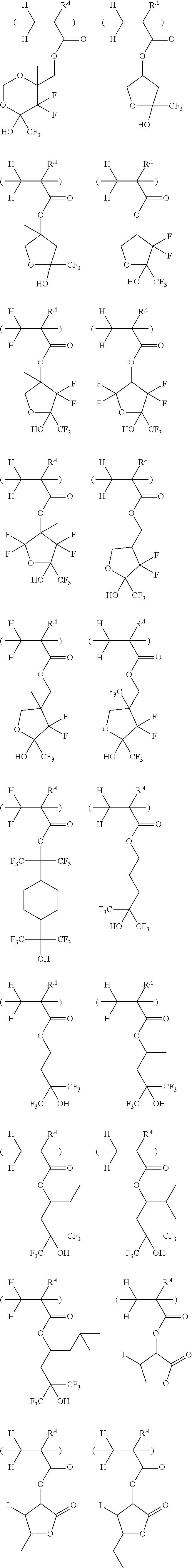

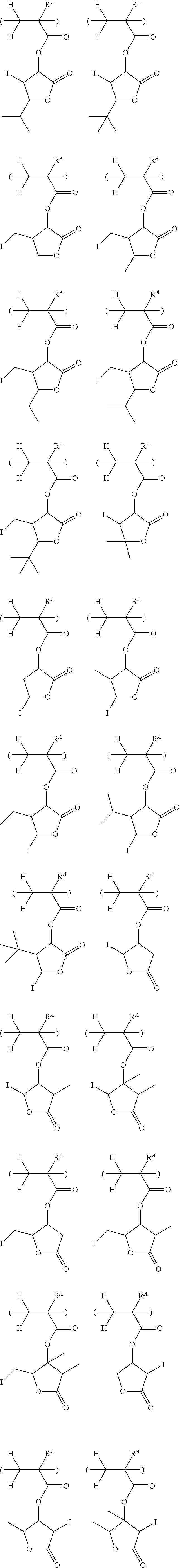

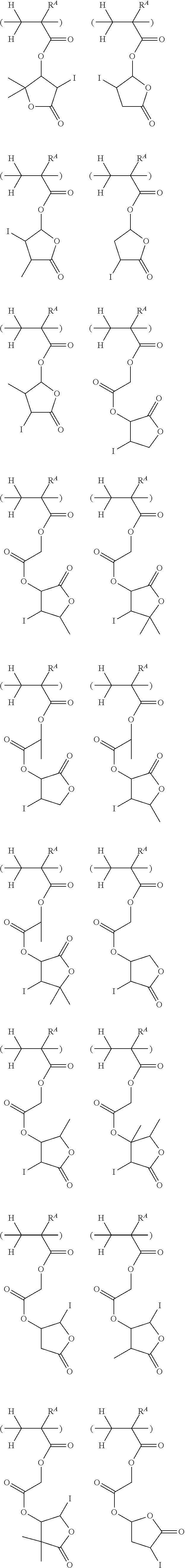

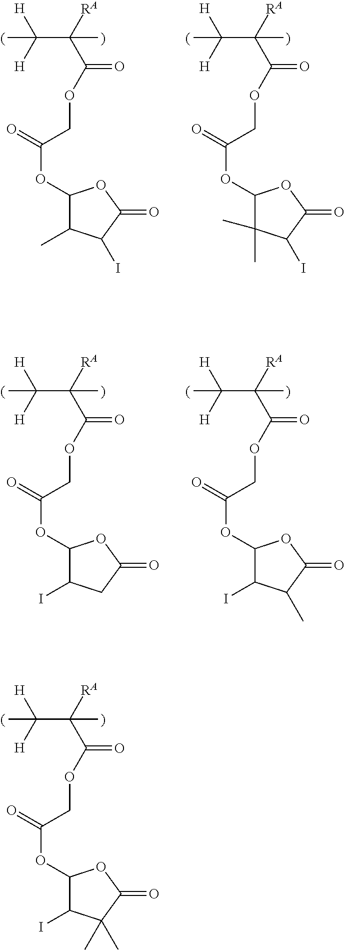

Example of the repeat unit (b2) are shown below, but not limited thereto. Herein R^{A} is as defined above.

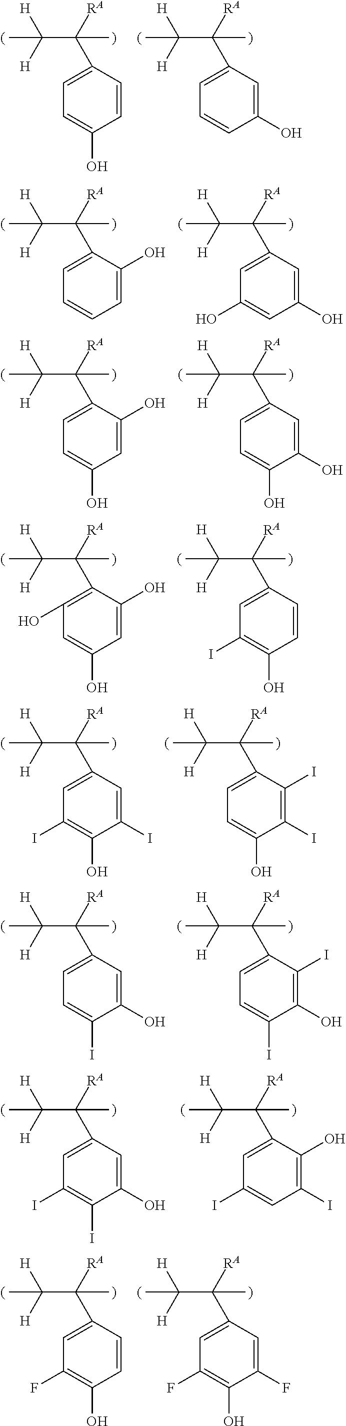

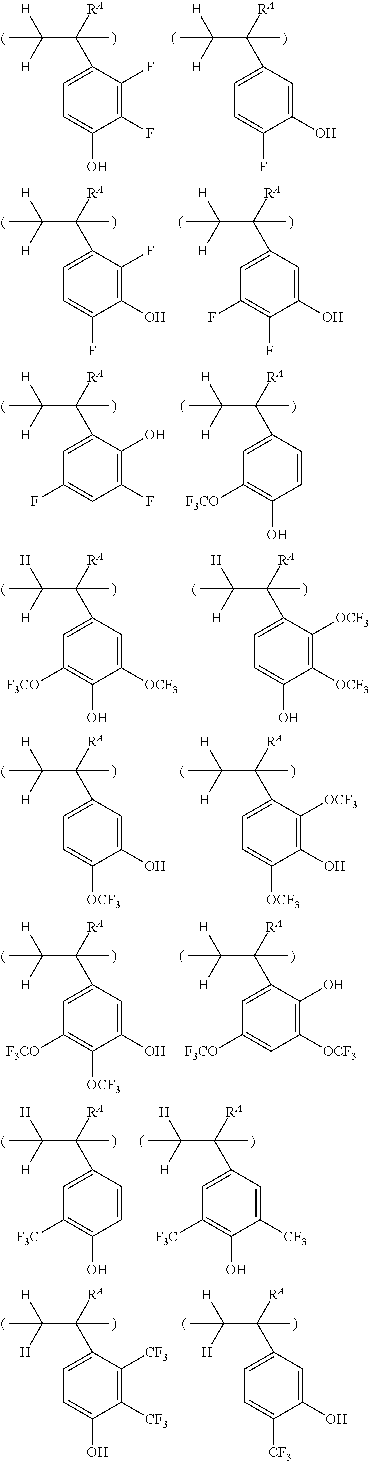

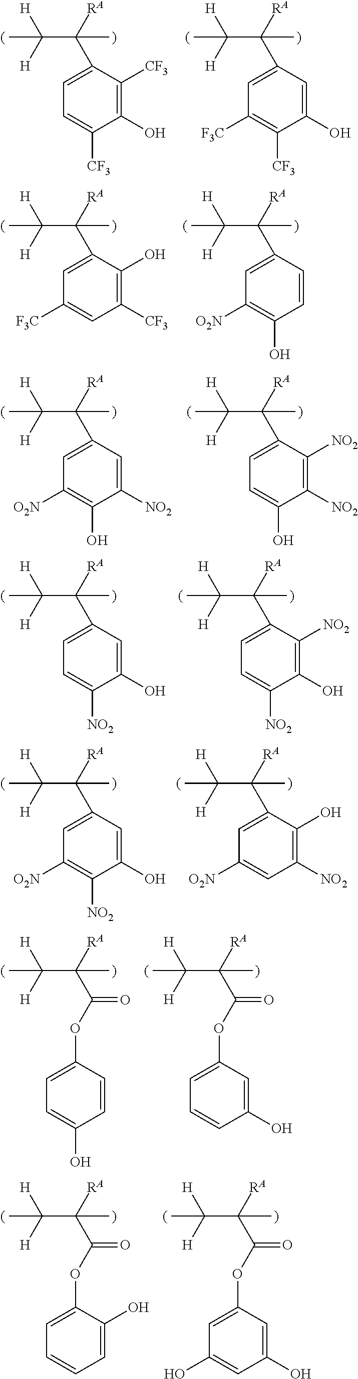

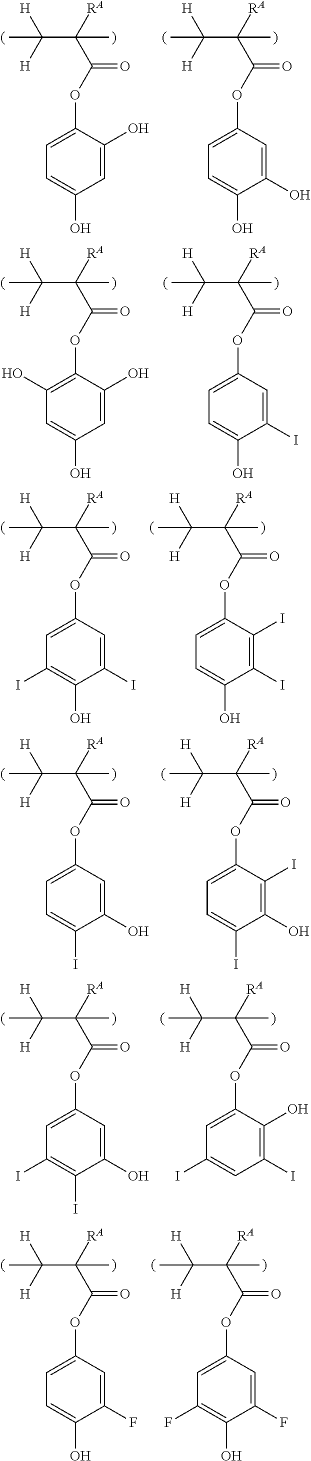

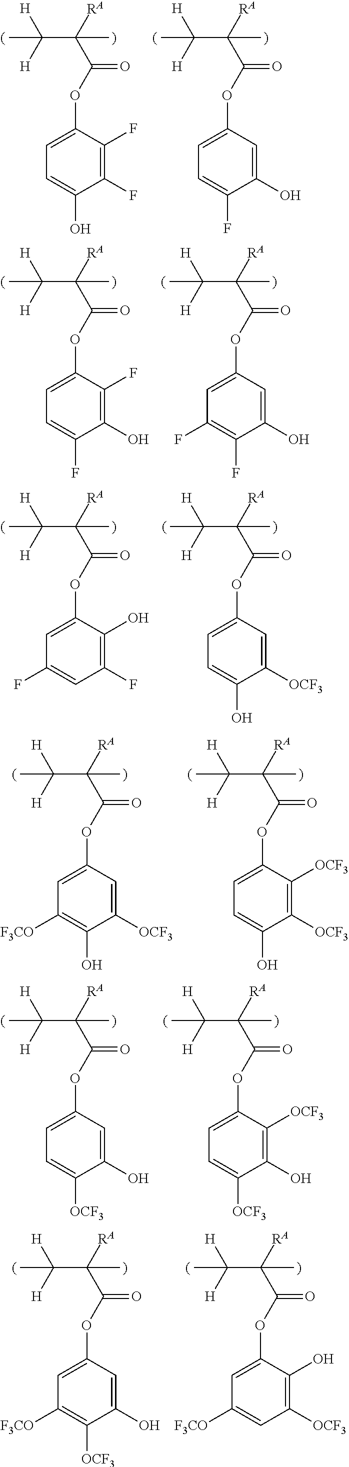

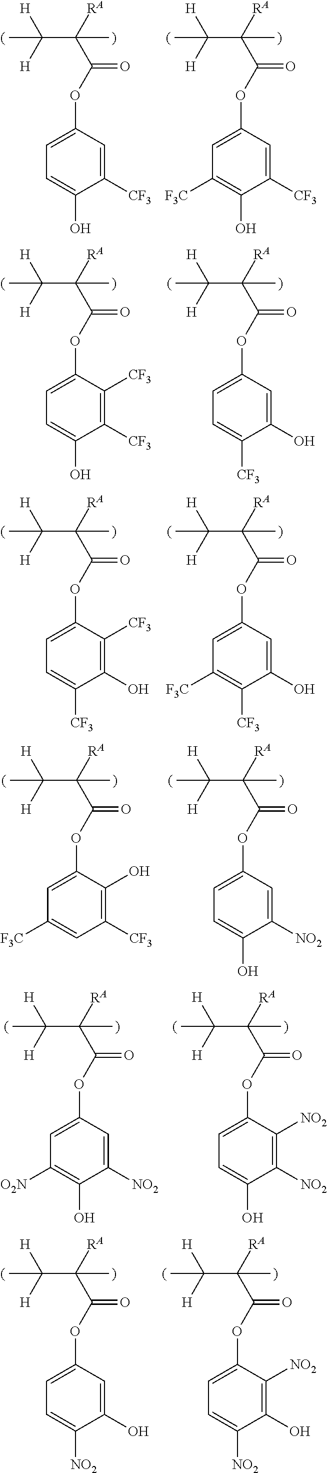

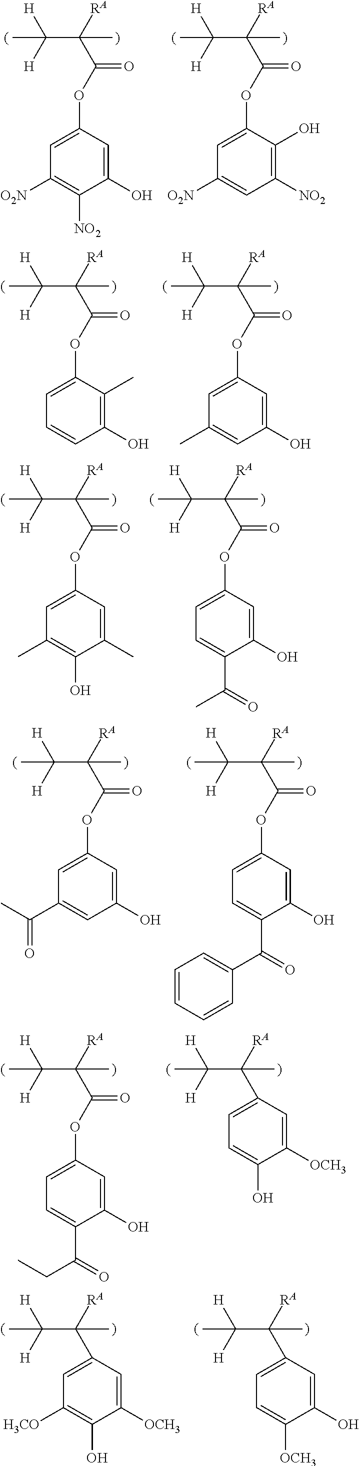

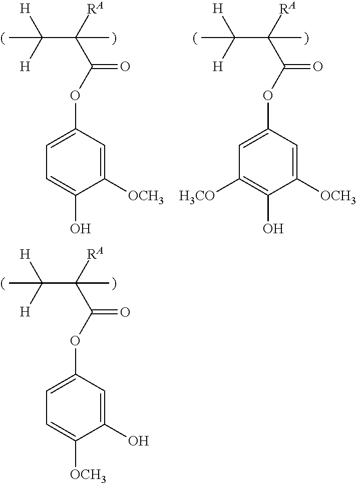

Of the repeat units (b1) and (b2), those units having a lactone ring as the polar group are preferred in the case of ArF lithography, and those units having a phenol site as the polar group are preferred in the case of KrF, EB or EUV lithography.

In a preferred embodiment, the base polymer further comprises repeat units of at least one type selected from repeat units having the formulae (c1) to (c4), which are also referred to as repeat units (c1) to (c4).

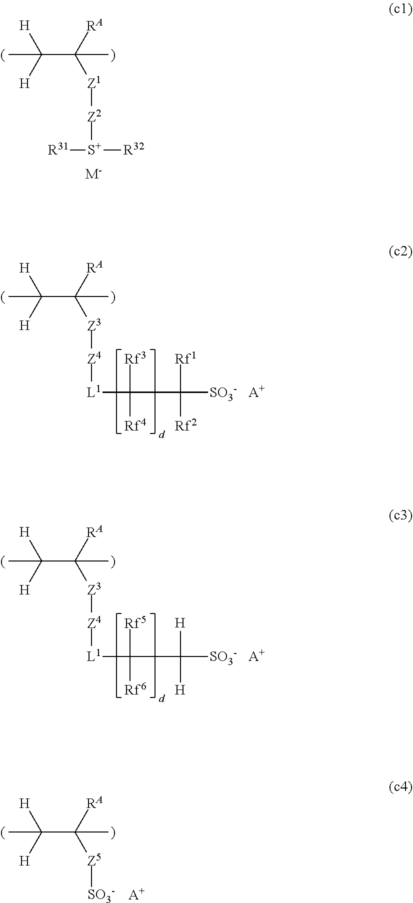

In formulae (c1) to (c4), R^{A} is each independently hydrogen, fluorine, methyl or trifluoromethyl. Z¹ is a single bond or phenylene group. Z² is *—C(═O)—O—Z²¹—, *—C(═O)—NH—Z²¹—, or*—O═Z²¹—, wherein Z²¹ is a C₁-C₆ aliphatic hydrocarbylene group, phenylene, or divalent group obtained by combining the foregoing, which may contain a carbonyl moiety, ester bond, ether bond or hydroxy moiety. Z³ is a single bond, phenylene group, naphthylene group or*—C(═O)—O—Z³¹—, wherein Z³¹ is a C₁-C₁₀ aliphatic hydrocarbylene group which may contain a hydroxy moiety, ether bond, ester bond or lactone ring, or a phenylene group or naphthylene group. Z⁴ is a single bond or*—Z⁴¹—C(═O)—O—, wherein Z⁴¹ is a C₁-C₂₀ hydrocarbylene group which may contain a heteroatom. Z⁵ is a single bond, methylene, ethylene, phenylene, fluorinated phenylene, trifluoromethyl-substituted phenylene. *—C(═O)—O—Z⁵¹—, *—C(═O)—N(H)—Z⁵¹— or*—O—Z⁵¹—, wherein Z⁵¹ is a C₁-C₆ aliphatic hydrocarbylene group, phenylene, fluorinated phenylene or trifluoromethyl-substituted phenylene group, which may contain a carbonyl moiety, ester bond, ether bond or hydroxy moiety. The asterisk (*) designates a point of attachment to the carbon atom in the backbone.

The aliphatic hydrocarbylene group represented by Z²¹, Z³¹ and Z⁵¹ may be straight, branched or cyclic. Examples thereof include alkanediyl groups such as methanediyl, ethane-1,1-diyl, ethane-1,2-diyl, propane-1,1-diyl, propane-1,2-diyl, propane-1,3-diyl, propane-2,2-diyl, butane-1,1-diyl, butane-1,2-diyl, butane-1,3-diyl, butane-2,3-diyl, butane-1,4-diyl, 1,1-dimethylethane-1,2-diyl, pentane-1,5-diyl, 2-methylbutane-1,2-diyl, and hexane-1,6-diyl; cycloalkanediyl groups such as cyclopropanediyl, cyclobutanediyl, cyclopentanediyl, and cyclohexanediyl; and combinations thereof.

The hydrocarbylene group Z⁴¹ may be saturated or unsaturated and straight, branched or cyclic. Examples thereof are shown below, but not limited thereto.

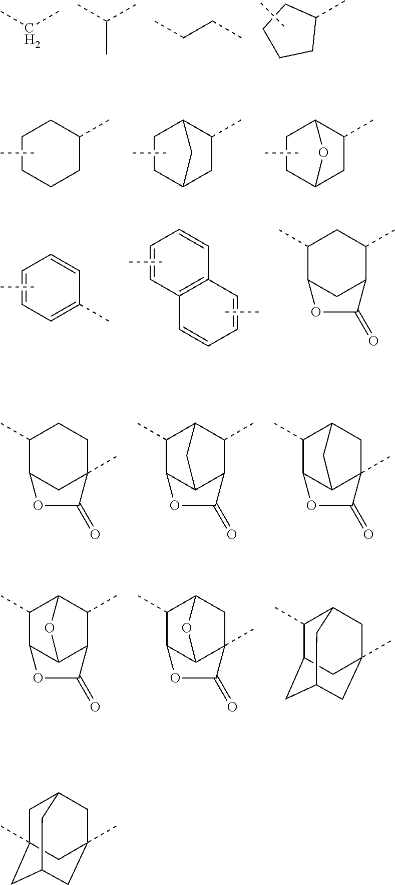

In formula (c1), R³¹ and R³² are each independently a C₁-C₂₀ hydrocarbyl group which may contain a heteroatom. The hydrocarbyl group may be saturated or unsaturated and straight, branched or cyclic. Examples thereof include C₁-C₂₀ alkyl groups such as methyl, ethyl, n-propyl, isopropyl, n-butyl, and tert-butyl: C₃-C₂₀ cyclic saturated hydrocarbyl groups such as cyclopropyl, cyclopentyl, cyclohexyl, cyclopropylmethyl. 4-methylcyclohexyl, cyclohexylmethyl, norbornyl, and adamantyl; C₂-C₂₀ alkenyl groups such as vinyl, allyl, propenyl, butenyl, and hexenyl; C₃-C₂₀ cyclic unsaturated hydrocarbyl groups such as cyclohexenyl; C₆-C₂₀ aryl groups such as phenyl, naphthyl, and thienyl; C₇-C₂₀ aralkyl groups such as benzyl, 1-phenylethyl and 2-phenylethyl; and combinations thereof. Inter alia, aryl groups are preferred. In the hydrocarbyl group, some or all of the hydrogen atoms may be substituted by a moiety containing a heteroatom such as oxygen, sulfur, nitrogen or halogen, or some constituent —CH₂— may be replaced by a moiety containing a heteroatom such as oxygen, sulfur or nitrogen, so that the group may contain a hydroxy, fluorine, chlorine, bromine, iodine, cyano, carbonyl, ether bond, ester bond, sulfonic ester bond, carbonate bond, lactone ring, sultone ring, carboxylic anhydride (—C(═O)—O—C(═O)—) or haloalkyl moiety.

Also, R³¹ and R³² may bond together to form a ring with the sulfur atom to which they are attached. Examples of the ring are as exemplified above for the ring that R^{f1} and R^{f2} in formula (cation-1), taken together, form with the sulfur atom to which they are attached.

Examples of the cation in repeat unit (c1) are shown below, but not limited thereto. Herein R^{A} is as defined above.

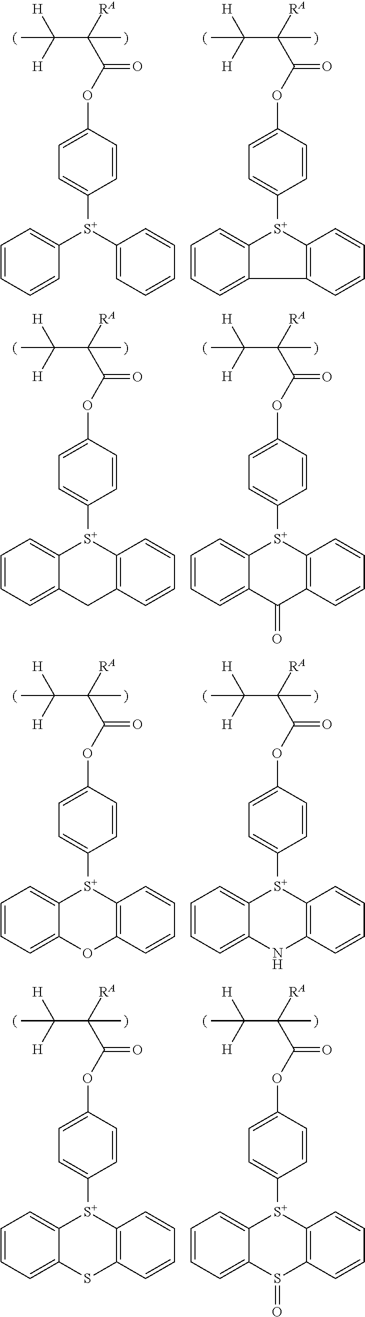

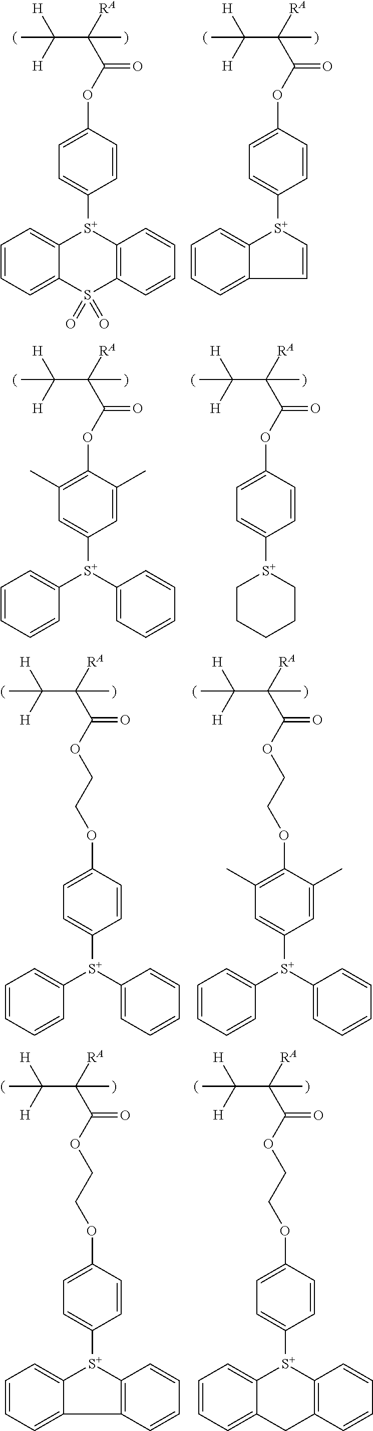

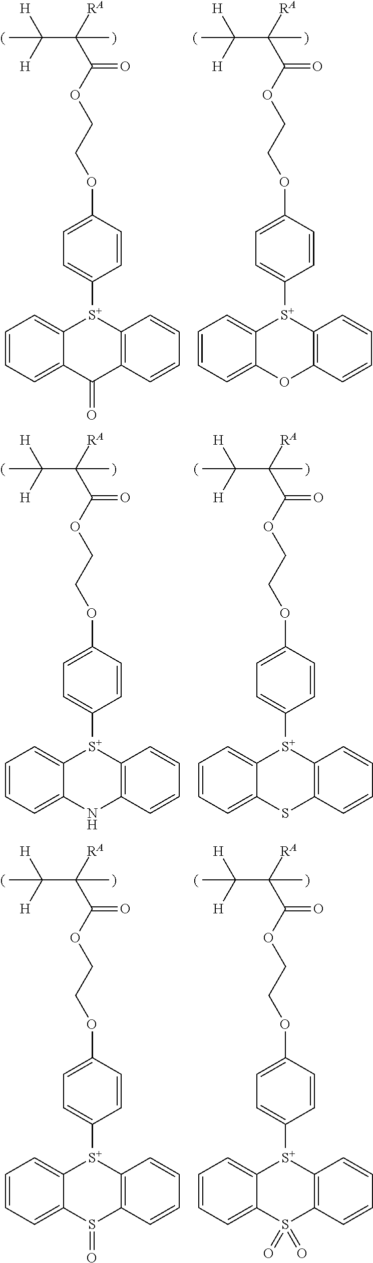

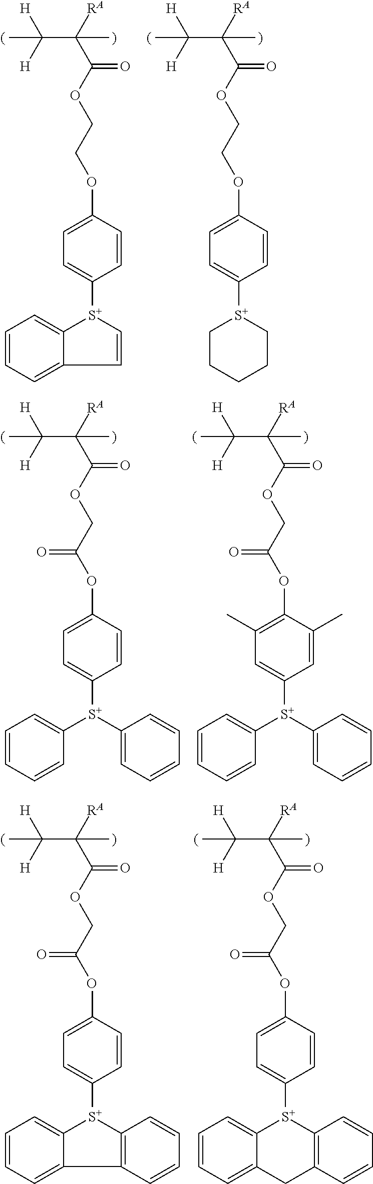

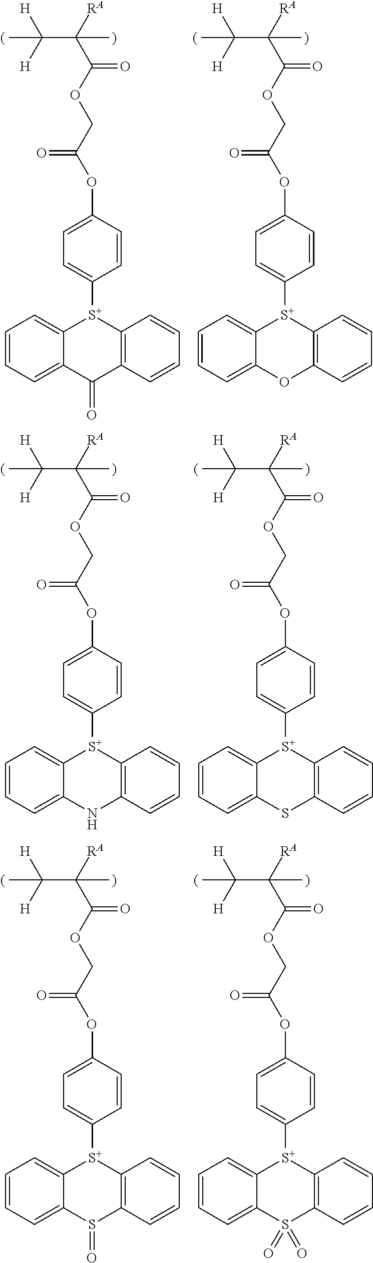

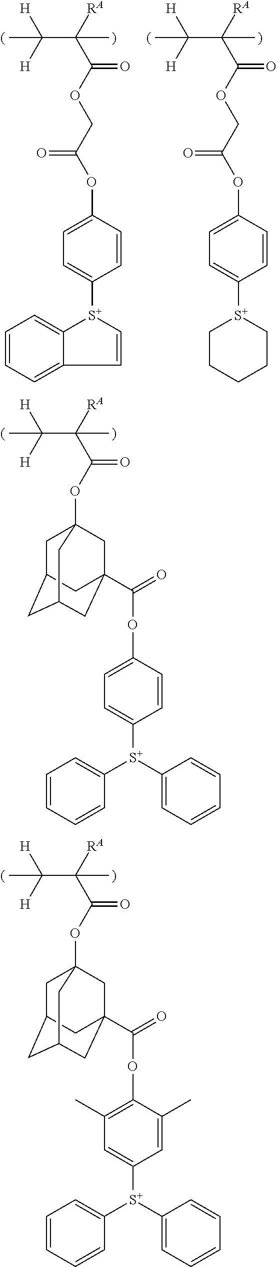

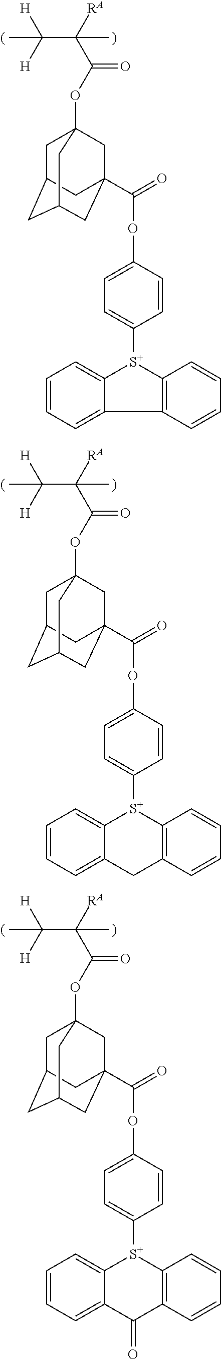

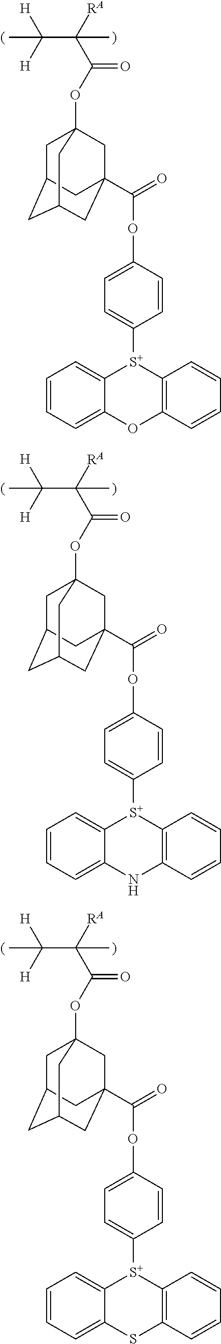

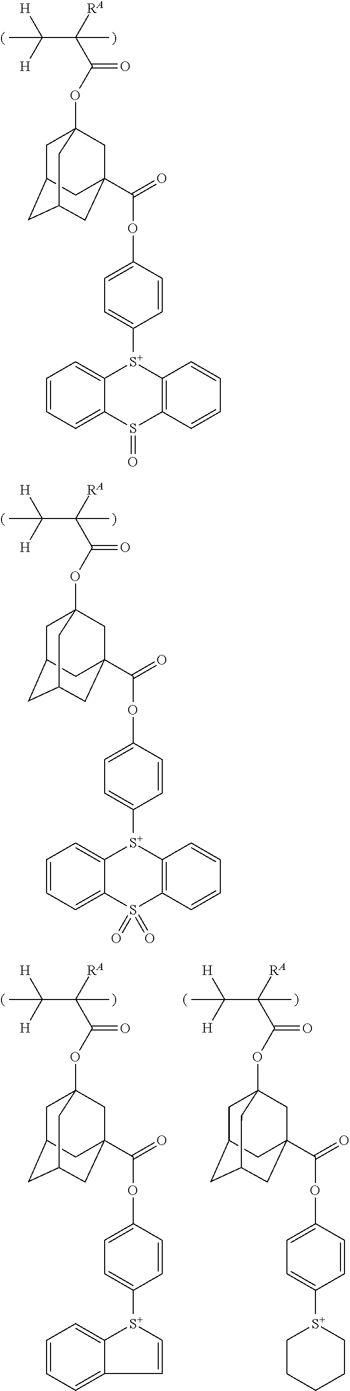

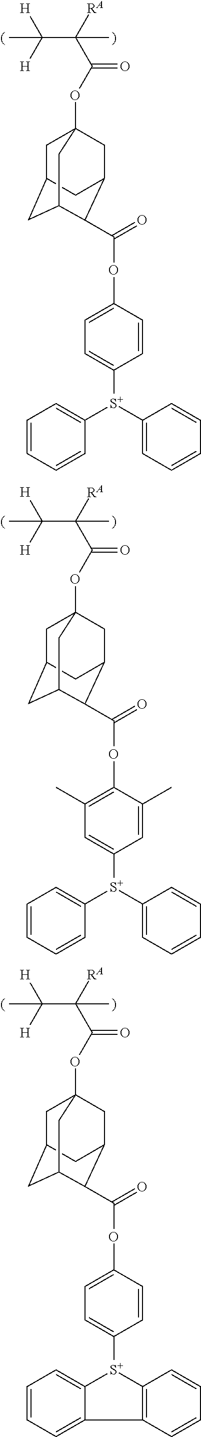

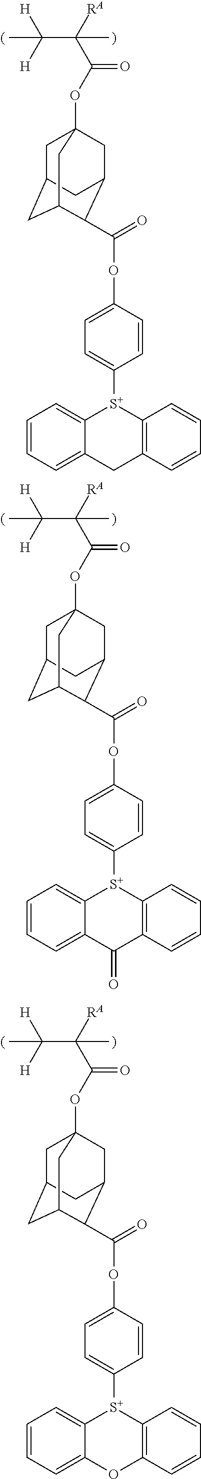

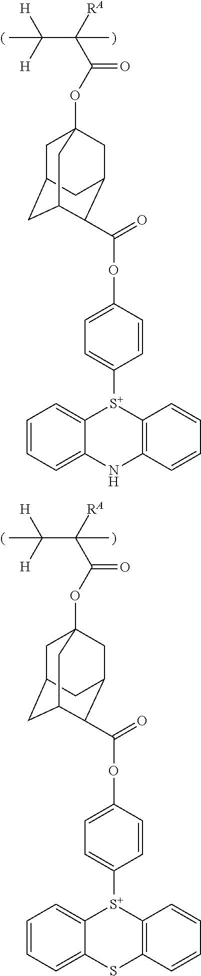

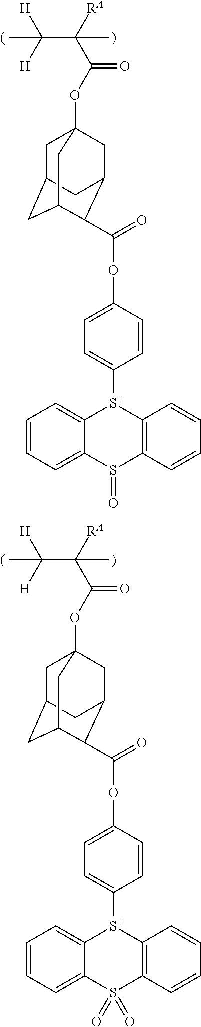

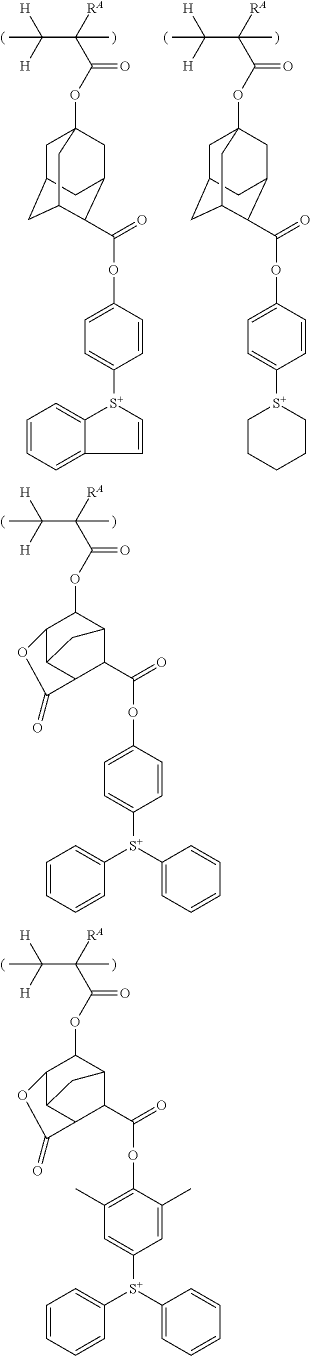

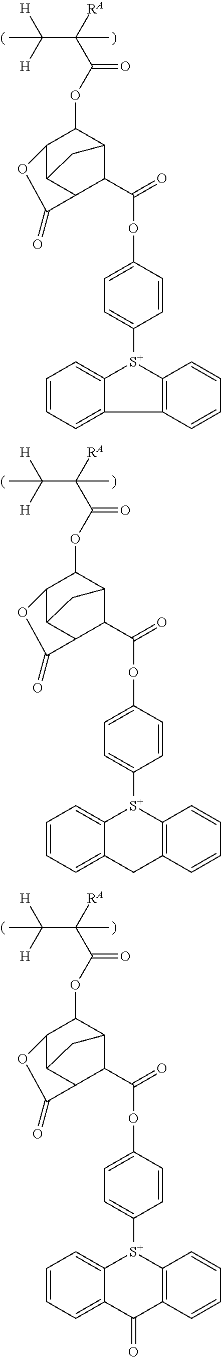

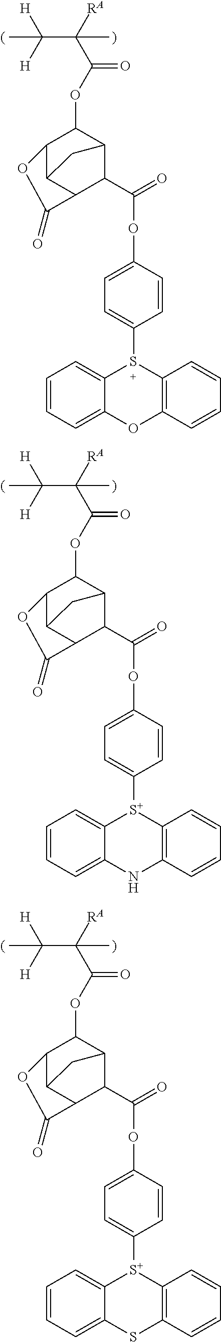

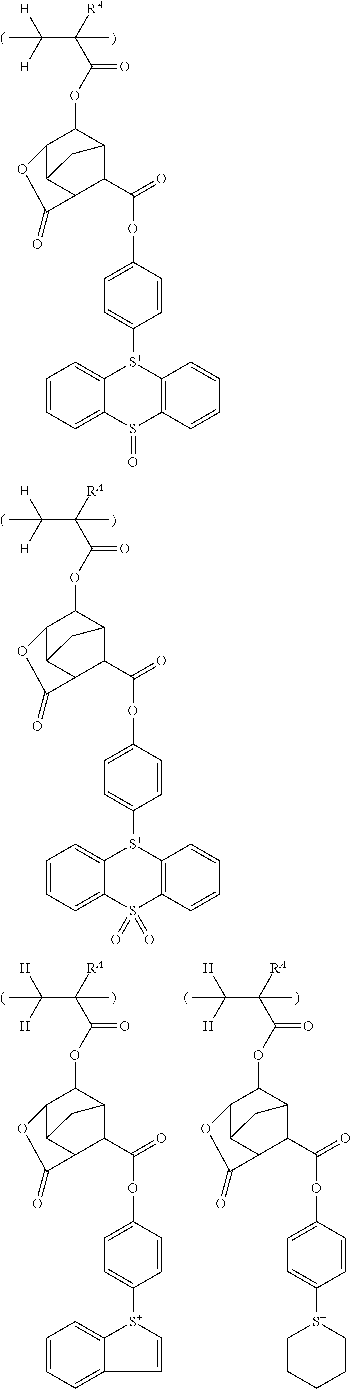

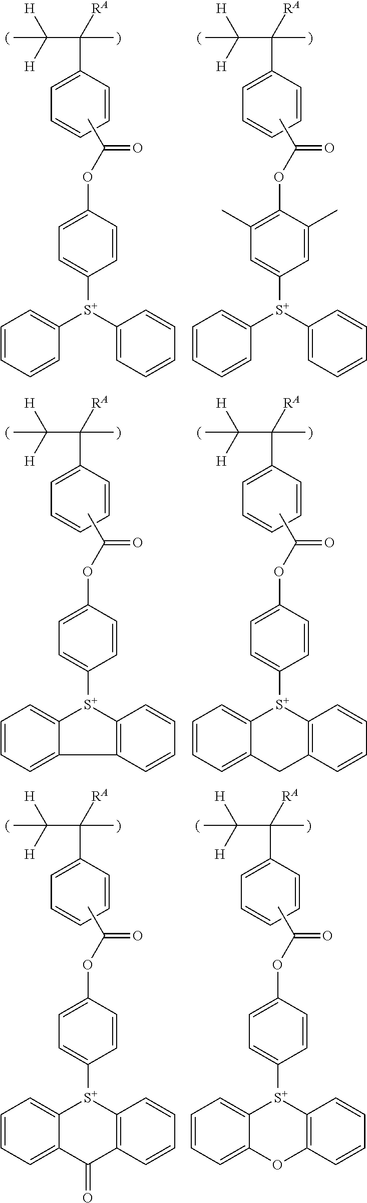

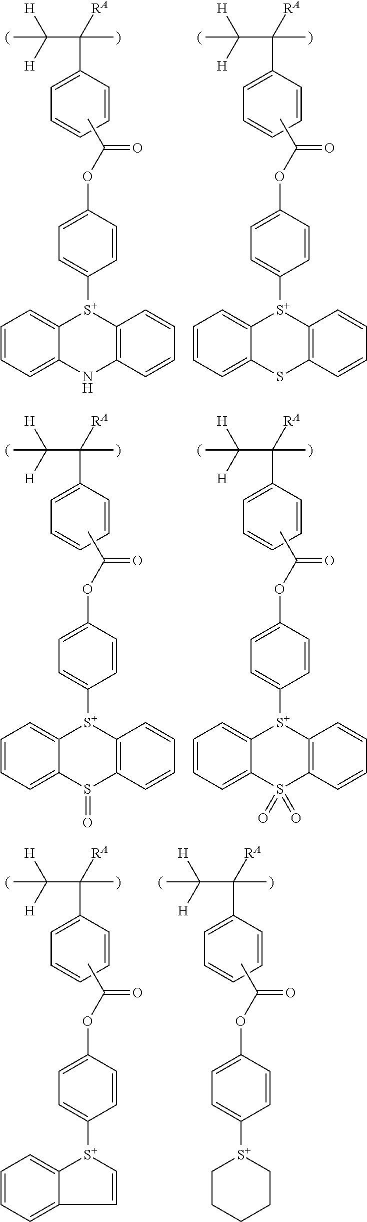

In formula (c1), M⁻ is a non-nucleophilic counter ion. Examples of the non-nucleophilic counter ion include halide ions such as chloride and bromide ions; fluoroalkylsulfonate ions such as triflate, 1,1,1-trifluoroethanesulfonate, and nonafluorobutanesulfonate; arylsulfonate ions such as tosylate, benzenesulfonate, 4-fluorobenzenesulfonate, and 1,2,3,4,5-pentafluorobenzenesulfonate; alkylsulfonate ions such as mesylate and butanesulfonate; imide ions such as bis(trifluoromethylsulfonyl)imide, bis(perfluoroethylsulfonyl)imide and bis(perfluorobutylsulfonyl)imide; methide ions such as tris(trifluoromethylsulfonyl)methide and tris(perfluoroethylsulfonyl)methide.

Also included are sulfonate anions having fluorine substituted at α-position as represented by the formula (c1-1) and sulfonate anions having fluorine substituted at α-position and trifluoromethyl at β-position as represented by the formula (c1-2).

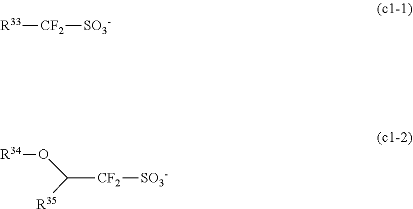

In formula (c1-1), R³³ is hydrogen, a C₁-C₃₀ hydrocarbyl group, C₂-C₃₀ hydrocarbylcarbonyloxy group, or C₂-C₃₀ hydrocarbyloxycarbonyl group. The hydrocarbyl group may contain halogen, ether bond, ester bond, carbonyl moiety, or lactone ring. The hydrocarbyl group and hydrocarbyl moiety in the hydrocarbylcarbonyloxy and hydrocarbyloxycarbonyl groups may be saturated or unsaturated and straight, branched or cyclic. Examples of the hydrocarbyl group are as will be exemplified later for R^{fa1} in formula (2A′).

In formula (c1-2), R³³ is hydrogen, or a C₁-C₃₀ hydrocarbyl group or C₂-C₃₀ hydrocarbylcarbonyl group, which may contain halogen, ether bond, ester bond, carbonyl moiety or lactone ring. R³⁵ is hydrogen, fluorine, or C₁-C₆ fluorinated alkyl group. The hydrocarbyl group and hydrocarbyl moiety in the hydrocarbylcarbonyl group may be saturated or unsaturated and straight, branched or cyclic. Examples of the hydrocarbyl group are as will be exemplified later for R^{fa1} in formula (2A′). R³⁵ is preferably trifluoromethyl.

Examples of the sulfonate anion having formula (c1-1) or (c1-2) are shown below, but not limited thereto. R³⁵ is as defined above.

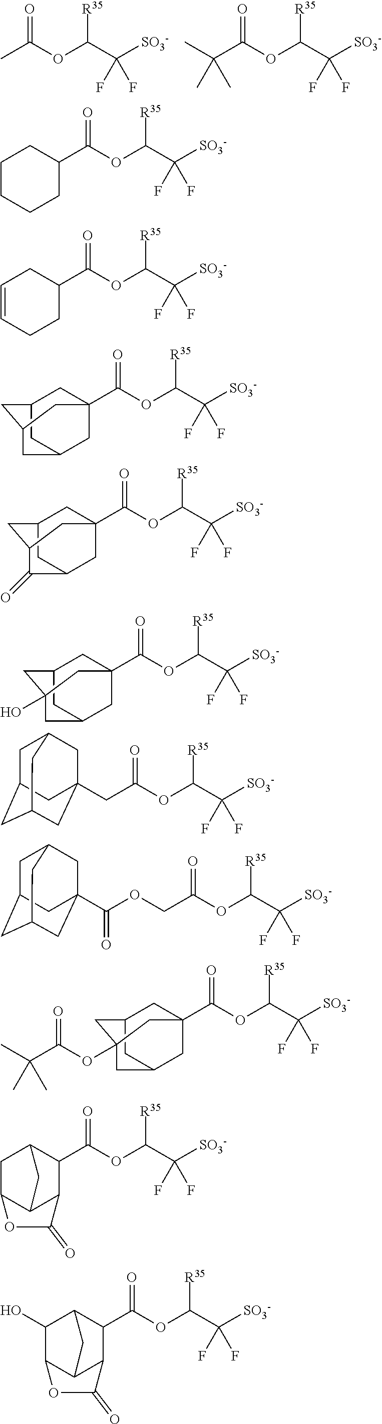

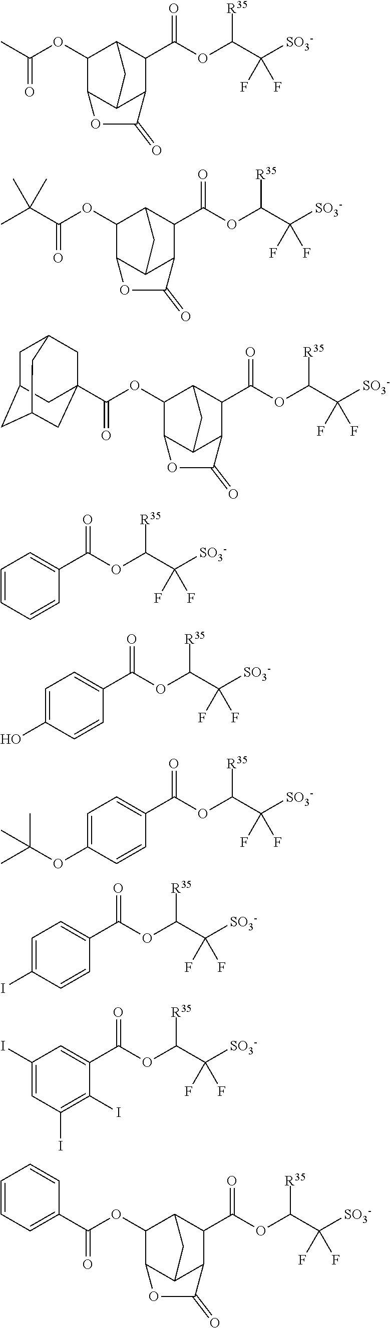

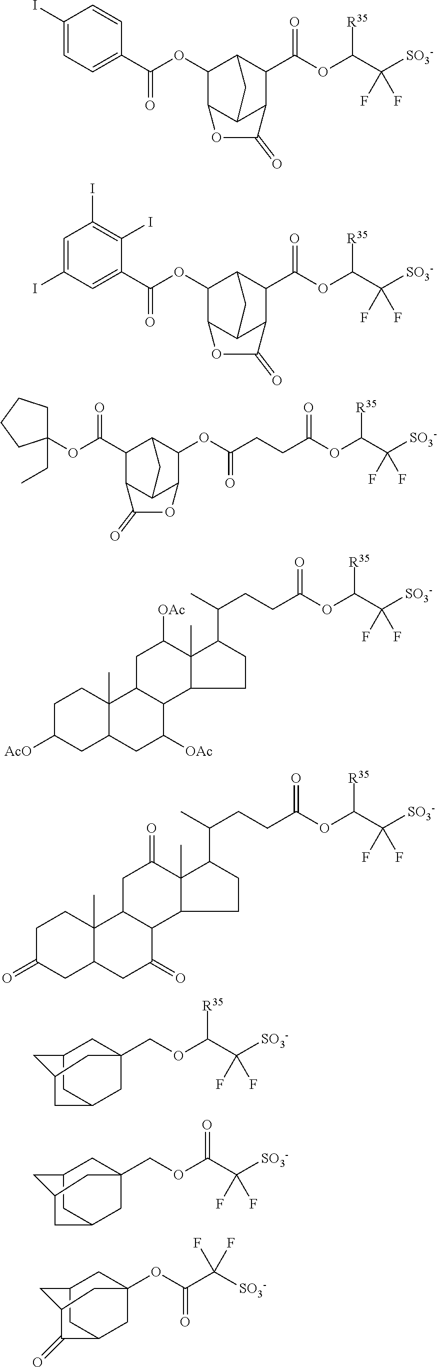

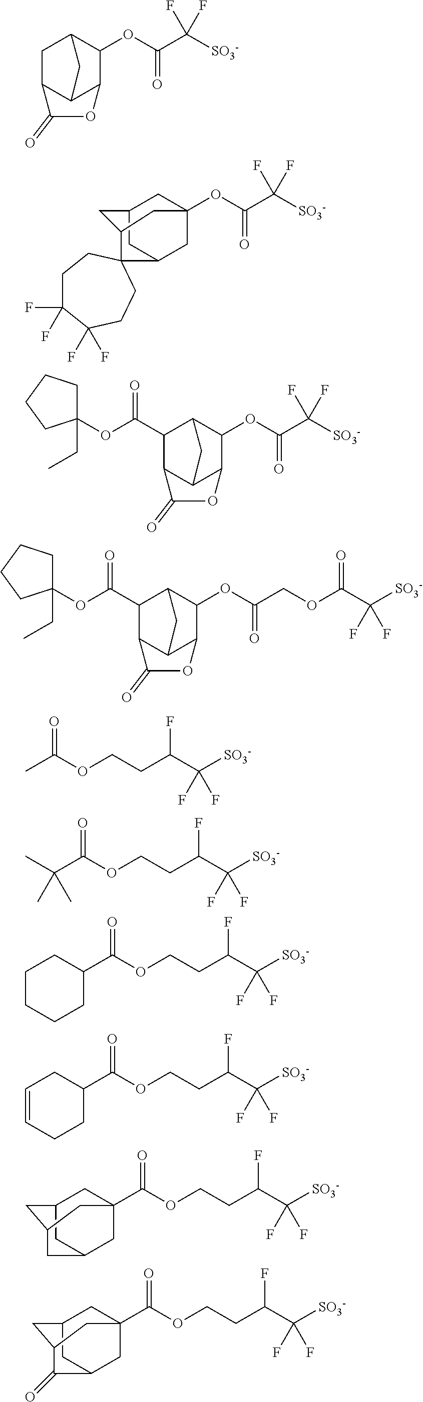

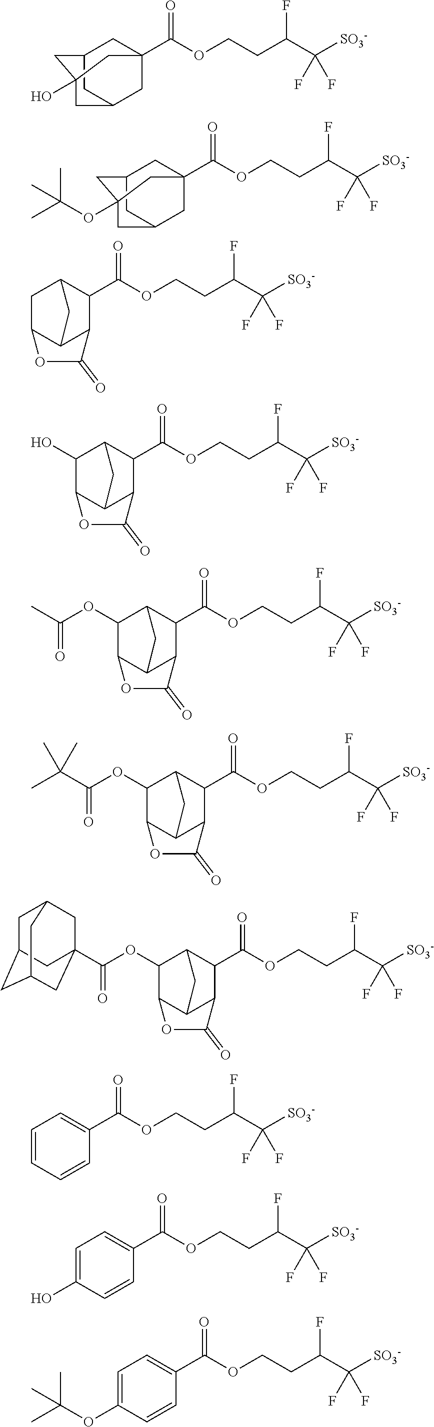

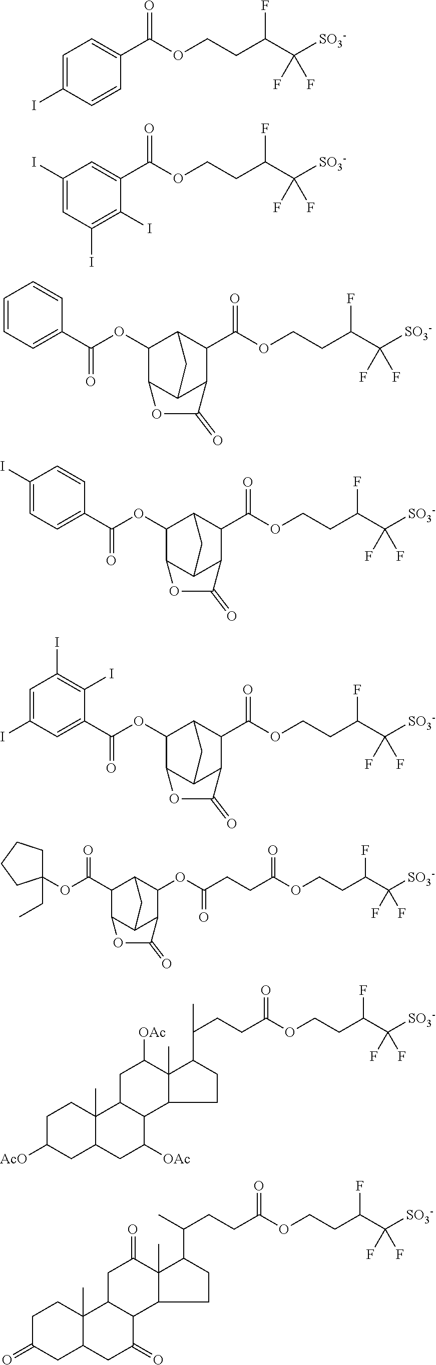

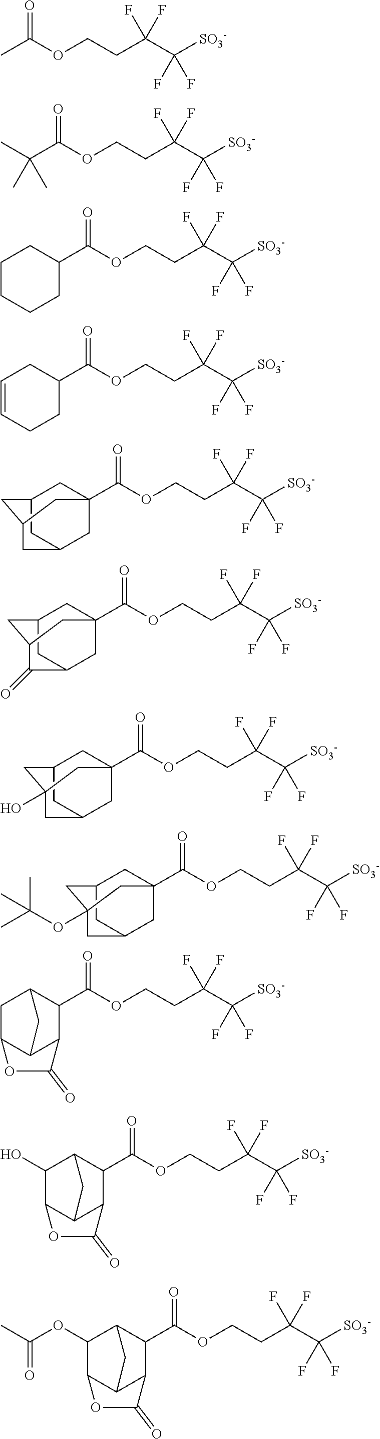

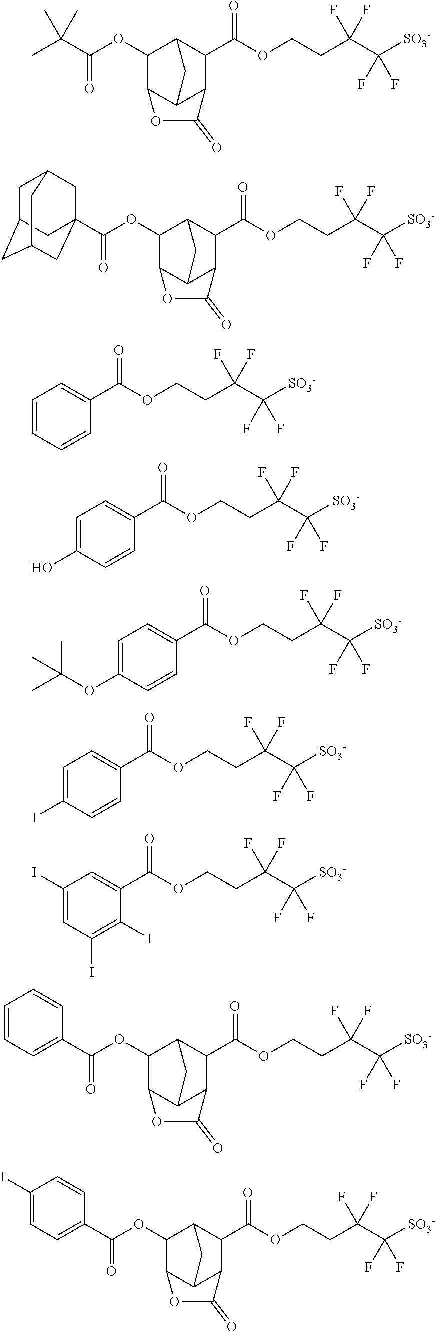

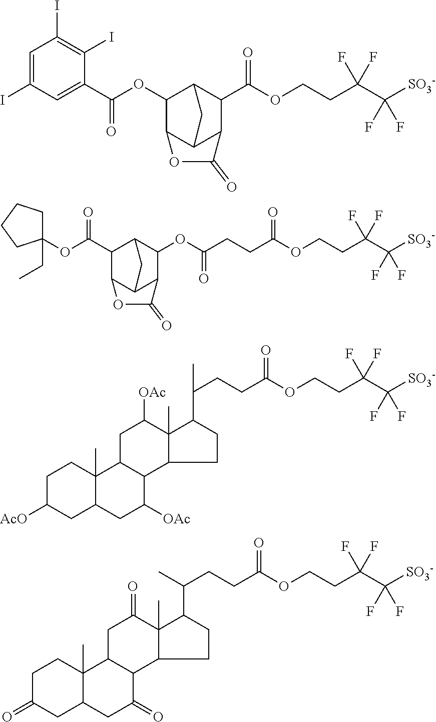

In formulae (c2) and (c3), L¹ is a single bond, ether bond, ester bond, carbonyl group, sulfonic ester bond, carbonate bond or carbamate bond. Of these, an ether bond, ester bond and carbonyl group are preferred from the aspect of synthesis, with an ester bond and carbonyl group being more preferred.

In formula (c2), Rf¹ and Rf² are each independently fluorine or a C₁-C₆ fluorinated alkyl group. It is preferred for enhancing the acid strength of the generated acid that both Rf¹ and Rf² be fluorine. Rf³ and Rf⁴ are each independently hydrogen, fluorine or a C₁-C⁶ fluorinated alkyl group. It is preferred for increasing solvent solubility that at least one of Rf³ and Rf⁴ be trifluoromethyl.

In formula (c3), Rf⁵ and Rf⁶ are each independently hydrogen, fluorine or a C₁-C₆ fluorinated alkyl group. It is noted that not all Rf⁵ and Rf⁶ are hydrogen at the same time. It is preferred for increasing solvent solubility that at least one of Rf⁵ and Rf⁶ be trifluoromethyl.

In formulae (c2) and (c3), d is an integer of 0 to 3, preferably 1.

Examples of the anion in repeat unit (c2) are shown below, but not limited thereto. R^{A} is as defined above.

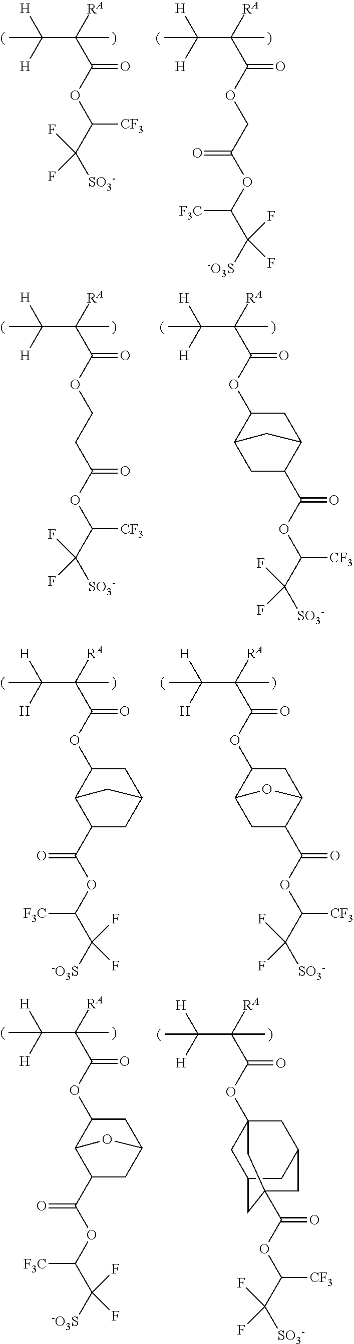

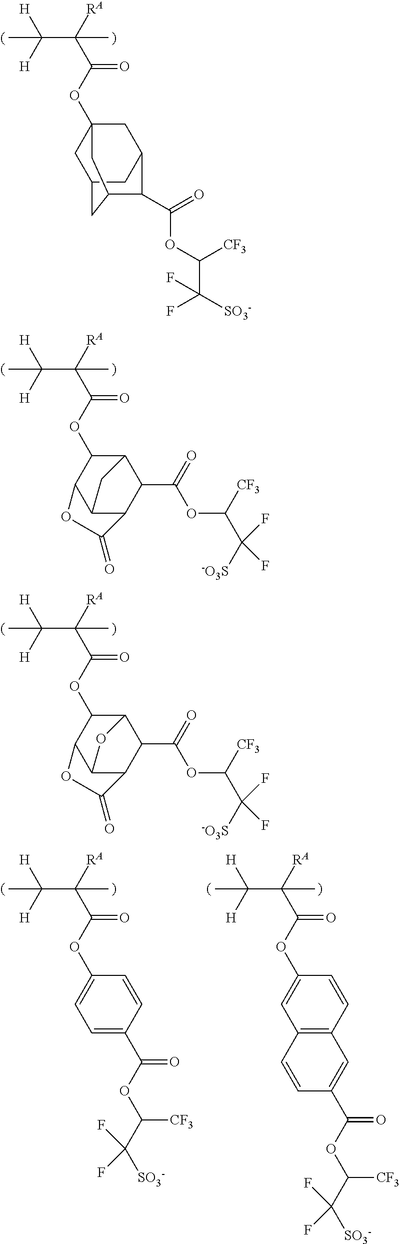

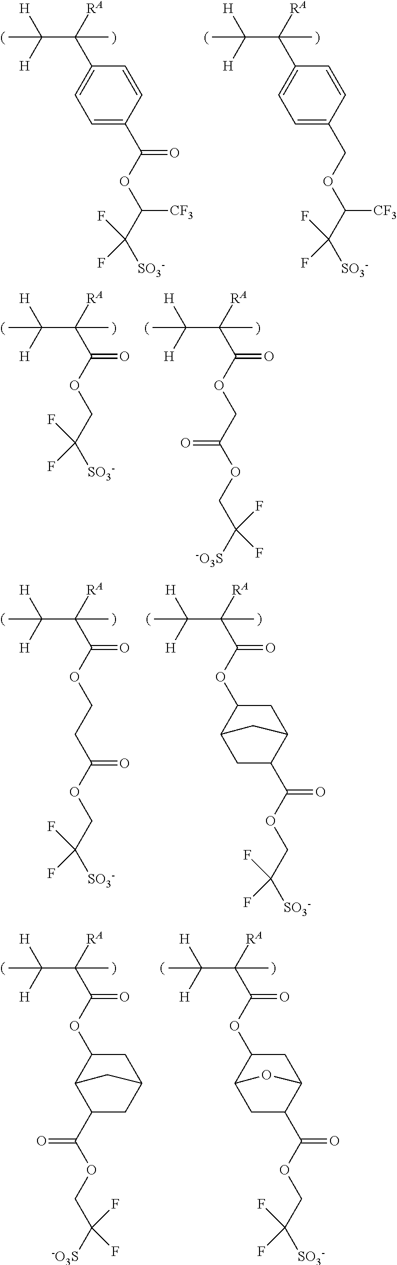

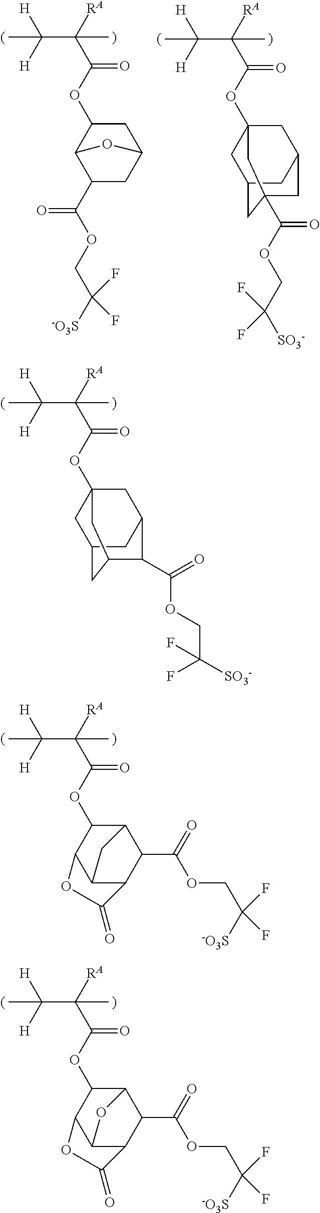

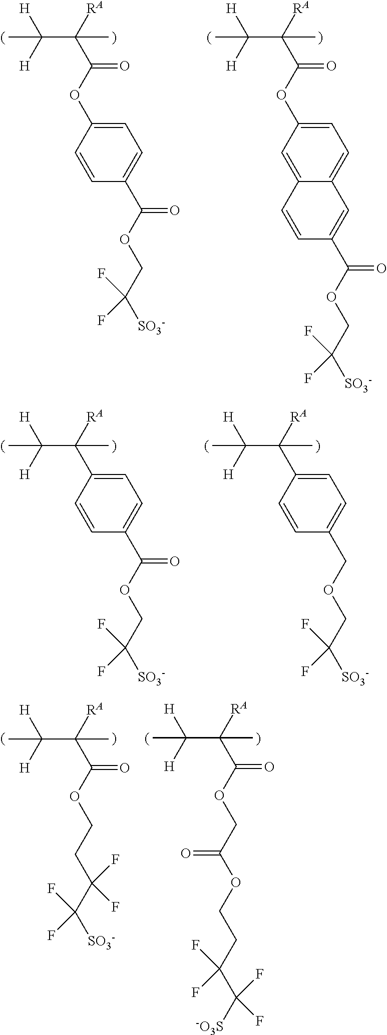

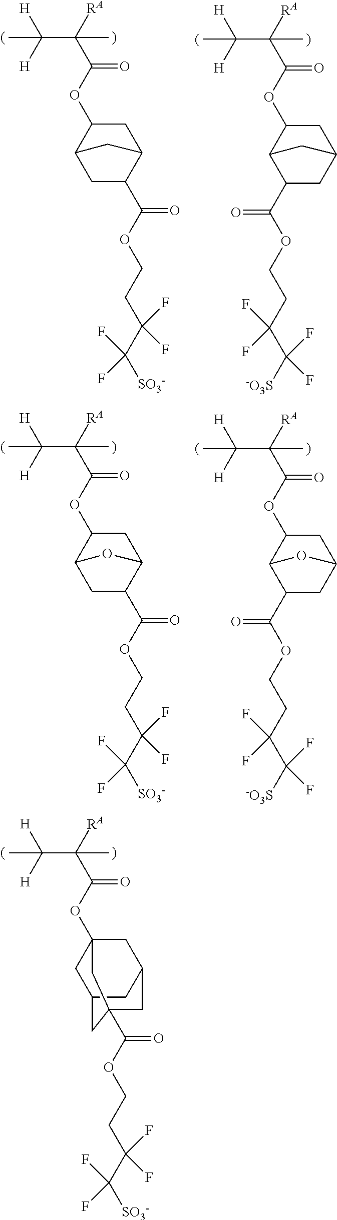

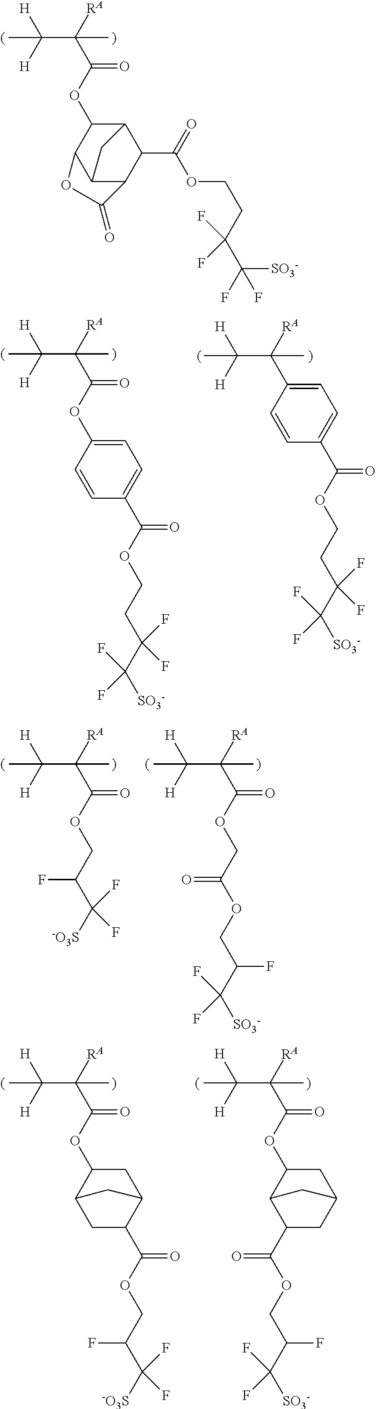

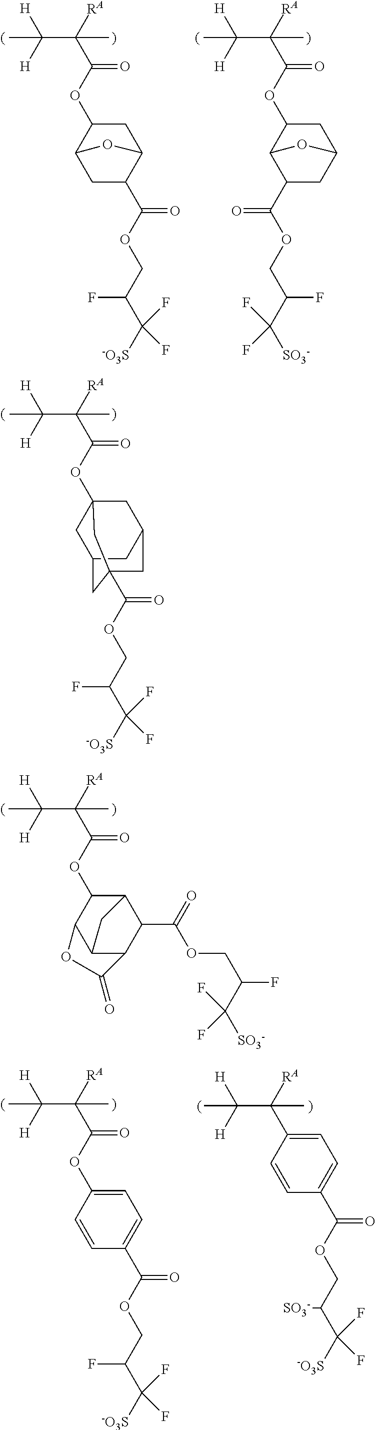

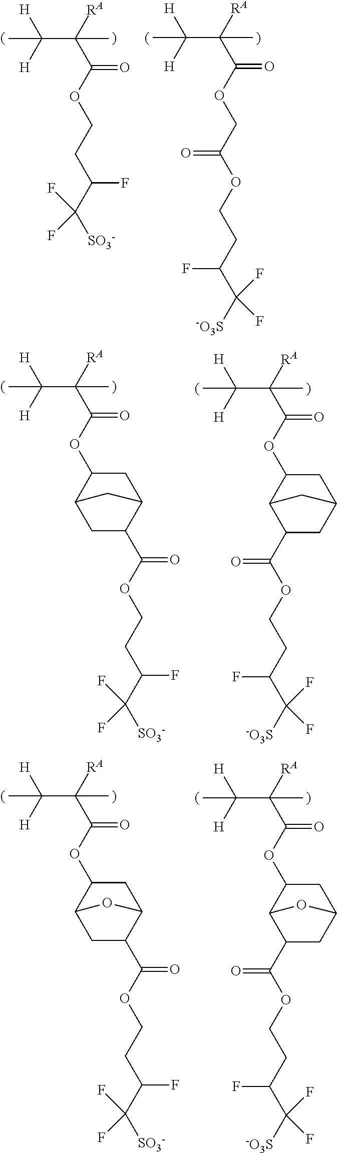

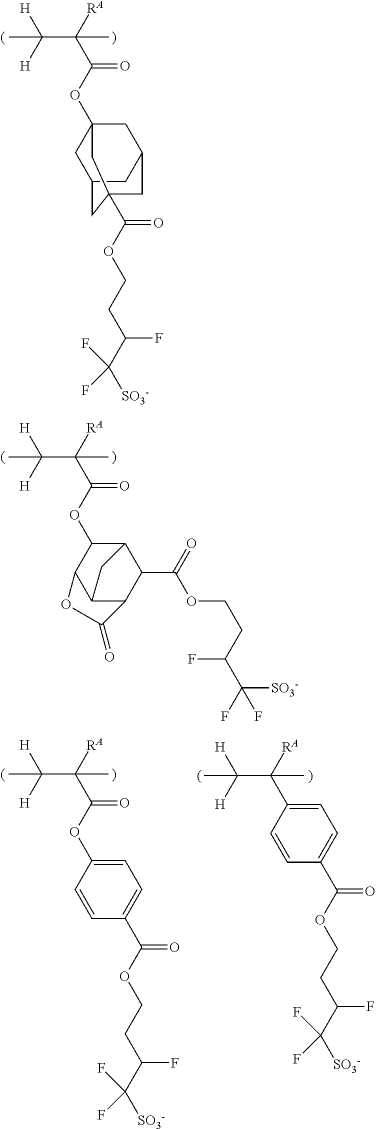

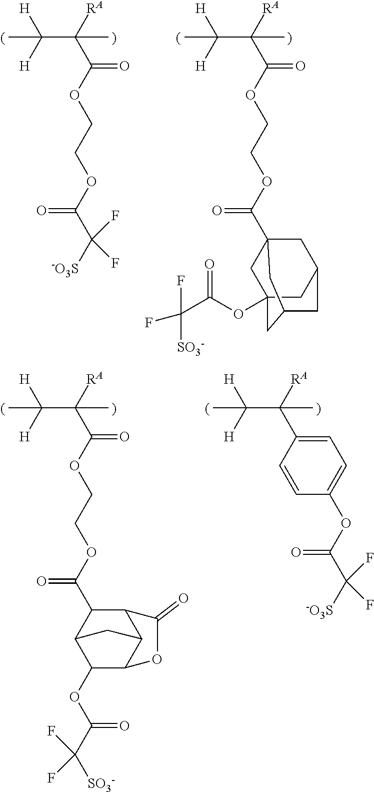

Examples of the anion in repeat unit (c3) are shown below, but not limited thereto. R^{A} is as defined above.

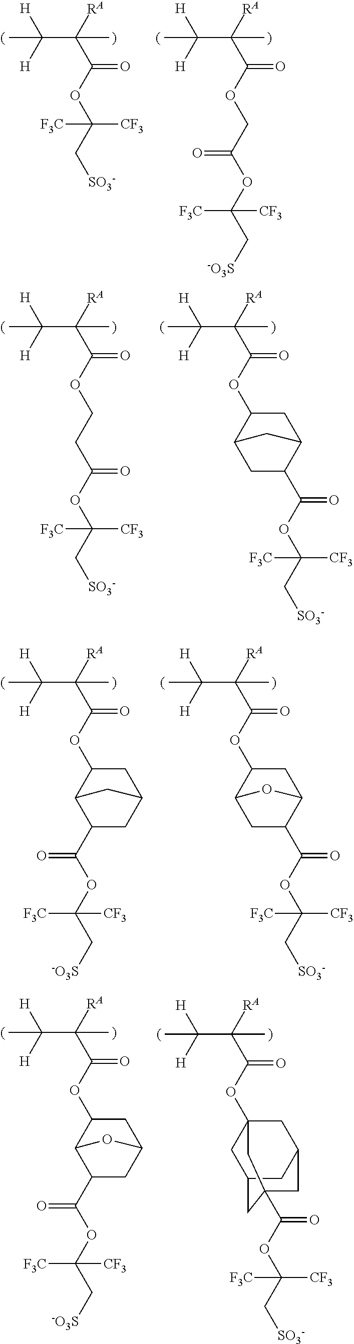

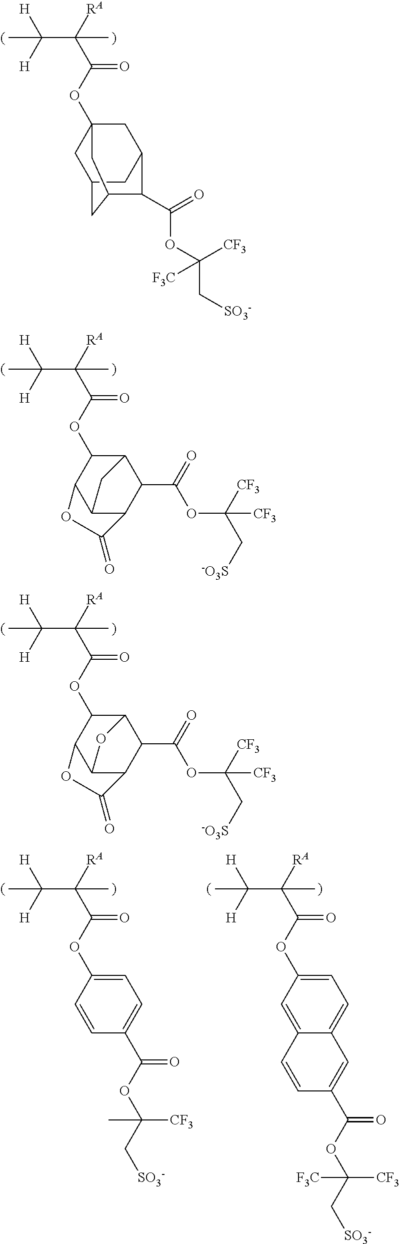

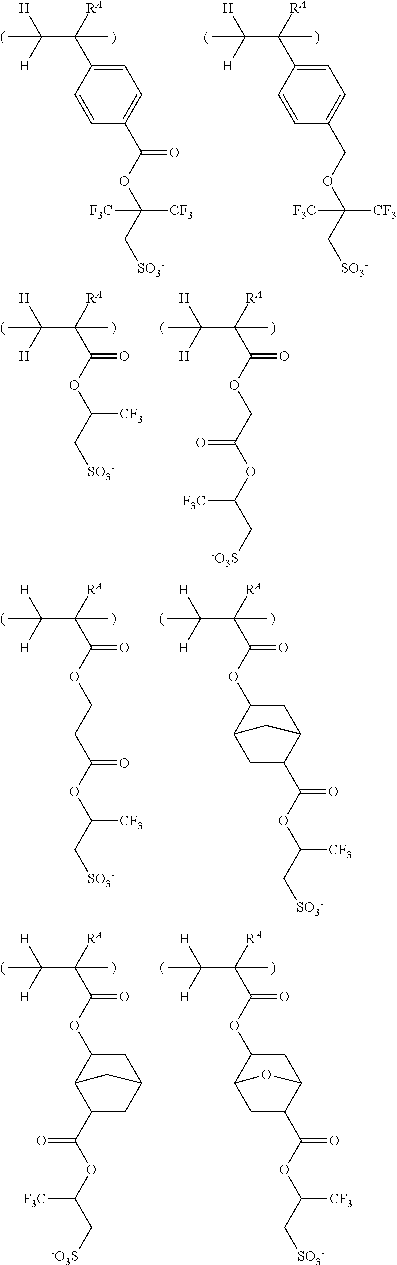

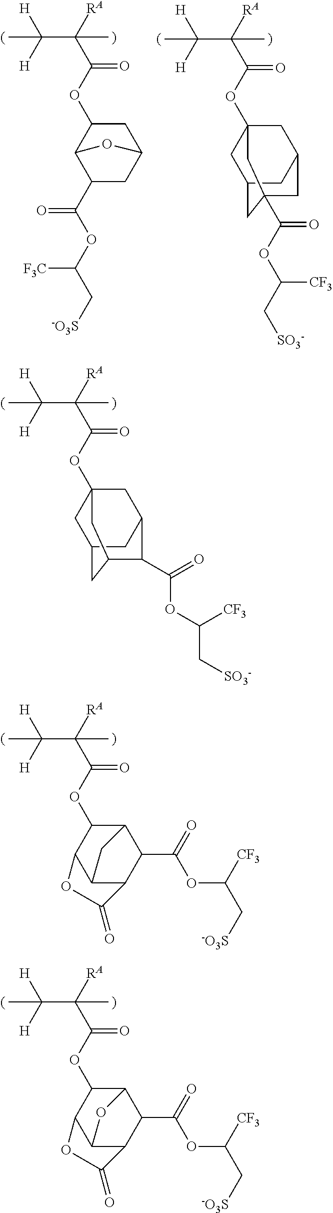

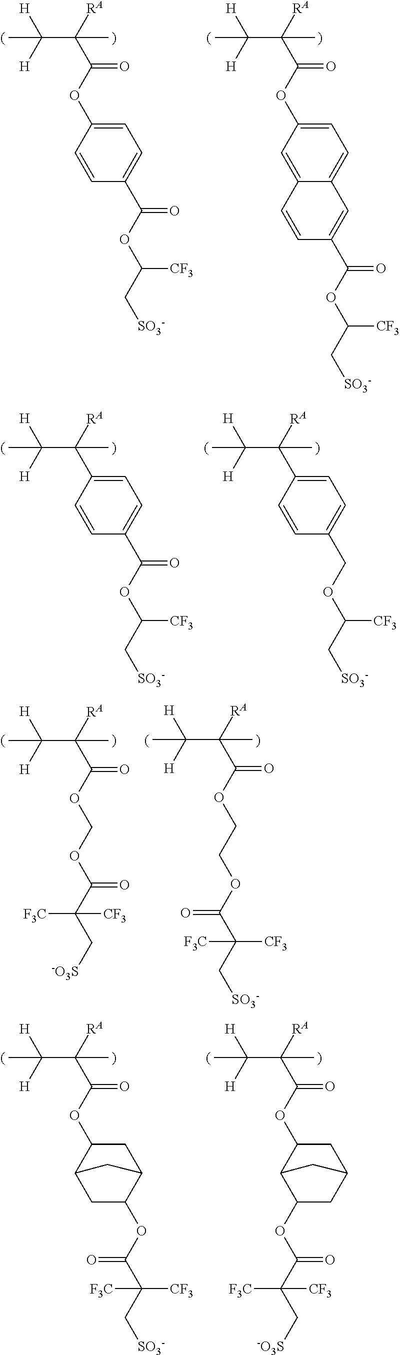

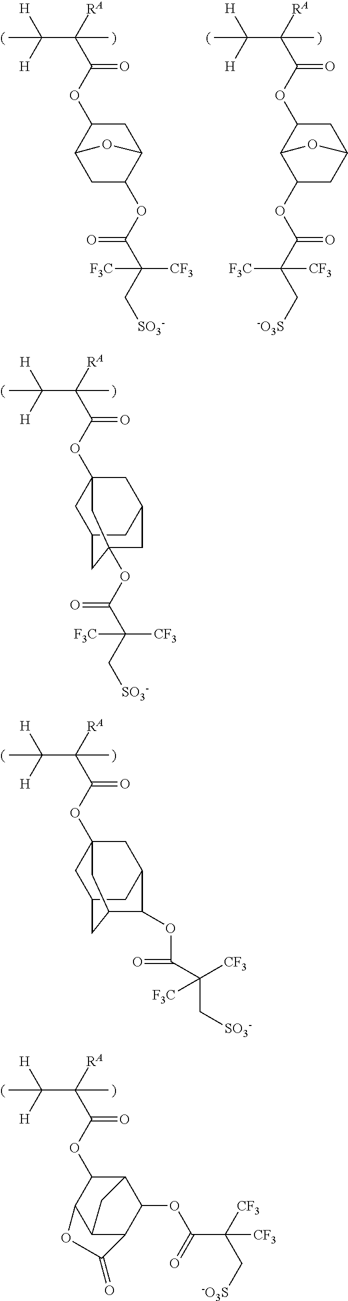

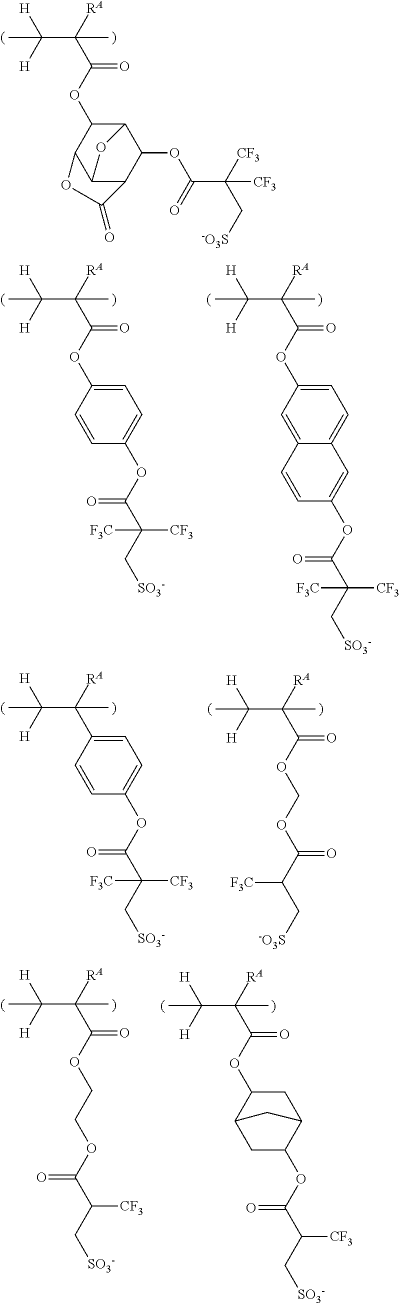

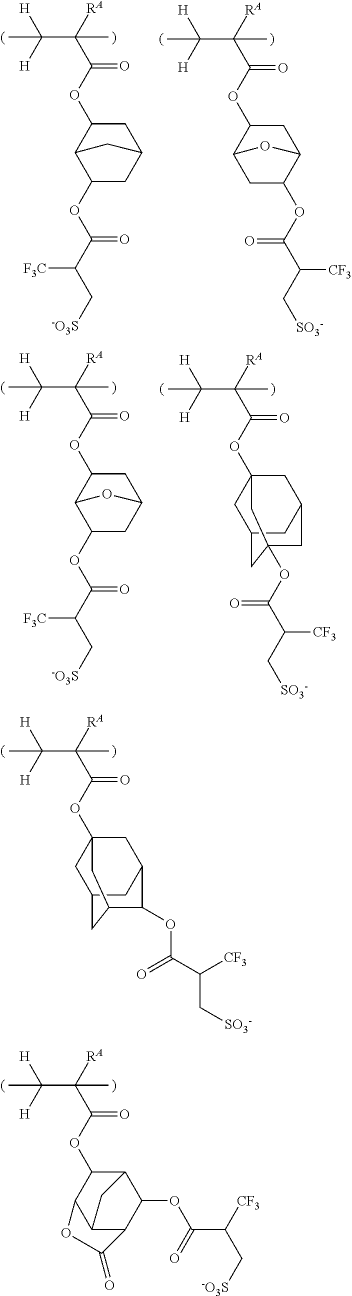

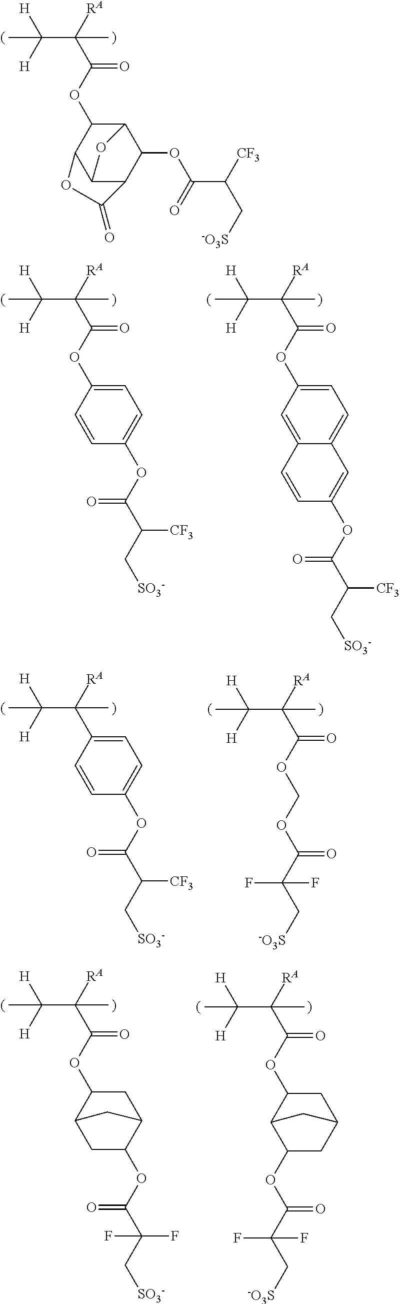

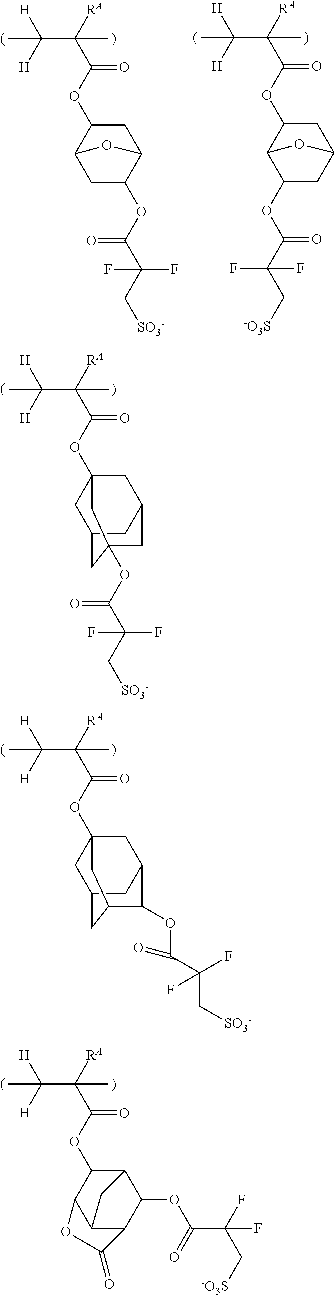

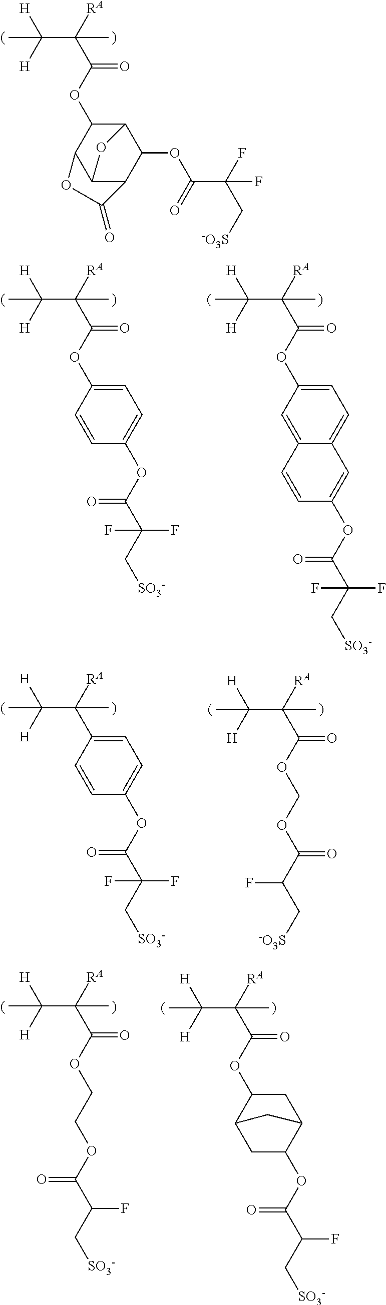

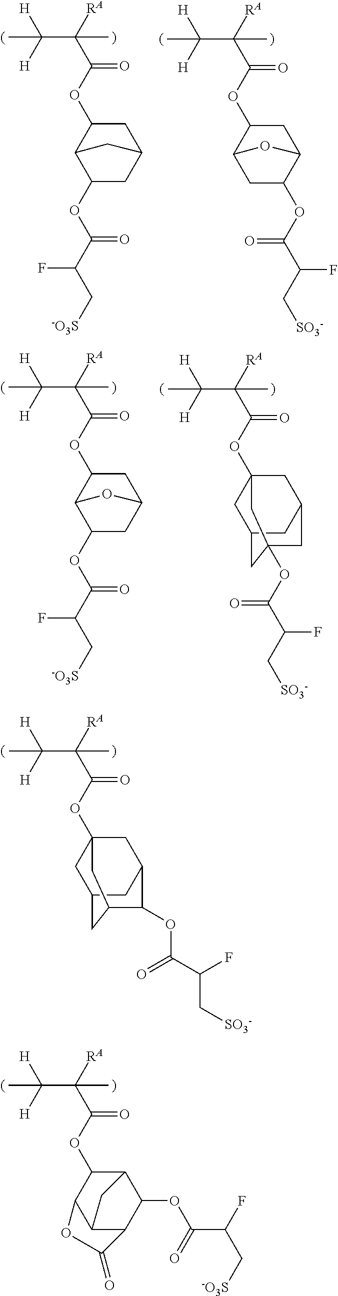

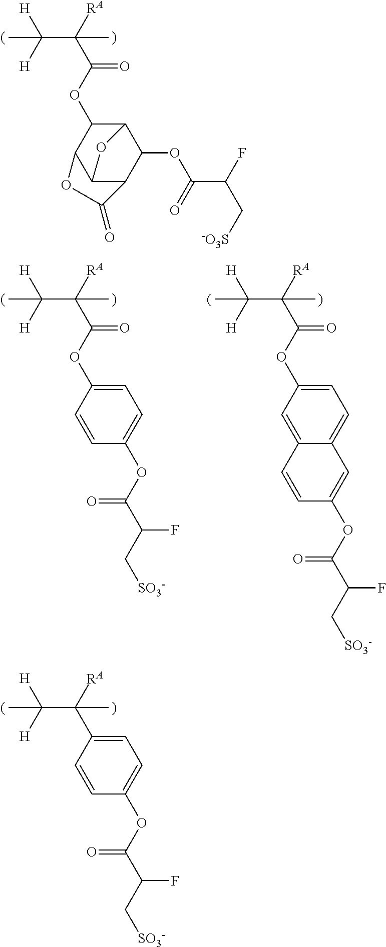

Examples of the anion in repeat unit (c4) are shown below, but not limited thereto. R^{A} is as defined above.

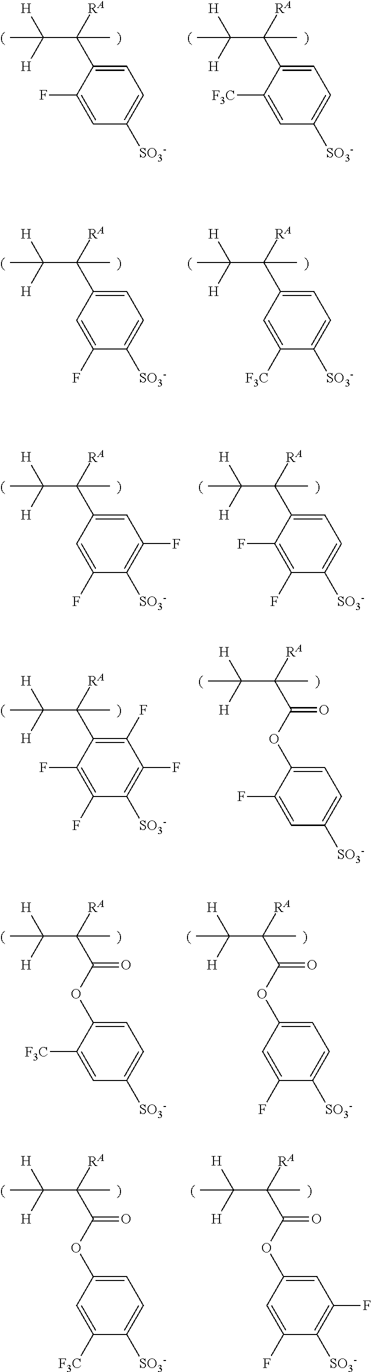

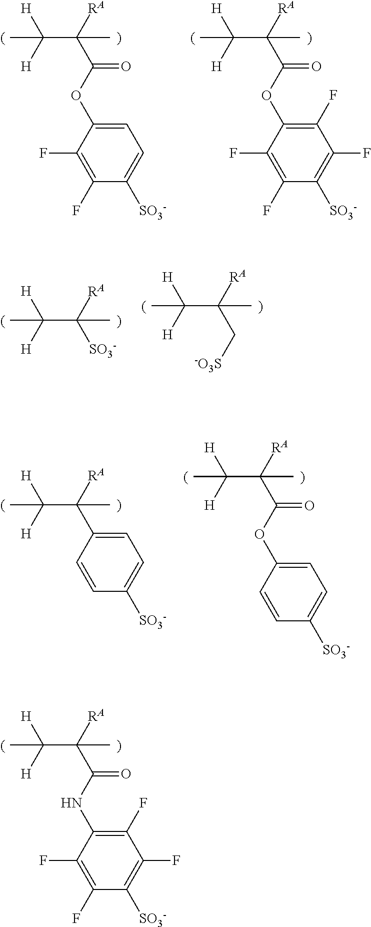

In formulae (c2) to (c4), A⁺ is an onium cation. Suitable onium cations include sulfonium, iodonium and ammonium cations, with sulfonium and iodonium cations being preferred. Specific structures thereof are as exemplified above for the cations having formulae (cation-1) to (cation-3).

The repeat units (c1) to (c4) function as a photoacid generator. Where a base polymer containing repeat units (c1) to (c4). i.e., polymer-bound acid generator is used, the resist composition may or may not contain (D) a photoacid generator to be described later.

The base polymer may further comprise repeat units (d) of a structure having a hydroxy group protected with an acid labile group. The repeat unit (d) is not particularly limited as long as the unit includes one or more structures having a hydroxy group protected with a protective group such that the protective group is decomposed to generate a hydroxy group under the action of acid. Repeat units having the formula (d1) are preferred.

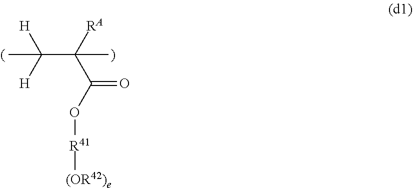

In formula (d1), R^{A} is as defined above. R⁴¹ is a C₁-C₃₀ (d+1)-valent hydrocarbon group which may contain a heteroatom. R⁴² is an acid labile group, and e is an integer of 1 to 4.

In formula (d1), the acid labile group R⁴² is deprotected under the action of acid so that a hydroxy group is generated. The structure of R⁴² is not particularly limited, an acetal structure, ketal structure, alkoxycarbonyl group and alkoxymethyl group having the following formula (d2) are preferred, with the alkoxymethyl group having formula (d2) being more preferred.

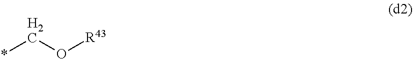

Herein * designates a valence bond and R⁴³ is a C₁-C₁₅ hydrocarbyl group.

Illustrative examples of the acid labile group R⁴², the alkoxymethyl group having formula (d2), and the repeat units (d) are as exemplified for the repeat units (d) in JP-A 2020-111564 (US 20200223796).

In addition to the foregoing units, the base polymer may further comprise repeat units (e) derived from indene, benzofuran, benzothiophene, acenaphthylene, chromone, coumarin, and norbornadiene, or derivatives thereof. Examples of the monomer from which repeat units (e) are derived are shown below, but not limited thereto.

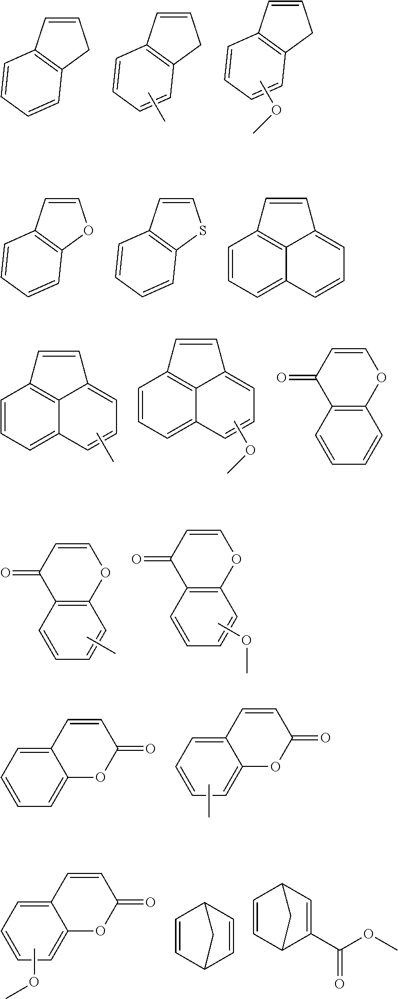

Furthermore, the base polymer may comprise repeat units (f) derived from indane, vinylpyridine, vinylcarbazole, or derivatives thereof.

In the base polymer, a fraction of units (a1), (a2), (b1), (b2), (c1) to (c4), (d), (e), and (f) is: preferably 0<a1≤0.8, 0≤a2≤0.8, 0≤b1≤0.6, 0≤b2≤0.6, 0≤c1≤0.4, 0≤c2≤0.4, 0≤c3≤0.4, 0≤c4≤0.4, 0≤d≤0.5, 0≤e≤0.3, and 0≤f≤0.3; more preferably 0<a1≤0.7, 0≤a2≤0.7, 0≤b1≤0.5, 0≤b2≤0.5, 0≤c1≤0.3, 0≤c2≤0.3, 0≤c3≤0.3, 0≤c4≤0.3, 0≤d≤0.3, 0≤e≤0.3, and 0≤f≤0.3.

The base polymer should preferably have a weight average molecular weight (Mw) in the range of 1,000 to 500,000, and more preferably 3,000 to 100,000. A Mw in the range ensures satisfactory etch resistance and eliminates the risk of resolution being lowered due to a failure to acquire a difference in dissolution rate before and after exposure. It is noted that Mw is as measured by GPC versus polystyrene standards using tetrahydrofuran (THF) or N,N-dimethylformamide (DMF) solvent

Since the influence of dispersity (Mw/Mn) becomes stronger as the pattern rule becomes finer, the base polymer should preferably have a narrow dispersity (Mw/Mn) of 1.0 to 2.0 in order to provide a resist composition suitable for micropatterning to a small feature size. A Mw/Mn in the range indicates smaller amounts of lower and higher molecular weight fractions and eliminates the risk of leaving foreign particles on the pattern or degrading the pattern profile after exposure and development.

The base polymer may be synthesized by any desired methods, for example, by dissolving one or more monomers selected from the monomers corresponding to the foregoing repeat units in an organic solvent, adding a radical polymerization initiator thereto, and heating for polymerization. Examples of the organic solvent which can be used for polymerization include toluene, benzene, tetrahydrofuran (THF), diethyl ether, dioxane, cyclohexane, cyclopentane, methyl ethyl ketone (MEK), PGMEA, and GBL. Examples of the polymerization initiator used herein include 2,2′-azobisisobutyronitrile (AIBN), 2,2′-azobis(2,4-dimethylvaleronitrile), dimethyl 2,2-azobis(2-methylpropionate), 1,1′-azobis(1-acetoxy-1-phenylethane), benzoyl peroxide, and lauroyl peroxide. The amount of the initiator added is preferably 0.01 to 25 mol % based on the total of monomers. The reaction temperature is preferably 50 to 150° C., more preferably 60 to 100° C. The reaction time is preferably 2 to 24 hours, a time of 2 to 12 hours being more preferred in view of production efficiency.

The polymerization initiator may be added to the monomer solution, which is fed to the reactor. Alternatively, a solution of the polymerization initiator is prepared separately from the monomer solution, and the monomer and initiator solutions be independently fed to the reactor. Since there is a possibility that the initiator generates a radical in the standby time, by which polymerization reaction takes place to form a ultrahigh molecular weight compound, it is preferred from the standpoint of quality control that the monomer solution and the initiator solution be independently prepared and added dropwise. The acid labile group that has been incorporated in the monomer may be kept as such, or the polymerization may be followed by protection or partial protection. Any of well-known chain transfer agents such as dodecylmercaptan and 2-mercaptoethanol may be used for the purpose of adjusting molecular weight. An appropriate amount of the chain transfer agent is 0.01 to 20 mol % based on the total of monomers to be polymerized.

Where a monomer having a hydroxy group is copolymerized, the hydroxy group may be replaced by an acetal group susceptible to deprotection with acid, typically ethoxyethoxy, prior to polymerization, and the polymerization be followed by deprotection with weak acid and water. Alternatively, the hydroxy group may be replaced by an acetyl, formyl, pivaloyl or similar group prior to polymerization, and the polymerization be followed by alkaline hydrolysis.

When hydroxystyrene or hydroxyvinylnaphthalene is copolymerized, an alternative method is possible. Specifically, acetoxystyrene or acetoxy vinylnaphthalene is used instead of hydroxystyrene or hydroxyvinylnaphthalene, and after polymerization, the acetoxy group is deprotected by alkaline hydrolysis, for thereby converting the polymer product to hydroxystyrene or hydroxyvinylnaphthalene. For alkaline hydrolysis, a base such as aqueous ammonia or triethylamine may be used. Preferably the reaction temperature is −20° C. to 100° C., more preferably 0° C. to 60¹C, and the reaction time is 0.2 to 100 hours, more preferably 0.5 to 20 hours.

The amounts of monomers in the monomer solution may be determined appropriate so as to provide the preferred fractions of repeat units as mentioned above.

It is described how to use the polymer obtained by the above preparation method. The reaction solution resulting from polymerization reaction may be used as the final product. Alternatively, the polymer may be recovered in powder form through a purifying step such as re-precipitation step of adding the reaction solution to a poor solvent and letting the polymer precipitate as powder, after which the polymer powder is used as the final product. It is preferred from the standpoints of operation efficiency and consistent quality to handle a polymer solution which is obtained by dissolving the powder polymer resulting from the purifying step in a solvent, as the final product.

The solvents which can be used herein are described in JP-A 2008-111103, paragraphs [0144]-[0145] (U.S. Pat. No. 7,537,880). Exemplary solvents include ketones such as cyclohexanone and methyl-2-n-pentyl ketone; alcohols such as 3-methoxybutanol, 3-methyl-3-methoxybutanol, 1-methoxy-2-propanol, 1-ethoxy-2-propanol; ethers such as PGME, ethylene glycol monomethyl ether, propylene glycol monoethyl ether, ethylene glycol monoethyl ether, propylene glycol dimethyl ether, and diethylene glycol dimethyl ether; esters such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate: lactones such as GBL; alcohols such as DAA: and high-boiling alcohols such as diethylene glycol, propylene glycol, glycerol, 1,4-butanediol, and 1,3-butanediol, which may be used alone or in admixture.

The polymer solution preferably has a polymer concentration of 0.01 to 30% by weight, more preferably 0.1 to 20% by weight.

Prior to use, the reaction solution or polymer solution is preferably filtered through a filter. Filtration is effective for consistent quality because foreign particles and gel which can cause defects are removed.

Suitable materials of which the filter is made include fluorocarbon, cellulose, nylon, polyester, and hydrocarbon base materials. Preferred for the filtration of a resist composition are filters made of fluorocarbons commonly known as Teflon®, hydrocarbons such as polyethylene and polypropylene, and nylon. While the pore size of the filter may be selected appropriate to comply with the desired cleanness, the filter preferably has a pore size of up to 100 nm, more preferably up to 20 nm. A single filter may be used or a plurality of filters may be used in combination. Although the filtering method may be single pass of the solution, preferably the filtering step is repeated by flowing the solution in a circulating manner. In the polymer preparation process, the filtering step may be carried out any times, in any order and in any stage. The reaction solution as polymerized or the polymer solution may be filtered, preferably both are filtered.

The base polymer (C) may be used alone or as a mixture of two or more polymers which are different in compositional ratio, Mw and/or Mw/Mn. In addition to the polymer defined above, the base polymer (C) may contain a hydrogenated product of ring-opening metathesis polymerization (ROMP) polymer, which is described in JP-A 2003-066612.

(D) Photoacid Generator

The resist composition may comprise (D) a photoacid generator. The PAG used herein may be any compound capable of generating an acid upon exposure to high-energy radiation. The preferred PAG has the formula (2).

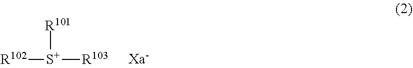

In formula (2), R¹⁰¹, R¹⁰² and R¹⁰³ are each independently a C₁-C₂₀ hydrocarbyl group which may contain a heteroatom. Any two of R¹⁰¹, R¹⁰² and R¹⁰³ may bond together to form a ring with the sulfur atom to which they are attached. Examples of the hydrocarbyl group are as exemplified above for the hydrocarbyl group represented by R¹¹ to R¹³ in formula (cation-1). Examples of the cation in the sulfonium salt having formula (2) are as exemplified above for the sulfonium cation having formula (cation-1).

In formula (2). Xa⁻ is an anion of the following formula (2A), (2B), (2C) or (2D).

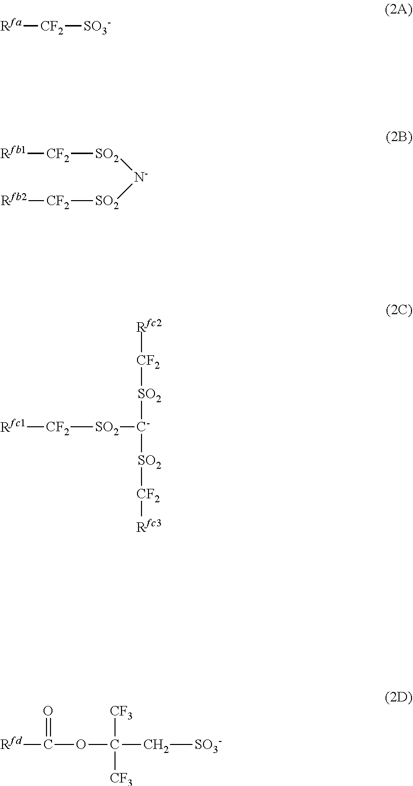

In formula (2A). R^{fa} is fluorine or a C₁-C₄₀ hydrocarbyl group which may contain a heteroatom. The hydrocarbyl group may be saturated or unsaturated and straight, branched or cyclic. Examples thereof are as will be exemplified later for hydrocarbyl group R^{fa1} in formula (2A′).

Of the anions of formula (2A), a structure having formula (2A′) is preferred.

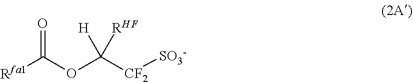

In formula (2A′), R^{HF} is hydrogen or trifluoromethyl, preferably trifluoromethyl.

R^{fa1} is a C₁-C₃₈ hydrocarbyl group which may contain a heteroatom. Suitable heteroatoms include oxygen, nitrogen, sulfur and halogen, with oxygen being preferred. Of the hydrocarbyl groups, those of 6 to 30 carbon atoms are preferred because a high resolution is available in fine pattern formation. The C₁-C₃₈ hydrocarbyl group R^{fa1} may be saturated or unsaturated and straight, branched or cyclic. Suitable hydrocarbyl groups include C₁-C₃₀ alkyl groups such as methyl, ethyl, n-propyl, isopropyl, n-butyl, isobutyl, sec-butyl, tert-butyl, pentyl, neopentyl, hexyl, heptyl, octyl, 2-ethylhexyl, nonyl, decyl, undecyl, dodecyl, tridecyl, pentadecyl, heptadecyl, icosanyl; C₃-C₃₀ cyclic saturated hydrocarbyl groups such as cyclopentyl, cyclohexyl, 1-adamantyl, 2-adamantyl, 1-adamantylmethyl, norbornyl, norbornylmethyl, tricyclodecanyl, tetracyclododecanyl, tetracyclododecanylmethyl, dicyclohexylmethyl; C₂-C₃₀ unsaturated aliphatic hydrocarbyl groups such as allyl and 3-cyclohexenyl; C₆-C₃₀ aryl groups such as phenyl, 1-naphthyl, 2-naphthyl; C₇-C₃₈ aralkyl groups such as benzyl and diphenylmethyl; and combinations thereof.

In the hydrocarbyl group, some or all of the hydrogen atoms may be substituted by a moiety containing a heteroatom such as oxygen, sulfur, nitrogen or halogen, or some constituent —CH₂— may be replaced by a moiety containing a heteroatom such as oxygen, sulfur or nitrogen, so that the group may contain a hydroxy, fluorine, chlorine, bromine, iodine, cyano, carbonyl, ether bond, ester bond, sulfonic ester bond, carbonate bond, lactone ring, sultone ring, carboxylic anhydride (—C(═O)—O—C(═O)—) or haloalkyl moiety. Examples of the heteroatom-containing hydrocarbyl group include tetrahydrofuryl, methoxymethyl, ethoxymethyl, methylthiomethyl, acetamidomethyl, trifluoroethyl, (2-methoxyethoxy)methyl, acetoxymethyl, 2-carboxy-1-cyclohexyl, 2-oxopropyl, 4-oxo-1-adamantyl, 5-hydroxy-1-adamantyl, 5-tert-butylcarbonyloxy-1-adamantyl, 4-oxatricyclo[4.2.1.0^{3,7}]nonan-5-on-2-yl, and 3-oxocyclohexyl.

With respect to the synthesis of the sulfonium salt having an anion of formula (2A′), reference is made to JP-A 2007-145797, JP-A 2008-106045, JP-A 2009-007327, and JP-A 2009-258695. Also useful are the sulfonium salts described in JP-A 2010-215608, JP-A 2012-041320, JP-A 2012-106986, and JP-A 2012-153644.

Examples of the anion having formula (2A) are as exemplified above for the anions having formulae (c1-1) and (c1-2).

In formula (2B), R^{fb1} and R^{fb2} are each independently fluorine or a C₁-C₄₀ hydrocarbyl group which may contain a heteroatom. The hydrocarbyl group may be saturated or unsaturated and straight, branched or cyclic. Suitable hydrocarbyl groups are as exemplified above for R^{fa1} in formula (2A′). Preferably R^{fb1} and R^{fb2} each are fluorine or a straight C₁-C₄ fluorinated alkyl group. A pair of R^{fb1} and R^{fb2} may bond together to form a ring with the linkage (—CF₂—SO₂—N⁻—SO₂—CF₂—) to which they are attached, and the ring-forming pair is preferably a fluorinated ethylene or fluorinated propylene group.

In formula (2C), R^{fc1}, R^{fc2} and R^{fc3} are each independently fluorine or a C₁-C₄₀ hydrocarbyl group which may contain a heteroatom. The hydrocarbyl group may be saturated or unsaturated and straight, branched or cyclic. Suitable hydrocarbyl groups are as exemplified above for R^{fa1} in formula (2A′). Preferably R^{fc1}, R^{fc2} and R^{fc3} each are fluorine or a straight C₁-C₄ fluorinated alkyl group. A pair of R^{fc1} and R^{fc2} may bond together to form a ring with the linkage (—CF₂—SO₂—C⁻—SO₂—CF₂—) to which they are attached, and the ring-forming pair is preferably a fluorinated ethylene or fluorinated propylene group.

In formula (2D), R^{fd} is a C₁-C₄₀ hydrocarbyl group which may contain a heteroatom. The hydrocarbyl group may be saturated or unsaturated and straight, branched or cyclic. Suitable hydrocarbyl groups are as exemplified above for R^{fa1}.

With respect to the synthesis of the sulfonium salt having an anion of formula (2D), reference is made to JP-A 2010-215608 and JP-A 2014-133723.

Examples of the anion having formula (2D) are shown below, but not limited thereto.

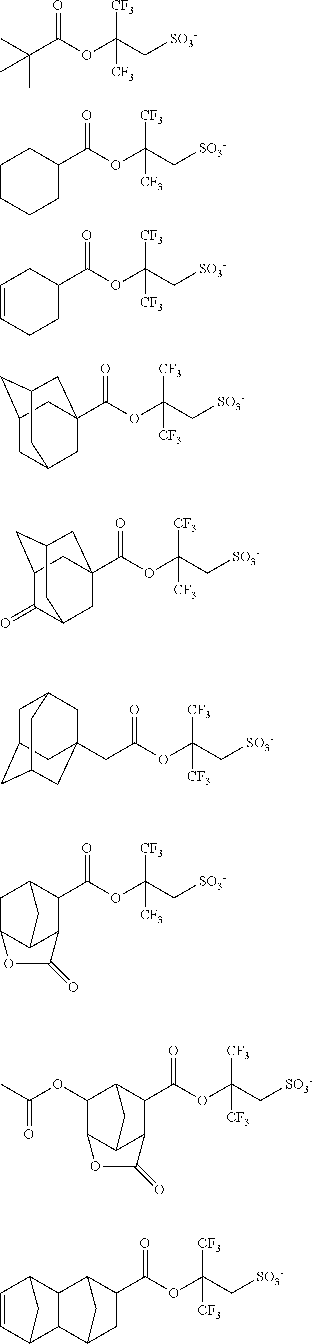

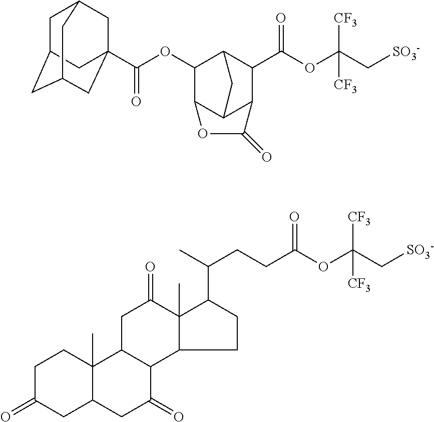

Another example of the non-nucleophilic counter ion is an anion having an iodine or bromine-substituted aromatic ring. The preferred anion has the formula (2E).

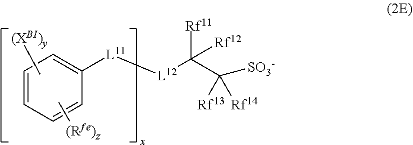

In formula (2E), x is an integer of 1 to 3, y is an integer of 1 to 5, z is an integer of 0 to 3, and y+z is from 1 to 5. Preferably, y is an integer of 1 to 3, more preferably 2 or 3, and z is an integer of 0 to 2.

In formula (2E), X^{BI} is iodine or bromine. When x and/or y is 2 or more, a plurality of X^{BI} may be the same or different.

In formula (2E), L¹¹ is a single bond, ether bond, ester bond, or a C₁-C₆ saturated hydrocarbylene group which may contain an ether bond or ester bond. The saturated hydrocarbylene group may be straight, branched or cyclic.

In formula (2E), L¹² is a single bond or a C₁-C₂₀ divalent linking group when x=1, and a C₁-C₂₀ (x+1)-valent linking group which may contain oxygen, sulfur or nitrogen when x=2 or 3.

In formula (2E), R^{fe} is hydroxy, carboxy, fluorine, chlorine, bromine, or amino, or a C₁-C₂₀ hydrocarbyl group, C₁-C₂₀ hydrocarbyloxy group, C₂-C₂₀ hydrocarbylcarbonyl group, C₂-C₂₀ hydrocarbyloxycarbonyl group, C₂-C₂₀ hydrocarbylcarbonyloxy group, or C₁-C₂₀ hydrocarbylsulfonyloxy group, which may contain fluorine, chlorine, bromine, hydroxy, amino or ether bond, or —N(R^{feA})(R^{feB}), —N(R^{feC})—C(═O)—R^{feD} or —N(R^{feC})—C(═O)—O—R^{feD}. R^{feA} and R^{feB} are each independently hydrogen or a C₁-C₆ saturated hydrocarbyl group. R^{feC} is hydrogen or a C₁-C₆ saturated hydrocarbyl group which may contain halogen, hydroxy, C₁-C₆ saturated hydrocarbyloxy, C₂-C₆ saturated hydrocarbylcarbonyl or C₂-C₆ saturated hydrocarbylcarbonyloxy moiety. R^{feD} is a C₁-C₁₆ aliphatic hydrocarbyl group, C₆-C₁₂ aryl group or C₇-C₁₅ aralkyl group, which may contain halogen, hydroxy. C₁-C₆ saturated hydrocarbyloxy, C₂-C₆ saturated hydrocarbylcarbonyl or C₂-C₆ saturated hydrocarbylcarbonyloxy moiety. The aliphatic hydrocarbyl group may be saturated or unsaturated and straight, branched or cyclic. The hydrocarbyl, hydrocarbyloxy, hydrocarbylcarbonyl, hydrocarbyloxycarbonyl, hydrocarbylcarbonyloxy and hydrocarbylsulfonyloxy groups may be straight, branched or cyclic. Groups R^{fe} may be the same or different when x and/or z is 2 or more. Of these, R^{fe} is preferably hydroxy, —N(R^{feC})—C(═O)—R^{feD}, —N(R^{feC})—C(═O)—O—R^{feD}, fluorine, chlorine, bromine, methyl or methoxy.

In formula (2E), Rf¹¹ to Rf¹⁴ are each independently hydrogen, fluorine or trifluoromethyl, at least one of Rf¹¹ to Rf¹⁴ is fluorine or trifluoromethyl. Rf¹¹ and Rf¹², taken together, may form a carbonyl group. Preferably, both Rf¹³ and Rf¹⁴ are fluorine.

Examples of the anion in the onium salt having formula (2E) are shown below, but not limited thereto. Herein X^{BI} is as defined above.

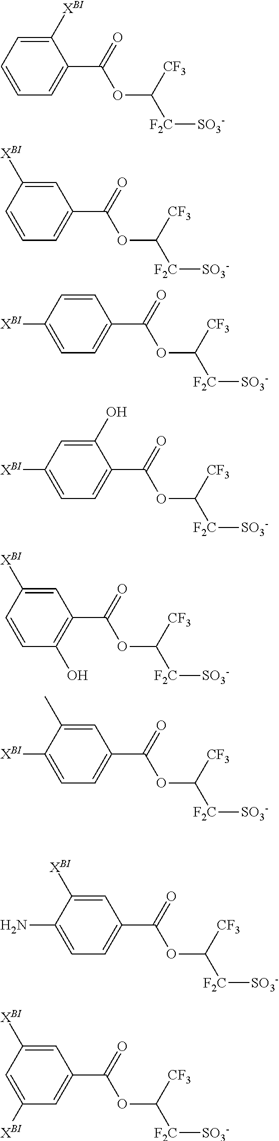

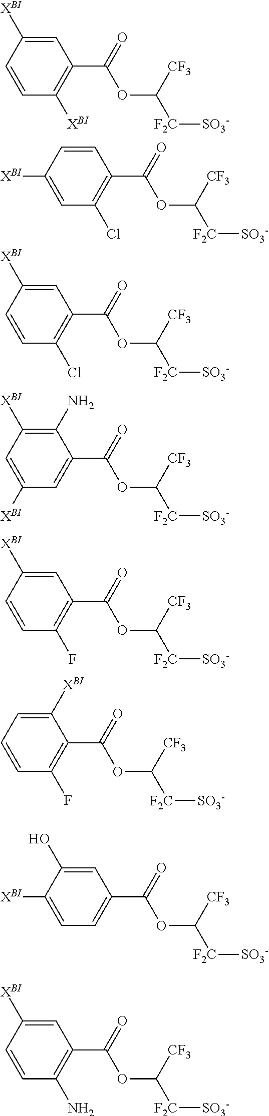

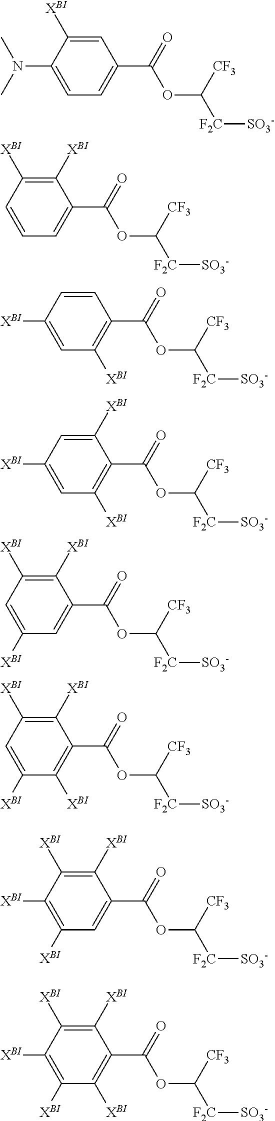

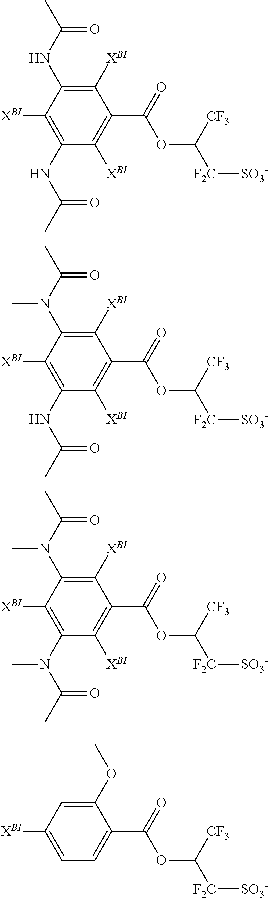

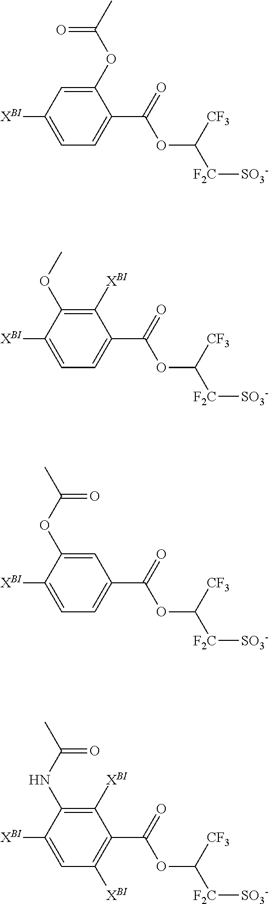

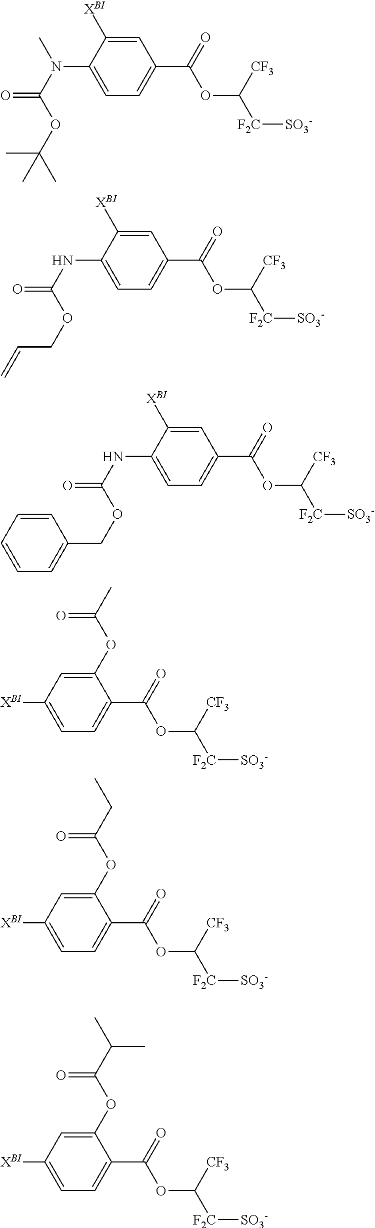

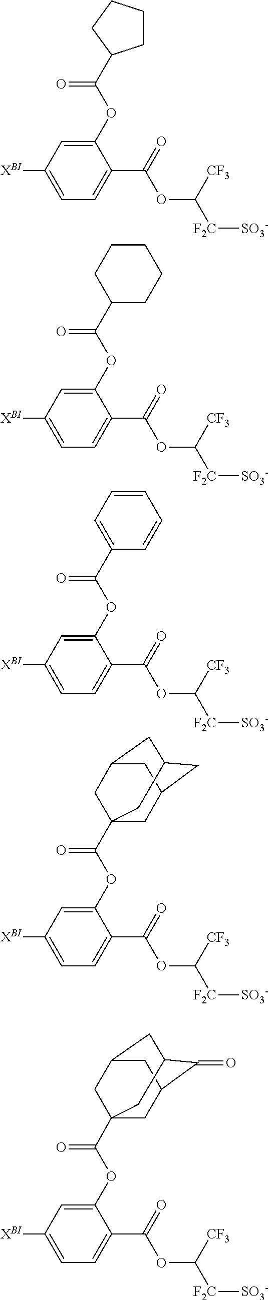

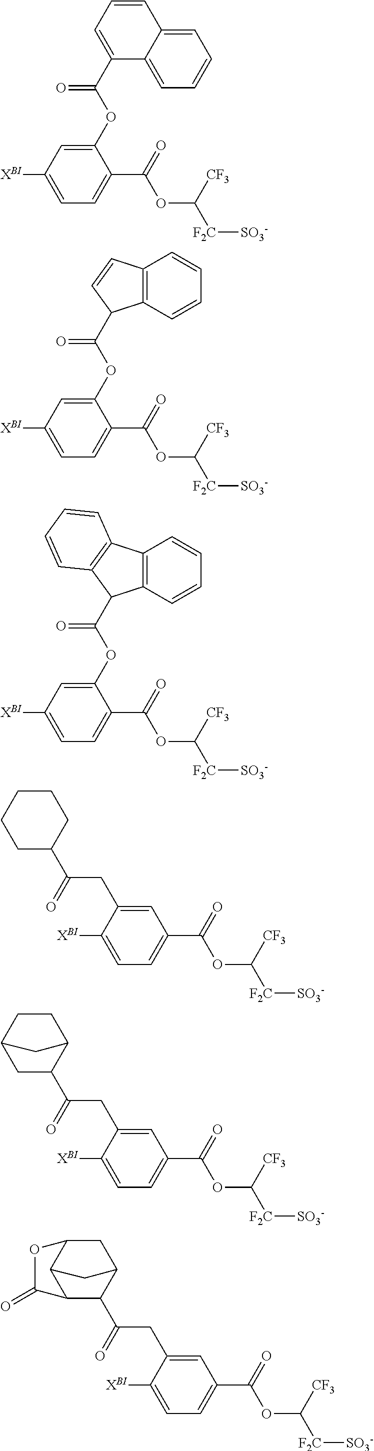

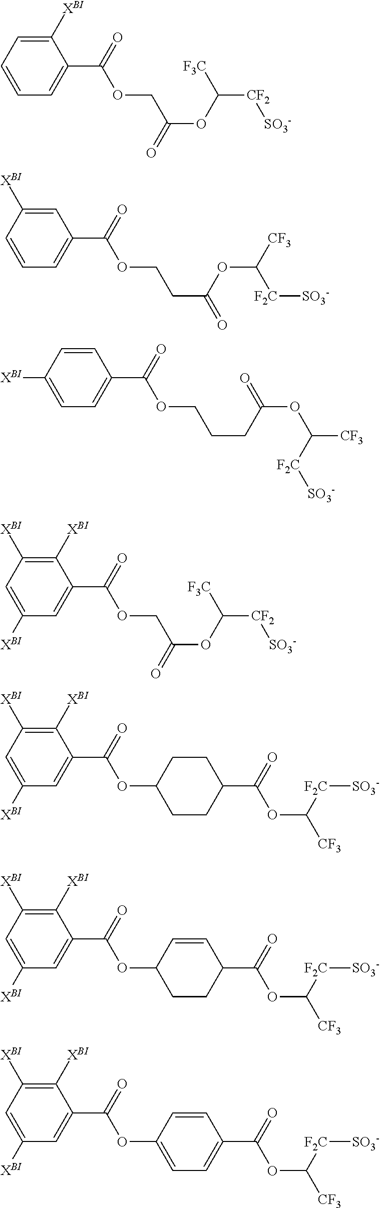

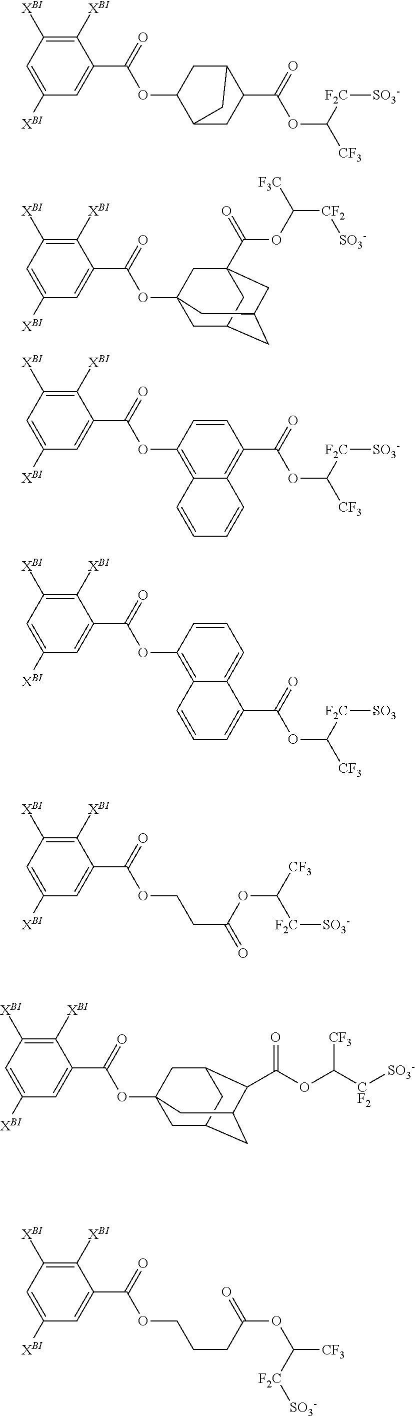

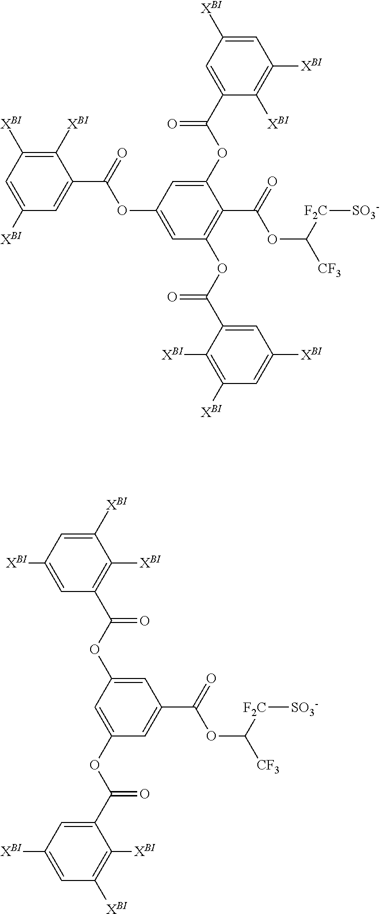

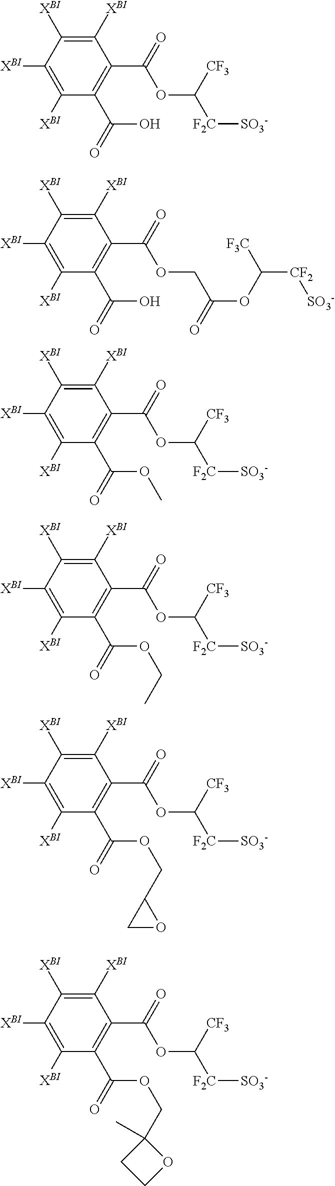

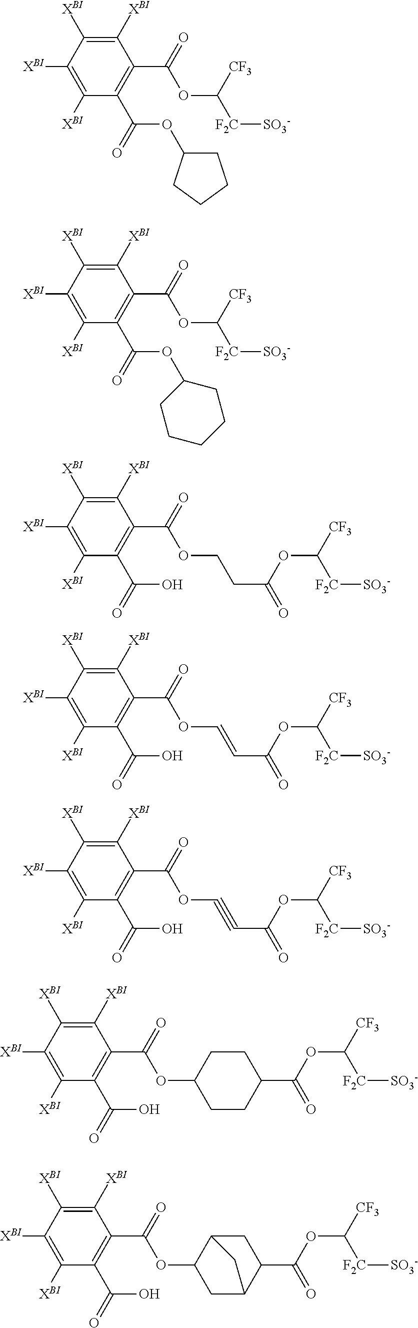

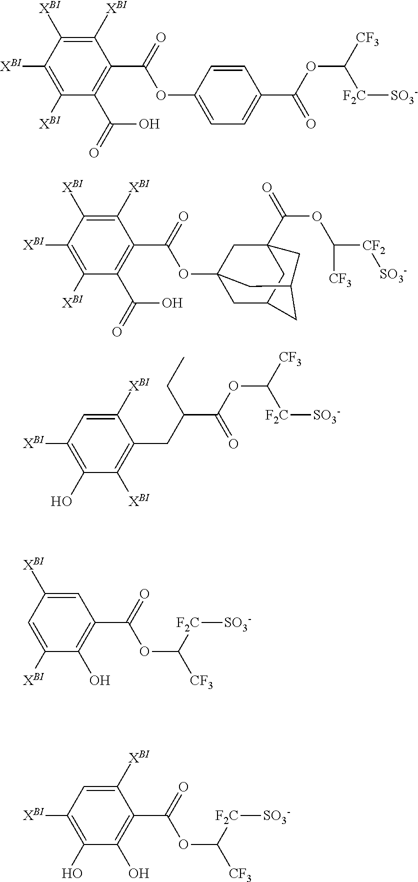

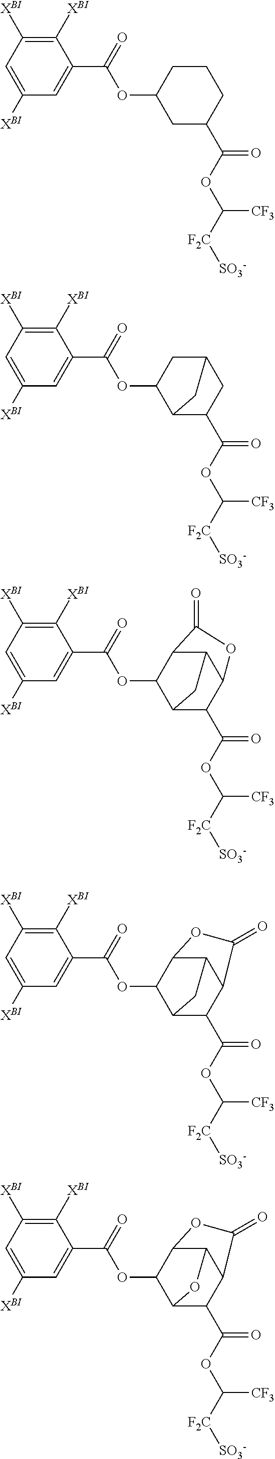

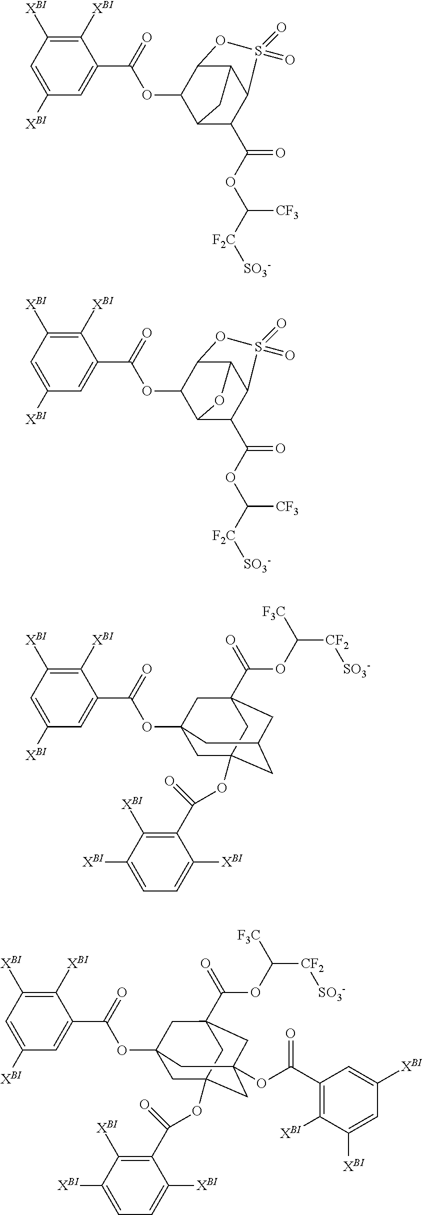

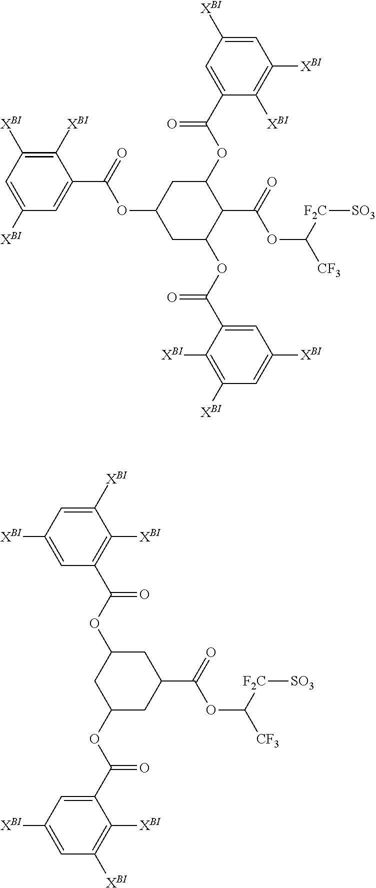

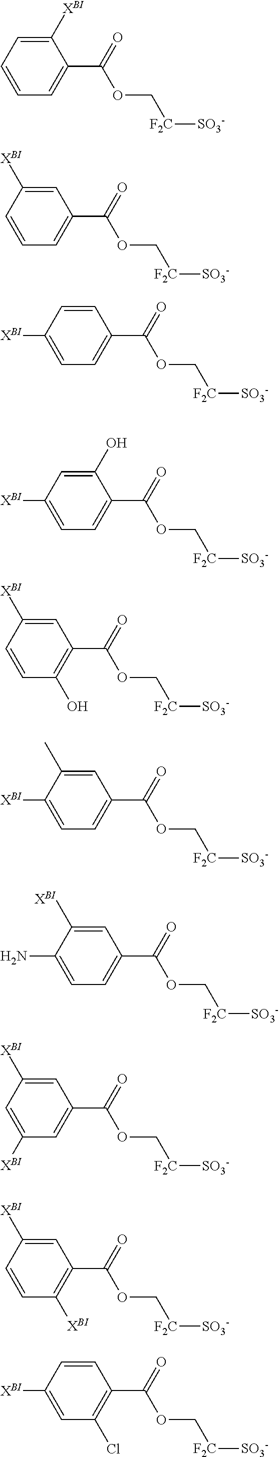

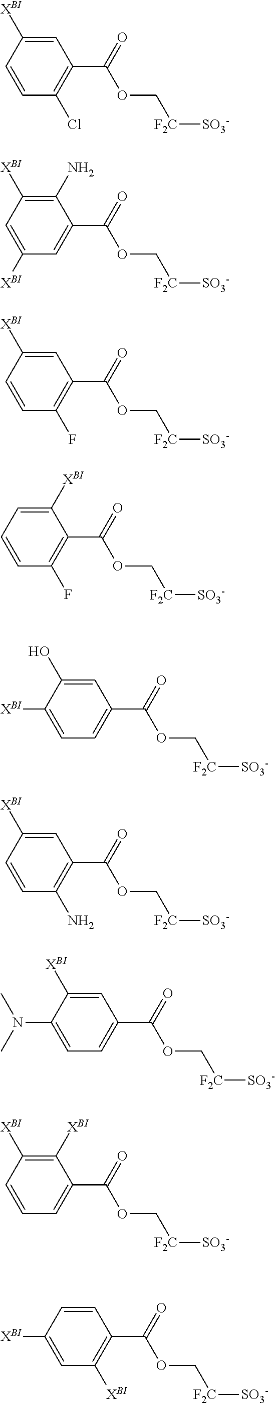

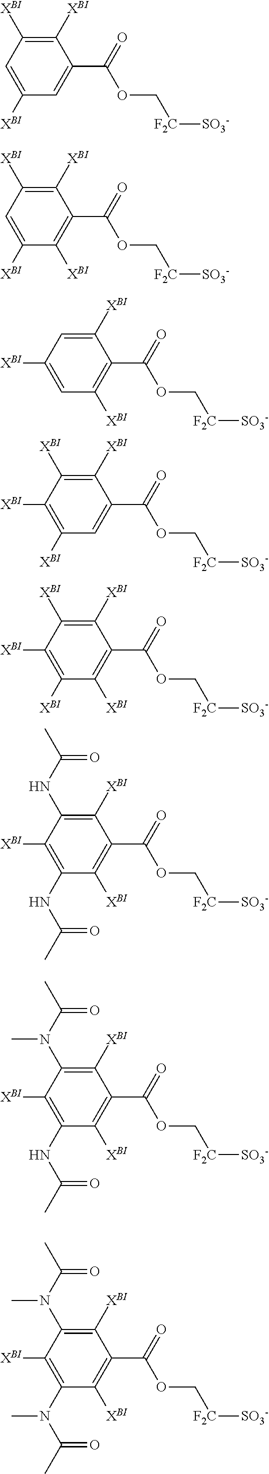

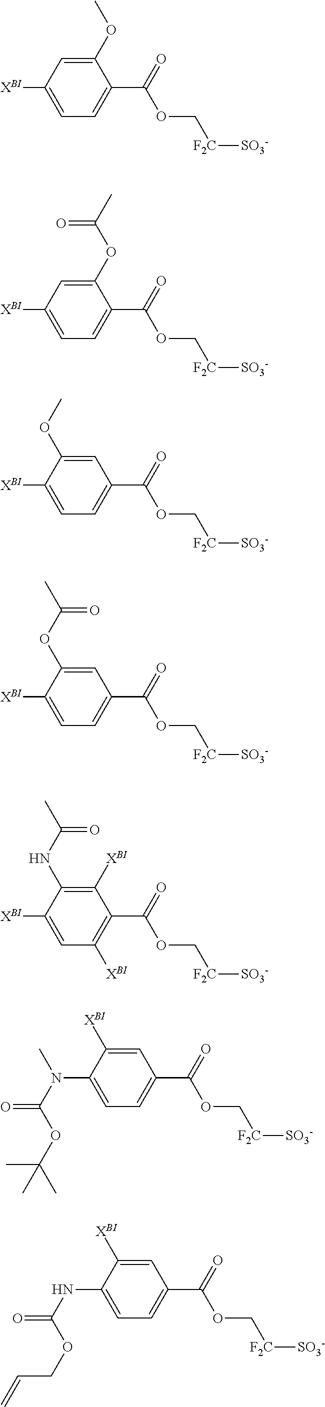

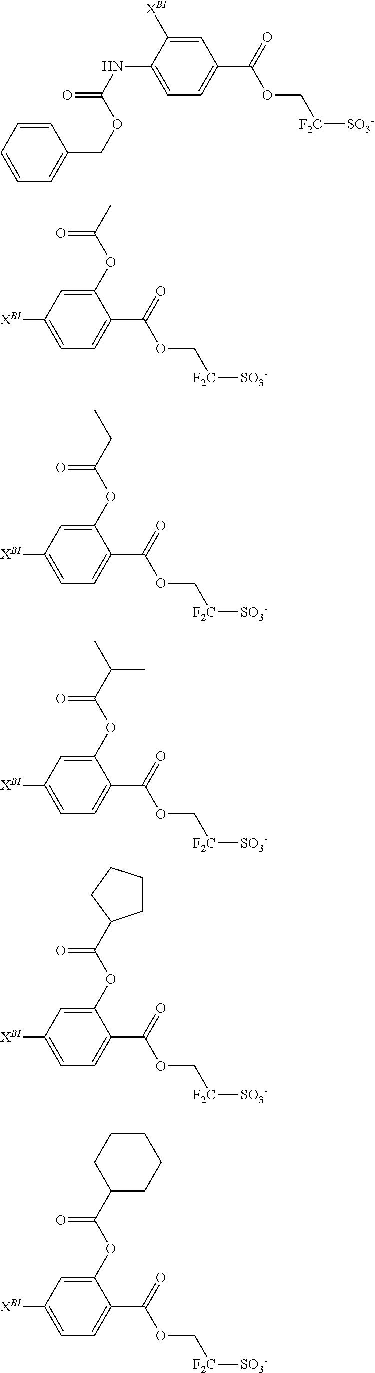

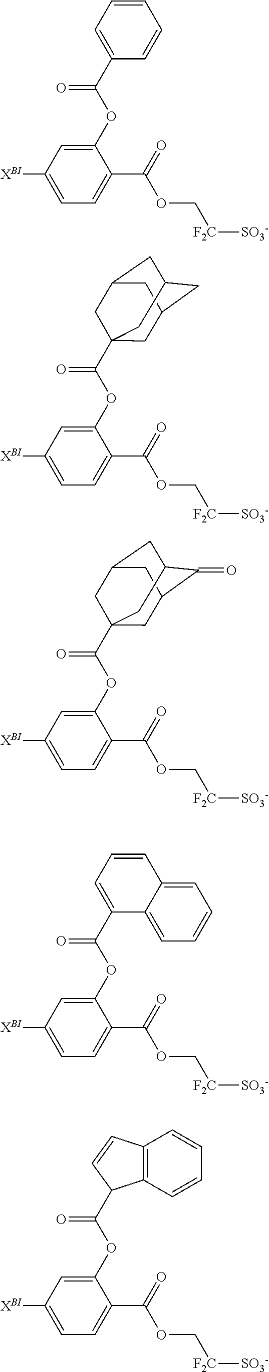

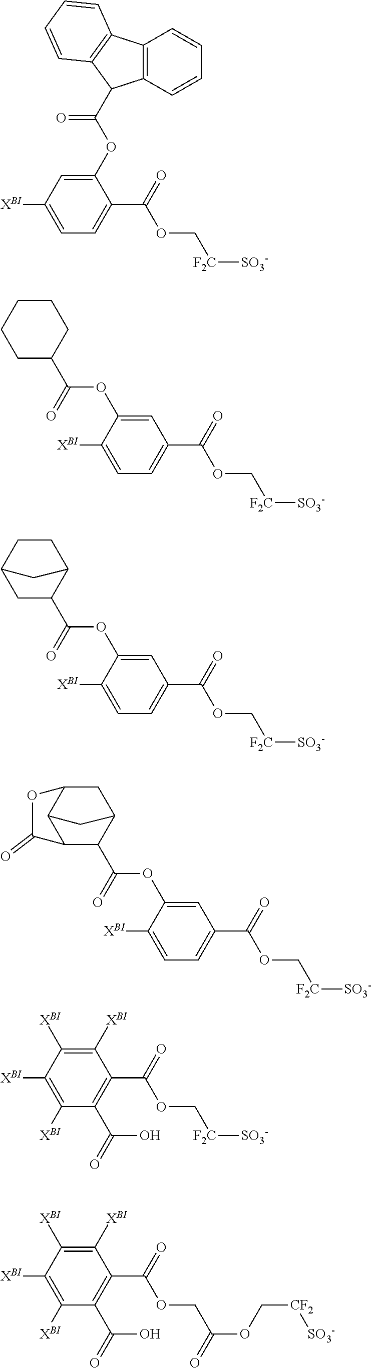

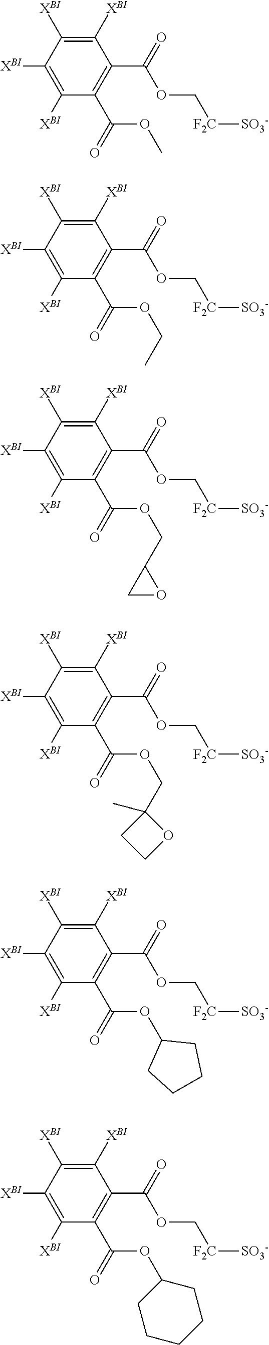

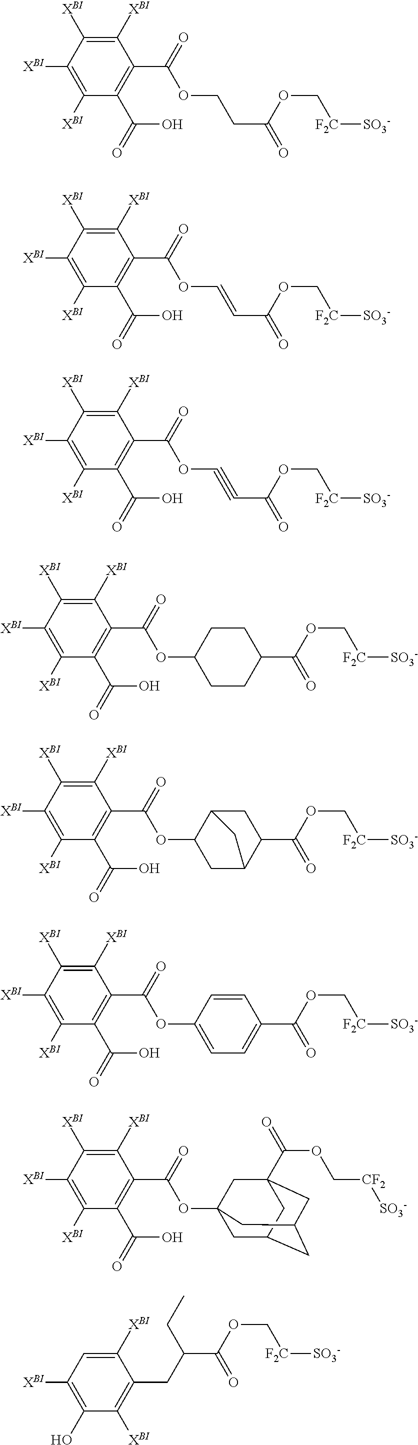

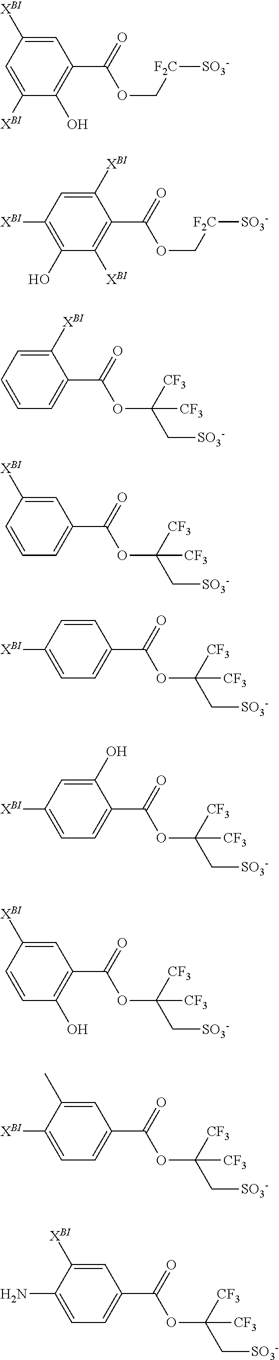

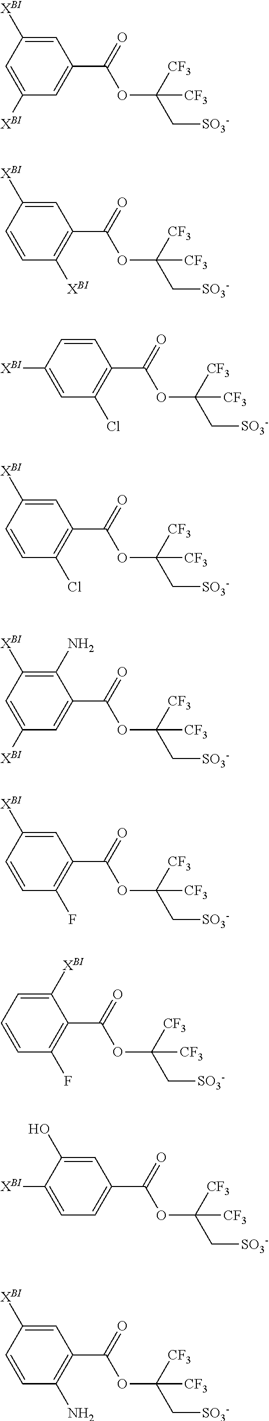

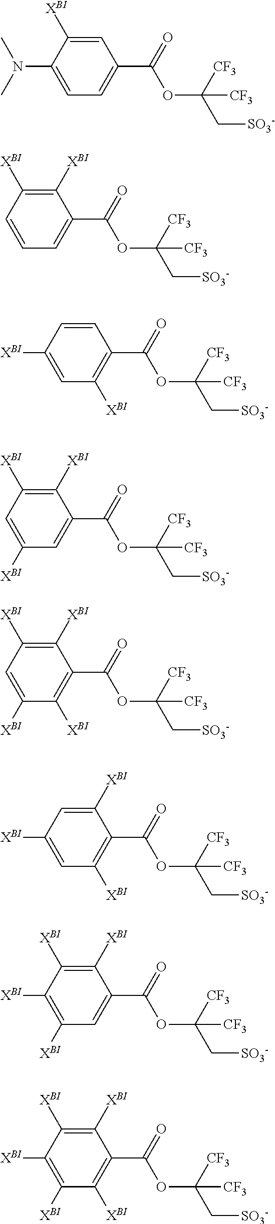

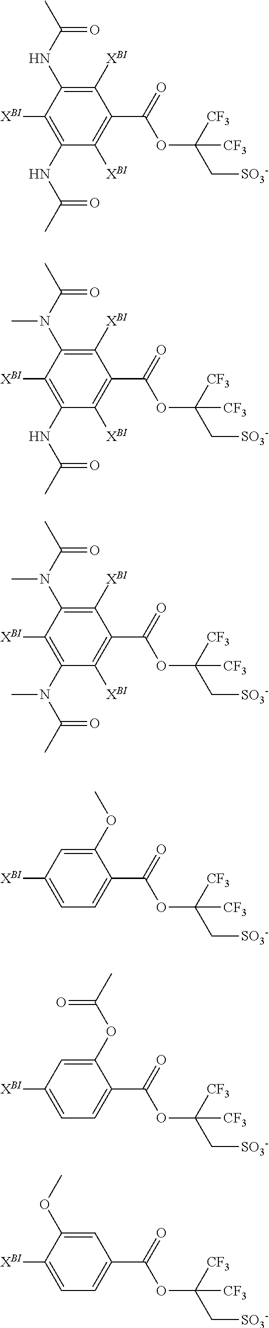

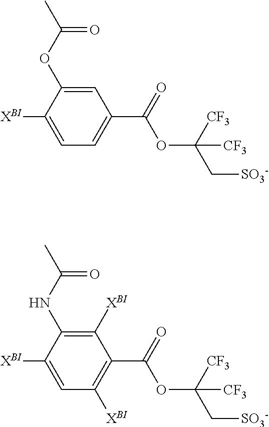

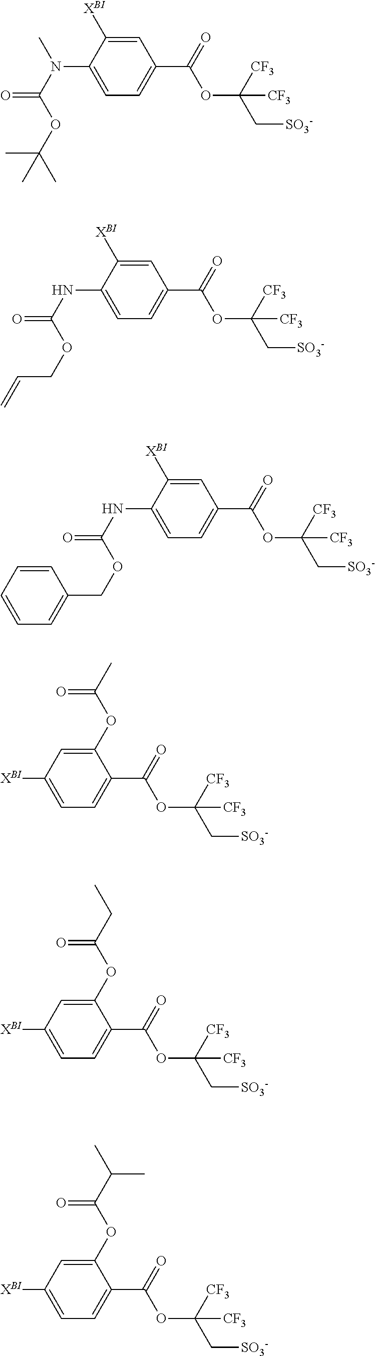

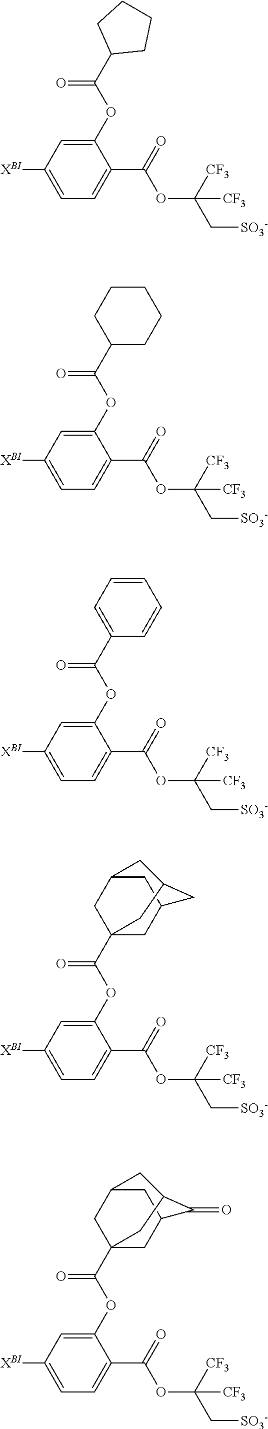

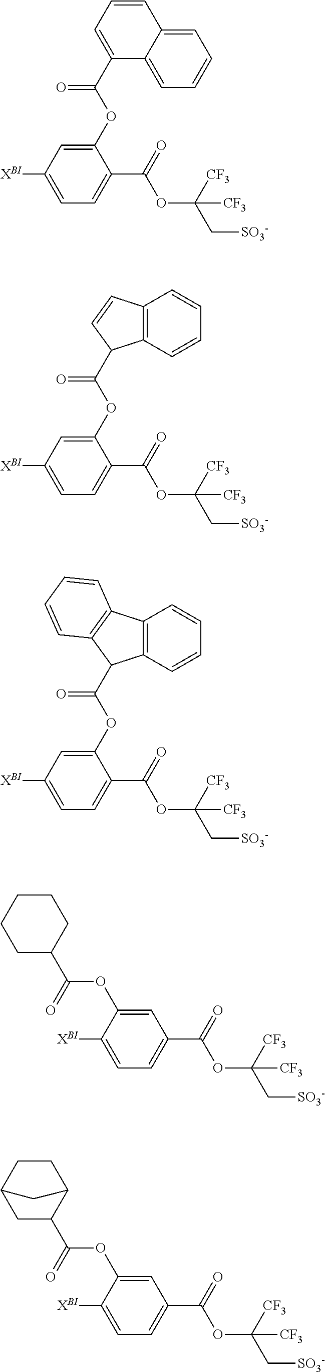

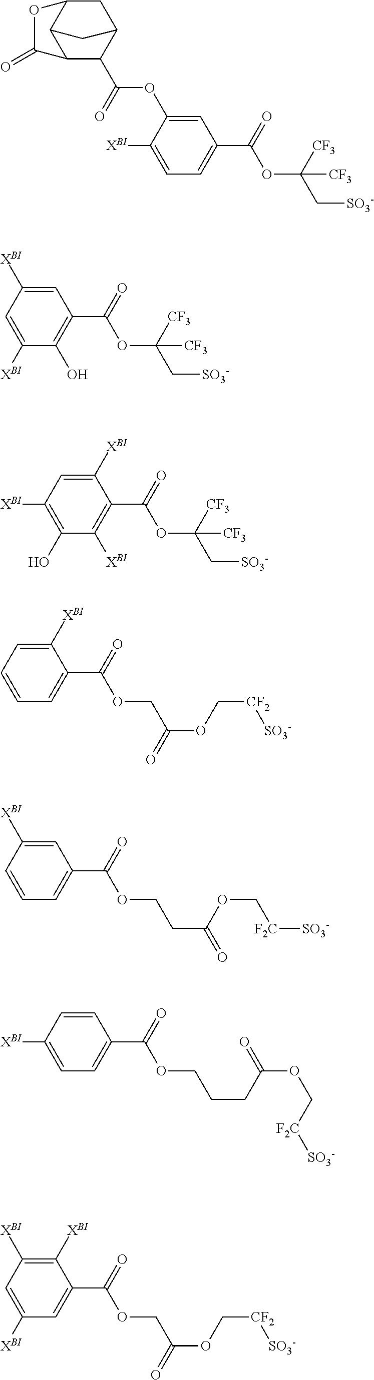

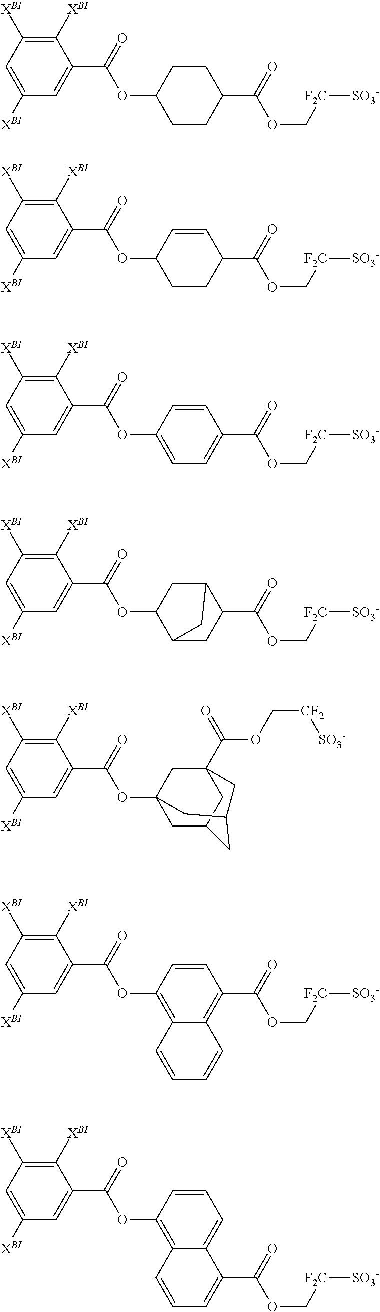

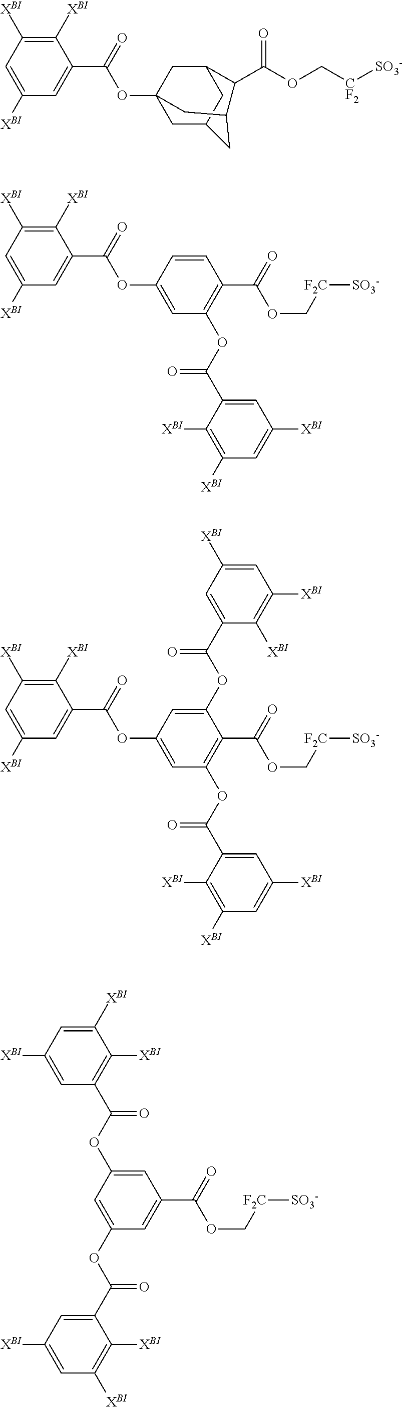

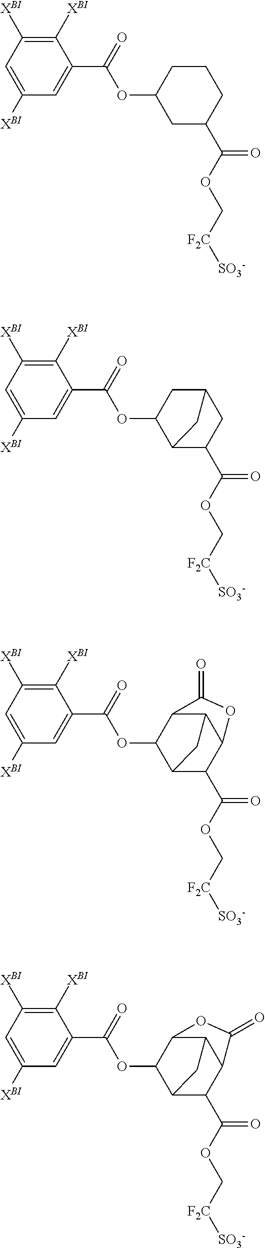

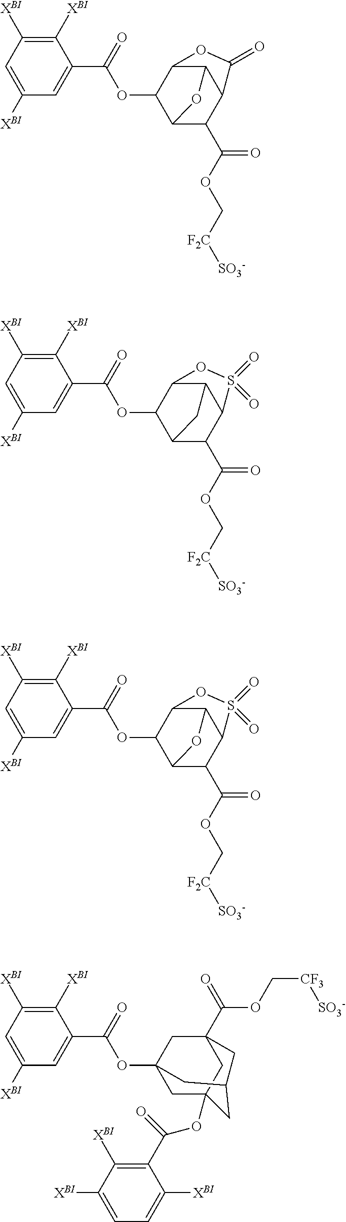

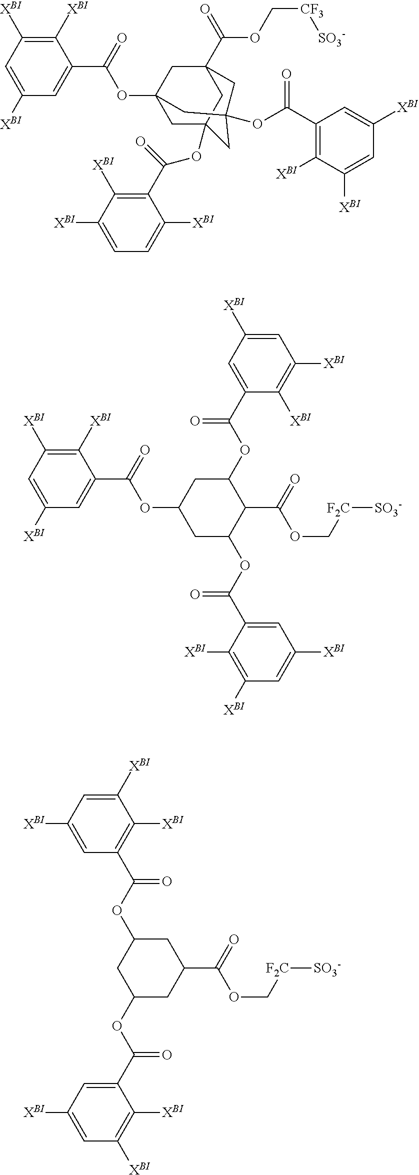

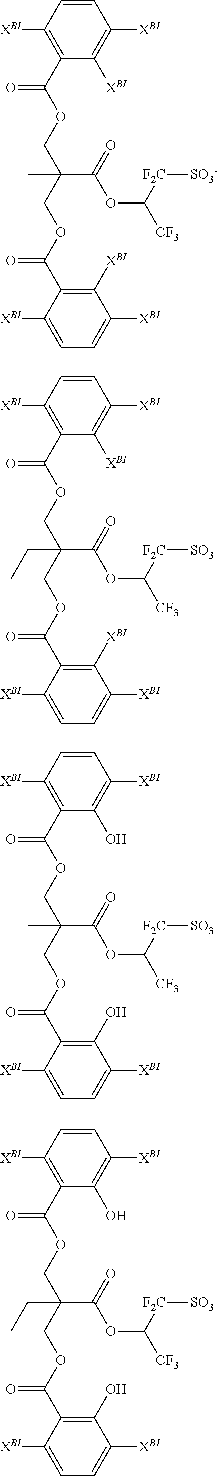

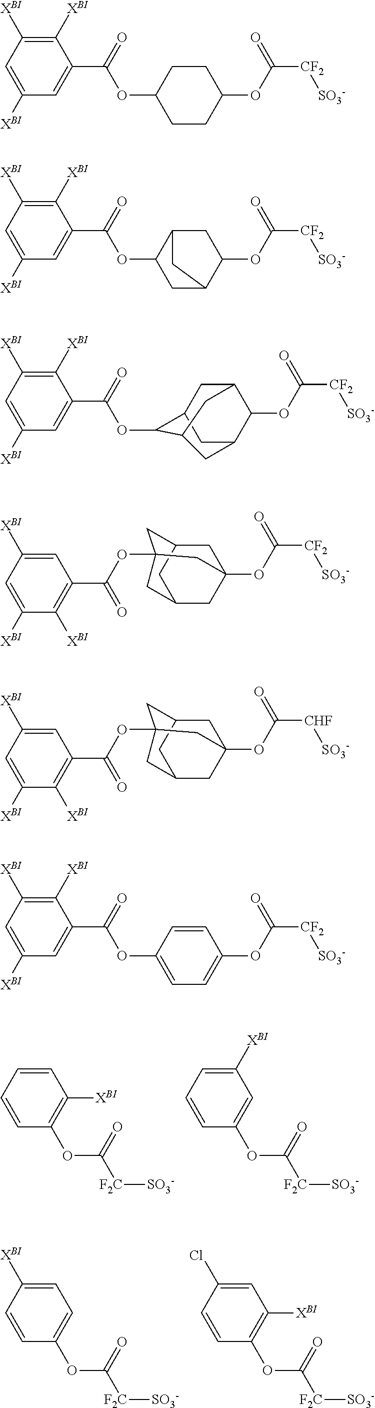

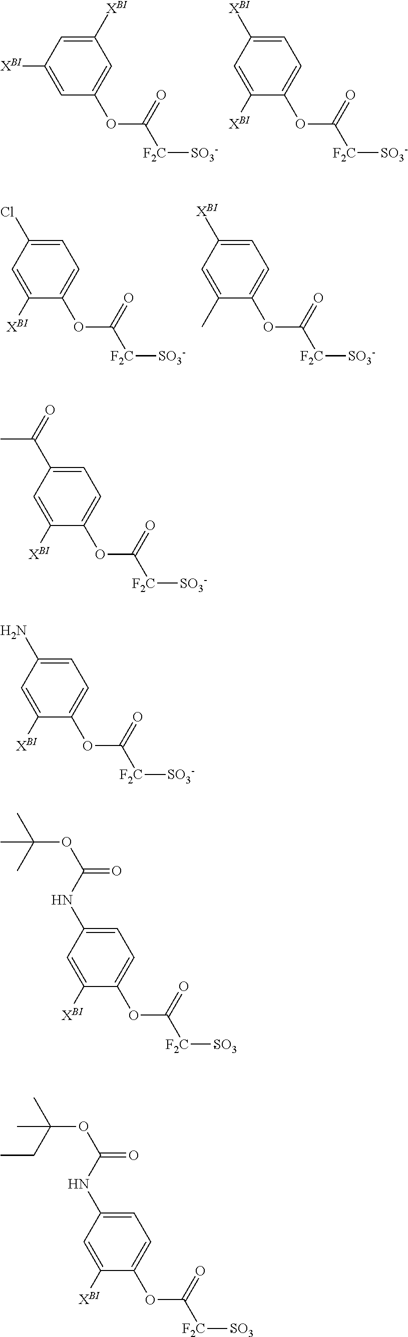

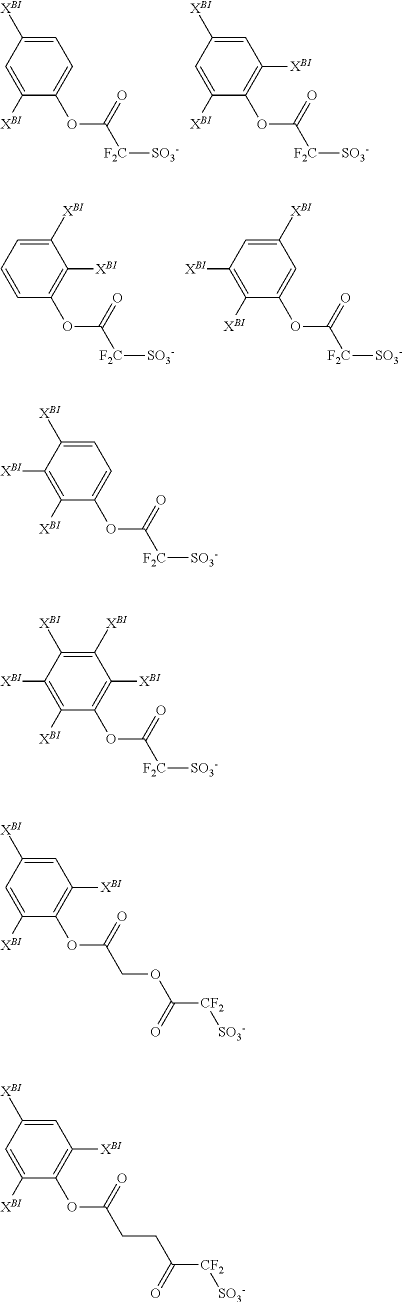

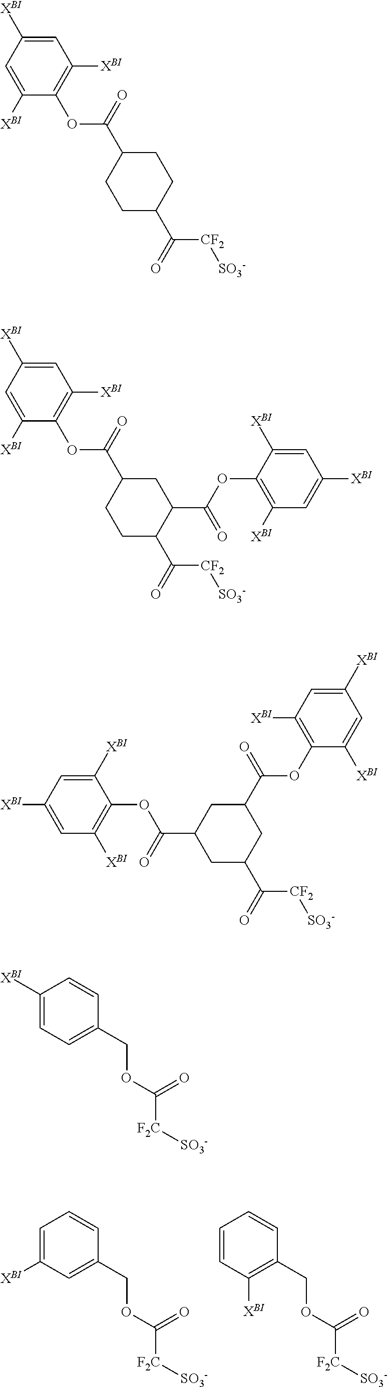

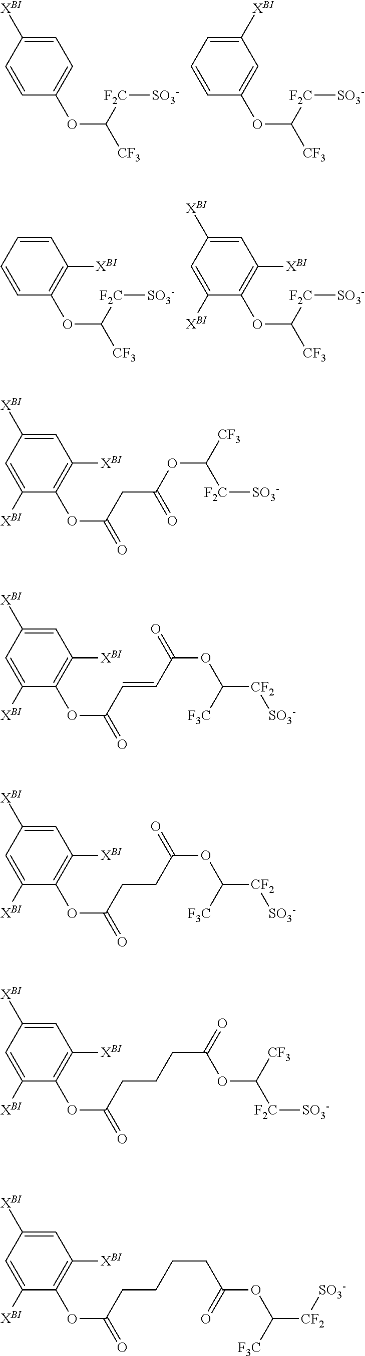

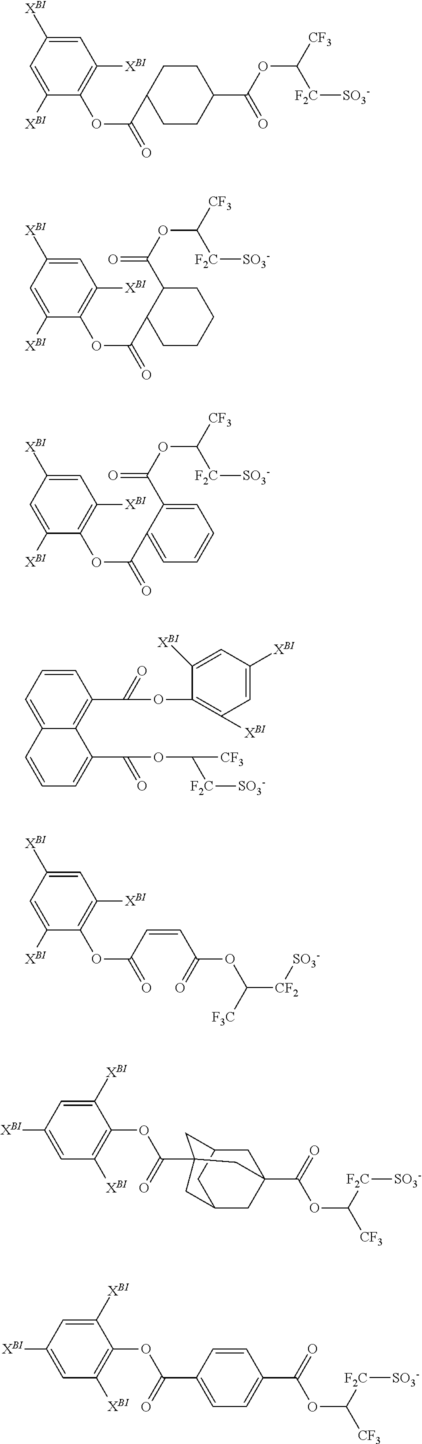

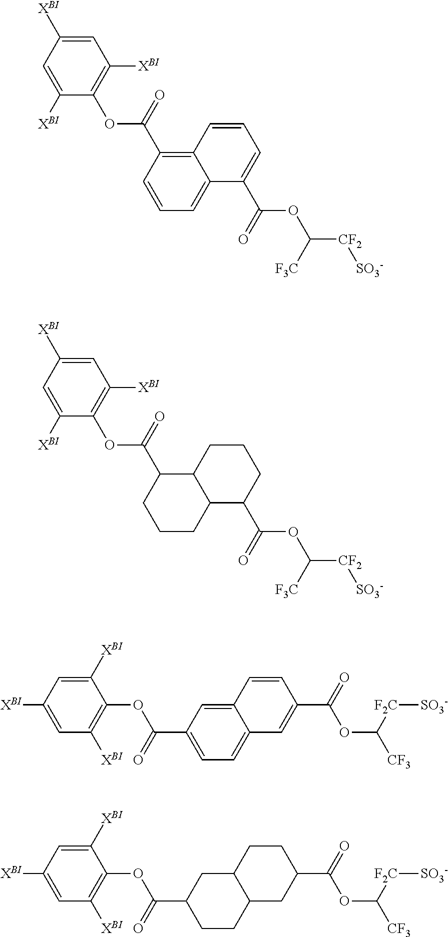

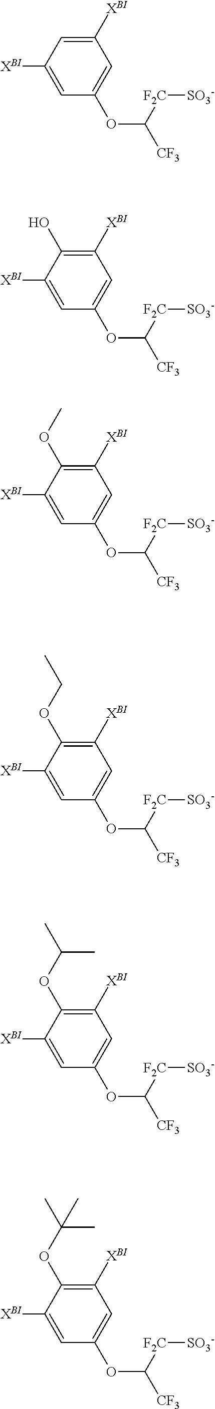

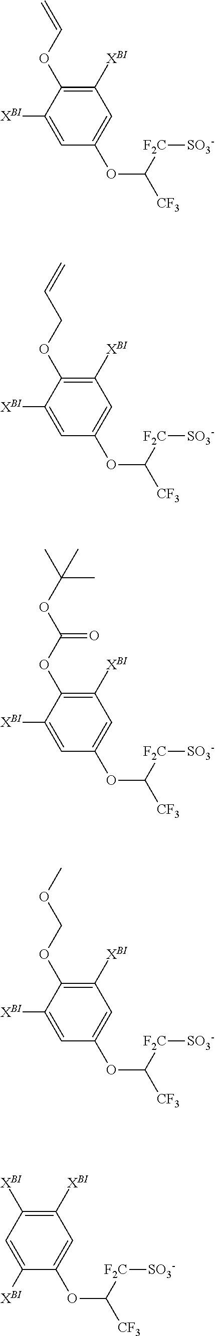

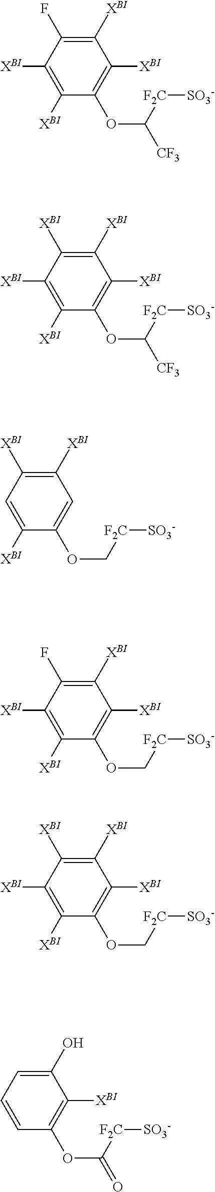

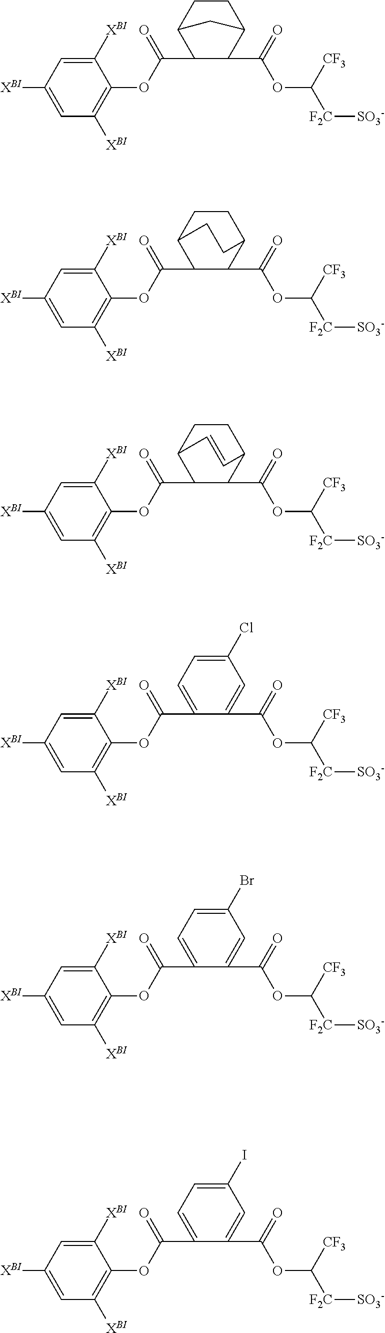

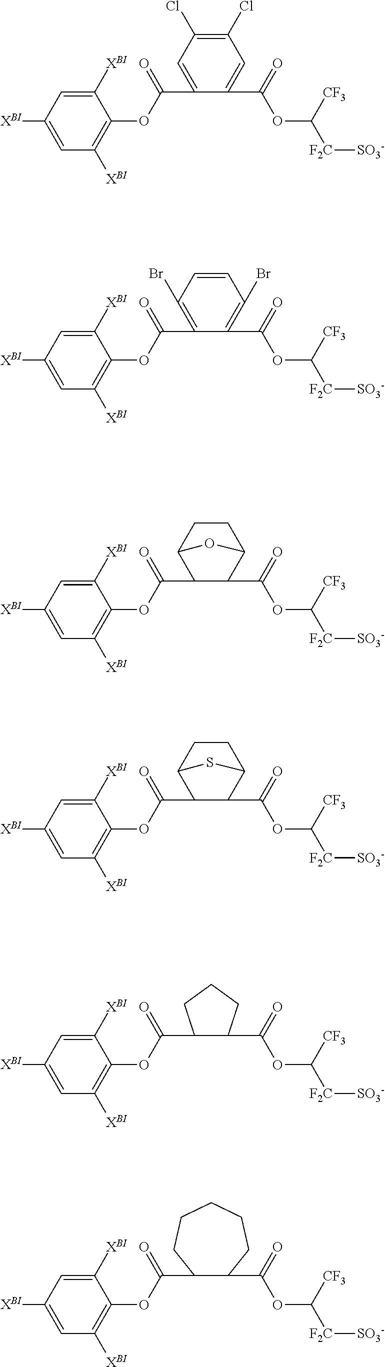

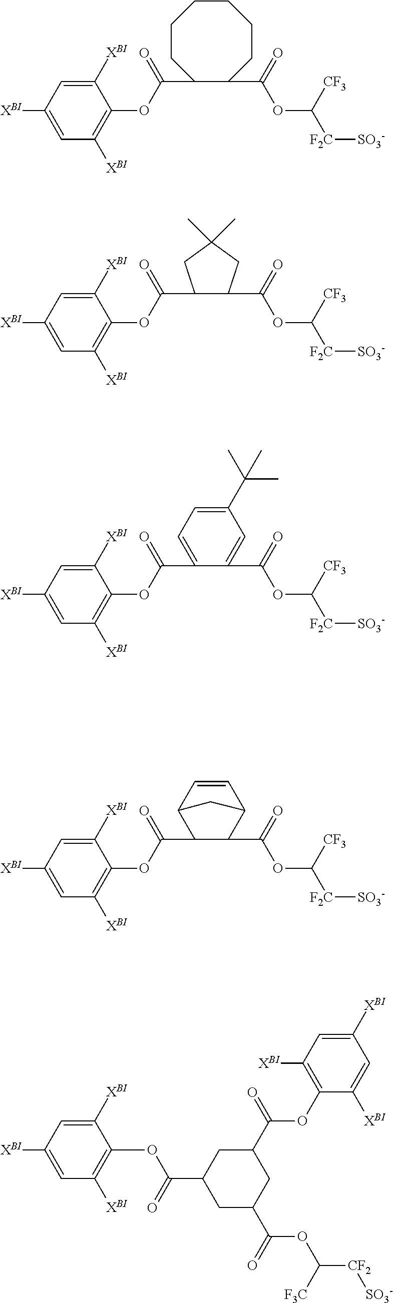

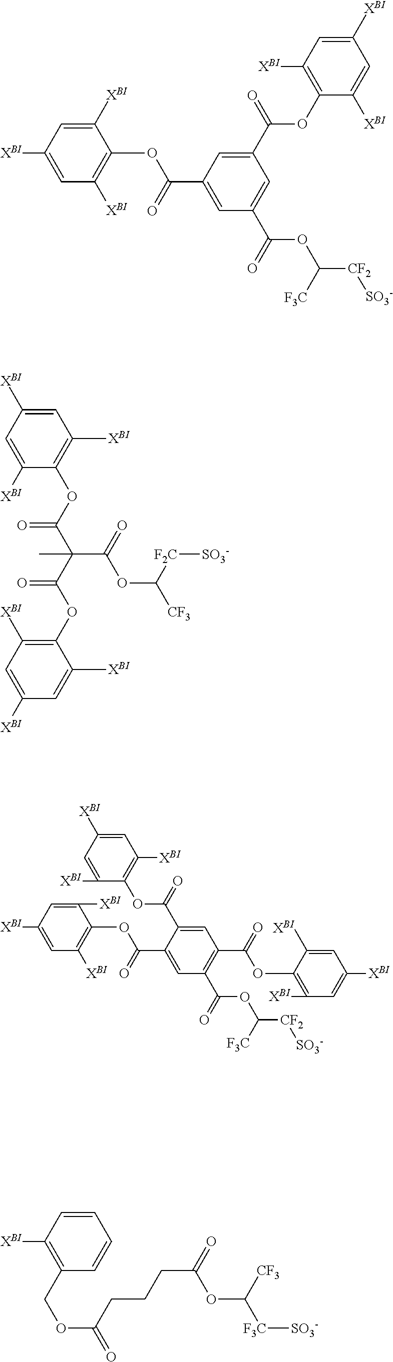

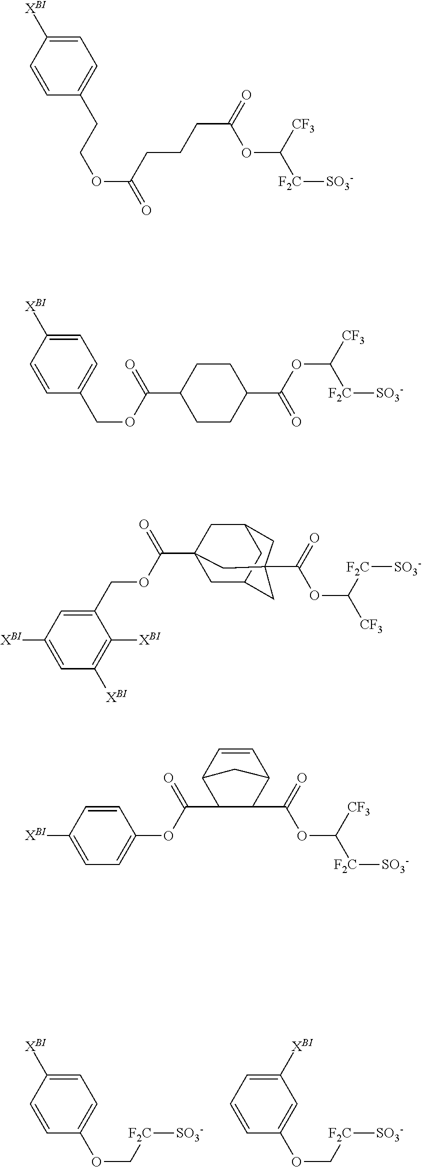

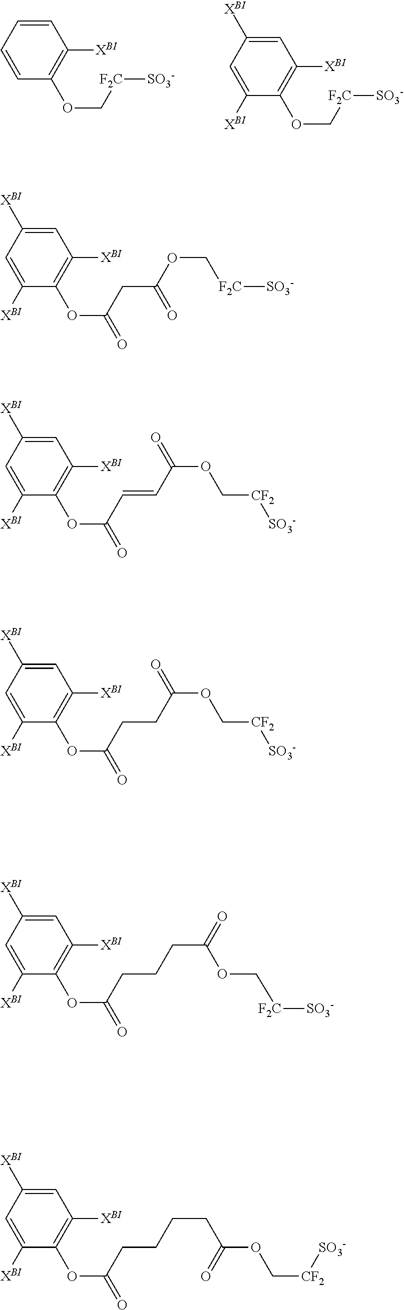

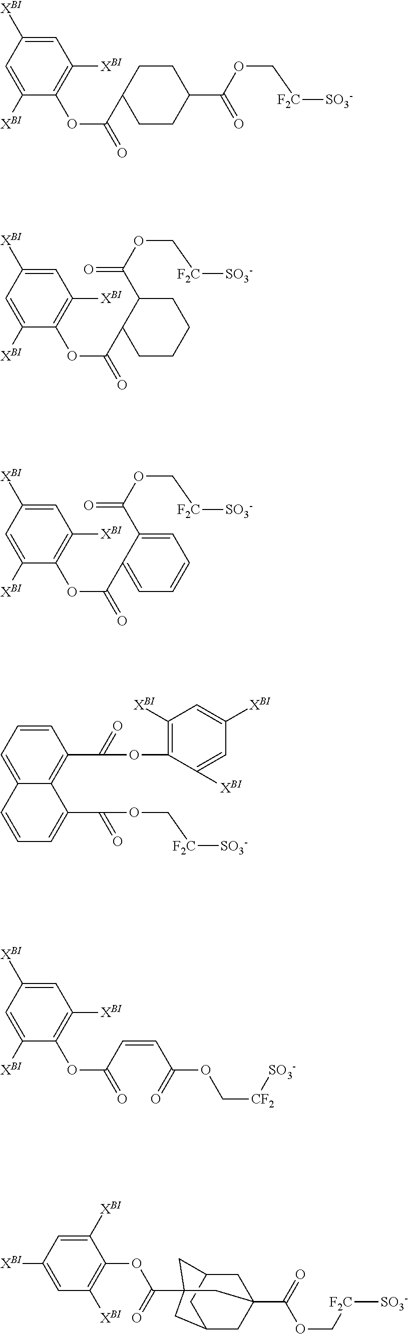

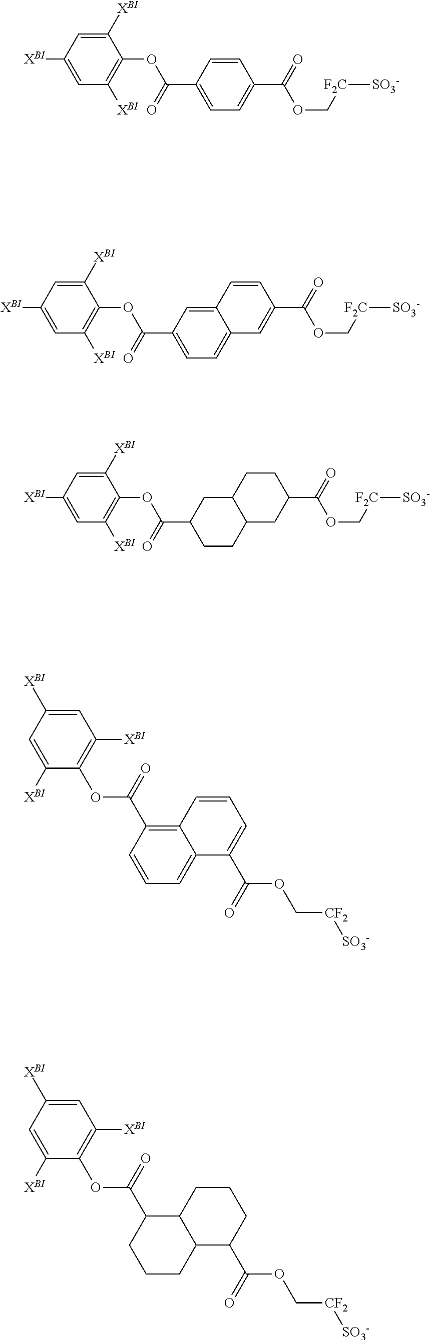

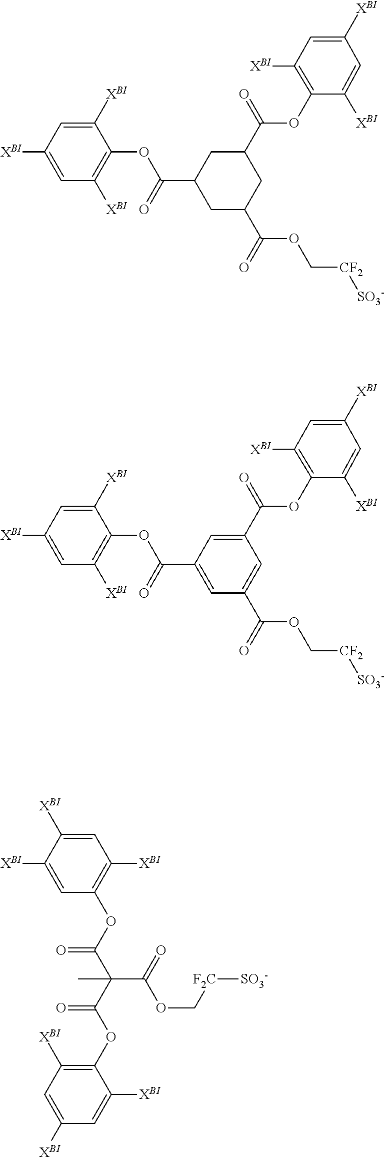

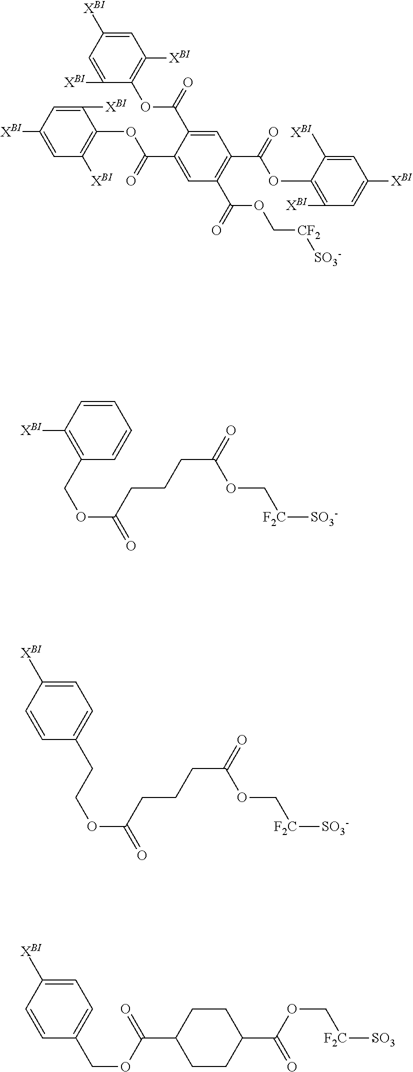

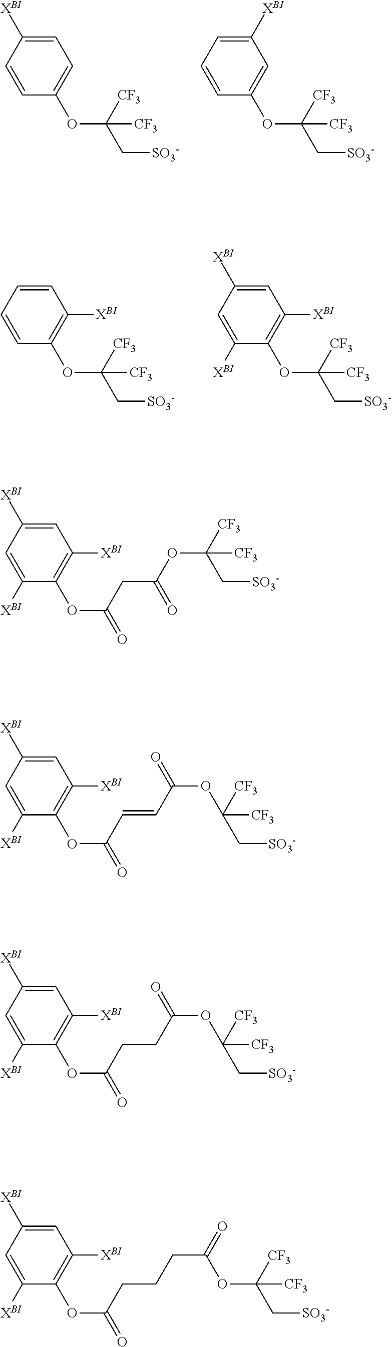

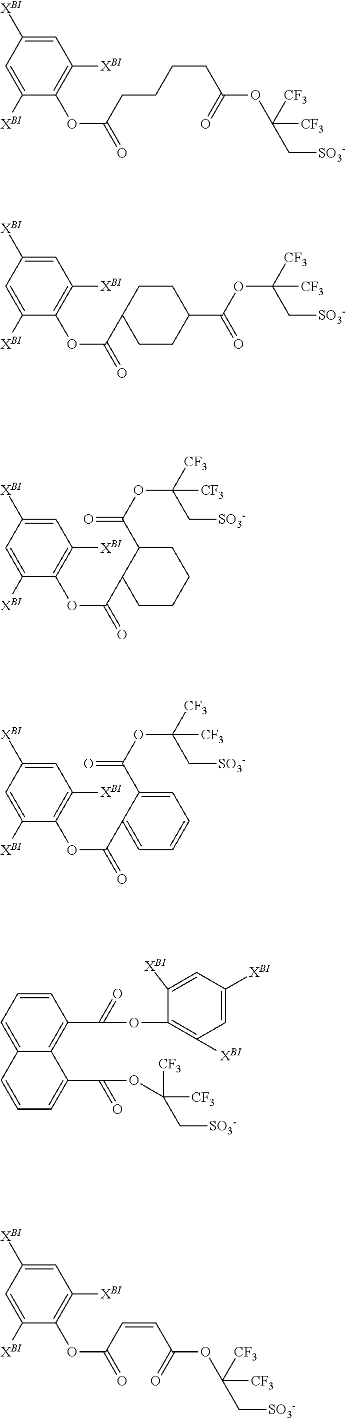

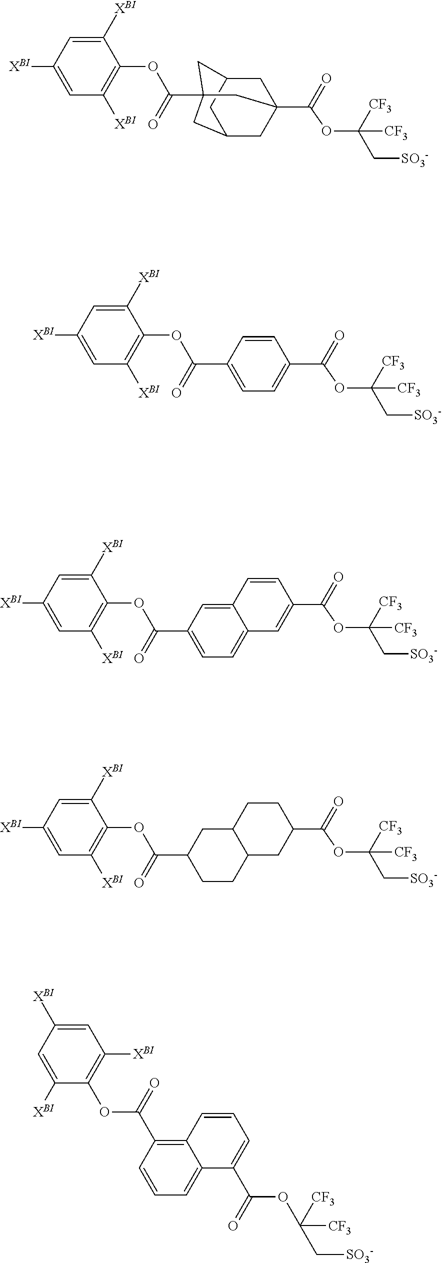

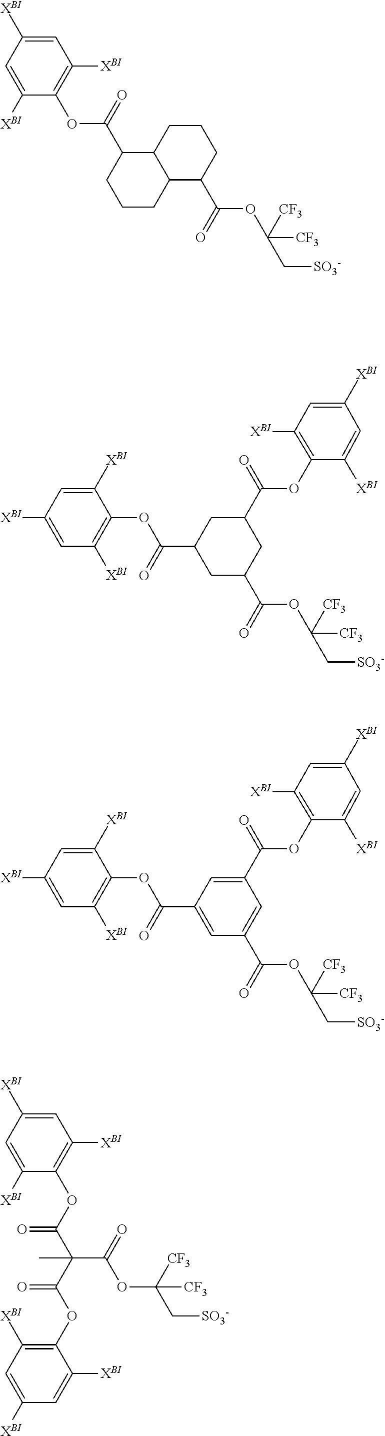

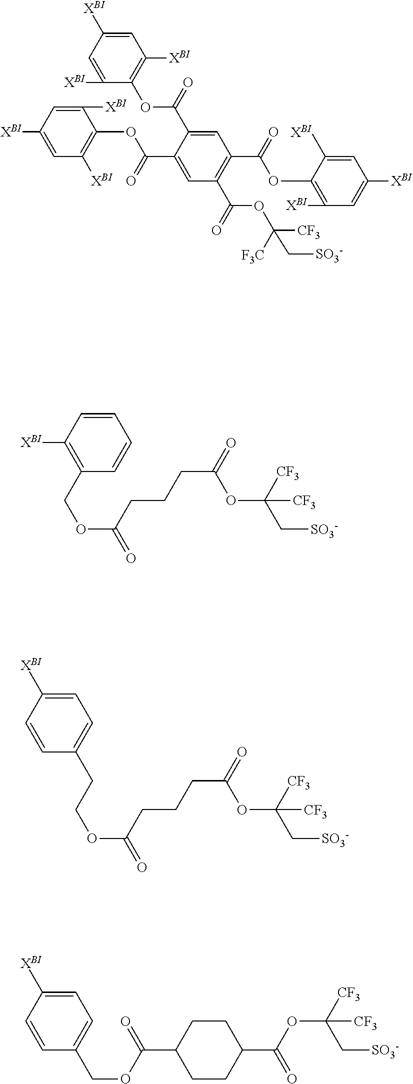

Other useful examples of the non-nucleophilic counter ion include fluorobenzenesulfinic acid anions having an iodized aromatic ring bonded thereto as described in JP6648726, anions having an acid-catalyzed decomposition mechanism as described in WO2021/200056 and JP-A 2021-070692, anions having a cyclic ether group as described in JP-A 2018-180525 and JP-A 2021-035935, and anions as described in JP-A 2018-092159.

Further useful examples of the non-nucleophilic counter ion include bulky fluorine-free benzenesulfonic acid anions as described in JP-A 2006-276759, JP-A 2015-117200, JP-A 2016-065016, and JP-A 2019-202974: fluorine-free benzenesulfonic acid or alkylsulfonic acid anions having an iodized aromatic group bonded thereto as described in JP 6645464.

Also useful are bissulfonic acid anions as described in JP-A 2015-206932, sulfonamide or sulfonimide anions having sulfonic acid side and different side as described in WO 2020/158366, and anions having a sulfonic acid side and a carboxylic acid side as described in JP-A 2015-024989.

Also compounds having the formula (3) are preferred as the PAG.

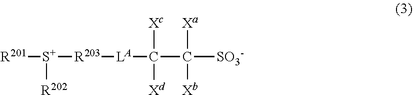

In formula (3), R²⁰¹ and R²⁰² are each independently a C₁-C₃₀ hydrocarbyl group which may contain a heteroatom. R²⁰³ is a C₁-C₃₀ hydrocarbylene group which may contain a heteroatom. Any two of R²⁰¹, R²⁰² and R²⁰³ may bond together to form a ring with the sulfur atom to which they are attached.

The C₁-C₃₀ hydrocarbyl groups R²⁰¹ and R²⁰² may be saturated or unsaturated and straight, branched or cyclic. Examples thereof include C₁-C₃₀ alkyl groups such as methyl, ethyl, n-propyl, isopropyl, n-butyl, sec-butyl, tert-butyl, n-pentyl, tert-pentyl, n-hexyl, n-octyl, 2-ethylhexyl, n-nonyl, and n-decyl; C₃-C₃₀ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, tricyclo[5.2.1.0^{2,6}]decanyl, and adamantyl; C₆-C₃₀ aryl groups such as phenyl, methylphenyl, ethylphenyl, n-propylphenyl, isopropylphenyl, n-butylphenyl, isobutylphenyl, sec-butylphenyl, tert-butylphenyl, naphthyl, methylnaphthyl, ethylnaphthyl, n-propylnaphthyl, isopropylnaphthyl, n-butylnaphthyl, isobutylnaphthyl, sec-butylnaphthyl, tert-butylnaphthyl, and anthracenyl; and combinations thereof. In these hydrocarbyl groups, some or all of the hydrogen atoms may be substituted by a moiety containing a heteroatom such as oxygen, sulfur, nitrogen or halogen, or some constituent —CH₂— may be replaced by a moiety containing a heteroatom such as oxygen, sulfur or nitrogen, so that the group may contain a hydroxy, cyano, fluorine, chlorine, bromine, iodine, carbonyl, ether bond, ester bond, sulfonic ester bond, carbonate bond, lactone ring, sultone ring, carboxylic anhydride (—C(═O)—O—C(═O)—) or haloalkyl moiety.

The C₁-C₃₀ hydrocarbylene group R²⁰³ may be saturated or unsaturated and straight, branched or cyclic. Examples thereof include C₁-C₃₀ alkanediyl groups such as methanediyl, ethane-1,1-diyl, ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, undecane-1,11-diyl, dodecane-1,12-diyl, tridecane-1,13-diyl, tetradecane-1,14-diyl, pentadecane-1,15-diyl, hexadecane-1,16-diyl, and heptadecane-1,17-diyl; C₃-C₃₀ cyclic saturated hydrocarbylene groups such as cyclopentanediyl, cyclohexanediyl, norbomanediyl and adamantanediyl; C₆-C₃₀ arylene groups such as phenylene, methylphenylene, ethylphenylene, n-propylphenylene, isopropylphenylene, n-butylphenylene, isobutylphenylene, sec-butylphenylene, tert-butylphenylene, naphthylene, methylnaphthylene, ethylnaphthylene, n-propyhiaphthylene, isopropylnaphthylene, n-butylnaphthylene, isobutylnaphthylene, sec-butylnaphthylene, and tert-butylnaphthylene; and combinations thereof. In these hydrocarbylene groups, some or all of the hydrogen atoms may be substituted by a moiety containing a heteroatom such as oxygen, sulfur, nitrogen or halogen, or some constituent —CH₂— may be replaced by a moiety containing a heteroatom such as oxygen, sulfur or nitrogen, so that the group may contain a hydroxy, cyano, fluorine, chlorine, bromine, iodine, carbonyl, ether bond, ester bond, sulfonic ester bond, carbonate bond, lactone ring, sultone ring, carboxylic anhydride (—C(═O)—O—C(═O)—) or haloalkyl moiety. Of the heteroatoms, oxygen is preferred.

In formula (3). L^{A} is a single bond, ether bond or a C₁-C₂₀ hydrocarbylene group which may contain a heteroatom. The hydrocarbylene group may be saturated or unsaturated and straight, branched or cyclic. Examples thereof are as exemplified above for R²⁰³.

In formula (3), X^{a}, X^{b}, X^{c} and X^{d} are each independently hydrogen, fluorine or trifluoromethyl, with the proviso that at least one of X^{a}, X^{b}, X^{c} and X^{d} is fluorine or trifluoromethyl.

Of the PAGs having formula (3), those having the formula (3′) are preferred.

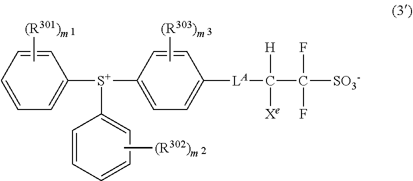

In formula (3′), L^{A} is as defined above. X^{e} is hydrogen or trifluoromethyl, preferably trifluoromethyl. R³⁰¹, R³⁰² and R³⁰³ are each independently hydrogen or a C₁-C₂₀ hydrocarbyl group which may contain a heteroatom. The hydrocarbyl group may be saturated or unsaturated and straight, branched or cyclic. Examples thereof are as exemplified above for the hydrocarbyl group R^{fa1} in formula (2A′). The subscripts m¹ and m² are each independently an integer of 0 to 5, and m³ is an integer of 0 to 4.

Examples of the PAG having formula (3) are as exemplified for the PAG having formula (2) in JP-A 2017-026980.

Of the foregoing PAGs, those having an anion of formula (2A′) or (2D) are especially preferred because of reduced acid diffusion and high solubility in the resist solvent. Also those having formula (3′) are especially preferred because of extremely reduced acid diffusion.

When the resist composition contains the PAG (D), it is preferably used in an amount of 0.1 to 40 parts, and more preferably 0.5 to 20 parts by weight per 80 parts by weight of the base polymer (C). An amount of the PAG (D) in the range ensures good resolution and eliminates the risk of leaving foreign particles after development or during separation of resist film. The PAG (D) may be used alone or in admixture of two or more. When the base polymer contains repeat units (c1) to (c4) and/or the resist composition contains the PAG (D), the resist composition functions as a chemically amplified resist composition.

(E) Nitrogen-Containing Compound

While the resist composition essentially contains the quencher (A), it may further contain a nitrogen-containing compound as another quencher. Suitable nitrogen-containing compounds include primary, secondary and tertiary amine compounds, specifically amine compounds having a hydroxy group, ether bond, ester bond, lactone ring, cyano group, or sulfonic ester bond, as described in JP-A 2008-111103, paragraphs [0146]-[0164], and primary and secondary amine compounds protected with a carbamate group, as described in JP 3790649.

Also a sulfonium salt of sulfonic acid having a nitrogen-containing substituent may be used as the nitrogen-containing compound (E). This compound functions as a quencher in the unexposed region, but as a so-called photo-degradable base in the exposed region because it loses the quencher function in the exposed region due to neutralization thereof with the acid generated by itself. Using a photo-degradable base, the contrast between exposed and unexposed regions can be further enhanced. With respect to the photo-degradable base, reference may be made to JP-A 2009-109595 and 2012-046501, for example.

When the resist composition contains the nitrogen-containing compound (E), it is preferably used in an amount of 0.001 to 12 parts by weight, more preferably 0.01 to 8 parts by weight per 80 parts by weight of the base polymer (C). The nitrogen-containing compound may be used alone or in admixture.

(F) Surfactant

The resist composition may further comprise (F) a surfactant. It is typically a surfactant which is insoluble or substantially insoluble in water and alkaline developer, or a surfactant which is insoluble or substantially insoluble in water and soluble in alkaline developer. For the surfactant, reference should be made to those compounds described in JP-A 2010-215608 and JP-A 2011-016746.

While many examples of the surfactant which is insoluble or substantially insoluble in water and alkaline developer are described in the patent documents cited herein, preferred examples are fluorochemical surfactants FC-4430 (3M). Olfine® E1004 (Nissin Chemical Co., Ltd.), Surflon® 5-381, KH-20 and KH-30 (AGC Seimi Chemical Co., Ltd.). Partially fluorinated oxetane ring-opened polymers having the formula (surf-1) are also useful.

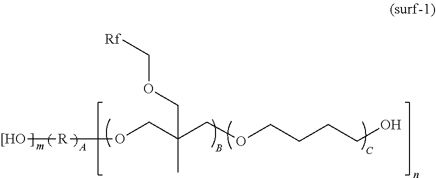

It is provided herein that R, Rf, A, B, C, n, and n are applied to only formula (surf-1), independent of their descriptions other than for the surfactant. R is a di- to tetra-valent C₂-C₅ aliphatic group. Exemplary divalent aliphatic groups include ethylene, 1,4-butylene, 1,2-propylene, 2,2-dimethyl-1,3-propylene and 1,5-pentylene. Exemplary tri- and tetra-valent groups are shown below.

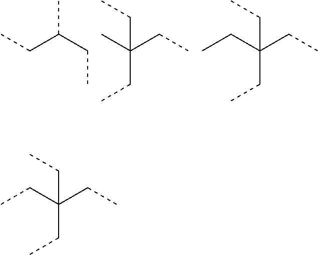

Herein the broken line denotes a valence bond. These formulae are partial structures derived from glycerol, trimethylol ethane, trimethylol propane, and pentaerythritol, respectively. Of these, 1,4-butylene and 2,2-dimethyl-1,3-propylene are preferred.

Rf is trifluoromethyl or pentafluoroethyl, and preferably trifluoromethyl. The subscript m is an integer of 0 to 3, n is an integer of 1 to 4, and the sum of m and n, which represents the valence of R, is an integer of 2 to 4. “A” is equal to 1, B is an integer of 2 to 25, and C is an integer of 0 to 10. Preferably, B is an integer of 4 to 20, and C is 0 or 1. Note that the formula (surf-1) does not prescribe the arrangement of respective constituent units while they may be arranged either blockwise or randomly. For the preparation of surfactants in the form of partially fluorinated oxetane ring-opened polymers, reference should be made to U.S. Pat. No. 5,650,483, for example.

The surfactant which is insoluble or substantially insoluble in water and soluble in alkaline developer is useful when ArF immersion lithography is applied to the resist composition in the absence of a resist protective film. In this embodiment, the surfactant has a propensity to segregate on the surface of a resist film for achieving a function of minimizing water penetration or leaching. The surfactant is also effective for preventing water-soluble components from being leached out of the resist film for minimizing any damage to the exposure tool. The surfactant becomes solubilized during alkaline development following exposure and PEB, and thus forms few or no foreign particles which become defects. The preferred surfactant is a polymeric surfactant which is insoluble or substantially insoluble in water, but soluble in alkaline developer, also referred to as “hydrophobic resin” in this sense, and especially which is water repellent and enhances water sliding.

Suitable polymeric surfactants include those containing repeat units of at least one type selected from the formulae (4A) to (4E).

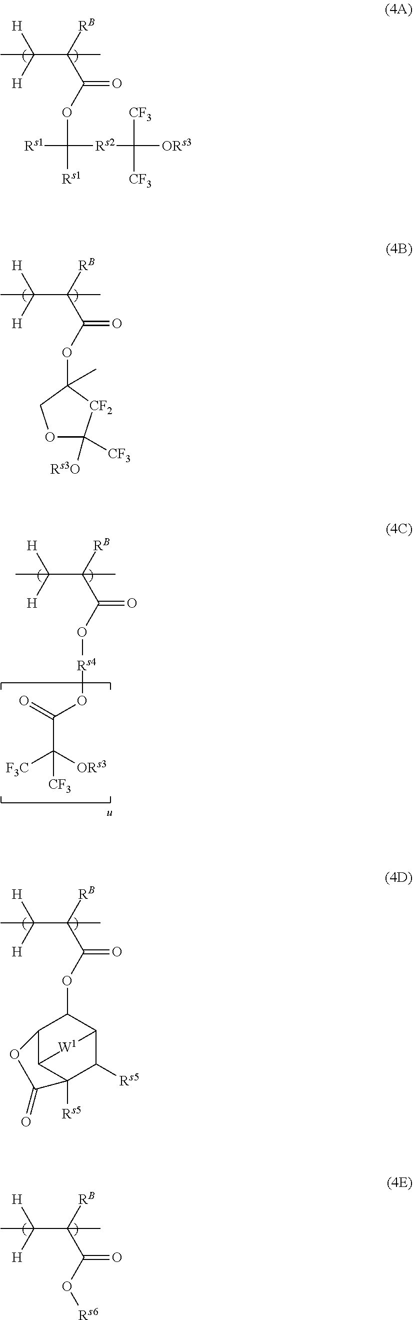

In formulae (4A) to (4E), RB is hydrogen, fluorine, methyl or trifluoromethyl. W¹ is —CH₂—, —CH₂CH₂— or —O—, or two separate —H. R^{s1} is each independently hydrogen or a C₁-C₁₀ hydrocarbyl group. R^{s2} is a single bond or a C₁-C₅ straight or branched hydrocarbylene group. R^{s3} is each independently hydrogen, a C₁-C₁₅ hydrocarbyl or fluorinated hydrocarbyl group, or an acid labile group. When R^{s3} is a hydrocarbyl or fluorinated hydrocarbyl group, an ether bond or carbonyl moiety may intervene in a carbon-carbon bond. R^{s4} is a C₁-C₂₀ (u+1)-valent hydrocarbon or fluorinated hydrocarbon group, and u is an integer of 1 to 3. R^{s5} is each independently hydrogen or a group: —C(═O)—O—R^{sa} wherein R^{sa} is a C₁-C₂₀ fluorinated hydrocarbyl group. R^{s6} is a C₁-C₁₅ hydrocarbyl or fluorinated hydrocarbyl group in which an ether bond or carbonyl moiety may intervene in a carbon-carbon bond.

The hydrocarbyl group R^{s1} is preferably saturated while it may be straight, branched or cyclic. Examples thereof include alkyl groups such as methyl, ethyl, n-propyl, isopropyl, n-butyl, isobutyl, sec-butyl, tert-butyl, n-pentyl, n-hexyl, n-heptyl, n-octyl, n-nonyl and n-decyl, and cyclic saturated hydrocarbyl groups such as cyclopropyl, cyclobutyl, cyclopentyl, cyclohexyl, adamantyl and norbornyl. Inter alia, C₁-C₆ groups are preferred.

The hydrocarbylene group R^{s2} is preferably saturated while it may be straight, branched or cyclic. Examples thereof include methylene, ethylene, propylene, butylene, and pentylene.

The hydrocarbyl group R^{s3} or R^{s6} may be saturated or unsaturated and straight, branched or cyclic. Examples thereof include saturated hydrocarbyl groups and aliphatic unsaturated hydrocarbyl groups such as alkenyl and alkynyl groups, with the saturated hydrocarbyl groups being preferred. Suitable saturated hydrocarbyl groups include those exemplified for the hydrocarbyl group represented by R^{s1} as well as n-undecyl, n-dodecyl, tridecyl, tetradecyl, and pentadecyl. Examples of the fluorinated hydrocarbyl group represented by R^{s3} or R^{s6} include the foregoing hydrocarbyl groups in which some or all carbon-bonded hydrogen atoms are substituted by fluorine atoms. In these groups, an ether bond or carbonyl moiety may intervene in a carbon-carbon bond as mentioned above.

Examples of the acid labile group represented by R^{s3} include groups of the above formulae (AL-3) to (AL-5), trialkylsilyl groups in which each alkyl moiety has 1 to 6 carbon atoms, and C₄-C₂₀ oxoalkyl groups.

The (u+1)-valent hydrocarbon or fluorinated hydrocarbon group represented by R^{s4} may be straight, branched or cyclic, and examples thereof include the foregoing hydrocarbyl or fluorinated hydrocarbyl groups from which “u” number of hydrogen atoms are eliminated.

The fluorinated hydrocarbyl group represented by R^{sa} is preferably saturated while it may be straight, branched or cyclic. Examples thereof include the foregoing hydrocarbyl groups in which some or all hydrogen atoms are substituted by fluorine atoms. Illustrative examples include trifluoromethyl, 2,2,2-trifluoroethyl, 3,3,3-trifluoro-1-propyl, 3,3,3-trifluoro-2-propyl, 2,2,3,3-tetrafluoropropyl, 1,1,1,3,3,3-hexafluoroisopropyl, 2,2,3,3,4,4,4-heptafluorobutyl, 2,2,3,3,4,4,5,5-octafluoropentyl, 2,2,3,3,4,4,5,5,6,6,7,7-dodecafluoroheptyl, 2-(perfluorobutyl)ethyl, 2-(perfluorohexyl)ethyl, 2-(perfluorooctyl)ethyl, and 2-(perfluorodecyl)ethyl.

Examples of the repeat units having formulae (4A) to (4E) are shown below, but not limited thereto. Herein R¹ is as defined above.

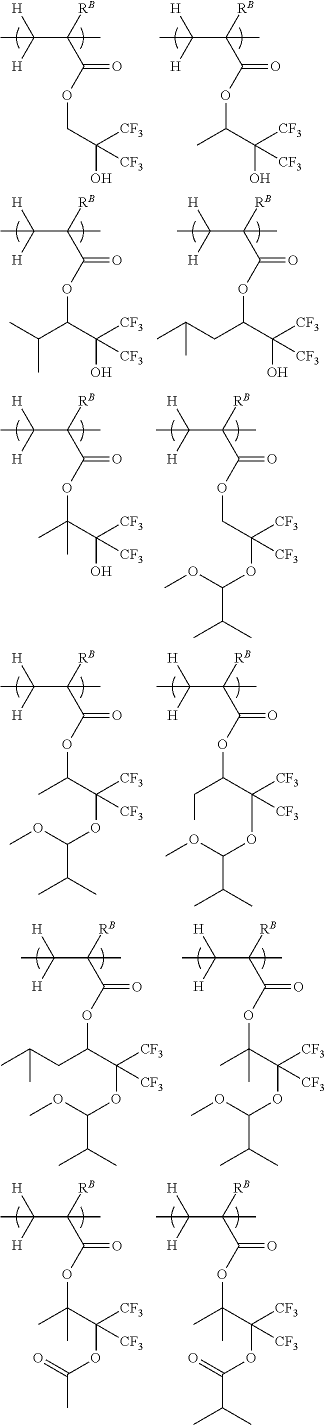

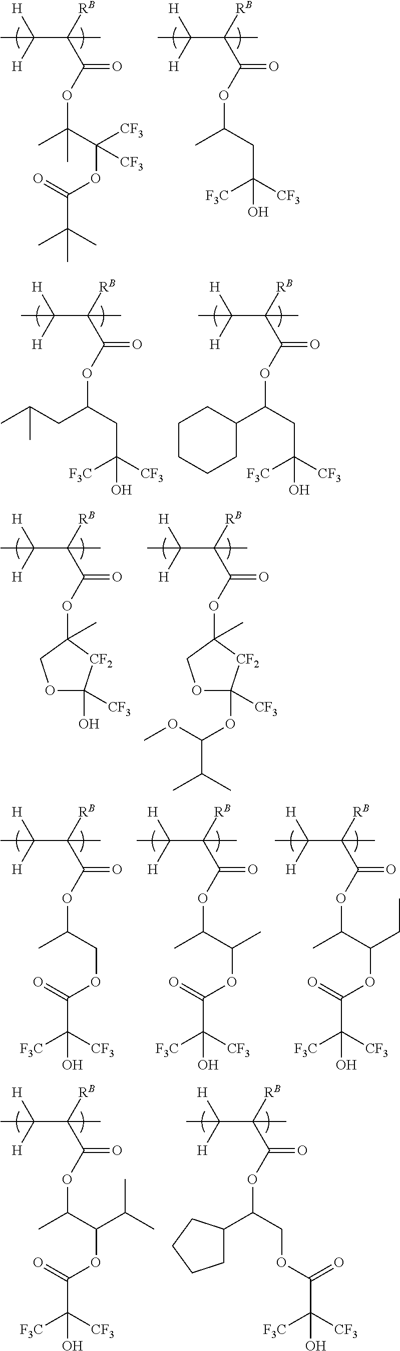

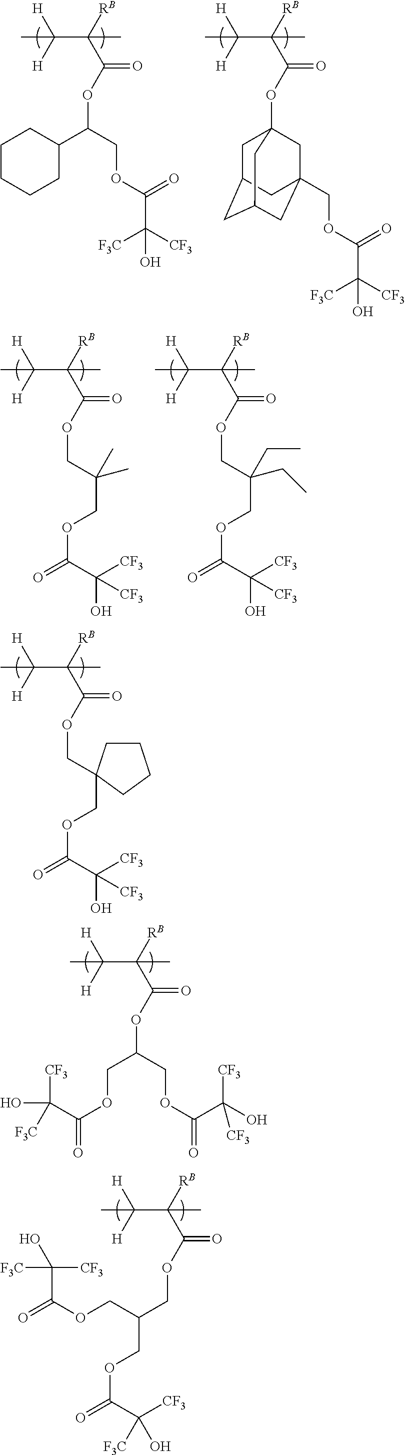

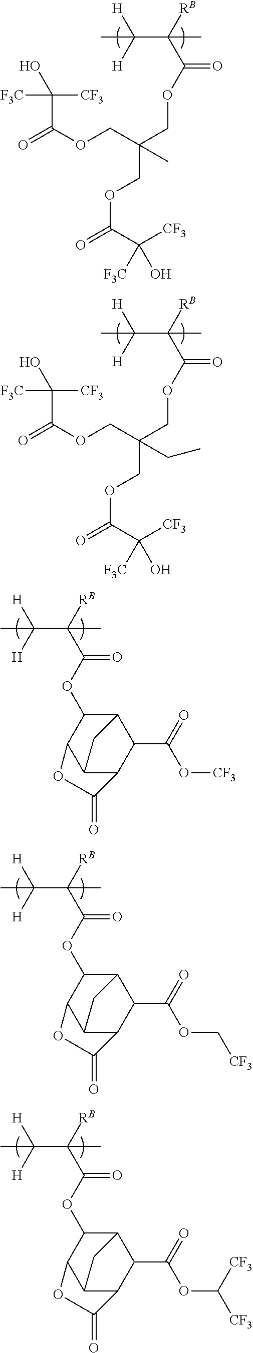

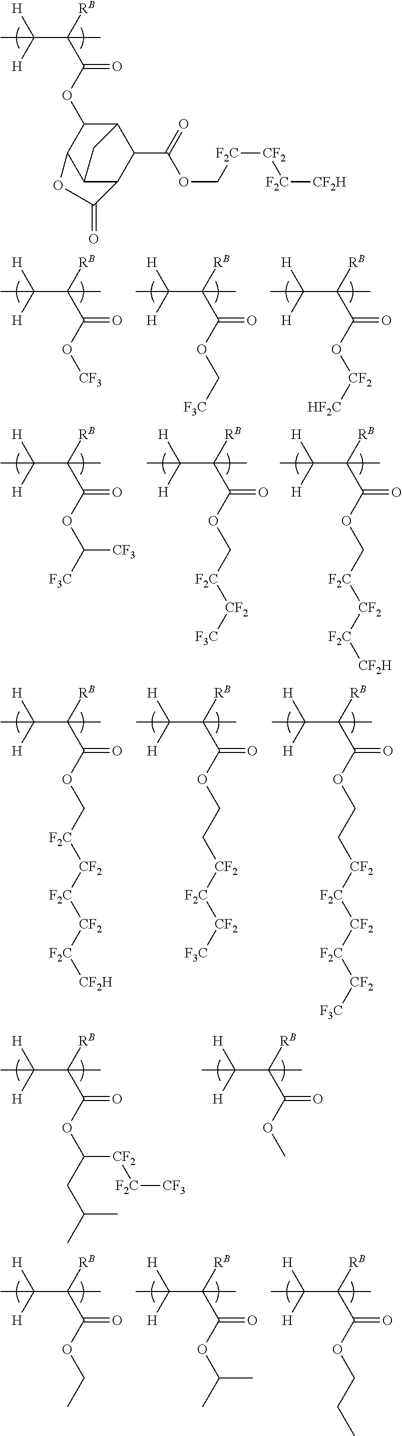

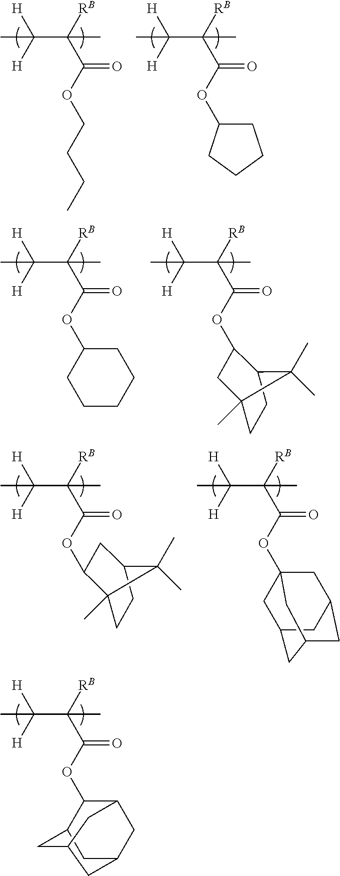

The polymeric surfactant may further contain repeat units other than the repeat units having formulae (4A) to (4E). Typical other repeat units are those derived from methacrylic acid and α-trifluoromethylacrylic acid derivatives. In the polymeric surfactant, the content of the repeat units having formulae (4A) to (4E) is preferably at least 20 mol %, more preferably at least 60 mol %, most preferably 100 mol % of the overall repeat units.

Preferably the polymeric surfactant has a Mw of 1,000 to 500,000, more preferably 3,000 to 100,000 and a Mw/Mn of 1.0 to 2.0, more preferably 1.0 to 1.6.

The polymeric surfactant may be synthesized, for example, by dissolving an unsaturated bond-containing monomer or monomers, from which repeat units having formulae (4A) to (4E) and optional other repeat units are derived, in an organic solvent, adding a radical initiator, and heating for polymerization. Suitable organic solvents used herein include toluene, benzene, THF, diethyl ether, and dioxane. Examples of the polymerization initiator used herein include AIBN, 2,2′-azobis(2,4-dimethylvaleronitrile), dimethyl 2,2-azobis(2-methylpropionate), benzoyl peroxide, and lauroyl peroxide. Preferably the reaction temperature is 50 to 100° C. and the reaction time is 4 to 24 hours. The acid labile group that has been incorporated in the monomer may be kept as such, or the polymer may be protected or partially protected therewith at the end of polymerization.

During the synthesis of the polymeric surfactant, any of well-known chain transfer agents such as dodecylmercaptan and 2-mercaptoethanol may be used for the purpose of adjusting molecular weight. An appropriate amount of the chain transfer agent is 0.01 to 10 mol % based on the total moles of monomers to be polymerized.

When the resist composition contains the surfactant (F), it is preferably used in an amount of 0.1 to 50 parts by weight, more preferably 0.5 to 10 parts by weight per 80 parts by weight of the base polymer (C). As long as the amount of the surfactant is at least 0.1 part by weight, the receding contact angle of resist film surface with water is fully improved. As long as the amount of the surfactant is up to 50 parts by weight, the dissolution rate of resist film surface in developer is so low that the resulting small-size pattern may maintain a sufficient height. The surfactant (F) may be used alone or in admixture.

(G) Other Components

The resist composition may further comprise other components, for example, a compound which is decomposed with an acid to generate another acid (i.e., acid amplifier compound), organic acid derivative, fluorinated alcohol, and a compound with Mw≤3,000 adapted to change its solubility in developer under the action of acid (i.e., dissolution inhibitor). Each of the other components may be used alone or in admixture.

The acid amplifier compound is described in JP-A 2009-269953 and JP-A 2010-15608. The acid amplifier compound is preferably used in an amount of 0 to 5 parts, more preferably 0 to 3 parts by weight per 80 parts by weight of the base polymer. An extra amount of the acid amplifier compound can make the acid diffusion control difficult and cause degradations to resolution and pattern profile. With respect to the organic acid derivative, fluorinated alcohol and dissolution inhibitor, reference should be made to JP-A 2009-269953 and JP-A 2010-215608.

Process

A further embodiment of the invention is a pattern forming process comprising the steps of applying the resist composition defined above onto a substrate to form a resist film thereon, exposing the resist film to high-energy radiation, baking the exposed resist film, and developing the baked resist film in a developer to form a resist pattern.

The substrate used herein may be selected from, for example, substrates for IC fabrication. e.g., Si, SiO₂, SiN, SiON, TiN, WSi, BPSG, SOG, and organic antireflective coating, and substrates for mask circuit fabrication, e.g., Cr, CrO, CrON, MoSi₂, and SiO₂.

The resist composition is first applied onto a substrate by a suitable coating technique such as spin coating. The coating is prebaked on a hotplate preferably at a temperature of 60 to 150° C. for 1 to 10 minutes, more preferably at 80 to 140° C. for 1 to 5 minutes to form a resist film of 0.05 to 2 μm thick.

Then the resist film is exposed patternwise to high-energy radiation, for example, KrF excimer laser, ArF excimer laser, EB or EUV. On use of KrF or ArF excimer laser or EUV, the resist film is exposed through a mask having the desired pattern, preferably in a dose of 1 to 200 mJ/cm², more preferably 10 to 100 mJ/cm². On use of EB, a pattern may be written directly or through a mask having the desired pattern, preferably in a dose of 1 to 300 μC/cm², more preferably 10 to 200 ρC/cm².

The exposure may be performed by conventional lithography whereas the immersion lithography of holding a liquid having a refractive index of at least 1.0, typically water between the resist film and the projection lens may be employed if desired. In the case of immersion lithography, a protective film which is insoluble in water may be formed on the resist film.

While the water-insoluble protective film serves to prevent any components from being leached out of the resist film and to improve water slippage at the film surface, it is generally divided into two types. The first type is an organic solvent-strippable protective film which must be stripped, prior to alkaline development, with an organic solvent in which the resist film is not dissolvable. The second type is an alkali-soluble protective film which is soluble in an alkaline developer so that it can be removed simultaneously with the removal of solubilized regions of the resist film. The protective film of the second type is preferably of a material comprising a polymer having a 1,1,1,3,3,3-hexafluoro-2-propanol residue (which is insoluble in water and soluble in an alkaline developer) as a base in an alcohol solvent of at least 4 carbon atoms, an ether solvent of 8 to 12 carbon atoms or a mixture thereof. Alternatively, the aforementioned surfactant which is insoluble in water and soluble in an alkaline developer may be dissolved in an alcohol solvent of at least 4 carbon atoms, an ether solvent of 8 to 12 carbon atoms or a mixture thereof to form a material from which the protective film of the second type is formed.

After the exposure, the resist film is baked (PEB), for example, on a hotplate preferably at 60 to 150° C. for 1 to 5 minutes, and more preferably at 80 to 140° C. for 1 to 3 minutes.

Finally, development is carried out using as the developer an aqueous alkaline solution, such as a 0.1 to 5 wt %, preferably 2 to 3 wt %, aqueous solution of tetramethylammonium hydroxide (TMAH), this being done by a conventional method such as dip, puddle, or spray development for a period of 0.1 to 3 minutes, and preferably 0.5 to 2 minutes. In this way the exposed region of resist film is dissolved away, forming the desired pattern on the substrate.

After formation of the photoresist film, deionized water rinsing may be carried out for extracting the acid generator and the like from the film surface or washing away particles, or after exposure, rinsing may be carried out for removing water droplets left on the resist film.

A pattern may also be formed by a double patterning process. The double patterning process includes a trench process of processing an underlay to a 1:3 trench pattern by a first step of exposure and etching, shifting the position, and forming a 1:3 trench pattern by a second step of exposure for forming a 1:1 pattern; and a line process of processing a first underlay to a 1:3 isolated left pattern by a first step of exposure and etching, shifting the position, processing a second underlay formed below the first underlay by a second step of exposure through the 1:3 isolated left pattern, for forming a half-pitch 1:1 pattern.

In the pattern forming process, an alkaline aqueous solution is often used as the developer. Instead, the negative tone development technique wherein the unexposed region of resist film is dissolved in an organic solvent developer is also applicable. In the organic solvent development, the organic solvent used as the developer is preferably selected from 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, isopentyl acetate, butenyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, methyl phenylacetate, ethyl phenylacetate, benzyl formate, phenylethyl formate, methyl 3-phenylpropionate, benzyl propionate, and 2-phenylethyl acetate. These organic solvents may be used alone or in admixture of two or more.

### EXAMPLES

Examples of the invention are given below by way of illustration and not by way of limitation. The abbreviation “pbw” is parts by weight. Analysis is made by IR spectroscopy, NMR spectroscopy, and time-of-flight mass spectrometry (TOF-MS) using analytic instruments as shown below.
IR: NICOLET 6700 by Thermo Fisher Scientific Inc.
¹H-NMR: ECA-500 by JEOL Ltd.
MALDI TOF-MS: S3000 by JEOL Ltd.

### [1] Synthesis of Onium Salts

### Example 1-1: Synthesis of Onium Salt SQ-1

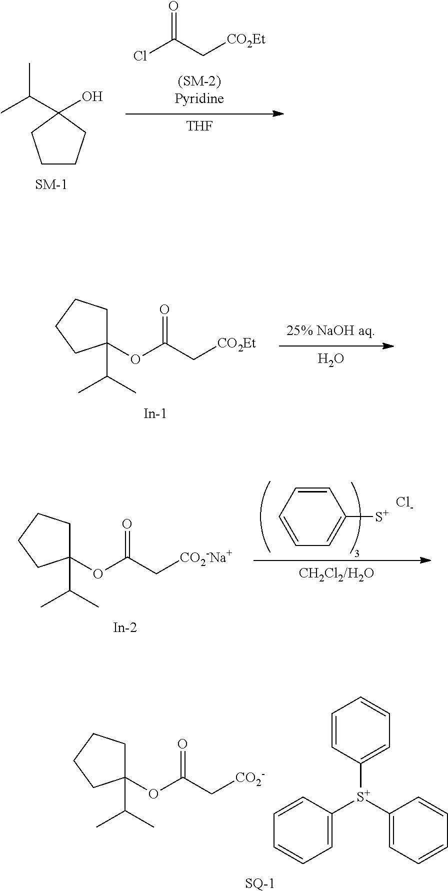

### (1) Synthesis of Intermediate In-1

In a reactor under nitrogen blanket, 57.8 g of Compound SM-1 and 42.7 g of pyridine were dissolved in 400 mL of THF, which was cooled in an ice bath. While the internal temperature was maintained below 20° C., 81.3 g of Compound SM-2 was added dropwise. At the end of addition, the reaction mixture was restored to room temperature and aged for 12 hours. The reaction solution was cooled in an ice bath, after which 300 mL of water was added to quench the reaction. The reaction product was extracted twice with 500 g of ethyl acetate, followed by ordinary aqueous work-up and solvent distillation. Subsequent purification by distillation gave 102.9 g (yield 94%) of Intermediate In-1 as colorless oily matter.

### (2) Synthesis of Intermediate In-2

In nitrogen atmosphere, 24.2 g of Intermediate In-1 was dissolved in 120 g of THF, to which 17.6 g of 25 wt % sodium hydroxide aqueous solution was added dropwise. At the end of addition, the reaction mixture was heated at 40° C. and aged for 4 hours. After the reaction system was cooled below 10° C., 100 g of diisopropyl ether and 100 g of water were added to wash the water layer. Then the water layer was taken out, which was an aqueous solution containing Intermediate In-2. The water layer taken out was used as such in the next step without further purification.

### (3) Synthesis of Onium Salt SQ-1

In nitrogen atmosphere, the aqueous solution containing Intermediate In-2 was mixed with 29.9 g of triphenylsulfonium chloride and 100 g of methylene chloride, which was stirred at room temperature for 2 hours. This was followed by ordinary aqueous work-up and solvent distillation, obtaining 34.8 g (yield 73%) of Onium Salt SQ-1 as oily matter.

The results of TOF-MS of Onium Salt SQ-1 are shown below.

MALDI TOF-MS:
positive M⁺ 263 (corresponding to C₁₈H₁₅S⁺)
negative M⁻ 213 (corresponding to C₁₁H₁₇O₄⁻)

### Example 1-2: Synthesis of Onium Salt SQ-2

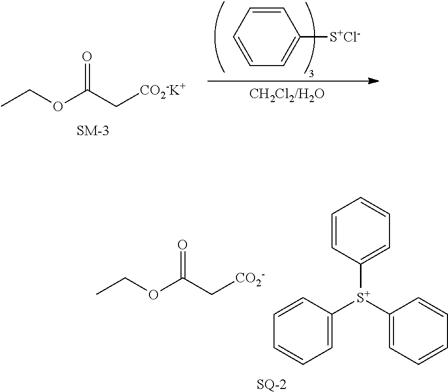

By the same procedure as in Example 1-1 (3) aside from using Compound SM-3 instead of Intermediate In-2, there was obtaining 7.7 g (yield 67%) of Onium Salt SQ-2 as oily matter.

The results of TOF-MS of Onium Salt SQ-2 are shown below.

MALDI TOF-MS:
positive M⁺ 263 (corresponding to C₁₈H₁₅S⁺)
negative M⁻ 131 (corresponding to C₅H₇O₄⁻)

### Examples 1-3 to 1-10: Synthesis of Onium Salts SQ-3 to SQ-10

Onium salts SQ-3 to SQ-10, shown below, were synthesized using the corresponding reactants and well-known organic chemistry reaction.

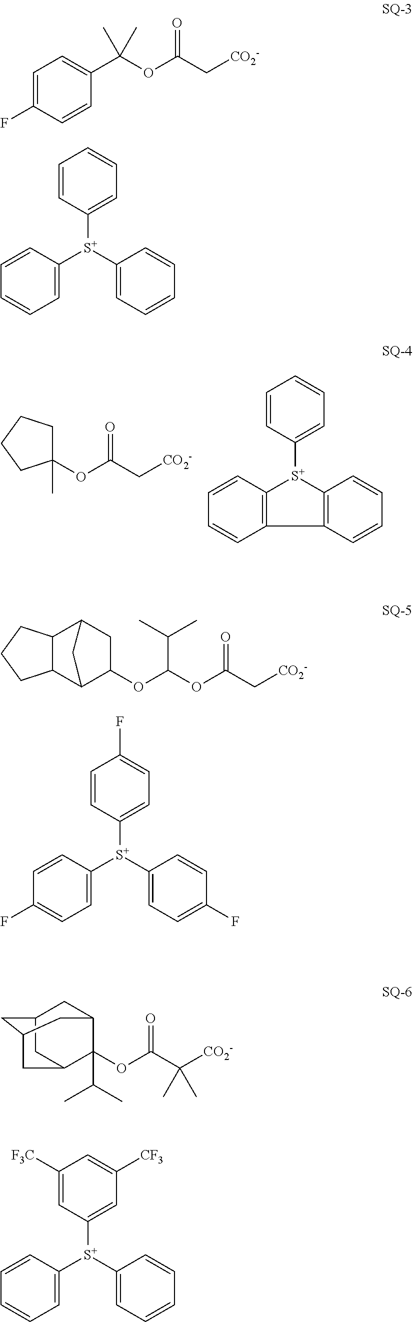

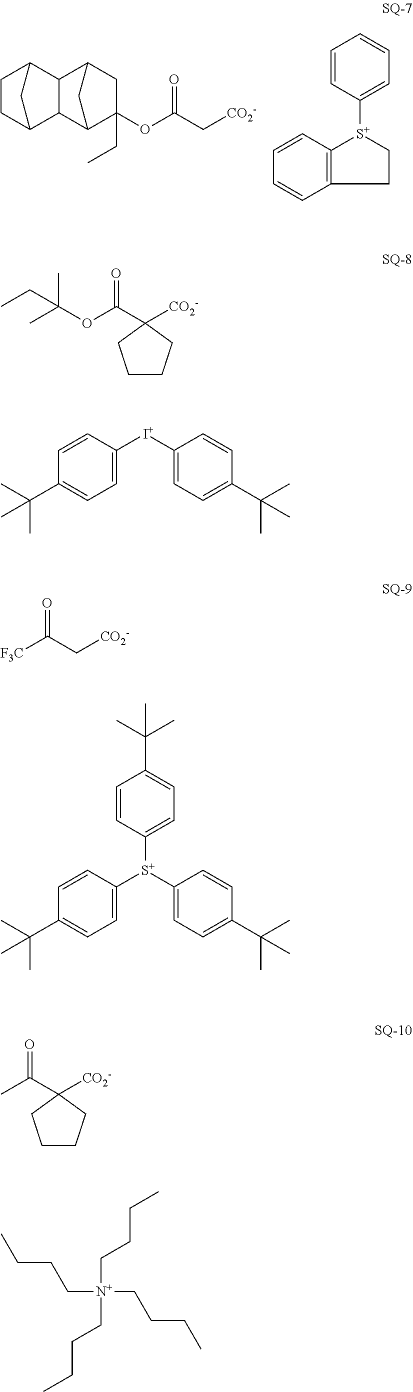

### [2] Synthesis of Base Polymer

Monomers of the structure shown below were used in the synthesis of base polymers.

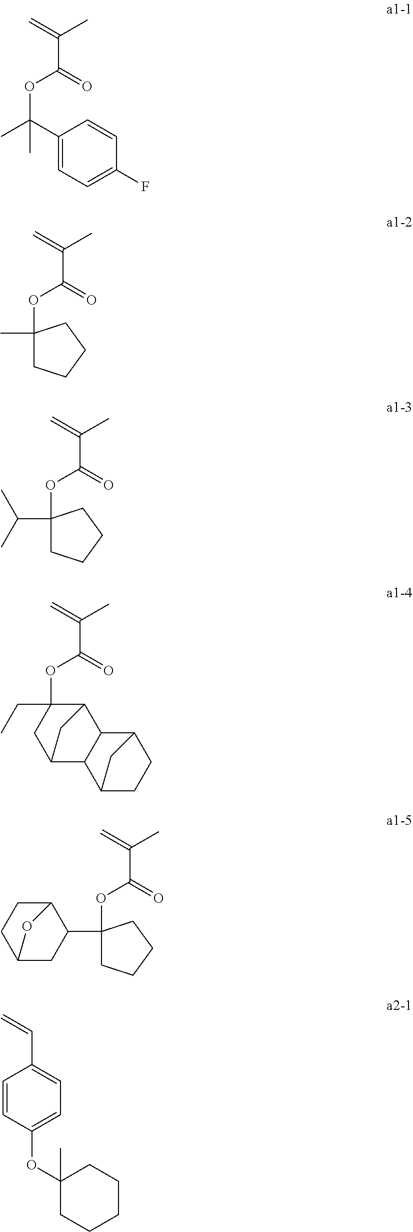

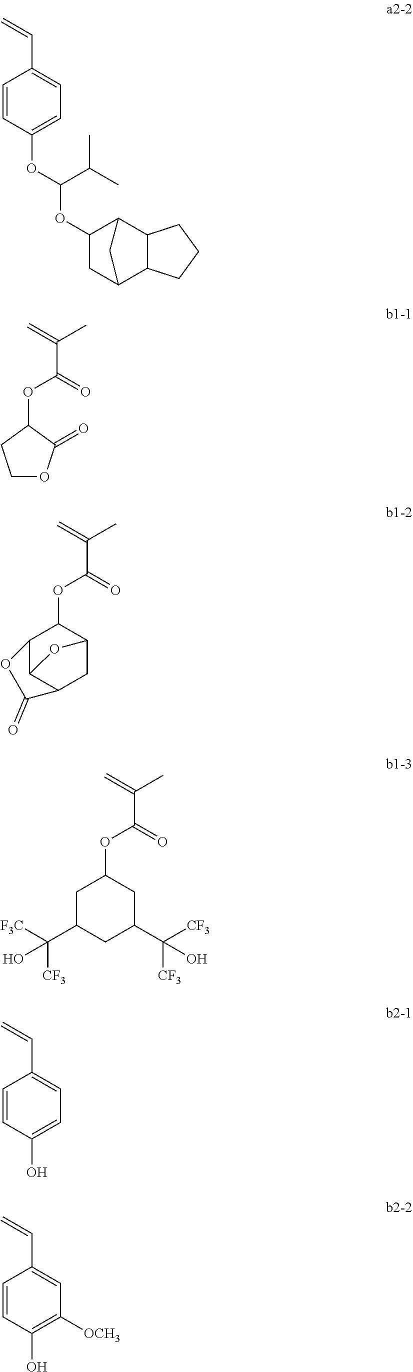

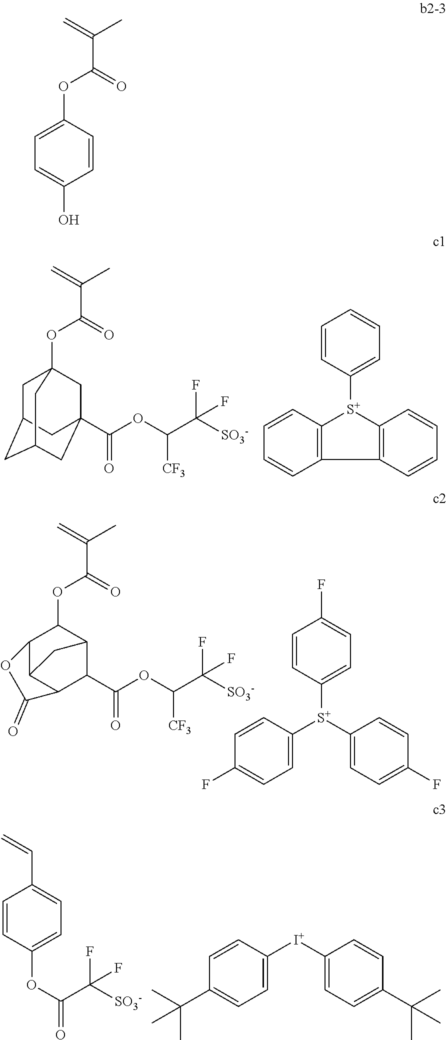

### Synthesis Example 1: Synthesis of Base Polymer P-1

A flask under nitrogen atmosphere was charged with 50.1 g of Monomer a1-1, 24.8 g of Monomer b2-1, 38.0 g of Monomer c1, 3.96 g of V-601 (dimethyl 2,2′-azobis(2-methylpropionate) by Fujifilm Wako Pure Chemical Corp.), and 127 g of MEK to form a monomer/initiator solution. Another flask under nitrogen atmosphere was charged with 46 g of MEK, which was heated at 80° C. with stirring. The monomer solution was added dropwise to the MEK over 4 hours. At the end of addition, the polymerization solution was continuously stirred for 2 hours while maintaining the temperature at 80° C. The polymerization solution was cooled to room temperature, after which it was added dropwise to 2,000 g of hexane with vigorous stirring. The solid precipitate was collected by filtration. The precipitate was washed twice with 600 g of hexane and vacuum dried at 50° C. for 20 hours, obtaining Base Polymer P-1 as white powder. Amount 98.1 g, yield 98%. Base Polymer P-1 had a Mw of 10,900 and a Mw/Mn of 1.82. It is noted that Mw is as measured by GPC versus polystyrene standards using DMF solvent.

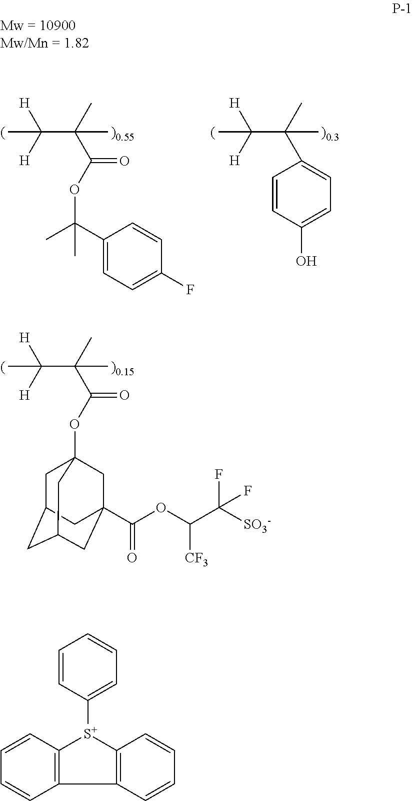

### Synthesis Examples 2 to 18: Synthesis of Base Polymers P-2 to P-18

Base Polymers P-2 to P-18, shown in Table 1, were synthesized by the same procedure as in Synthesis Example 1 except that the type and amount (blending ratio) of monomers were changed.

TABLE 1

| | | Incorpo- | | Incorpo- | | Incorpo- | | Incorpo- | | Incorpo- | | |
|---|---|---|---|---|---|---|---|---|---|---|---|---|
| | | ration | | ration | | ration | | ration | | ration | | |
| Base | Unit | ratio | Unit | ratio | Unit | ratio | Unit | ratio | Unit | ratio | | |
| Polymer | a1 | (mol %) | a2 | (mol %) | b1 | (mol %) | b2 | (mol %) | c | (mol %) | Mw | Mw/Mn |
| P-1 | a1-1 | 55 | — | — | — | — | b2-1 | 30 | c1 | 15 | 10,900 | 1.82 |
| P-2 | a1-2 | 55 | — | — | — | — | b2-1 | 30 | c1 | 15 | 10,700 | 1.81 |
| P-3 | a1-1 | 25 | — | — | — | — | b2-1 | 35 | c2 | 15 | 10,100 | 1.79 |
| | a1-3 | 25 | | | | | | | | | | |
| P-4 | a1-3 | 35 | — | — | — | — | b2-3 | 35 | c3 | 15 | 9,800 | 1.77 |
| | a1-5 | 15 | | | | | | | | | | |
| P-5 | a1-4 | 10 | a2-1 | 30 | b1-2 | 30 | b2-3 | 20 | c2 | 10 | 10,700 | 1.81 |
| P-6 | a1-1 | 50 | — | — | — | — | b2-1 | 25 | c2 | 10 | 10,700 | 1.81 |
| | | | | | | | b2-2 | 15 | | | | |
| P-7 | a1-3 | 50 | — | — | — | — | b2-1 | 35 | c2 | 15 | 9,900 | 1.77 |
| P-8 | a1-1 | 25 | — | — | — | — | b2-1 | 35 | c3 | 15 | 9,700 | 1.84 |
| | a1-3 | 25 | | | | | | | | | | |
| P-9 | a1-3 | 35 | a2-2 | 15 | — | — | b2-1 | 35 | c1 | 15 | 10,200 | 1.78 |
| P-10 | a1-1 | 30 | a2-1 | 20 | b1-3 | 10 | b2-1 | 20 | c2 | 20 | 10,000 | 1.82 |
| P-11 | a1-1 | 20 | a2-1 | 30 | — | — | b2-2 | 30 | c3 | 20 | 9,800 | 1.81 |
| P-12 | a1-1 | 35 | — | — | — | — | b2-1 | 35 | c2 | 15 | 9,600 | 1.79 |
| | a1-5 | 15 | | | | | | | | | | |
| P-13 | a1-1 | 35 | — | — | b1-3 | 10 | b2-3 | 30 | c1 | 10 | 9,900 | 1.81 |
| | a1-4 | | | | | | | | | | | |
| P-14 | a1-2 | 60 | — | — | — | — | b2-1 | 40 | — | — | 7,300 | 1.76 |
| P-15 | a1-1 | 50 | — | — | b1-1 | 20 | b2-1 | 20 | — | — | 7,400 | 1.74 |
| | | | | | b1-3 | 10 | | | | | | |
| P-16 | a1-1 | 50 | — | — | — | — | b2-1 | 50 | — | — | 7,700 | 1.78 |
| P-17 | a1-1 | 25 | a2-2 | 25 | b1-1 | 20 | b2-3 | 30 | — | — | 7,500 | 1.73 |
| P-18 | a1-3 | 35 | a2-2 | 15 | b1-3 | 10 | b2-2 | 25 | — | — | 8,100 | 1.74 |
| | | | | | | | b2-3 | 15 | | | | |

### [3] Preparation of Resist Composition

### Examples 2-1 to 2-30 and Comparative Examples 1-1 to 1-30

A resist composition was prepared by dissolving an inventive onium salt (SQ-1 to SQ-10) or comparative quencher (SQ-A to SQ-H, AQ-A, AQ-B), base polymer (P-1 to P-18), and photoacid generator (PAG-X, PAG-Y) in a solvent containing 100 ppm of surfactant FC-4430 (3M) in accordance with the formulation shown in Tables 2 to 5, and filtering the solution through a Teflon® t filter with a pore size of 0.2 μm.

The components in Tables 2 to 5 are identified below.

Organic Solvent
PGMEA: propylene glycol monomethyl ether acetate
DAA: diacetone alcohol

Photoacid generators PAG-X and PAG-Y

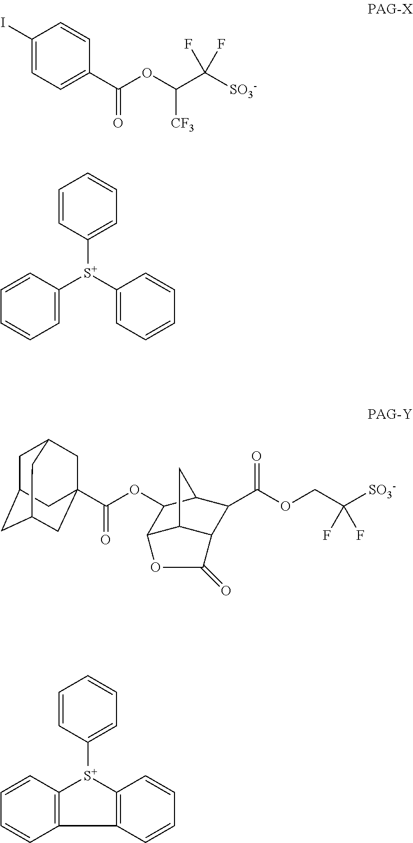

Comparative Quenchers SQ-A to SQ-H, AQ-A and AQ-B

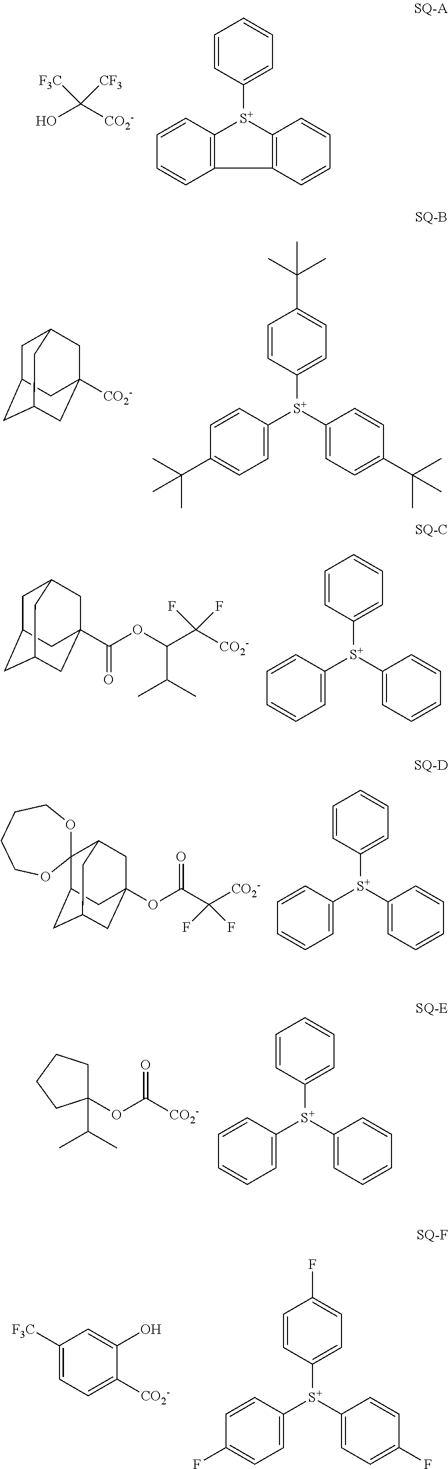

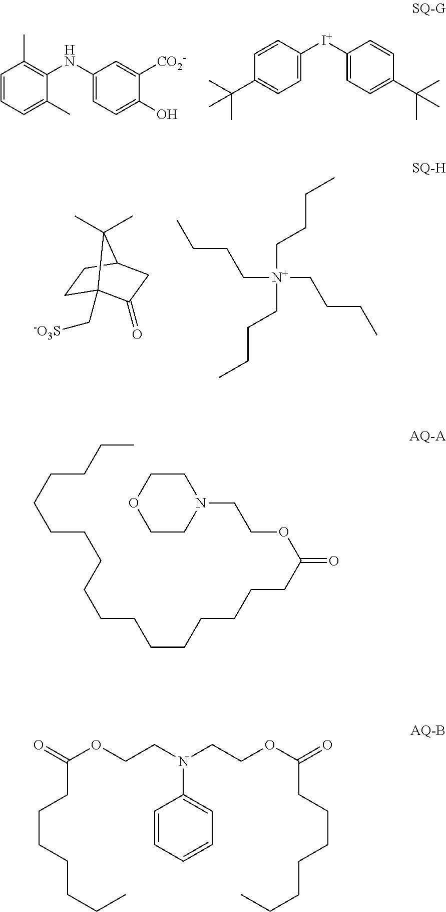

TABLE 2

| | | | Base | Photoacid | | | |
|---|---|---|---|---|---|---|---|
| | | Resist | polymer | generator | Quencher | Solvent 1 | Solvent 2 |
| | | composition | (pbw) | (pbw) | (pbw) | (pbw) | (pbw) |
| Example | 2-1 | R-1 | P-1 | — | SQ-1 | PGMEA | DAA |
| | | | (80) | | (7.8) | (2200) | (900) |
| | 2-2 | R-2 | P-1 | — | SQ-2 | PGMEA | DAA |
| | | | (80) | | (7.6) | (2200) | (900) |
| | 2-3 | R-3 | P-1 | — | SQ-3 | PGMEA | DAA |
| | | | (80) | | (7.8) | (2200) | (900) |
| | 2-4 | R-4 | P-1 | — | SQ-4 | PGMEA | DAA |
| | | | (80) | | (7.8) | (2200) | (900) |
| | 2-5 | R-5 | P-1 | — | SQ-5 | PGMEA | DAA |
| | | | (80) | | (7.8) | (2200) | (900) |
| | 2-6 | R-6 | P-1 | — | SQ-6 | PGMEA | DAA |
| | | | (80) | | (7.4) | (2200) | (900) |
| | 2-7 | R-7 | P-1 | — | SQ-7 | PGMEA | DAA |
| | | | (80) | | (7.8) | (2200) | (900) |
| | 2-8 | R-8 | P-1 | — | SQ-8 | PGMEA | DAA |
| | | | (80) | | (7.8) | (2200) | (900) |
| | 2-9 | R-9 | P-1 | — | SQ-9 | PGMEA | DAA |
| | | | (80) | | (7.7) | (2200) | (900) |
| | 2-10 | R-10 | P-1 | — | SQ-10 | PGMEA | DAA |
| | | | (80) | | (7.8) | (2200) | (900) |
| | 2-11 | R-11 | P-2 | — | SQ-1 | PGMEA | DAA |
| | | | (80) | | (7.8) | (2200) | (900) |
| | 2-12 | R-12 | P-3 | — | SQ-3 | PGMEA | DAA |
| | | | (80) | | (8.8) | (2200) | (900) |
| | 2-13 | R-13 | P-4 | — | SQ-4 | PGMEA | DAA |
| | | | (80) | | (7.8) | (2200) | (900) |
| | 2-14 | R-14 | P-5 | — | SQ-5 | PGMEA | DAA |
| | | | (80) | | (7.8) | (2200) | (900) |
| | 2-15 | R-15 | P-6 | PAG-X | SQ-6 | PGMEA | DAA |
| | | | (80) | (12) | (7.8) | (2200) | (900) |

TABLE 3

| | | | Base | Photoacid | | | |
|---|---|---|---|---|---|---|---|
| | | Resist | polymer | generator | Quencher | Solvent 1 | Solvent 2 |
| | | composition | (pbw) | (pbw) | (pbw) | (pbw) | (pbw) |
| Example | 2-16 | R-16 | P-7 | — | SQ-7 | PGMEA | DAA |
| | | | (80) | | (6.8) | (2200) | (900) |
| | 2-17 | R-17 | P-8 | — | SQ-8 | PGMEA | DAA |
| | | | (80) | | (7.8) | (2200) | (900) |
| | 2-18 | R-18 | P-9 | — | SQ-9 | PGMEA | DAA |
| | | | (80) | | (7.8) | (2200) | (900) |
| | 2-19 | R-19 | P-10 | — | SQ-1 (4.8) | PGMEA | DAA |
| | | | (80) | | SQ-10 (3.6) | (2200) | (900) |
| | 2-20 | R-20 | P-11 | — | SQ-2 | PGMEA | DAA |
| | | | (80) | | (7.8) | (2200) | (900) |
| | 2-21 | R-21 | P-12 | — | SQ-1 (4.8) | PGMEA | DAA |
| | | | (80) | | AQ-A (2.4) | (2200) | (900) |
| | 2-22 | R-22 | P-13 | — | SQ-5 | PGMEA | DAA |
| | | | (80) | | (6.8) | (2200) | (900) |
| | 2-23 | R-23 | P-14 | PAG-X | SQ-1 | PGMEA | DAA |
| | | | (80) | (20) | (6.2) | (2200) | (900) |
| | 2-24 | R-24 | P-15 | PAG-Y | SQ-3 | PGMEA | DAA |
| | | | (80) | (24) | (7.2) | (2200) | (900) |
| | 2-25 | R-25 | P-16 | PAG-X | SQ-5 | PGMEA | DAA |
| | | | (80) | (20) | (7.4) | (2200) | (900) |
| | 2-26 | R-26 | P-17 | PAG-Y | SQ-7 | PGMEA | DAA |
| | | | (80) | (18) | (7.8) | (2200) | (900) |
| | 2-27 | R-27 | P-18 | PAG-X | SQ-9 | PGMEA | DAA |
| | | | (80) | (20) | (7.2) | (2200) | (900) |
| | 2-28 | R-28 | P-1 | PAG-X | SQ-3 | PGMEA | DAA |
| | | | (80) | (8) | (8.2) | (2200) | (900) |
| | 2-29 | R-29 | P-3 | — | SQ-3 (4.0) | PGMEA | DAA |
| | | | (80) | | SQ-7 (3.4) | (2200) | (900) |
| | 2-30 | R-30 | P-11 | — | SQ-2 | PGMEA | DAA |
| | | | (80) | | (7.8) | (2200) | (900) |

TABLE 4

| | | | Base | Photoacid | | | |
|---|---|---|---|---|---|---|---|
| | | Resist | polymer | generator | Quencher | Solvent 1 | Solvent 2 |
| | | composition | (pbw) | (pbw) | (pbw) | (pbw) | (pbw) |
| Comparative | 1-1 | CR-1 | P-1 | — | SQ-A | PGMEA | DAA |
| Example | | | (80) | | (7.8) | (2200) | (900) |
| | 1-2 | CR-2 | P-1 | — | SQ-B | PGMEA | DAA |
| | | | (80) | | (7.6) | (2200) | (900) |
| | 1-3 | CR-3 | P-1 | — | SQ-C | PGMEA | DAA |
| | | | (80) | | (7.5) | (2200) | (900) |
| | 1-4 | CR-4 | P-1 | — | SQ-D | PGMEA | DAA |
| | | | (80) | | (7.6) | (2200) | (900) |
| | 1-5 | CR-5 | P-1 | — | SQ-E | PGMEA | DAA |
| | | | (80) | | (7.8) | (2200) | (900) |
| | 1-6 | CR-6 | P-1 | — | SQ-F | PGMEA | DAA |
| | | | (80) | | (7.6) | (2200) | (900) |
| | 1-7 | CR-7 | P-1 | — | SQ-G | PGMEA | DAA |
| | | | (80) | | (7.6) | (2200) | (900) |
| | 1-8 | CR-8 | P-1 | — | SQ-H | PGMEA | DAA |
| | | | (80) | | (8.8) | (2200) | (900) |
| | 1-9 | CR-9 | P-1 | — | AQ-A | PGMEA | DAA |
| | | | (80) | | (7.8) | (2200) | (900) |
| | 1-10 | CR-10 | P-1 | — | AQ-B | PGMEA | DAA |
| | | | (80) | | (7.6) | (2200) | (900) |
| | 1-11 | CR-11 | P-2 | — | SQ-C | PGMEA | DAA |
| | | | (80) | | (7.5) | (2200) | (900) |
| | 1-12 | CR-12 | P-3 | — | SQ-B | PGMEA | DAA |
| | | | (80) | | (7.6) | (2200) | (900) |
| | 1-13 | CR-13 | P-4 | — | SQ-E | PGMEA | DAA |
| | | | (80) | | (7.8) | (2200) | (900) |
| | 1-14 | CR-14 | P-5 | — | SQ-F | PGMEA | DAA |
| | | | (80) | | (7.4) | (2200) | (900) |
| | 1-15 | CR-15 | P-6 | PAG-X | SQ-D | PGMEA | DAA |
| | | | (80) | (12) | (7.6) | (2200) | (900) |

TABLE 5

| | | | Base | Photoacid | | | |
|---|---|---|---|---|---|---|---|
| | | Resist | polymer | generator | Quencher | Solvent 1 | Solvent 2 |
| | | composition | (pbw) | (pbw) | (pbw) | (pbw) | (pbw) |
| Comparative | 1-16 | CR-16 | P-7 | — | SQ-G | PGMEA | DAA |
| Example | | | (80) | | (7.2) | (2200) | (900) |
| | 1-17 | CR-17 | P-8 | — | SQ-F | PGMEA | DAA |
| | | | (80) | | (7.2) | (2200) | (900) |
| | 1-18 | CR-18 | P-9 | — | SQ-H | PGMEA | DAA |
| | | | (80) | | (7.8) | (2200) | (900) |
| | 1-19 | CR-19 | P-10 | — | SQ-A | PGMEA | DAA |
| | | | (80) | | (7.8) | (2200) | (900) |
| | 1-20 | CR-20 | P-11 | — | SQ-B | PGMEA | DAA |
| | | | (80) | | (7.6) | (2200) | (900) |
| | 1-21 | CR-21 | P-12 | — | AQ-A | PGMEA | DAA |
| | | | (80) | | (7.8) | (2200) | (900) |
| | 1-22 | CR-22 | P-13 | — | AQ-B | PGMEA | DAA |
| | | | (80) | | (7.6) | (2200) | (900) |
| | 1-23 | CR-23 | P-14 | PAG-X | AQ-A | PGMEA | DAA |
| | | | (80) | (20) | (7.8) | (2200) | (900) |
| | 1-24 | CR-24 | P-15 | PAG-Y | SQ-D | PGMEA | DAA |
| | | | (80) | (24) | (7.6) | (2200) | (900) |
| | 1-25 | CR-25 | P-16 | PAG-X | SQ-E | PGMEA | DAA |
| | | | (80) | (20) | (7.8) | (2200) | (900) |
| | 1-26 | CR-26 | P-17 | PAG-Y | SQ-F | PGMEA | DAA |
| | | | (80) | (18) | (7.2) | (2200) | (900) |
| | 1-27 | CR-27 | P-18 | PAG-X | AQ-A | PGMEA | DAA |
| | | | (80) | (20) | (7.8) | (2200) | (900) |
| | 1-28 | CR-28 | P-1 | PAG-X | SQ-E | PGMEA | DAA |
| | | | (80) | (8) | (7.8) | (2200) | (900) |
| | 1-29 | CR-29 | P-3 | — | SQ-C (4.8) | PGMEA | DAA |
| | | | (80) | | AQ-A (2.4) | (2200) | (900) |
| | 1-30 | CR-30 | P-11 | — | SQ-D | PGMEA | DAA |
| | | | (80) | | (7.4) | (2200) | (900) |

### [4] EUV Lithography Test

### Examples 3-1 to 3-30 and Comparative Examples 2-1 to 2-30

Each of the resist compositions (R-1 to R-30, CR-1 to CR-30) was spin coated on a silicon substrate having a 20-nm coating of silicon-containing spin-on hard mask SHB-A940 (Shin-Etsu Chemical Co., Ltd., silicon content 43 wt %) and prebaked on a hotplate at 100° C. for 60 seconds to form a resist film of 50 nm thick. Using an EUV scanner NXE3300 (ASML, NA 0.33. σ0.9/0.6, dipole illumination), the resist film was exposed to EUV through a mask bearing a line-and-space (LS) pattern having a width of 18 nm (on-wafer size) and a pitch of 36 nm while changing the dose at a pitch of 1 mJ/cm² and the focus at a pitch of 0.020 μm. The resist film was baked (PEB) at the temperature shown in Tables 6 and 7 for 60 seconds. This was followed by puddle development in a 2.38 wt % TMAH aqueous solution for 30 seconds, rinsing with a surfactant-containing rinse fluid, and spin drying. A positive LS pattern was obtained.

The LS pattern was observed under CD-SEM (CG6300, Hitachi High-Technologies Corp.) and evaluated for sensitivity, exposure latitude (EL), LWR, depth of focus (DOF), and collapse limit by the following methods. The results are shown in Tables 6 and 7.

Evaluation of Sensitivity

The optimum dose (Eop, mJ/cm²) which provided an LS pattern with a line width of 18 nm and a pitch of 36 nm was determined and reported as sensitivity.

Evaluation of EL

The exposure dose which provided a LS pattern with a space width of 18 nm±10% (i.e., 16.2 to 19.8 nm) was determined. EL (%) is calculated from the exposure doses according to the following equation:

EL (%)=(|E1−E2|/Eop)×100

wherein E1 is an optimum exposure dose which provides a LS pattern with a line width of 16.2 nm and a pitch of 36 nm, E2 is an optimum exposure dose which provides a LS pattern with a line width of 19.8 nm and a pitch of 36 nm, and Eop is an optimum exposure dose which provides a LS pattern with a line width of 18 nm and a pitch of 36 mu. A larger value indicates better performance.

Evaluation of LWR

For the LS pattern formed by exposure at the optimum dose Eop, the line width was measured at 10 longitudinally spaced apart points, from which a 3-fold value (3σ) of the standard deviation (σ) was determined and reported as LWR. A smaller value of 3a indicates a pattern having small roughness and uniform line width.

Evaluation of DOF

As an index of DOF, a range of focus which provided a LS pattern with a size of 18 nm±10% (i.e., 16.2 to 19.8 nm) was determined. A greater value indicates a wider DOF.

Evaluation of Collapse Limit of Line Pattern

For the LS pattern formed by exposure at the dose corresponding to the optimum focus, the line width was measured at 10 longitudinally spaced apart points. The minimum line size above which lines could be resolved without collapse was determined and reported as collapse limit. A smaller value indicates better collapse limit.

TABLE 6

| | | | | | | | | Collapse |
|---|---|---|---|---|---|---|---|---|
| | | Resist | PEB temp. | Eop | EL | LWR | DOF | limit |
| | | composition | (° C.) | (mJ/cm²) | (%) | (nm) | (nm) | (nm) |
| Example | 3-1 | R-1 | 105 | 37 | 19 | 2.6 | 130 | 10.1 |
| | 3-2 | R-2 | 100 | 38 | 19 | 2.9 | 120 | 10.4 |
| | 3-3 | R-3 | 110 | 38 | 18 | 2.7 | 110 | 10.2 |
| | 3-4 | R-4 | 105 | 38 | 18 | 2.8 | 120 | 10.4 |
| | 3-5 | R-5 | 105 | 39 | 17 | 2.7 | 120 | 10.3 |
| | 3-6 | R-6 | 100 | 36 | 19 | 2.8 | 110 | 10.4 |
| | 3-7 | R-7 | 110 | 38 | 18 | 2.8 | 100 | 10.5 |
| | 3-8 | R-8 | 105 | 40 | 17 | 2.9 | 120 | 11.1 |
| | 3-9 | R-9 | 105 | 38 | 18 | 3 | 120 | 10.6 |
| | 3-10 | R-10 | 110 | 39 | 18 | 2.8 | 120 | 10.9 |
| | 3-11 | R-11 | 100 | 37 | 19 | 2.9 | 110 | 10.5 |
| | 3-12 | R-12 | 105 | 38 | 19 | 2.8 | 120 | 10.6 |
| | 3-13 | R-13 | 110 | 38 | 17 | 2.8 | 100 | 11.1 |
| | 3-14 | R-14 | 110 | 37 | 18 | 2.7 | 120 | 10.8 |
| | 3-15 | R-15 | 105 | 36 | 18 | 3 | 130 | 11.3 |
| | 3-16 | R-16 | 100 | 39 | 19 | 2.7 | 120 | 10.7 |
| | 3-17 | R-17 | 105 | 40 | 17 | 2.9 | 110 | 11.1 |
| | 3-18 | R-18 | 100 | 38 | 19 | 2.9 | 110 | 10.7 |
| | 3-19 | R-19 | 105 | 36[ | 18 | 2.8 | 100 | 10.4 |
| | 3-20 | R-20 | 105 | 37 | 17 | 2.8 | 120 | 10.3 |
| | 3-21 | R-21 | 100 | 41 | 19 | 2.9 | 120 | 11 |
| | 3-22 | R-22 | 110 | 38 | 18 | 2.7 | 110 | 11.2 |
| | 3-23 | R-23 | 105 | 37 | 18 | 2.8 | 100 | 10.8 |
| | 3-24 | R-24 | 100 | 39 | 17 | 3 | 110 | 10.2 |
| | 3-25 | R-25 | 110 | 37 | 18 | 2.8 | 120 | 10.4 |
| | 3-26 | R-26 | 105 | 39 | 17 | 2.9 | 120 | 11.1 |
| | 3-27 | R-27 | 100 | 37 | 17 | 2.7 | 110 | 10.6 |
| | 3-28 | R-28 | 105 | 38 | 18 | 2.8 | 100 | 10.2 |
| | 3-29 | R-29 | 110 | 38 | 19 | 2.9 | 100 | 10.2 |
| | 3-30 | R-30 | 110 | 37 | 18 | 2.8 | 120 | 10.3 |

TABLE 7

| | | | | | | | | Collapse |
|---|---|---|---|---|---|---|---|---|
| | | Resist | PEB temp. | Eop | EL | LWR | DOF | limit |
| | | composition | (° C.) | (mJ/cm²) | (%) | (nm) | (nm) | (nm) |
| Comparative | 2-1 | CR-1 | 100 | 41 | 17 | 3.7 | 80 | 14.5 |
| Example | 2-2 | CR-2 | 105 | 43 | 16 | 3.5 | 90 | 15.3 |
| | 2-3 | CR-3 | 110 | 42 | 17 | 3.3 | 70 | 13.4 |
| | 2-4 | CR-4 | 104 | 44 | 18 | 3.8 | 100 | 14.8 |
| | 2-5 | CR-5 | 100 | 41 | 17 | 4 | 80 | 16.3 |
| | 2-6 | CR-6 | 110 | 42 | 16 | 3.9 | 90 | 16.2 |
| | 2-7 | CR-7 | 105 | 43 | 15 | 3.5 | 70 | 14 |
| | 2-8 | CR-8 | 100 | 41 | 16 | 3.7 | 100 | 14.6 |
| | 2-9 | CR-9 | 105 | 39 | 14 | 3.6 | 90 | 13.4 |
| | 2-10 | CR-10 | 105 | 41 | 15 | 3.5 | 80 | 15.1 |
| | 2-11 | CR-11 | 100 | 40 | 16 | 3.9 | 90 | 14.5 |
| | 2-12 | CR-12 | 110 | 38 | 15 | 3.5 | 90 | 13.4 |
| | 2-13 | CR-13 | 110 | 40 | 17 | 4.1 | 80 | 15.4 |
| | 2-14 | CR-14 | 105 | 41 | 14 | 3.4 | 70 | 14.8 |
| | 2-15 | CR-15 | 100 | 42 | 18 | 3.5 | 100 | 14.5 |
| | 2-16 | CR-16 | 105 | 43 | 15 | 3.6 | 90 | 14.2 |
| | 2-17 | CR-17 | 105 | 39 | 17 | 3.4 | 90 | 15.4 |
| | 2-18 | CR-18 | 110 | 41 | 14 | 3.8 | 80 | 15.3 |
| | 2-19 | CR-19 | 105 | 45 | 15 | 3.6 | 70 | 13.4 |
| | 2-20 | CR-20 | 100 | 42 | 17 | 3.7 | 90 | 13.6 |
| | 2-21 | CR-21 | 110 | 41 | 16 | 3.8 | 80 | 14.1 |
| | 2-22 | CR-22 | 105 | 40 | 17 | 3.7 | 70 | 14.2 |
| | 2-23 | CR-23 | 110 | 41 | 18 | 3.5 | 80 | 13.9 |
| | 2-24 | CR-24 | 105 | 42 | 16 | 3.6 | 90 | 14.1 |
| | 2-25 | CR-25 | 100 | 43 | 15 | 3.8 | 100 | 13.8 |
| | 2-26 | CR-26 | 105 | 40 | 17 | 3.6 | 90 | 14.8 |
| | 2-27 | CR-27 | 110 | 39 | 15 | 3.4 | 70 | 14.3 |
| | 2-28 | CR-28 | 110 | 41 | 16 | 3.5 | 80 | 14.8 |
| | 2-29 | CR-29 | 105 | 42 | 15 | 3.6 | 90 | 15.1 |
| | 2-30 | CR-30 | 110 | 43 | 15 | 3.5 | 70 | 14.2 |

It is demonstrated in Tables 6 and 7 that resist compositions within the scope of the invention exhibit a high sensitivity, improved lithography properties, and resistance to pattern collapse.

Japanese Patent Application No. 2022-095416 is incorporated herein by reference. Although some preferred embodiments have been described, many modifications and variations may be made thereto in light of the above teachings. It is therefore to be understood that the invention may be practiced otherwise than as specifically described without departing from the scope of the appended claims.

1. An onium salt having an anion moiety whose conjugate acid is decomposed under the action of acid and heat into carbon dioxide and an organic compound of up to 12 carbon atoms.

2. The onium salt of claim 1 having the formula (1): 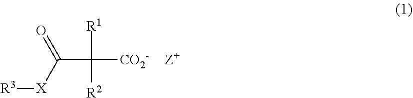
wherein X is a single bond, —O— or —S—,
R¹ and R² are each independently hydrogen or a C₁-C₁₀ hydrocarbyl group in which some constituent —CH₂— may be replaced by —O— or —C(═O)—, R¹ and R² may bond together to form a ring with the carbon atom to which they are attached,
R³ is hydrogen or a C₁-C₁₀ hydrocarbyl group when X is a single bond or —S—, and hydrogen, a C₁-C₁₀ hydrocarbyl group other than an acid labile group, or an acid labile group when X is —O—, some or all of the hydrogen atoms in the hydrocarbyl group may be substituted by halogen, some constituent —CH₂— in the hydrocarbyl group may be replaced by —O— or —C(═O)—, R¹ and R³ may bond together to form a ring with the atoms to which they are attached and intervenient atom, with the proviso that the number of carbon atoms within R¹ to R³ is up to 10 when R³ is other than the acid labile group, and
Z⁺ is an onium cation.

3. The onium salt of claim 2 wherein X is —O—.

4. The onium salt of claim 3 wherein R³ is an acid labile group.

5. The onium salt of claim 4 wherein the acid labile group has the formula (AL-1) or (AL-2): 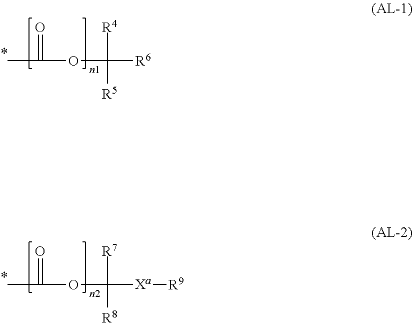
wherein X^{a} is —O— or —S—,
R⁴, R⁵ and R⁶ are each independently a C₁-C₁₂ hydrocarbyl group, some constituent —CH₂— in the hydrocarbyl group may be replaced by —O— or —S—, and when the hydrocarbyl group contains an aromatic ring, some or all of the hydrogen atoms on the aromatic ring may be substituted by halogen, cyano, nitro, optionally halogenated C₁-C₄ alkyl moiety, or optionally halogenated C₁-C₄ alkoxy moiety, any two of⁴, R⁵ and R⁶ may bond together to form a ring, some constituent —CH₂— in the ring may be replaced by —O— or —S—,
R⁷ and R⁸ are each independently hydrogen or a C₁-C₁₀ hydrocarbyl group, R⁹ is a C₁-C₂₀ hydrocarbyl group in which some constituent —CH₂— may be replaced by —O— or —S—, R⁸ and R⁹ may bond together to form a C₃-C₂₀ heterocycle with the carbon atom and X^{a} to which they are attached, some constituent —CH₂— in the heterocycle may be replaced by —O— or —S—,
n1 and n2 are each independently 0 or 1, and
designates a point of attachment to the adjacent —O—.

6. The onium salt of claim 2 wherein Z⁺is an onium cation having any one of the formulae (cation-1) to (cation-3): 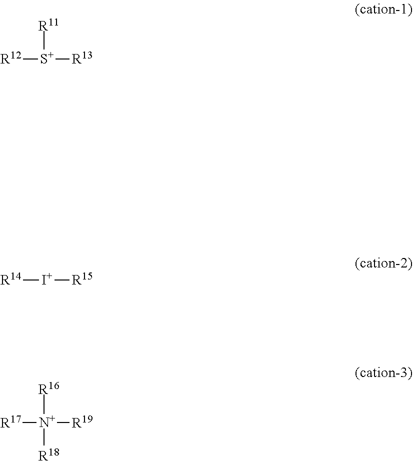
wherein R¹¹ to R¹⁹ are each independently a C₁-C₃₀ hydrocarbyl group which may contain a heteroatom, R¹¹ and R¹² may bond together to form a ring with the sulfur atom to which they are attached.

7. A quencher comprising the onium salt of claim 1.

8. A resist composition comprising the quencher of claim 7.

9. The resist composition of claim 8, further comprising an organic solvent.

10. The resist composition of claim 8, further comprising a base polymer comprising repeat units having the formula (a1): 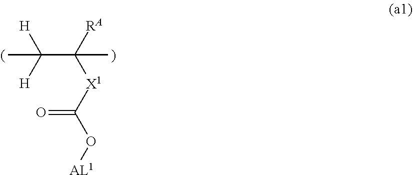
wherein R^{A} is hydrogen, fluorine, methyl or trifluoromethyl,
X¹ is a single bond, phenylene group, naphthylene group or*—C(═O)—O—X¹¹—, the phenylene group and naphthylene group may be substituted with an optionally fluorinated C₁-C₁₀ alkoxy moiety or halogen, X¹¹ is a C₁-C₁₀ saturated hydrocarbylene group which may contain a hydroxy moiety, ether bond, ester bond or lactone ring, a phenylene group or naphthylene group, * designates a point of attachment to the carbon atom in the backbone, and
AL¹ is an acid labile group.

11. The resist composition of claim 10 wherein the base polymer further comprises repeat units having the formula (a2): 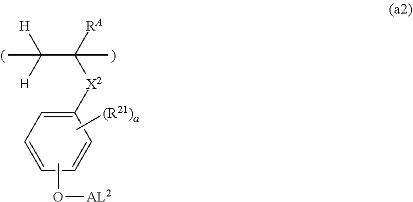
wherein R^{A} is hydrogen, fluorine, methyl or trifluoromethyl,
X² is a single bond or*—C(═O)—O—, wherein * designates a point of attachment to the carbon atom in the backbone,
R²¹ is halogen, cyano, a C₁-C₂₀ hydrocarbyl group which may contain a heteroatom, C₁-C₂₀ hydrocarbyloxy group which may contain a heteroatom, C₂-C₂₀ hydrocarbylcarbonyl group which may contain a heteroatom, C₂-C₂₀ hydrocarbylcarbonyloxy group which may contain a heteroatom, or C₂-C₂₀ hydrocarbyloxycarbonyl group which may contain a heteroatom,
AL² is an acid labile group, and
a is an integer of 0 to 4.

12. The resist composition of claim 10 wherein the base polymer further comprises repeat units having the formula (b1) or (b2): 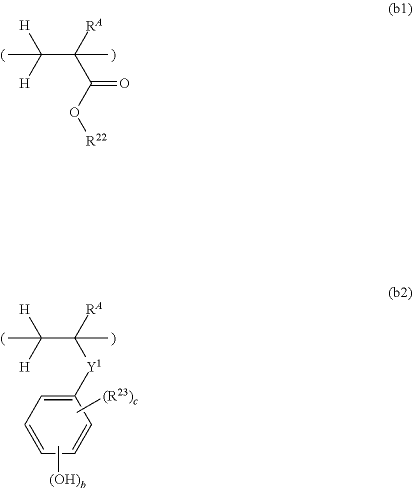
wherein R^{A} is each independently hydrogen, fluorine, methyl or trifluoromethyl,
Y¹ is a single bond or*—C(═O)—O—,
R²² is hydrogen, or a C₁-C₂₀ group containing at least one moiety selected from hydroxy moiety other than phenolic hydroxy, cyano moiety, carbonyl moiety, carboxy moiety, ether bond, ester bond, sulfonic ester bond, carbonate bond, lactone ring, sultone ring, and carboxylic anhydride (—C(═O)—O—C(═O)—),
R²³ is halogen, hydroxy, nitro, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom,
b is an integer of 1 to 4, c is an integer of 0 to 4, and b+c is from 1 to 5.

13. The resist composition of claim 10 wherein the base polymer further comprises repeat units of at least one type selected from repeat units having the formulae (c1) to (c4): 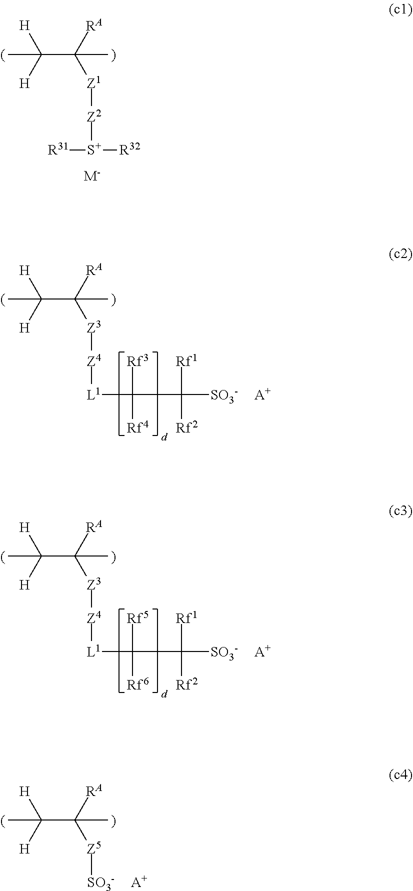
wherein R^{A} is each independently hydrogen, fluorine, methyl or trifluoromethyl,
Z¹ is a single bond or phenylene group,
Z² is *—C(═O)—O—Z²¹—, *C(═O)—NH—Z²¹—, or*—O—Z²¹—, wherein Z²¹ is a C₁-C₆ aliphatic hydrocarbylene group, phenylene, or divalent group obtained by combining the foregoing, which may contain a carbonyl moiety, ester bond, ether bond or hydroxy moiety,
Z³ is a single bond, phenylene group, naphthylene group or*—C(═O)—O—Z³¹—, wherein Z³¹ is a C₁-C₁₀ aliphatic hydrocarbylene group which may contain a hydroxy moiety, ether bond, ester bond or lactone ring, or a phenylene group or naphthylene group,
Z⁴ is a single bond or*—Z⁴¹—C(═(=)—O—, wherein Z⁴¹ is a C₁-C₂₀ hydrocarbylene group which may contain a heteroatom,
Z⁵ is a single bond, methylene, ethylene, phenylene, fluorinated phenylene, trifluoromethyl-substituted phenylene, *—C(═O)—O—Z⁵¹—, *—C(═O)—N(H)—Z⁵¹— or*—O—Z⁵¹—, wherein Z⁵¹ is a C₁-C₆ aliphatic hydrocarbylene group, phenylene, fluorinated phenylene or trifluoromethyl-substituted phenylene group, which may contain a carbonyl moiety, ester bond, ether bond or hydroxy moiety,
the asterisk (*) designates a point of attachment to the carbon atom in the backbone,
R³¹ and R³² are each independently a C₁-C₂₀ hydrocarbyl group which may contain a heteroatom, R³¹ and R³² may bond together to form a ring with the sulfur atom to which they are attached,
L¹ is a single bond, ether bond, ester bond, carbonyl group, sulfonic ester bond, carbonate bond or carbamate bond,
Rf¹ and Rf² are each independently fluorine or a C₁-C₆ fluorinated alkyl group,
Rf³ and Rf⁴ are each independently hydrogen, fluorine or a C₁-C₆ fluorinated alkyl group,
Rf⁵ and Rf⁶ are each independently hydrogen, fluorine or a C₁-C₆ fluorinated alkyl group, excluding that all Rf⁵ and Rf⁶ are hydrogen at the same time,
M⁻ is a non-nucleophilic counter ion,
A⁺ is an onium cation, and
d is an integer of 0 to 3.

14. The resist composition of claim 8, further comprising a photoacid generator.

15. The resist composition of claim 8, further comprising an amine compound.

16. The resist composition of claim 8, further comprising a surfactant.

17. A process for forming a pattern comprising the steps of applying the resist composition of claim 8 to a substrate to form a resist film thereon, exposing the resist film to high-energy radiation, baking the resist film, and developing the PEB resist film in a developer.

18. The process of claim 17 wherein the high-energy radiation is KrF excimer laser, ArF excimer laser, EB, or EUV of wavelength 3 to 15 nm.
